(19) United States
(12) Patent Application Publication
(10) Pub. No.: US 2003/0064946 A1
(43) Pub. Date: Apr. 3, 2003

(54) Method and reagent for the inhibition of calcium activated chloride channel-1 (CLCA-1)

(76) Inventors: James McSwiggen, Boulder, CO (US); James Thompson, Lafayette, CO (US); Timothy McKenzie, Aurora, CO (US); David Ayers, Broomfield, CO (US); David E. Szymkowski, Mountain View, CA (US); Andrew Grupe, Redwood City, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/927,046

(22) Filed: Aug. 9, 2001

Related U.S. Application Data
(60) Provisional application No. 60/224,383, filed on Aug. 9, 2000.

(51) Int. Cl. A61K 48/00; C07H 21/02; C07H 21/04; C12N 15/87

(52) U.S. Cl. 514/44; 536/23.1; 435/320.1; 435/455

(57) ABSTRACT

Nucleic acid molecules, including antisense and enzymatic nucleic acid molecules, such as hammerhead ribozymes, DNAzymes, and GeneBlocs, which modulate the expression of calcium activated chloride channels (CLCA1, CLCA2, CLCA3, and CLCA4).

### BACKGROUND OF THE INVENTION

The present invention concerns compounds, compositions, and methods for the study, diagnosis, and treatment of conditions and diseases related to the expression of CLCA (Cl⁻ Channel Ca²⁺-Activated) genes.

The following is a brief description of the current understanding of CLCAs.

The discussion is not meant to be complete and is provided only for understanding the invention that follows. The summary is not an admission that any of the work described below is prior art to the claimed invention.

CLCA proteins are emerging as a new class of channel proteins that mediate Ca²⁺-activated Cl⁻ conductance in a variety of tissues. Members of the CLCA family have been cloned, isolated, and partially characterized from human, bovine, and murine species. These proteins demonstrate a high degree of homology in their size, sequence, and predicted structure yet can vary considerably in tissue distribution. Bovine CLCA1 (bCLCA1 or CaCC) was the first reported CLCA homolog. The bCLCA1 protein, which was isolated from and is exclusively detected in trachial epithelial cells, functions as a Ca²⁺-activated Cl⁻ channel (Ran and Benos, 1992, J Biol. Chem., 267, 3618-3625; Cunningham et al., 1995, J. Biol. Cherm, 270, 31016-31026). Another bovine homolog, bovine lung-endothelial cell adhesion molecule-1 (Lu-ECAM-1), appears to have involvement in the preferential metastasis of melanoma cells to the lung. Lu-ECAM-1 shares 92% nucleotide identity to bCLCA1 and is expressed in vascular endothelial cells (Elble et al., 1997, J. Biol. Chem., 272, 27853-27861). It has been shown that Lu-ECAM-1, can mediate the binding of lung-metastatic mouse B 16F 10 melanoma cells to endothelial cells (Zhu et al., 1992, J. Clin. Invest., 89, 1718-1724), however, due to sequence similarity to bCLCA1, the role of Lu-ECAM-1 as a chloride channel has been suggested (Elble et al., supra). The mouse homolog, mCLCA1, appears to have an expression pattern similar to the cystic fibrosis transmembrane conductance regulator (CFTR), with expression seen in various secretory epithelial cells, squamous epithelia, and in some lymphocytes (Gruber et al., 1998, Histochem. Cell Biol., 110,43-49).

The three human CLCA homologs (hCLCA1, hCLCA2, and hCLCA3) thus far cloned, isolated, and partially characterized, all retain sequence homology, similar cDNA length, and are all located on the short arm of chromosome 1 (tp22-p31). Human CLCA proteins show a restricted pattern of expression in differing secretory tissues. Human CLCA1 was the first reported calcium activated chloride channel in humans. The 31,902-bp hCLCA1 gene is located on chromosome 1p22-p31, contains 14 introns, and is preceded by a canonic promoter region that contains an LI transposable element. Expression of hCLCA1 is predominant in intestinal basal crypt epithelia and goblet cells. A protein processing model has been proposed for hCLCA1 in which the primary translation product (125-kDa) is cleaved to a 90-kDa and a group of 37- to 41-kDa proteins, the latter apparently representing different glycosylation products of the same polypeptide (Gruber et al., 1998, Genomics, 54, 200-214). Transient expression of hCLCA1 cDNA in HEK 293 cells is associated with an increase in whole-cell Ca²⁺-activated Cl⁻ conductance that is susceptible to inhibition with anion channel blocking compounds. Cell attached patch recordings of transfected cells in this study revealed single channels with a slope conductance of 13.4 pS (Gruber et al., supra).

The hCLCA2 homolog is processed in a similar manner as is hCLCA1, resulting in the formation of a heterodimer consisting of a 90-kDa amino terminal and an approximately 35-kDa carboxy terminal subunit with anchorage to the plasma membrane via four or five transmembrane domains. Expression of hCLCA2 is somewhat less restricted than that of hCLCA1, being expressed from human lung, trachea, and breast tissue (Gruber et al., 1999, Am. J. Physiol., 276, C1261-C1270). Human CLCA2 is expressed in normal breast epithelium but not in breast tumors of different stages of progression, suggesting that hCLCA2 may act as a tumor suppressor in breast cancer (Gruber et al., 1999, Cancer Res., 59, 5488-5491). Human CLCA3 is a truncated, secreted member of the CLCA family which is expressed in numerous tissues including lung, trachea, spleen, thymus, and breast tissue. Unlike hCLCA1 and hCLCA2 which are processed into heterodimers, hCLCA3 mRNA encodes a 37-kDa glycoprotein that corresponds to the N-terminal extracellular domain of its homologs. When hCLCA3 is expressed in HEK 293 or CHO cells, the 37-kDa glycoprotein is secreted (Gruber and Pauli, 1999, Biochem. Biophys. Acta, 1444, 418-423).

Holroyd et al., International PCT publication No. WO/9944620, describe a calcium-activated chloride channel that is induced by IL-9.

### SUMMARY OF THE INVENTION

The invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups] and methods for their use to modulate the expression of CLCA (Cl⁻ Channel Ca²⁺-Activated) genes.

In a preferred embodiment, the invention features the use of one or more of the nucleic acid-based techniques independently or in combination to inhibit the expression of the genes encoding hCLCA1, hCLCA2, hCLCA3, and hCLCA4. Specifically, the invention features the use of nucleic acid-based techniques to specifically inhibit the expression of CLCA1 (GenBank accession Nos. NM_{—}001285, AF039400, AF039401, AF127036), CLCA2 (GenBank accession No. NM_{—}006536), CLCA3 (GenBank accession No. NM_{—}004921), and CLCA4 (GenBank accession No. NM_{—}012128) genes. In yet another preferred embodiment, the invention features the inhibition of CLCA1 gene using the nucleic acid-based techniques of the instant invention.

In another preferred embodiment, the invention features the use of an enzymatic nucleic acid molecule, preferably in the hammerhead, NCH (Inozyme), G-cleaver, amberzyme, zinzyme and/or DNAzyme motif, to inhibit the expression of CLCA genes.

By “inhibit” it is meant that the activity of CLCA1 or level of RNAs or equivalent RNAs encoding one or more protein subunits of CLCA1 is reduced below that observed in the absence of the nucleic acid molecules of the invention. In one embodiment, inhibition with enzymatic nucleic acid molecules preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition of CLCA1 genes with the nucleic acid molecule of the instant invention is greater than in the presence of the nucleic acid molecule than in its absence, or the presence of a control, irrelevant, or non-inhibitory oligonucleotide.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

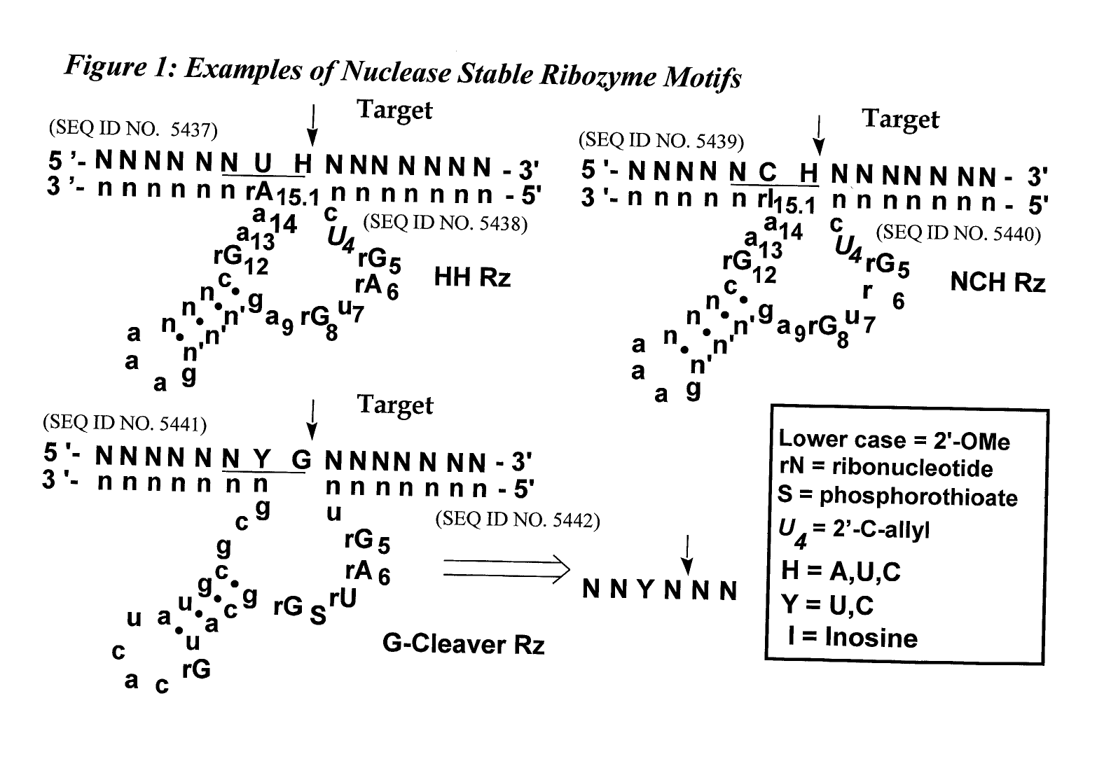

By “enzymatic portion” or “catalytic domain” is meant that portion/region of the enzymatic nucleic acid molecule essential for cleavage of a nucleic acid substrate (for example, see FIGS. 1-4).

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired. Examples of such arms are shown generally in FIGS. 1-4. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides and of sufficient length to stably interact with the target RNA; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “NCH” or “Inozyme” motif is meant, an enzymatic nucleic acid molecule comprising a motif as described in Ludwig et al., U.S. Ser. No. 09/406,643, filed Sep. 27, 1999, entitled “COMPOSITIONS HAVING RNA CLEAVING ACTIVITY”, and International PCT publication Nos. WO 98/58058 and WO 98/58057, all incorporated by reference herein in their entirety including the drawings.

By “G-cleaver” motif is meant, an enzymatic nucleic acid molecule comprising a motif as described in Eckstein et al., International PCT publication No. WO 99/16871, incorporated by reference herein in its entirety including the drawings.

By “zinzyme” motif is meant, a class II enzymatic nucleic acid molecule comprising a motif as described in Beigelman et al., International PCT publication No. WO 99/55857, incorporated by reference herein in its entirety including the drawings. Zinzymes represent a non-limiting example of an enzymatic nucleic acid molecule that does not require a ribonucleotide (2′-OH) group within its own nucleic acid sequence for activity.

By “amberzyme” motif is meant, a class I enzymatic nucleic acid molecule comprising a motif as described in Beigelman et al., International PCT publication No. WO 99/55857, incorporated by reference herein in its entirety including the drawings. Amberzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a ribonucleotide (2′-OH) group within the DNAzyme molecule for its activity. In particular embodiments the enzymatic nucleic acid molecule may have an attached linker(s) or other attached or associated groups, moieties, or chains containing one or more nucleotides with 2′-OH groups.

DNAzyme can be synthesized chemically or expressed endogenously in vivo, by means of a single stranded DNA vector or equivalent thereof.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant, interaction of the oligonucleotides with target nucleic acid (e.g., by forming hydrogen bonds with complementary nucleotides in the target under physiological conditions).

By “equivalent” RNA to CLCA1 is meant to include those naturally occurring RNA molecules having homology (partial or complete) to CLCA1 proteins or encoding for proteins with similar function as CLCA1 in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 25 Science 261, 1004 and Woolf et al, U.S. Pat. No. 5,849,902). Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. Antisense DNA can be synthesized chemically or expressed via the use of a single stranded DNA expression vector or equivalent thereof.

By “2-5A antisense chimera” it is meant, an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

By “triplex DNA” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504).

By “gene” it is meant a nucleic acid that encodes an RNA.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix inhibition. Determination of binding free energies for nucleic acid molecules is well known in the art (see, e.g., Turner et al., 1987, CSHSymp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

At least seven basic varieties of naturally occurring enzymatic nucleic acids are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in CLCA1-specific RNAs represent a novel therapeutic approach to treat Chronic Obstructive Pulmonary Diseases (COPDs), chronic bronchitis, asthma, cystic fibrosis, obstructive bowel syndrome, and other indications that may respond to the level of CLCA1.

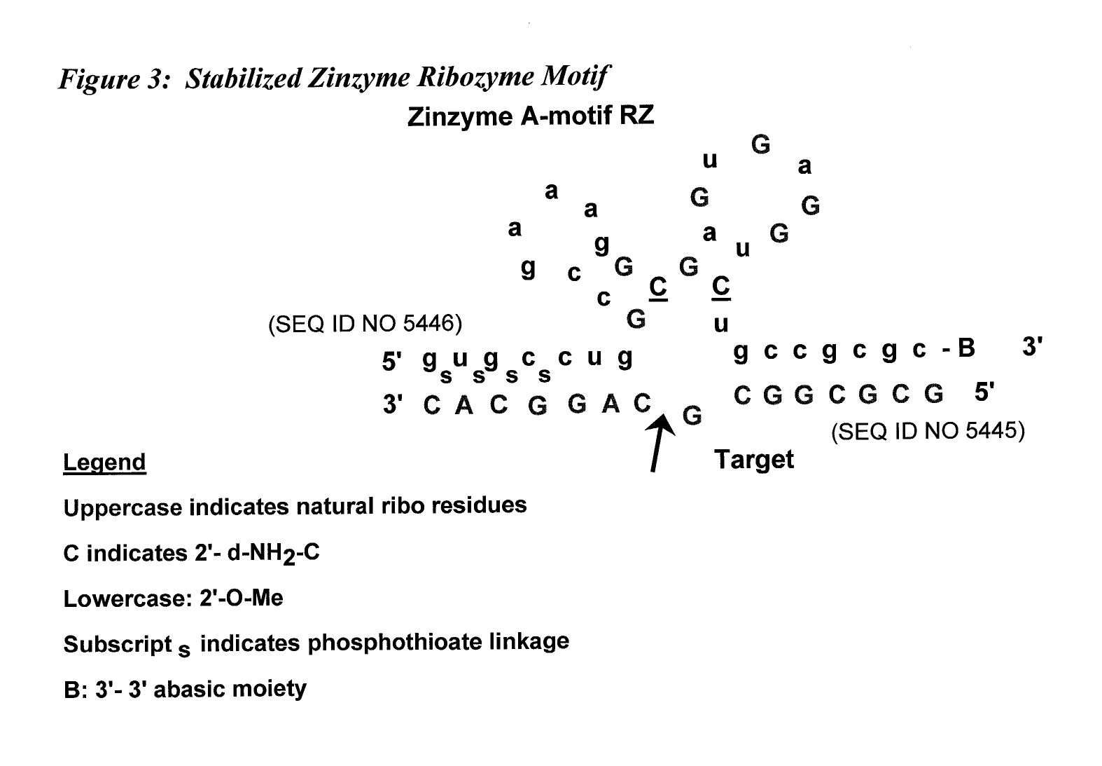

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; Examples of hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nucleic Acids Res. 18, 299; Chowrira & McSwiggen, U.S. Pat. No. 5,631,359. The hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; Li and Altman, 1996, Nucleic Acids Res. 24, 835. Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363). Group II introns are described by Griffin et al., 1995, Chem. Biol. 2, 761; Michels and Pyle, 1995, Biochemistry 34, 2965; Pyle et al., International PCT Publication No. WO 96/22689. The Group I intron is described by Cech et al., U.S. Pat. No. 4,987,071. DNAzymes are described by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., International PCT publication No. WO 99/55857) and Zinzyme (Beigelman et al., International PCT publication No. WO 99/55857), all these references are incorporated by reference herein in their totalities, including drawings and can also be used in the present invention. These specific motifs are not limiting in the invention, and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding CLCA proteins (for example, CLCA1, CLCA2, CLCA3 and/or CLCA4) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

In a preferred embodiment, the invention features the use of nucleic acid-based inhibitors of the invention to specifically target genes that share homology with the CLCA1 gene.

As used herein “cell” is used in its usual biological sense, and does not refer to an entire multicellular organism, e.g., specifically does not refer to a human. The cell may be present in a non-human multicellular organism, e.g., birds, plants and mammals such as cows, sheep, apes, monkeys, swine, dogs, and cats.

By “CLCA proteins” is meant, a protein or a mutant protein derivative thereof, comprising a calcium activated chloride channel protein.

By “highly conserved sequence region” is meant, a nucleotide sequence of one or more regions in a target gene does not vary significantly from one generation to the other or from one biological system to the other.

The nucleic acid-based inhibitors of CLCA1 expression are useful for the prevention and/or treatment of diseases and conditions including Chronic Obstructive Pulmonary Disease (COPD), chronic bronchitis, asthma, cystic fibrosis, obstructive bowel syndrome, and any other diseases or conditions that are related to or will respond to the levels of CLCA1 in a cell or tissue, alone or in combination with other therapies.

By “related” is meant that the reduction of CLCA1 expression (specifically CLCA1 gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to IX. Examples of such enzymatic nucleic acid molecules also are shown in Tables III to IX. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to IX. Such nucleic acid molecules can include sequences as shown for the binding arms of the enzymatic nucleic acid molecules in Tables III to VIII and sequences shown as GeneBloc™ sequences in Table IX. Similarly, triplex molecules can be provided targeted to the corresponding DNA target regions, and containing the DNA equivalent of a target sequence or a sequence complementary to the specified target (substrate) sequence. Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables III, IV and VIII can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 5450), or any other Stem II region known in the art.

In another aspect of the invention, ribozymes or antisense molecules that interact with target RNA molecules and inhibit CLCA1 (specifically CLCA1 gene) activity are expressed from transcription units inserted into DNA or RNA vectors. The recombinant vectors are preferably DNA plasmids or viral vectors. Ribozyme or antisense expressing viral vectors could be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed endogenously via the use of a single stranded DNA intracellular expression vector.

By RNA is meant a molecule comprising at least one ribonucleotide residue. By “ribonucleotide” is meant a nucleotide with a hydroxyl group at the 2′ position of a β-D-ribo-faranose moiety.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

The nucleic acid molecules of the instant invention, individually, or in combination or in conjunction with other drugs, can be used to treat diseases or conditions discussed above. For example, to treat a disease or condition associated with the levels of CLCA1, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat Chronic Obstructive Pulmonary Diseases (COPDs), chronic bronchitis, asthma, cystic fibrosis, obstructive bowel syndrome, and/or other disease states or conditions which respond to the modulation of CLCA1 expression.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., CLCA1) capable of progression and/or maintenance of Chronic Obstructive Pulmonary Diseases (COPDs), chronic bronchitis, asthma, cystic fibrosis, obstructive bowel syndrome, and/or other disease states or conditions which respond to the modulation of CLCA1 expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the And listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The foregoing description of the various aspects and embodiments is provided with reference to the exemplary calcium activated chloride channel gene CLCA1, which is also referred to as CaCC1 or ICACC-1. However, the various aspects and embodiments are also directed to other genes which express CLCA1 or CaCC1-like proteins (for example hCLCA2, hCLCA3, hCLCA4, CaCC2, and CaCC3). Those additional genes can be analyzed for target sites using the methods described for CLCA1. Thus, the inhibition and the effects of such inhibition of the other genes can be performed as described herein.

Other features and advantages of the invention will be apparent from the following description of the preferred embodiments thereof, and from the claims.

### DESCRIPTION OF THE PREFERRED EMBODIMENTS

First the drawings will be described briefly.

Drawings

FIG. 1 shows examples of chemically stabilized ribozyme motifs. HH Rz, 5 represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

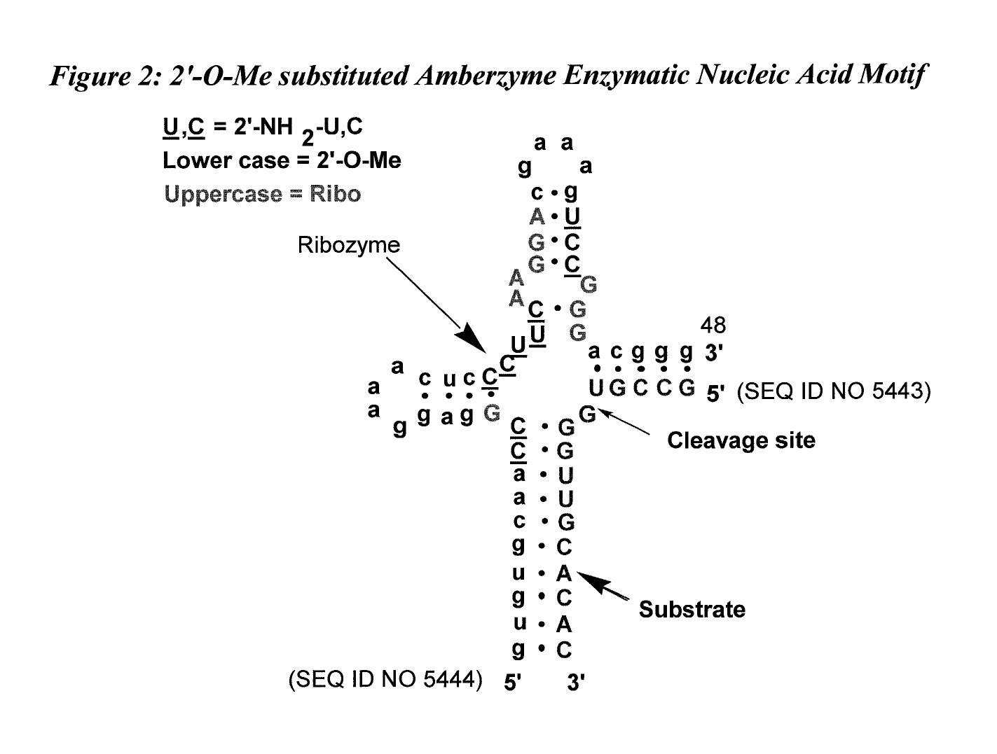

FIG. 2 shows an example of the Amberzyme ribozyme motif that is chemically stabilized (see, for example, Beigelman et al., International PCT publication No. WO 99/55857, incorporated by reference herein; also referred to as Class I Motif). The Amberzyme motif is a class of enzymatic nucleic molecules that do not require the presence of a ribonucleotide (2′-OH) group for its activity.

FIG. 3 shows an example of the Zinzyme A ribozyme motif that is chemically stabilized (Beigelman et al., International PCT publication No. WO 99/55857, incorporated by reference herein; also referred to as Class A or Class II Motif). The Zinzyme motif is a class of enzymatic nucleic molecules that do not require the presence of a ribonucleotide (2′-OH) group for its activity.

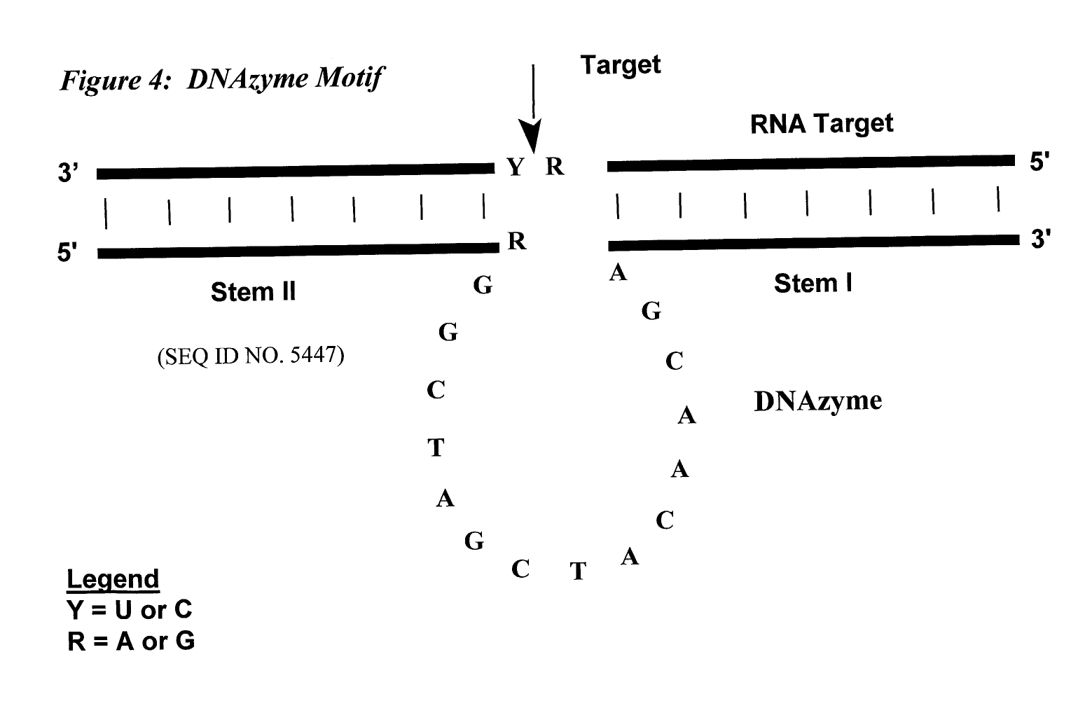

FIG. 4 shows an example of a DNAzyme motif described by Santoro et al., 1997, PNAS, 94, 4262.

FIGS. 5A and 5B are diagrammatic schemes representative of the process used for Target Discovery in the instant invention. The process for Target Discovery is described in Jarvis et al., International PCT publication No. WO 98/50530, incorporated by reference herein in its entirety including the Figures.

### MECHANISM OF ACTION OF NUCLEIC ACID MOLECULES OF THE INVENTION

Antisense:

Antisense molecules may be modified or unmodified RNA, DNA, or mixed polymer oligonucleotides which primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on September 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed endogenously in vivo via the use of a single stranded DNA intracellular expression vector or equivalents and variations thereof.

Triplex Forming Oligonucleotides (TFO):

Single stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth, supra).

2-5A Antisense Chimera:

The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-SA synthetases require double stranded RNA to form 2′-5′ oligoadenylates (2-SA). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures may be covalently linked to antisense molecules to form chimeric oligonucleotides capable of RNA cleavage (Torrence, supra). These molecules putatively bind and activate a 2-SA dependent RNase, the oligonucleotide/enzyme complex then binds to a target RNA molecule which can then be cleaved by the RNase enzyme.

Enzymatic Nucleic Acid:

Seven basic varieties of naturally occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent CLCA1 protein expression and can be used to treat disease or diagnose disease associated with the levels of CLCA1.

The enzymatic nature of a ribozyme has significant advantages, such as the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986 ; Uhlenbeck, 1987 Nature 328, 596; Kim et al., 84 Proc. Natl. Acad. Sci. USA 8788, 1987; Dreyfus, 1988, Einstein Quart. J Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes show promise as therapeutic agents for human disease (Usman and McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

The nucleic acid molecules of the instant invention are also referred to as GeneBloc reagents, which are essentially nucleic acid molecules (e.g.; ribozymes, antisense) capable of down-regulating gene expression.

GeneBlocs are modified oligonucleotides including ribozymes and modified antisense oligonucleotides that bind to and target specific mRNA molecules. Because GeneBlocs can be designed to target any specific mRNA, their potential applications are quite broad. Traditional antisense approaches have often relied heavily on the use of phosphorothioate modifications to enhance stability in biological samples, leading to a myriad of specificity problems stemming from non-specific protein binding and general cytotoxicity (Stein, 1995, Nature Medicine, 1, 1119). In contrast, GeneBlocs contain a number of modifications that confer nuclease resistance while making minimal use of phosphorothioate linkages, which reduces toxicity, increases binding affinity and minimizes non-specific effects compared with traditional antisense oligonucleotides. Similar reagents have recently been utilized successfully in various cell culture systems (Vassar, et al., 1999, Science, 286, 735) and in vivo (Jarvis et al., manuscript in preparation). In addition, novel cationic lipids can be utilized to enhance cellular uptake in the presence of serum. Since ribozymes and antisense oligonucleotides regulate gene expression at the RNA level, the ability to maintain a steady-state dose of GeneBloc over several days was important for target protein and phenotypic analysis. The advances in resistance to nuclease degradation and prolonged activity in vitro have supported the use of GeneBlocs in target validation applications.

Target Sites

Targets for useful ribozymes and antisense nucleic acids can be determined as disclosed in Draper et al, WO 93/23569; Sullivan et al., WO 93/23057; Thompson et al., WO 94/02595; Draper et al., WO 95/04818; McSwiggen et al., U.S. Pat. No. 5,525,468. All of these publications are hereby incorporated by reference herein in their totality. Other examples include the following PCT applications, which concern inactivation of expression of disease-related genes: WO 95/23225, WO 95/13380, WO 94/02595, all of which are incorporated by reference herein. Rather than repeat the guidance provided in those documents here, specific examples of such methods are provided herein, not limiting to those in the art. Ribozymes and antisense to such targets are designed as described in those applications and synthesized to be tested in vitro and in vivo, as also described. The sequences of human CLCA1 RNAs were screened for optimal enzymatic nucleic acid and antisense target sites using a computer-folding algorithm. Antisense, hammerhead, DNAzyme, NCH, amberzyme, zinzyme, or G-Cleaver ribozyme binding/cleavage sites were identified. These sites are shown in Tables III to IX (all sequences are 5′ to 3′ in the tables; the underlined region can be any base-paired sequence, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, amberzyme, zinzymne or G-Cleaver ribozyme binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (e.g.; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, US patent No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 Rmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 Rmol) of 2′-O-methyl phosphoramidite and a 105-fold excess of S-ethyl tetrazole (60 μL of 0.25 M =15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M =10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to -20° C., the supernatant is removed from the polymer support.

The support is washed three times with 1.0 mL of EtOH:MeCN:H20/3 :1: 1, vortexed and the supernatant is then added to the first supernatant. The combined supernatants, containing the oligoribonucleotide, are dried to a white powder. The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684 and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 lmol) of 2′-O-methyl phosphoramidite and a 75-fold excess of S-ethyl tetrazole (60 μL of 0.25 M =15 μmol) can be used in each coupling cycle of 2′-O-methyl residues relative to polymer-bound 5′-hydroxyl. A 66-fold excess (120 μL of 0.11 M =13.2 μmol) of alkylsilyl (ribo) protected phosphoramidite and a 150-fold excess of S-ethyl tetrazole (120 μL of 0.25 M =30 gmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. 10 synthesizer include; detritylation solution is 3% TCA in methylene chloride (ABI);

capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVET). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to -20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min.

The vial is brought to r.t. TEA3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G5 and a U for A14 (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the examples described above including but not limited to 96-well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., supra, the totality of which is hereby incorporated herein by reference) and are re-suspended in water.

The sequences of the ribozymes and antisense constructs that are chemically synthesized, useful in this study, are shown in Tables III to IX. Those in the art will recognize that these sequences are representative only of many more such sequences where the enzymatic portion of the ribozyme (all but the binding arms) is altered to affect activity. The ribozyme and antisense construct sequences listed in Tables III to IX may be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases may increase their potency (see e.g., Eckstein et al., International Publication No.

WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; 35 Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into ribozymes without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of these the nucleic acid-based molecules of the invention will lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules may also include combinations of different types of nucleic acid molecules.

Therapeutic nucleic acid molecules (e.g., enzymatic nucleic acid molecules and antisense nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

By “enhanced enzymatic activity” is meant to include activity measured in cells and/or in vivo where the activity is a reflection of both catalytic activity and ribozyme stability. In this invention, the product of these properties is increased or not significantly (less than 10-fold) decreased in vivo compared to an all RNA ribozyme or all DNA enzyme.

In yet another preferred embodiment, nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity of an all RNA ribozyme.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both termini. In non-limiting examples the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details, see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH3)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated π electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlmann & Peyman, 1990, Chemical Reviews, 90, 4, 544-579, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, fornacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of P-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which may be modified or unmodified. Such modified groups are described, for example, in Eckstein et al., U.S. Pat. No. 5,672,695 and Matulic-Adamic et al., WO 98/28317, respectively, which are both incorporated by reference herein in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules may be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al, PCT WO93/23569, Beigelman et al., PCT W099/05094, and Klimuk et al., PCT WO99/04819 all of which have been incorporated by reference herein.

In addition, the nucleic acid molecules of the instant invention, used to treat pulmonary diseases and disorders, may be administered directly to the lungs via pulmonary delivery. The pulmonary delivery of oligonucleotides is described by Bennett et al., International PCT publication Nos. WO/9960166 and WO/9960010; Danahay et al., 1999, Pharm. Res., 16(10), 1542-1549; Metzger and Nyce, 1999, J. Allergy Clin. Immunol., 104(2, Pt. 1), 260-266; Nicklin et al., 1998, Pharm. Res., 15(4), 583-591; Illum and Watts, International PCT publication No. WO/9735562; and Nyce, 1997, Expert Opin. Invest. Drugs, 6(9), 1149-1156.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and other compositions known in the art. The present invention also includes pharmaceutically acceptable formulations of the compounds described. These formulations include salts of the above compounds, e.g., acid addition salts, including salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble.

Other factors are known in the art, and include considerations such as toxicity and forms which prevent the composition or formulation from exerting its effect. By “systemic administration” is meant in vivo systemic absorption or accumulation of drugs in the blood stream followed by distribution throughout the entire body. Administration routes that lead to systemic absorption include, without limitations: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity.

Non-limiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011 ). All incorporated by reference herein. Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). All incorporated by reference herein. The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen.

In addition, the invention features the use of methods to deliver the nucleic acid molecules of the instant invention to hematopoietic cells, including monocytes and lymphocytes. These methods are described in detail by Hartmann et al., 1998, J. Phamacol. Exp. Ther., 285(2), 920-928; Kronenwett et al., 1998, Blood, 91(3), 852-862; Filion and Phillips, 1997, Biochim. Biophys. Acta., 1329(2), 345-356; Ma and Wei, 1996, Leuk. Res., 20(11/12), 925-930; and Bongartz et al., 1994, Nucleic Acids Research, 22(22), 4681-8. Such methods, as described above, include the use of free oligonucleotide, cationic lipid formulations, liposome formulations including pH sensitive liposomes and immunoliposomes, and bioconjugates including oligonucleotides conjugated to fusogenic peptides, for the transfection of hematopoietic cells with oligonucleotides.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects. Oxygen therapy, bronchodilators, corticosteroids, antibacterials, vaccinations, acetylcysteine, mucokinetic agents, and DNase (Pulmozyme) are non-limiting examples of compounds and/or methods that can be combined with or used in conjunction with the nucleic acid molecules (e.g. ribozymes and antisense molecules) of the instant invention. Those skilled in the art will recognize that other drug compounds and therapies can be similarly and readily combined with the nucleic acid molecules of the instant invention (e.g. ribozymes and antisense molecules) and are, therefore, within the scope of the instant invention. Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intramuscular administration, by administration to target cells explanted from the patient followed by reintroduction into the patient, or by any other means that would allow for introduction into the desired target cell (for a review, see Couture et al., 1996, TIG., 12,510).

In one aspect, the invention features an expression vector comprising a nucleic acid sequence encoding at least one of the nucleic acid molecules disclosed in the instant invention. The nucleic acid sequence encoding the nucleic acid molecule of the instant invention is operably linked in a manner which allows expression of that nucleic acid molecule.

In another aspect, the invention features an expression vector comprising: a) a transcription initiation region (e.g., eukaryotic pol I, II or Ell initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol HI promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein.

Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; and Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al, 1994, Nucleic Acid Res., 22, 2830; Noonberg et al, U.S. Pat. No. 5,624,803; Good et al., 1997, Gene Ther., 4, 45; and Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review, see Couture and Stinchcomb, 1996, supra).

In yet another aspect, the invention features an expression vector comprising a nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another preferred embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule.

In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within CLCA1 RNA.

### Example 1

### Reporter System

Applicant used a target discovery and target validation approach to finding genes that are involved in chronic mucous hypersecretion. In order to discover genes playing a role in the expression of mucins, a readily assayable reporter system was devised. The reporter system consists of a plasmid construct, termed pMUC5AC-EGFP, bearing a gene coding for Green Fluorescent Protein (GFP). The promoter region of the GFP gene is replaced by a portion of the Mucin 5AC promoter sufficient to direct efficient transcription of the GFP gene. The plasmid also contains the neomycin drug resistance gene.

### Example 2

### Host Cell Line for Target Discovery

The cell line selected as host for these studies, NCI-H292 (ATCC CRL-1848), is derived from a human lung mucoepidermoid carcinoma. The cells retain mucoepidermoid characteristics in culture and endogenously express mucin 5AC and mucin 2. The pMUC5AC-EGFP plasmid was transfected into NCI-H292 using a cationic lipid formulation. Following transfection, the cells were subjected to limiting dilution cloning under selection by 600 μg/mL Geneticin. Cells retaining the pMUC5AC-EGFP plasmid survive the Geneticin treatment and form colonies derived from single surviving cells. The resulting clonal cell lines were screened by flow cytometry for the capacity to upregulate GFP production directed by the Mucin 5AC promoter. Treating the cells with sterilized M9 bacterial medium in which Pseudomonas aeruginosa had been cultured (Pseudomonas conditioned medium, PCM) induced the mucin promoter. The PCM is supplemented with phorbol myristate acetate (PMA).

A clonal cell line highly responsive to mucin promoter induction, designated H292/MUC5AC/EGFP Clone8 (H292 Clone 8) was selected as the reporter line for subsequent studies. The process for Target Discovery is described in Jarvis et al., International PCT publication No. WO 98/50530, incorporated by reference herein in its entirety including the Figures.

### Example 3

### Ribozyme Library Construction

A ribozyme library was constructed with oligonucletides containing ribozymes with two randomized regions comprising six-nucleotide binding “arms” (Stem I and Stem HI of a ribozyme-substrate complex). Oligo sequence 5′ and 3′ of the ribozyme contains restriction endonuclease cleavage sites for cloning. The 3′ trailing sequence forms a stem-loop for priming DNA polymerase extension to form a double stranded molecule. The double-stranded ribozyme library was cloned into the U6+27 transcription unit located in the 5′ LTR region of a retroviral vector containing the human nerve growth factor receptor (hNGFr) reporter gene.

Positioning the U6+27/ribozyme transcription unit in the 5′ LTR results in a duplication of the transcription unit when the vector integrates into the host cell genome. As a result, the ribozyme is transcribed by RNA polymerase HI from U6+27 and by RNA polymerase II activity directed by the 5′ LTR. The ribozyme library was packaged into retroviral particles that were used to infect and transduce H292 Clone 8 cells. Assay of the hNGFr reporter indicated that 50% to 60% of Clone 8 cells incorporated the ribozyme construct. FIGS. 5A and 5B describe the generalized scheme used in the ribozyme library construction and target discovery. By “randomized region” is meant a region of completely random sequence and/or partially random sequence. By completely random sequence is meant a sequence wherein theoretically there is equal representation of A, T, G and C nucleotides or modified derivatives thereof, at each position in the sequence. By partially random sequence is meant a sequence wherein there is an unequal representation of A, T, G and C nucleotides or modified derivatives thereof, at each position in the sequence. A partially random sequence can therefore have one or more positions of complete randomness and one or more positions with defined nucleotides.

### Example 4

### Enriching for Non-responders to Mucin Induction

Sorting of ribozyme library-containing cells was performed to enrich for cells that produce less GFP after treatment with PCM and PMA. Lower GFP production may be due to ribozyme action upon genes involved in the activation of the mucin promoter. Alternatively, ribozymes may directly target the mucin/GFP transcript resulting in reduced GFP expression.

Cells were seeded at a density of 1×10⁶ per 150 cm² style cell culture flasks. After 72 hours the standard cell culture medium was replaced with medium without fetal bovine serum. After 24 hours of serum deprivation the cells were treated with serum-containing medium supplemented with PCM (to 40%) and PMA (to 50 nM) to induced GFP production via the mucin promoter. After 20 to 22 hours, cells were monitored for GFP level on a FACStar Plus cell sorter.

Sorting was performed if 90% of ribozyme library cells from an unsorted control sample were induced to produce GFP above background levels. Two cell fractions were collected in each round of sorting.

In the initial sort the M1 gate collected cells in luminescence channels 1 to 4.5; those cells with the lowest GFP signal (5% of the induced population). The M2 sort gate collected cells in luminescence channels 4.5 to 20; cells with low GFP signal (10% of the induced population). The M1 and M2 fractions together represented the 15% of the induced population responding least to the GFP induction treatment. In order to assure that the diversity of the ribozyme library was represented 2.3×10⁶ cells were collected in the M1 fraction and 4.6×10⁶ cells were collected in the M2 fraction. The M1 and M2 fractions wee cultured separately and representative portions of each were cryopreserved after each round of sorting.

When treated with PCM and PMA prior to a second round of sorting, cells from both the M1 and M2 fractions responded as before with >90% of the cells producing elevated levels of GFP. The same sorting criteria and sort gates were used in the second round. As in the first round of sorting the M1 sort gate collected 5% of the treated cells (those with little or no GFP) and the M2 gate collected 10% of the cells. Two more rounds of sorting were performed using the same sorting criteria.

Prior to the third round of sorting the M1 fraction showed a three-fold enrichment of GFP negative cells. Prior to the fourth round of sorting both the M1 and M2 fractions were significantly enriched in cells unresponsive to the GFP induction treatment.

Following the third round of sorting the M1 fraction was selected to generate a database of ribozymes present in the sorted cells.

### Example 5

### Recovery of Ribozyme Sequence from Sorted Cells

Genomic DNA was obtained from sorted ribozyme library cells by standard methods. Nested polymerase chain reaction (PCR) primers (Sequence ID Nos. 5468 and 5469) that hybridized to the retroviral vector 5′ and 3′ of the ribozyme were used to recover and amplify the ribozyme sequences from the Clone 8 library cell DNA. The PCR product was ligated into a bacterial cloning vector. Two methods were developed to use the recovered ribozyme library, in plasmid form, to generate a database of ribozyme binding arm sequences. In the first approach the library was cloned into E. coli. DNA was prepared by plasmid isolation from bacterial colonies or by direct colony PCR and ribozyme arm sequence was determined. Over 450 sequences have been obtained by this method. A second method used the ribozyme library to transfect H292 Clone 8 cells. Clonal lines of stably transfected cells were established and induced with PCM and PMA. Those lines which failed to respond to GFP induction were probed by PCR for single ribozyme integration events. Over 300 sequences were obtained in this manner. The unique ribozyme sequences obtained by both methods were added to a Target Sequence Tag (TST) database.

### Example 6

### Bioinformatics

After sequencing 760 recovered ribozymes 171 unique sequences were found. Of the unique sequences, 91 have been recovered once and 80 have been found multiple times. Most of the repeated sequences have been found 2 to 11 times. One sequence has been recovered 145 times. The diversity of the sequences obtained indicates that the sorted cells are a promising source of information leading to target discovery.

Ribozyme binding arm sequences were compared to public and private gene data banks. Gene matches were compiled according to perfect and imperfect matches. Potential gene targets were categorized by the number of different ribozyme sequences matching each gene. Multiple ribozyme matches have been found for 180 genes. Genes with more than one perfect ribozyme match were given close attention. A total of 34 genes have been verified to date to have multiple perfect ribozyme matches. Of those at least 17 have protein products of known function.

Two perfect ribozyme matches were found for human calcium activated chloride channel-1 (hCLCA1). Each ribozyme matches at two sites in the hCLCA1 gene. A third sorted library ribozyme sequence “hits” hCLCA1 but has a single nucleotide mismatch.

### Example 7

### Selection of hCLCA1 for Validation

The selection of hCLCA1 as a candidate for target validation was based on bioinformatics and on emerging data in murine models of mucous hypersecretion in the trachea and lung. Two ribozymes (Seq. ID Nos. 2332 and 2273) recovered from cells that no longer respond to mucin promoter/GFP induction match perfectly to hCLCA1. A third has a single mismatch. Evidence from two murine models indicates a correlation between mucous hypersecretion in the lung and strong upregulation of gob-5 (GenBank ABO17156), a murine homologue of hCLCA1.

### Example 8

### Validation of hCLCA1

To validate hCLCA1 as a regulator of MUC5AC expression, GeneBloc reagents were designed (Table IX) to the hCLCA1 cDNA sequence (GenBank AF039400). GeneBloc reagents are complexed with a cationic lipid formulation prior to administration to H292/MUC5AC/GFP Clone 8 cells. Concentrations of the GeneBloc reagents administered range from 30 nM to 120 nM at cationic lipid concentrations of 4-6 μg/mL. Cells are treated with GeneBloc reagents for 72 to 96 30 hours. Before the termination of GeneBloc treatment, PCM (to 40 %) and PMA (to 50 nM) are added to induce the MUC5AC promoter. After twenty hours of induction the cells are harvested and assayed for phenotypic and molecular parameters. Reduced GFP expression in GeneBloc treated cells (measured by flow cytometry) is taken as evidence for validation of hCLCA1. Knockdown of hCLCA1 RNA in GeneBloc treated cells can correlate with reduced endogenous MUC5AC RNA and reduced GFP RNA (from the MUC5AC/GFP construct) to complete validation of hCLCA1.

### Example 9

### Identification of Potential Target Sites in Human CLCA1 RNA

The sequence of human CLCA1 is screened for accessible sites using a computer-folding algorithm. Regions of the RNA are identified that do not form secondary folding structures. These regions contain potential ribozyme and/or antisense binding/cleavage sites. The sequences of these binding/cleavage sites are shown in Tables III-IX.

### Example 10

### Selection of Enzymatic Nucleic Acid Cleavage Sites in Human CLCA1 RNA

Ribozyme target sites are chosen by analyzing sequences of Human CLCA1 (GenBank accession numbers: NM_{—}001285 and AF039400) and prioritizing the sites on the basis of folding. Ribozymes are designed that could bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 11

### Chemical Synthesis and Purification of Ribozymes and Antisense for Efficient Cleavage and/or blocking of CLCA1 RNA

Ribozymes and antisense constructs are designed to anneal to various sites in the RNA message. The binding arms of the ribozymes are complementary to the target site sequences described above, while the antisense constructs are fully complimentary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Ribozymes and antisense constructs are also synthesized from DNA templates using bacteriophage T7 RNA polymerase (Milligan and Uhlenbeck, 1989, Methods Enzymol. 180, 51). Ribozymes and antisense constructs are purified by gel electrophoresis using general methods or are purified by high pressure liquid chromatography (HPLC; see Wincott et al., supra; the totality of which is hereby incorporated herein by reference) and are resuspended in water. The sequences of the chemically synthesized ribozymes and antisense constructs used in this study are shown below in Table III-IX.

Indications

Particular conditions and disease states that can be associated with CLCA1 expression modulation include but are not limited to Chronic Obstructive Pulmonary Disease (COPD), chronic bronchitis, asthma, cystic fibrosis, obstructive bowel syndrome, and any other diseases or conditions that are related to or will respond to the levels of CLCA1 in a cell or tissue, alone or in combination with other therapies.

The present body of knowledge in CLCA1 research indicates the need for methods to assay CLCA1 activity and for compounds that can regulate CLCA1 expression for research, diagnostic, and therapeutic use.

The nucleic acid molecules of the present invention may also be administered to a patient in combination with other therapeutic compounds to increase the overall therapeutic effect. The use of multiple compounds to treat an indication may increase the beneficial effects while reducing the presence of side effects. Oxygen therapy, bronchodilators, corticosteroids, antibacterials, vaccinations, acetylcysteine, mucokinetic agents, and DNase (Pulmozyme), are non-limiting examples of methods and/or treatments that can be used in combination with nucleic acid molecules of the invention. Those skilled in the art will recognize that other drug compounds and therapies can be similarly and readily combined with the nucleic acid molecules of the instant invention (e.g. ribozymes and antisense molecules) and are, therefore, within the scope of the instant invention.

Cell Culture

The cell culture system described in Example 8 can be used to evaluate nucleic acid molecules of the invention for efficacy in CLCA1 and mucin modulation.

Animal Models

Numerous reports can be found which describe animal models relevant to disease states such as COPD and cystic fibrosis. These models can be used to determine efficacy of the nucleic acid molecules of the instant invention targeting such disease states or conditions. Animal models for chronic pulmonary disease (COPD) are described by Shapiro, 2000, Am. J. Respir. Cell Mol. BioL, 22(1), 4-7; Hogg, 1998, Ika Daigaku Zasshi, 56(3), 429-432; and Garssen et al., 1997, Inhalation Toxicol., 9(6), 581-599. Animal models for cystic fibrosis are described by Kent et al., 1997, J. Clin. Invest., 100(12), 3060-3069; Hill et al., 1997, 62(1), 113-122; Grubb and Gabriel, 1997, Am. J. Physiol., 272, G258-G266; Rozmahel, 1996, From: Diss. Abstr. Int. B 1997, 57(8), 4863; Van Doominck et al., 1995, EMBOJ., 14(18), 4403-11; and Zeiher et al., 1995, J. Clin. Invest., 96(4), 2051-64.

Diagnostic Uses

The nucleic acid molecules of this invention (e.g., ribozymes) may be used as diagnostic tools to examine genetic drift and mutations within diseased cells or to detect the presence of CLCA1 RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with CLCA1 -related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA will be cleaved by both ribozymes to demonstrate the relative ribozyme efficiencies in the reactions and the absence of cleavage of the “non-targeted” RNA species. The cleavage products from the synthetic substrates will also serve to generate size markers for the analysis of wild-type and mutant RNAs in the sample population. Thus, each analysis can require two ribozymes, two substrates and one unknown sample, which will be combined into six reactions. The presence of cleavage products will be determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., CLCA1) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the . levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintainance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic
comparisons, mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{viii},^{ix}].
Chemical modification investigation of important residues well established
[^{x},^{xi}]
The small (4-6 nt) binding site may make this ribozyme too non-specific
for targeted RNA cleavage, however, the Tetrahymena group I intron has
been used to repair a “defective” □-galactosidase message by the ligation
of new □-galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage
products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS) to
the target RNA [^{xiv},^{xv}]
Important phosphate and 2′-OH contacts recently identified [^{xvi},^{xvii}]
Group II Introns
Size: >1000 nucleotides.
Trans cleavage of target RNAs recently demonstrated [^{xviii},^{xix}].
Sequence requirements not fully determined.
Reaction mechanism: 2′-OH of an internal adenosine generates cleavage
products with 3′-OH and a “lariat” RNA containing a 3′-5′ and a 2′-5′
branch point.
Only natural ribozyme with demonstrated participation in DNA cleavage
[^{xx},^{xxi}] in addition to RNA cleavage and ligation.
Major structural features largely established through phylogenetic
comparisons [^{xxii}].
Important 2′-OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal structures
[^{xxvi},^{xxvii}]
Minimal ligation activity demonstrated (for engineering through in vitro
selection) [^{xxviii}]
Complete kinetic framework established for two or more ribozymes [^{xxix}].
Chemical modification investigation of important residues well established
[^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi},^{xxxii},^{xxxiii},^{xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues begun
[^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{xl}].
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability
[^{xli}]
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R., Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R., Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H., A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H., Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H., The time dependence of chemical modification reveals slow steps in the folding of a group I ribozyme. Biochemistry (1995), 34(19), 6504-12.
^{ix}Zarrinkar, Patrick P.; Williamson, James R., The P9.1-P9.2 peripheral extension helps guide folding of the Tetrahymena ribozyme. Nucleic Acids Res. (1996), 24(5), 854-8.
^{x}Strobel, Scott A.; Cech, Thomas R., Minor groove recognition of the conserved G.cntdot.U Pair at the Tetrahymena ribozyme reaction site. Science (Washington, D. C.) (1995), 267(5198), 675-9.
^{xi}Strobel, Scott A.; Cech, Thomas R., Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii}Sullenger, Bruce A.; Cech, Thomas R., Ribozyme-mediated repair of defective mRNA by targeted trans-splicing. Nature (London) (1994), 371(6498), 619-22.
^{xiii}Robertson, H.D.; Altman, S.; Smith, J. D. J. Biol. Chem., 247, 5243-5251 (1972).
^{xiv}Forster, Anthony C.; Altman, Sidney. External guide sequences for an RNA enzyme. Science (Washington, D. C., 1883-) (1990), 249(4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R., Identification of phosphates involved in catalysis by the ribozyme RNase P RNA. RNA (1995), 1(2), 210-18.
^{xvii}Pan, Tao; Lana, Andrew; Zhong, Kun. Probing of tertiary interactions in RNA: 2-hydroxyl-base contacts between the RNase P RNA and pre-tRNA. Proc. Natl. Acad. Sci. U.S.A. (1995), 92(26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M., A group II intron RNA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2-hydroxyl groups. Chem. Biol. (1995), 2(11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C. T.; Collins, Richard A.. Efficient trans-cleavage of a stem-loop RNA substrate by a ribozyme derived from Neurospora VS RNA. EMBO J. (1995), 14(2), 368-76.
^{xxvi}Scott, W. G., Finch, J. T., Aaron, K. The crystal structure of an all RNA hammerhead ribozyme: A proposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K. J., Herschlag, D., Uhlenbeck, O. A kinetic and thermodynamic framework for the hammerhead ribozyme reaction. Biochemistry, (1994) 33, 3374-3385. Beigelman, L., et aL, Chemical modifications of hammerhead ribozymes. J. Biol. Chem., (1995) 270, 25702-25708.
^{xxx}Beigelman, L., et al, Chemical modifications of hammerhead ribozymes. J. Biol. Chem., (1995) 270, 25702-25708.
^{xxxi}Hampel, Arnold; Tritz, Richard; Hicks, Margaret; Cruz, Philip. ‘Hairpin’ catalytic RNA model: evidence for helixes and sequence requirement for substrate RNA. Nucleic Acids Res. (1990), 18(2), 299-304.
^{xxxii}Chownira, Bharat M.; Berzal-Herranz, Alfredo; Burke, John M., Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M., Essential nucleotide sequences and secondary structure elements of the hairpin ribozyme. EMBO J. (1993), 12(6), 2567-73.
^{xxxiv}Joseph, Simpson; Berzal-Herranz, Alfredo; Chownira, Bharat M.; Butcher, Samuel E., Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M., In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J., Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J., Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ukik S.; Gait, Michael J., Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D., Cleavage of oligoribonucleotides by a ribozyme derived from the hepatitis delta. virus RNA sequence. Biochemistry (1992), 31(1), 16-21.
^{xl}Perrotta, Anne T.; Been, Michael D., A pseudoknot-like structure required for efficient self-cleavage of hepatitis delta virus RNA. Nature (London) (1991), 350(6317), 434-6.
^{xli}Puttaraju, M.; Perrotta, Anne T.; Been, Michael D., A circular trans-acting hepatitis delta virus ribozyme. Nucleic Acids Res. (1993), 21(18), 4253-8.

TABLE II

| | | | | | |
|---|---|---|---|---|---|
| | | | Wait | Wait | Wait |
| | Equiva- | | Time* | Time* | Time* |
| Reagent | lents | Amount | DNA | 2′-O-methyl | RNA |

A. 2.5 μmol Synthesis Cycle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equiva- | | | | |
|---|---|---|---|---|---|
| | lents | Amount | | Wait | |
| | DNA/2′- | DNA/2′- | Wait | Time* | Wait |
| | O-methyl/ | O-methyl/ | Time* | 2′-O- | Time* |
| Reagent | Ribo | Ribo | DNA | methyl | Ribo |
| Phosphoramidites | 22/33/66 | 40/60/ | 60 sec | 180 sec | 360 sec |
| | | 120 μL | | | |
| S-Ethyl Tetrazole | 70/105/ | 40/60/ | 60 sec | 180 min | 360 sec |
| | 210 | 120 μL | | | |
| Acetic Anhydride | 265/265/ | 50/50/ | 10 sec | 10 sec | 10 sec |
| | 265 | 50 μL | | | |
| N-Methyl | 502/502/ | 50/50/ | 10 sec | 10 sec | 10 sec |
| Imidazole | 502 | 50 μL | | | |
| TCA | 238/475/ | 250/500/ | 15 sec | 15 sec | 15 sec |
| | 475 | 500 μL | | | |
| Iodine | 6.8/6.8/ | 80/80/ | 30 sec | 30 sec | 30 sec |
| | 6.8 | 80 μL | | | |
| Beaucage | 34/51/51 | 80/120/120 | 100 sec | 200 sec | 200 sec |
| Acetonitrile | NA | 1150/1150/ | NA | NA | NA |
| | | 1150 μL | | | |

*Wait time does not include contact time during delivery.

TABLE III
Human CLCA1 Hammerhead Ribozyme and Target Sequence 249.021

| | | | | |
|---|---|---|---|---|
| | | | | Rz |
| | | Seq ID | | Seq ID |
| Pos | Substrate | No. | Ribozyme | No. |

| | | | | | |
|---|---|---|---|---|---|
| 11 | CUAAUGCU U UUGGUACA | 1 | UGUACCAA CUGAUGAG GCCGUUAGGC CGAA AGCAUUAG | 2190 | |
| 12 | UAAUGCUU U UGGUACAA | 2 | UUGUACCA CUGAUGAG GCCGUUAGGC CGAA AAGCAUUA | 2191 | |
| 13 | AAUGCUUU U GGUACAAA | 3 | UUUGUACC CUGAUGAG GCCGUUAGGC CGAA AAAGCAUU | 2192 | |
| 17 | CUUUUGGU A CAAAUGGA | 4 | UCCAUUUG CUGAUGAG GCCGUUAGGC CGAA ACCAAAAG | 2193 | |
| 34 | UGUGGAAU A UAAUUGAA | 5 | UUCAAUUA CUGAUGAG GCCGUUAGGC CGAA AUUCCACA | 2194 | |
| 36 | UGGAAUAU A AUUGAAUA | 6 | UAUUCAAU CUGAUGAG GCCGUUAGGC CGAA AUAUUCCA | 2195 | |
| 39 | AAUAUAAU U GAAUAUUU | 7 | AAAUAUUC CUGAUGAG GCCGUUAGGC CGAA AUUAUAUU | 2196 | |
| 44 | AAUUGAAU A UUUUCUUG | 8 | CAAGAAAA CUGAUGAG GCCGUUAGGC CGAA AUUCAAUU | 2197 | |
| 46 | UUGAAUAU U UUCUUGUU | 9 | AACAAGAA CUGAUGAG GCCGUUAGGC CGAA AUAUUCAA | 2198 | |
| 47 | UGAAUAUU U UCUUGUUU | 10 | AAACAAGA CUGAUGAG GCCGUUAGGC CGAA AAUAUUCA | 2199 | |
| 48 | GAAUAUUU U CUUGUUUA | 11 | UAAACAAG CUGAUGAG GCCGUUAGGC CGAA AAAUAUUC | 2200 | |
| 49 | AAUAUUUU C UUGUUUAA | 12 | UUAAACAA CUGAUGAG GCCGUUAGGC CGAA AAAAUAUU | 2201 | |
| 51 | UAUUUUCU U GUUUAAGG | 13 | CCUUAAAC CUGAUGAG GCCGUUAGGC CGAA AGAAAAUA | 2202 | |
| 54 | UUUCUUGU U UAAGGGGA | 14 | UCCCCUUA CUGAUGAG GCCGUUAGGC CGAA ACAAGAAA | 2203 | |
| 55 | UUCUUGUU U AAGGGGAG | 15 | CUCCCCUU CUGAUGAG GCCGUUAGGC CGAA AACAAGAA | 2204 | |
| 56 | UCUUGUUU A AGGGGAGC | 16 | GCUCCCCU CUGAUGAG GCCGUUAGGC CGAA AAACAAGA | 2205 | |
| 77 | AGAGGUGU U GAGGUUAU | 17 | AUAACCUC CUGAUGAG GCCGUUAGGC CGAA ACACCUCU | 2206 | |
| 83 | GUUGAGGU U AUGUCAAG | 18 | CUUGACAU CUGAUGAG GCCGUUAGGC CGAA ACCUCAAC | 2207 | |
| 84 | UUGAGGUU A UGUCAAGC | 19 | GCUUGACA CUGAUGAG GCCGUUAGGC CGAA AACCUCAA | 2208 | |
| 88 | GGUUAUGU C AAGCAUCU | 20 | AGAUGCUU CUGAUGAG GCCGUUAGGC CGAA ACAUAACC | 2209 | |
| 95 | UCAAGCAU C UGGCACAG | 21 | CUGUGCCA CUGAUGAG GCCGUUAGGC CGAA AUGCUUGA | 2210 | |
| 122 | AUGGAAAU A UUUACAAG | 22 | CUUGUAAA CUGAUGAG GCCGUUAGGC CGAA AUUUCCAU | 2211 | |
| 124 | GGAAAUAU U UACAAGUA | 23 | UACUUGUA CUGAUGAG GCCGUUAGGC CGAA AUAUUUCC | 2212 | |
| 125 | GAAAUAUU U ACAAGUAC | 24 | GUACUUGU CUGAUGAG GCCGUUAGGC CGAA AAUAUUUC | 2213 | |
| 126 | AAAUAUUU A CAAGUACG | 25 | CGUACUUG CUGAUGAG GCCGUUAGGC CGAA AAAUAUUU | 2214 | |
| 132 | UUACAAGU A CGCAAUUU | 26 | AAAUUGCG CUGAUGAG GCCGUUAGGC CGAA ACUUGUAA | 2215 | |
| 139 | UACGCAAU U UGAGACUA | 27 | UAGUCUCA CUGAUGAG GCCGUUAGGC CGAA AUUGCGUA | 2216 | |
| 140 | ACGCAAUU U GAGACUAA | 28 | UUAGUCUC CUGAUGAG GCCGUUAGGC CGAA AAUUGCGU | 2217 | |
| 147 | UUGAGACU A AGAUAUUG | 29 | CAAUAUCU CUGAUGAG GCCGUUAGGC CGAA AGUCUCAA | 2218 | |
| 152 | ACUAAGAU A UUGUUAUC | 30 | GAUAACAA CUGAUGAG GCCGUUAGGC CGAA AUCUUAGU | 2219 | |
| 154 | UAAGAUAU U GUUAUCAU | 31 | AUGAUAAC CUGAUGAG GCCGUUAGGC CGAA AUAUCUUA | 2220 | |
| 157 | GAUAUUGU U AUCAUUCU | 32 | AGAAUGAU CUGAUGAG GCCGUUAGGC CGAA ACAAUAUC | 2221 | |
| 158 | AUAUUGUU A UCAUUCUC | 33 | GAGAAUGA CUGAUGAG GCCGUUAGGC CGAA AACAAUAU | 2222 | |
| 160 | AUUGUUAU C AUUCUCCU | 34 | AGGAGAAU CUGAUGAG GCCGUUAGGC CGAA AUAACAAU | 2223 | |
| 163 | GUUAUCAU U CUCCUAUU | 35 | AAUAGGAG CUGAUGAG GCCGUUAGGC CGAA AUGAUAAC | 2224 | |
| 164 | UUAUCAUU C UCCUAUUG | 36 | CAAUAGGA CUGAUGAG GCCGUUAGGC CGAA AAUGAUAA | 2225 | |
| 166 | AUCAUUCU C CUAUUGAA | 37 | UUCAAUAG CUGAUGAG GCCGUUAGGC CGAA AGAAUGAU | 2226 | |
| 169 | AUUCUCCU A UUGAAGAC | 38 | GUCUUCAA CUGAUGAG GCCGUUAGGC CGAA AGGAGAAU | 2227 | |
| 171 | UCUCCUAU U GAAGACAA | 39 | UUGUCUUC CUGAUGAG GCCGUUAGGC CGAA AUAGGAGA | 2228 | |
| 187 | AGAGCAAU A GUAAAACA | 40 | UGUUUUAC CUGAUGAG GCCGUUAGGC CGAA AUUGCUCU | 2229 | |
| 190 | GCAAUAGU A AAACACAU | 41 | AUGUGUUU CUGAUGAG GCCGUUAGGC CGAA ACUAUUGC | 2230 | |
| 199 | AAACACAU C AGGUCAGG | 42 | CCUGACCU CUGAUGAG GCCGUUAGGC CGAA AUGUGUUU | 2231 | |
| 204 | CAUCAGGU C AGGGGGUU | 43 | AACCCCCU CUGAUGAG GCCGUUAGGC CGAA ACCUGAUG | 2232 | |
| 212 | CAGGGGGU U AAAGACCU | 44 | AGGUCUUU CUGAUGAG GCCGUUAGGC CGAA ACCCCCUG | 2233 | |
| 213 | AGGGGGUU A AAGACCUG | 45 | CAGGUCUU CUGAUGAG GCCGUUAGGC CGAA AACCCCCU | 2234 | |
| 226 | CCUGUGAU A AACCACUU | 46 | AAGUGGUU CUGAUGAG GCCGUUAGGC CGAA AUCACAGG | 2235 | |
| 234 | AAACCACU U CCGAUAAG | 47 | CUUAUCGG CUGAUGAG GCCGUUAGGC CGAA AGUGGUUU | 2236 | |
| 235 | AACCACUU C CGAUAAGU | 48 | ACUUAUCG CUGAUGAG GCCGUUAGGC CGAA AAGUGGUU | 2237 | |
| 240 | CUUCCGAU A AGUUGGAA | 49 | UUCCAACU CUGAUGAG GCCGUUAGGC CGAA AUCGGAAG | 2238 | |
| 244 | CGAUAAGU U GGAAACGU | 50 | ACGUUUCC CUGAUGAG GCCGUUAGGC CGAA ACUUAUCG | 2239 | |
| 257 | ACGUGUGU C UAUAUUUU | 51 | AAAAUAUA CUGAUGAG GCCGUUAGGC CGAA ACACACGU | 2240 | |
| 259 | GUGUGUCU A UAUUUUCA | 52 | UGAAAAUA CUGAUGAG GCCGUUAGGC CGAA AGACACAC | 2241 | |
| 261 | GUGUCUAU A UUUUCAUA | 53 | UAUGAAAA CUGAUGAG GCCGUUAGGC CGAA AUAGACAC | 2242 | |
| 263 | GUCUAUAU U UUCAUAUC | 54 | GAUAUGAA CUGAUGAG GCCGUUAGGC CGAA AUAUAGAC | 2243 | |
| 264 | UCUAUAUU U UCAUAUCU | 55 | AGAUAUGA CUGAUGAG GCCGUUAGGC CGAA AAUAUAGA | 2244 | |
| 265 | CUAUAUUU U CAUAUCUG | 56 | CAGAUAUG CUGAUGAG GCCGUUAGGC CGAA AAAUAUAG | 2245 | |
| 266 | UAUAUUUU C AUAUCUGU | 57 | ACAGAUAU CUGAUGAG GCCGUUAGGC CGAA AAAAUAUA | 2246 | |
| 269 | AUUUUCAU A UCUGUAUA | 58 | UAUACAGA CUGAUGAG GCCGUUAGGC CGAA AUGAAAAU | 2247 | |
| 271 | UUUCAUAU C UGUAUAUA | 59 | UAUAUACA CUGAUGAG GCCGUUAGGC CGAA AUAUGAAA | 2248 | |
| 275 | AUAUCUGU A UAUAUAUA | 60 | UAUAUAUA CUGAUGAG GCCGUUAGGC CGAA ACAGAUAU | 2249 | |
| 277 | AUCUGUAU A UAUAUAAU | 61 | AUUAUAUA CUGAUGAG GCCGUUAGGC CGAA AUACAGAU | 2250 | |
| 279 | CUGUAUAU A UAUAAUGG | 62 | CCAUUAUA CUGAUGAG GCCGUUAGGC CGAA AUAUACAG | 2251 | |
| 281 | GUAUAUAU A UAAUGGUA | 63 | UACCAUUA CUGAUGAG GCCGUUAGGC CGAA AUAUAUAC | 2252 | |
| 283 | AUAUAUAU A AUGGUAAA | 64 | UUUACCAU CUGAUGAG GCCGUUAGGC CGAA AUAUAUAU | 2253 | |
| 289 | AUAAUGGU A AAGAAAGA | 65 | UCUUUCUU CUGAUGAG GCCGUUAGGC CGAA ACCAUUAU | 2254 | |
| 303 | AGACACCU U CGUAACCC | 66 | GGGUUACG CUGAUGAG GCCGUUAGGC CGAA AGGUGUCU | 2255 | |
| 304 | GACACCUU C GUAACCCG | 67 | CGGGUUAC CUGAUGAG GCCGUUAGGC CGAA AAGGUGUC | 2256 | |
| 307 | ACCUUCGU A ACCCGCAU | 68 | AUGCGGGU CUGAUGAG GCCGUUAGGC CGAA ACGAAGGU | 2257 | |
| 326 | ACCCGCAU U UUCCAAAG | 69 | CUUUGGAA CUGAUGAG GCCGUUAGGC CGAA AUGCGGGU | 2258 | |
| 317 | CCCGCAUU U UCCAAAGA | 70 | UCUUUGGA CUGAUGAG GCCGUUAGGC CGAA AAUGCGGG | 2259 | |
| 318 | CCGCAUUU U CCAAAGAG | 71 | CUCUUUGG CUGAUGAG GCCGUUAGGC CGAA AAAUGCGG | 2260 | |
| 319 | CGCAUUUU C CAAAGAGA | 72 | UCUCUUUG CUGAUGAG GCCGUUAGGC CGAA AAAAUGCG | 2261 | |
| 333 | AGAGGAAU C ACAGGGAG | 73 | CUCCCUGU CUGAUGAG GCCGUUAGGC CGAA AUUCCUCU | 2262 | |
| 346 | GGAGAUGU A CAGCAAUG | 74 | CAUUGCUG CUGAUGAG GCCGUUAGGC CGAA ACAUCUCC | 2263 | |
| 362 | GGGGCCAU U UAAGAGUU | 75 | AACUCUUA CUGAUGAG GCCGUUAGGC CGAA AUGGCCCC | 2264 | |
| 363 | GGGCCAUU U AAGAGUUC | 76 | GAACUCUU CUGAUGAG GCCGUUAGGC CGAA AAUGGCCC | 2265 | |
| 364 | GGCCAUUU A AGAGUUCU | 77 | AGAACUCU CUGAUGAG GCCGUUAGGC CGAA AAAUGGCC | 2266 | |
| 370 | UUAAGAGU U CUGUGUUC | 78 | GAACACAG CUGAUGAG GCCGUUAGGC CGAA ACUCUUAA | 2267 | |
| 371 | UAAGAGUU C UGUGUUCA | 79 | UGAACACA CUGAUGAG GCCGUUAGGC CGAA AACUCUUA | 2268 | |
| 377 | UUCUGUGU U CAUCUUGA | 80 | UCAAGAUG CUGAUGAG GCCGUUAGGC CGAA ACACAGAA | 2269 | |
| 378 | UCUGUGUU C AUCUUGAU | 81 | AUCAAGAU CUGAUGAG GCCGUUAGGC CGAA AACACAGA | 2270 | |
| 381 | GUGUUCAU C UUGAUUCU | 82 | AGAAUCAA CUGAUGAG GCCGUUAGGC CGAA AUGAACAC | 2271 | |
| 383 | GUUCAUCU U GAUUCUUC | 83 | GAAGAAUC CUGAUGAG GCCGUUAGGC CGAA AGAUGAAC | 2272 | |
| 387 | AUCUUGAU U CUUCACCU | 84 | AGGUGAAG CUGAUGAG GCCGUUAGGC CGAA AUCAAGAU | 2273 | |
| 388 | UCUUGAUU C UUCACCUU | 85 | AAGGUGAA CUGAUGAG GCCGUUAGGC CGAA AAUCAAGA | 2274 | |
| 390 | UUGAUUCU U CACCUUCU | 86 | AGAAGGUG CUGAUGAG GCCGUUAGGC CGAA AGAAUCAA | 2275 | |
| 391 | UGAUUCUU C ACCUUCUA | 87 | UAGAAGGU CUGAUGAG GCCGUUAGGC CGAA AAGAAUCA | 2276 | |
| 396 | CUUCACCU U CUAGAAGG | 88 | CCUUCUAG CUGAUGAG GCCGUUAGGC CGAA AGGUGAAG | 2277 | |
| 397 | UUCACCUU C UAGAAGGG | 89 | CCCUUCUA CUGAUGAG GCCGUUAGGC CGAA AAGGUGAA | 2278 | |
| 399 | CACCUUCU A GAAGGGGC | 90 | GCCCCUUC CUGAUGAG GCCGUUAGGC CGAA AGAAGGUG | 2279 | |
| 415 | CCCUGAGU A AUUCACUC | 91 | GAGUGAAU CUGAUGAG GCCGUUAGGC CGAA ACUCAGGG | 2280 | |
| 418 | UGAGUAAU U CACUCAUU | 92 | AAUGAGUG CUGAUGAG GCCGUUAGGC CGAA AUUACUCA | 2281 | |
| 419 | GAGUAAUU C ACUCAUUC | 93 | GAAUGAGU CUGAUGAG GCCGUUAGGC CGAA AAUUACUC | 2282 | |
| 423 | AAUUCACU C AUUCAGCU | 94 | AGCUGAAU CUGAUGAG GCCGUUAGGC CGAA AGUGAAUU | 2283 | |
| 426 | UCACUCAU U CAGCUGAA | 95 | UUCAGCUG CUGAUGAG GCCGUUAGGC CGAA AUGAGUGA | 2284 | |
| 427 | CACUCAUU C AGCUGAAC | 96 | GUUCAGCU CUGAUGAG GCCGUUAGGC CGAA AAUGAGUG | 2285 | |
| 446 | CAAUGGCU A UGAAGGCA | 97 | UGCCUUCA CUGAUGAG GCCGUUAGGC CGAA AGCCAUUG | 2286 | |
| 456 | GAAGGCAU U GUCGUUGC | 98 | GCAACGAC CUGAUGAG GCCGUUAGGC CGAA AUGCCUUC | 2287 | |
| 459 | GGCAUUGU C GUUGCAAU | 99 | AUUGCAAC CUGAUGAG GCCGUUAGGC CGAA ACAAUGCC | 2288 | |
| 462 | AUUGUCGU U GCAAUCGA | 100 | UCGAUUGC CUGAUGAG GCCGUUAGGC CGAA ACGACAAU | 2289 | |
| 468 | GUUGCAAU C GACCCCAA | 101 | UUGGGGUC CUGAUGAG GCCGUUAGGC CGAA AUUGCAAC | 2290 | |
| 498 | GAAACACU C AUUCAACA | 102 | UGUUGAAU CUGAUGAG GCCGUUAGGC CGAA AGUGUUUC | 2291 | |
| 501 | ACACUCAU U CAACAAAU | 103 | AUUUGUUG CUGAUGAG GCCGUUAGGC CGAA AUGAGUGU | 2292 | |
| 502 | CACUCAUU C AACAAAUA | 104 | UAUUUGUU CUGAUGAG GCCGUUAGGC CGAA AAUGAGUG | 2293 | |
| 510 | CAACAAAU A AAGGACAU | 105 | AUGUCCUU CUGAUGAG GCCGUUAGGC CGAA AUUUGUUG | 2294 | |
| 533 | CCAGGCAU C UCUGUAUC | 106 | GAUACAGA CUGAUGAG GCCGUUAGGC CGAA AUGCCUGG | 2295 | |
| 535 | AGGCAUCU C UGUAUCUG | 107 | CAGAUACA CUGAUGAG GCCGUUAGGC CGAA AGAUGCCU | 2296 | |
| 539 | AUCUCUGU A UCUGUUUG | 108 | CAAACAGA CUGAUGAG GCCGUUAGGC CGAA ACAGAGAU | 2297 | |
| 541 | CUCUGUAU C UGUUUGAA | 109 | UUCAAACA CUGAUGAG GCCGUUAGGC CGAA AUACAGAG | 2298 | |
| 545 | GUAUCUGU U UGAAGCUA | 110 | UAGCUUCA CUGAUGAG GCCGUUAGGC CGAA ACAGAUAC | 2299 | |
| 546 | UAUCUGUU U GAAGCUAC | 111 | GUAGCUUC CUGAUGAG GCCGUUAGGC CGAA AACAGAUA | 2300 | |
| 553 | UUGAAGCU A CAGGAAAG | 112 | CUUUCCUG CUGAUGAG GCCGUUAGGC CGAA AGCUUCAA | 2301 | |
| 566 | AAAGCGAU U UUAUUUCA | 113 | UGAAAUAA CUGAUGAG GCCGUUAGGC CGAA AUCGCUUU | 2302 | |
| 567 | AAGCGAUU U UAUUUCAA | 114 | UUGAAAUA CUGAUGAG GCCGUUAGGC CGAA AAUCGCUU | 2303 | |
| 568 | AGCGAUUU U AUUUCAAA | 115 | UUUGAAAU CUGAUGAG GCCGUUAGGC CGAA AAAUCGCU | 2304 | |
| 569 | GCGAUUUU A UUUCAAAA | 116 | UUUUGAAA CUGAUGAG GCCGUUAGGC CGAA AAAAUCGC | 2305 | |
| 571 | GAUUUUAU U UCAAAAAU | 117 | AUUUUUGA CUGAUGAG GCCGUUAGGC CGAA AUAAAAUC | 2306 | |
| 572 | AUUUUAUU U CAAAAAUG | 118 | CAUUUUUG CUGAUGAG GCCGUUAGGC CGAA AAUAAAAU | 2307 | |
| 573 | UUUUAUUU C AAAAAUGU | 119 | ACAUUUUU CUGAUGAG GCCGUUAGGC CGAA AAAUAAAA | 2308 | |
| 582 | AAAAAUGU U GCCAUUUU | 120 | AAAAUGGC CUGAUGAG GCCGUUAGGC CGAA ACAUUUUU | 2309 | |
| 588 | GUUGCCAU U UUGAUUCC | 121 | GGAAUCAA CUGAUGAG GCCGUUAGGC CGAA AUGGCAAC | 2310 | |
| 589 | UUGCCAUU U UGAUUCCU | 122 | AGGAAUCA CUGAUGAG GCCGUUAGGC CGAA AAUGGCAA | 2311 | |
| 590 | UGCCAUUU U GAUUCCUG | 123 | CAGGAAUC CUGAUGAG GCCGUUAGGC CGAA AAAUGGCA | 2312 | |
| 594 | AUUUUGAU U CCUGAAAC | 124 | GUUUCAGG CUGAUGAG GCCGUUAGGC CGAA AUCAAAAU | 2313 | |
| 595 | UUUUGAUU C CUGAAACA | 125 | UGUUUCAG CUGAUGAG GCCGUUAGGC CGAA AAUCAAAA | 2314 | |
| 623 | GGCUGACU A UGUGAGAC | 126 | GUCUCACA CUGAUGAG GCCGUUAGGC CGAA AGUCAGCC | 2315 | |
| 639 | CCAAAACU U GAGACCUA | 127 | UAGGUCUC CUGAUGAG GCCGUUAGGC CGAA AGUUUUGG | 2316 | |
| 647 | UGAGACCU A CAAAAAUG | 128 | CAUUUUUG CUGAUGAG GCCGUUAGGC CGAA AGGUCUCA | 2317 | |
| 663 | GCUGAUGU U CUGGUUGC | 129 | GCAACCAG CUGAUGAG GCCGUUAGGC CGAA ACAUCAGC | 2318 | |
| 664 | CUGAUGUU C UGGUUGCU | 130 | AGCAACCA CUGAUGAG GCCGUUAGGC CGAA AACAUCAG | 2319 | |
| 669 | GUUCUGGU U GCUGAGUC | 131 | GACUCAGC CUGAUGAG GCCGUUAGGC CGAA ACCAGAAC | 2320 | |
| 677 | UGCUGAGU C UACUCCUC | 132 | GAGGAGUA CUGAUGAG GCCGUUAGGC CGAA ACUCAGCA | 2321 | |
| 679 | CUGAGUCU A CUCCUCCA | 133 | UGGAGGAG CUGAUGAG GCCGUUAGGC CGAA AGACUCAG | 2322 | |
| 682 | AGUCUACU C CUCCAGGU | 134 | ACCUGGAG CUGAUGAG GCCGUUAGGC CGAA AGUAGACU | 2323 | |
| 685 | CUACUCCU C CAGGUAAU | 135 | AUUACCUG CUGAUGAG GCCGUUAGGC CGAA AGGAGUAG | 2324 | |
| 691 | CUCCAGGU A AUGAUGAA | 136 | UUCAUCAU CUGAUGAG GCCGUUAGGC CGAA ACCUGGAG | 2325 | |
| 704 | UGAACCCU A CACUGAGC | 137 | GCUCAGUG CUGAUGAG GCCGUUAGGC CGAA AGGGUUCA | 2326 | |
| 747 | GAAAGGAU C CACCUCAC | 138 | GUGAGGUG CUGAUGAG GCCGUUAGGC CGAA AUCCUUUC | 2327 | |
| 753 | AUCCACCU C ACUCCUGA | 139 | UCAGGAGU CUGAUGAG GCCGUUAGGC CGAA AGGUGGAU | 2328 | |
| 757 | ACCUCACU C CUGAUUUC | 140 | GAAAUCAG CUGAUGAG GCCGUUAGGC CGAA AGUGAGGU | 2329 | |
| 763 | CUCCUGAU U UCAUUGCA | 141 | UGCAAUGA CUGAUGAG GCCGUUAGGC CGAA AUCAGGAG | 2330 | |
| 764 | UCCUGAUU U CAUUGCAG | 142 | CUGCAAUG CUGAUGAG GCCGUUAGGC CGAA AAUCAGGA | 2331 | |
| 765 | CCUGAUUU C AUUGCAGG | 143 | CCUGCAAU CUGAUGAG GCCGUUAGGC CGAA AAAUCAGG | 2332 | |
| 768 | GAUUUCAU U GCAGGAAA | 144 | UUUCCUGC CUGAUGAG GCCGUUAGGC CGAA AUGAAAUC | 2333 | |
| 782 | AAAAAAGU U AGCUGAAU | 145 | AUUCAGCU CUGAUGAG GCCGUUAGGC CGAA ACUUUUUU | 2334 | |
| 783 | AAAAAGUU A GCUGAAUA | 146 | UAUUCAGC CUGAUGAG GCCGUUAGGC CGAA AACUUUUU | 2335 | |
| 791 | AGCUGAAU A UGGACCAC | 147 | GUGGUCCA CUGAUGAG GCCGUUAGGC CGAA AUUCAGCU | 2336 | |
| 805 | CACAAGGU A AGGCAUUU | 148 | AAAUGCCU CUGAUGAG GCCGUUAGGC CGAA ACCUUGUG | 2337 | |
| 812 | UAAGGCAU U UGUCCAUG | 149 | CAUGGACA CUGAUGAG GCCGUUAGGC CGAA AUGCCUUA | 2338 | |
| 813 | AAGGCAUU U GUCCAUGA | 150 | UCAUGGAC CUGAUGAG GCCGUUAGGC CGAA AAUGCCUU | 2339 | |
| 816 | GCAUUUGU C CAUGAGUG | 151 | CACUCAUG CUGAUGAG GCCGUUAGGC CGAA ACAAAUGC | 2340 | |
| 829 | AGUGGGCU C AUCUACGA | 152 | UCGUAGAU CUGAUGAG GCCGUUAGGC CGAA AGCCCACU | 2341 | |
| 832 | GGGCUCAU C UACGAUGG | 153 | CCAUCGUA CUGAUGAG GCCGUUAGGC CGAA AUGAGCCC | 2342 | |
| 834 | GCUCAUCU A CGAUGGGG | 154 | CCCCAUCG CUGAUGAG GCCGUUAGGC CGAA AGAUGAGC | 2343 | |
| 846 | UGGGGAGU A UUUGACGA | 155 | UCGUCAAA CUGAUGAG GCCGUUAGGC CGAA ACUCCCCA | 2344 | |
| 848 | GGGAGUAU U UGACGAGU | 156 | ACUCGUCA CUGAUGAG GCCGUUAGGC CGAA AUACUCCC | 2345 | |
| 849 | GGAGUAUU U GACGAGUA | 157 | UACUCGUC CUGAUGAG GCCGUUAGGC CGAA AAUACUCC | 2346 | |
| 857 | UGACGAGU A CAAUAAUG | 158 | CAUUAUUG CUGAUGAG GCCGUUAGGC CGAA ACUCGUCA | 2347 | |
| 862 | AGUACAAU A AUGAUGAG | 159 | CUCAUCAU CUGAUGAG GCCGUUAGGC CGAA AUUGUACU | 2348 | |
| 875 | UGAGAAAU U CUACUUAU | 160 | AUAAGUAG CUGAUGAG GCCGUUAGGC CGAA AUUUCUCA | 2349 | |
| 876 | GAGAAAUU C UACUUAUC | 161 | GAUAAGUA CUGAUGAG GCCGUUAGGC CGAA AAUUUCUC | 2350 | |
| 878 | GAAAUUCU A CUUAUCCA | 162 | UGGAUAAG CUGAUGAG GCCGUUAGGC CGAA AGAAUUUC | 2351 | |
| 881 | AUUCUACU U AUCCAAUG | 163 | CAUUGGAU CUGAUGAG GCCGUUAGGC CGAA AGUAGAAU | 2352 | |
| 882 | UUCUACUU A UCCAAUGG | 164 | CCAUUGGA CUGAUGAG GCCGUUAGGC CGAA AAGUAGAA | 2353 | |
| 884 | CUACUUAU C CAAUGGAA | 165 | UUCCAUUG CUGAUGAG GCCGUUAGGC CGAA AUAAGUAG | 2354 | |
| 897 | GGAAGAAU A CAAGCAGU | 166 | ACUGCUUG CUGAUGAG GCCGUUAGGC CGAA AUUCUUCC | 2355 | |
| 906 | CAAGCAGU A AGAUGUUC | 167 | GAACAUCU CUGAUGAG GCCGUUAGGC CGAA ACUGCUUG | 2356 | |
| 913 | UAAGAUGU U CAGCAGGU | 168 | ACCUGCUG CUGAUGAG GCCGUUAGGC CGAA ACAUCUUA | 2357 | |
| 914 | AAGAUGUU C AGCAGGUA | 169 | UACCUGCU CUGAUGAG GCCGUUAGGC CGAA AACAUCUU | 2358 | |
| 922 | CAGCAGGU A UUACUGGU | 170 | ACCAGUAA CUGAUGAG GCCGUUAGGC CGAA ACCUGCUG | 2359 | |
| 924 | GCAGGUAU U ACUGGUAC | 171 | GUACCAGU CUGAUGAG GCCGUUAGGC CGAA AUACCUGC | 2360 | |
| 925 | CAGGUAUU A CUGGUACA | 172 | UGUACCAG CUGAUGAG GCCGUUAGGC CGAA AAUACCUG | 2361 | |
| 931 | UUACUGGU A CAAAUGUA | 173 | UACAUUUG CUGAUGAG GCCGUUAGGC CGAA ACCAGUAA | 2362 | |
| 939 | ACAAAUGU A GUAAAGAA | 174 | UUCUUUAC CUGAUGAG GCCGUUAGGC CGAA ACAUUUGU | 2363 | |
| 942 | AAUGUAGU A AAGAAGUG | 175 | CACUUCUU CUGAUGAG GCCGUUAGGC CGAA ACUACAUU | 2364 | |
| 952 | AGAAGUGU C AGGGAGGC | 176 | GCCUCCCU CUGAUGAG GCCGUUAGGC CGAA ACACUUCU | 2365 | |
| 967 | GCAGCUGU U ACACCAAA | 177 | UUUGGUGU CUGAUGAG GCCGUUAGGC CGAA ACAGCUGC | 2366 | |
| 968 | CAGCUGUU A CACCAAAA | 178 | UUUUGGUG CUGAUGAG GCCGUUAGGC CGAA AACAGCUG | 2367 | |
| 986 | AUGCACAU U CAAUAAAG | 179 | CUUUAUUG CUGAUGAG GCCGUUAGGC CGAA AUGUGCAU | 2368 | |
| 987 | UGCACAUU C AAUAAAGU | 180 | ACUUUAUU CUGAUGAG GCCGUUAGGC CGAA AAUGUGCA | 2369 | |
| 991 | CAUUCAAU A AAGUUACA | 181 | UGUAACUU CUGAUGAG GCCGUUAGGC CGAA AUUGAAUG | 2370 | |
| 996 | AAUAAAGU U ACAGGACU | 182 | AGUCCUGU CUGAUGAG GCCGUUAGGC CGAA ACUUUAUU | 2371 | |
| 997 | AUAAAGUU A CAGGACUC | 183 | GAGUCCUG CUGAUGAG GCCGUUAGGC CGAA AACUUUAU | 2372 | |
| 1005 | ACAGGACU C UAUGAAAA | 184 | UUUUCAUA CUGAUGAG GCCGUUAGGC CGAA AGUCCUGU | 2373 | |
| 1007 | AGGACUCU A UGAAAAAG | 185 | CUUUUUCA CUGAUGAG GCCGUUAGGC CGAA AGAGUCCU | 2374 | |
| 1025 | AUGUGAGU U UGUUCUCC | 186 | GGAGAACA CUGAUGAG GCCGUUAGGC CGAA ACUCACAU | 2375 | |
| 1026 | UGUGAGUU U GUUCUCCA | 187 | UGGAGAAC CUGAUGAG GCCGUUAGGC CGAA AACUCACA | 2376 | |
| 1029 | GAGUUUGU U CUCCAAUC | 188 | GAUUGGAG CUGAUGAG GCCGUUAGGC CGAA ACAAACUC | 2377 | |
| 1030 | AGUUUGUU C UCCAAUCC | 189 | GGAUUGGA CUGAUGAG GCCGUUAGGC CGAA AACAAACU | 2378 | |
| 1032 | UUUGUUCU C CAAUCCCG | 190 | CGGGAUUG CUGAUGAG GCCGUUAGGC CGAA AGAACAAA | 2379 | |
| 1037 | UCUCCAAU C CCGCCAGA | 191 | UCUGGCGG CUGAUGAG GCCGUUAGGC CGAA AUUGGAGA | 2380 | |
| 1057 | AGAAGGCU U CUAUAAUG | 192 | CAUUAUAG CUGAUGAG GCCGUUAGGC CGAA AGCCUUCU | 2381 | |
| 1058 | GAAGGCUU C UAUAAUGU | 193 | ACAUUAUA CUGAUGAG GCCGUUAGGC CGAA AAGCCUUC | 2382 | |
| 1060 | AGGCUUCU A UAAUGUUU | 194 | AAACAUUA CUGAUGAG GCCGUUAGGC CGAA AGAAGCCU | 2383 | |
| 1062 | GCUUCUAU A AUGUUUGC | 195 | GCAAACAU CUGAUGAG GCCGUUAGGC CGAA AUAGAAGC | 2384 | |
| 1067 | UAUAAUGU U UGCACAAC | 196 | GUUGUGCA CUGAUGAG GCCGUUAGGC CGAA ACAUUAUA | 2385 | |
| 1068 | AUAAUGUU U GCACAACA | 197 | UGUUGUGC CUGAUGAG GCCGUUAGGC CGAA AACAUUAU | 2386 | |
| 1080 | CAACAUGU U GAUUCUAU | 198 | AUAGAAUC CUGAUGAG GCCGUUAGGC CGAA ACAUGUUG | 2387 | |
| 1084 | AUGUUGAU U CUAUAGUU | 199 | AACUAUAG CUGAUGAG GCCGUUAGGC CGAA AUCAACAU | 2388 | |
| 1085 | UGUUGAUU C UAUAGUUG | 200 | CAACUAUA CUGAUGAG GCCGUUAGGC CGAA AAUCAACA | 2389 | |
| 1087 | UUGAUUCU A UAGUUGAA | 201 | UUCAACUA CUGAUGAG GCCGUUAGGC CGAA AGAAUCAA | 2390 | |
| 1089 | GAUUCUAU A GUUGAAUU | 202 | AAUUCAAC CUGAUGAG GCCGUUAGGC CGAA AUAGAAUC | 2391 | |
| 1092 | UCUAUAGU U GAAUUCUG | 203 | CAGAAUUC CUGAUGAG GCCGUUAGGC CGAA ACUAUAGA | 2392 | |
| 1097 | AGUUGAAU U CUGUACAG | 204 | CUGUACAG CUGAUGAG GCCGUUAGGC CGAA AUUCAACU | 2393 | |
| 1098 | GUUGAAUU C UGUACAGA | 205 | UCUGUACA CUGAUGAG GCCGUUAGGC CGAA AAUUCAAC | 2394 | |
| 1102 | AAUUCUGU A CAGAACAA | 206 | UUGUUCUG CUGAUGAG GCCGUUAGGC CGAA ACAGAAUU | 2395 | |
| 1129 | AAGAAGCU C CAAACAAG | 207 | CUUGUUUG CUGAUGAG GCCGUUAGGC CGAA AGCUUCUU | 2396 | |
| 1144 | AGCAAAAU C AAAAAUGC | 208 | GCAUUUUU CUGAUGAG GCCGUUAGGC CGAA AUUUUGCU | 2397 | |
| 1156 | AAUGCAAU C UCCGAAGC | 209 | GCUUCGGA CUGAUGAG GCCGUUAGGC CGAA AUUGCAUU | 2398 | |
| 1158 | UGCAAUCU C CGAAGCAC | 210 | GUGCUUCG CUGAUGAG GCCGUUAGGC CGAA AGAUUGCA | 2399 | |
| 1179 | GAAGUGAU C CGUGAUUC | 211 | GAAUCACG CUGAUGAG GCCGUUAGGC CGAA AUCACUUC | 2400 | |
| 1186 | UCCGUGAU U CUGAGGAC | 212 | GUCCUCAG CUGAUGAG GCCGUUAGGC CGAA AUCACGGA | 2401 | |
| 1187 | CCGUGAUU C UGAGGACU | 213 | AGUCCUCA CUGAUGAG GCCGUUAGGC CGAA AAUCACGG | 2402 | |
| 1196 | UGAGGACU U UAAGAAAA | 214 | UUUUCUUA CUGAUGAG GCCGUUAGGC CGAA AGUCCUCA | 2403 | |
| 1197 | GAGGACUU U AAGAAAAC | 215 | GUUUUCUU CUGAUGAG GCCGUUAGGC CGAA AAGUCCUC | 2404 | |
| 1198 | AGGACUUU A AGAAAACC | 216 | GGUUUUCU CUGAUGAG GCCGUUAGGC CGAA AAAGUCCU | 2405 | |
| 1210 | AAACCACU C CUAUGACA | 217 | UGUCAUAG CUGAUGAG GCCGUUAGGC CGAA AGUGGUUU | 2406 | |
| 1213 | CCACUCCU A UGACAACA | 218 | UGUUGUCA CUGAUGAG GCCGUUAGGC CGAA AGGAGUGG | 2407 | |
| 1234 | CACCAAAU C CCACCUUC | 219 | GAAGGUGG CUGAUGAG GCCGUUAGGC CGAA AUUUGGUG | 2408 | |
| 1241 | UCCCACCU U CUCAUUGC | 220 | GCAAUGAG CUGAUGAG GCCGUUAGGC CGAA AGGUGGGA | 2409 | |
| 1242 | CCCACCUU C UCAUUGCU | 221 | AGCAAUGA CUGAUGAG GCCGUUAGGC CGAA AAGGUGGG | 2410 | |
| 1244 | CACCUUCU C AUUGCUGC | 222 | GCAGCAAU CUGAUGAG GCCGUUAGGC CGAA AGAAGGUG | 2411 | |
| 1247 | CUUCUCAU U GCUGCAGA | 223 | UCUGCAGC CUGAUGAG GCCGUUAGGC CGAA AUGAGAAG | 2412 | |
| 1257 | CUGCAGAU U GGACAAAG | 224 | CUUUGUCC CUGAUGAG GCCGUUAGGC CGAA AUCUGCAG | 2413 | |
| 1269 | CAAAGAAU U GUGUGUUU | 225 | AAACACAC CUGAUGAG GCCGUUAGGC CGAA AUUCUUUG | 2414 | |
| 1276 | UUGUGUGU U UAGUCCUU | 226 | AAGGACUA CUGAUGAG GCCGUUAGGC CGAA ACACACAA | 2415 | |
| 1277 | UGUGUGUU U AGUCCUUG | 227 | CAAGGACU CUGAUGAG GCCGUUAGGC CGAA AACACACA | 2416 | |
| 1278 | GUGUGUUU A GUCCUUGA | 228 | UCAAGGAC CUGAUGAG GCCGUUAGGC CGAA AAACACAC | 2417 | |
| 1281 | UGUUUAGU C CUUGACAA | 229 | UUGUCAAG CUGAUGAG GCCGUUAGGC CGAA ACUAAACA | 2418 | |
| 1284 | UUAGUCCU U GACAAAUC | 230 | GAUUUGUC CUGAUGAG GCCGUUAGGC CGAA AGGACUAA | 2419 | |
| 1292 | UGACAAAU C UGGAAGCA | 231 | UGCUUCCA CUGAUGAG GCCGUUAGGC CGAA AUUUGUCA | 2420 | |
| 1312 | CGACUGGU A ACCGCCUC | 232 | GAGGCGGU CUGAUGAG GCCGUUAGGC CGAA ACCAGUCG | 2421 | |
| 1320 | AACCGCCU C AAUCGACU | 233 | AGUCGAUU CUGAUGAG GCCGUUAGGC CGAA AGGCGGUU | 2422 | |
| 1324 | GCCUCAAU C GACUGAAU | 234 | AUUCAGUC CUGAUGAG GCCGUUAGGC CGAA AUUGAGGC | 2423 | |
| 1333 | GACUGAAU C AAGCAGGC | 235 | GCCUGCUU CUGAUGAG GCCGUUAGGC CGAA AUUCAGUC | 2424 | |
| 1347 | GGCCAGCU U UUCCUGCU | 236 | AGCAGGAA CUGAUGAG GCCGUUAGGC CGAA AGCUGGCC | 2425 | |
| 1348 | GCCAGCUU U UCCUGCUG | 237 | CAGCAGGA CUGAUGAG GCCGUUAGGC CGAA AAGCUGGC | 2426 | |
| 1349 | CCAGCUUU U CCUGCUGC | 238 | GCAGCAGG CUGAUGAG GCCGUUAGGC CGAA AAAGCUGG | 2427 | |
| 1350 | CAGCUUUU C CUGCUGCA | 239 | UGCAGCAG CUGAUGAG GCCGUUAGGC CGAA AAAAGCUG | 2428 | |
| 1365 | CAGACAGU U GAGCUGGG | 240 | CCCAGCUC CUGAUGAG GCCGUUAGGC CGAA ACUGUCUG | 2429 | |
| 1376 | GCUGGGGU C CUGGGUUG | 241 | CAACCCAG CUGAUGAG GCCGUUAGGC CGAA ACCCCAGC | 2430 | |
| 1383 | UCCUGGGU U GGGAUGGU | 242 | ACCAUCCC CUGAUGAG GCCGUUAGGC CGAA ACCCAGGA | 2431 | |
| 1397 | GGUGACAU U UGACAGUG | 243 | CACUGUCA CUGAUGAG GCCGUUAGGC CGAA AUGUCACC | 2432 | |
| 1398 | GUGACAUU U GACAGUGC | 244 | GCACUGUC CUGAUGAG GCCGUUAGGC CGAA AAUGUCAC | 2433 | |
| 1416 | GCCCAUGU A CAAAGUGA | 245 | UCACUUUG CUGAUGAG GCCGUUAGGC CGAA ACAUGGGC | 2434 | |
| 1428 | AGUGAACU C AUACAGAU | 246 | AUCUGUAU CUGAUGAG GCCGUUAGGC CGAA AGUUCACU | 2435 | |
| 1431 | GAACUCAU A CAGAUAAA | 247 | UUUAUCUG CUGAUGAG GCCGUUAGGC CGAA AUGAGUUC | 2436 | |
| 1437 | AUACAGAU A AACAGUGG | 248 | CCACUGUU CUGAUGAG GCCGUUAGGC CGAA AUCUGUAU | 2437 | |
| 1464 | GACACACU C GCCAAAAG | 249 | CUUUUGGC CUGAUGAG GCCGUUAGGC CGAA AGUGUGUC | 2438 | |
| 1475 | CAAAAGAU U ACCUGCAG | 250 | CUGCAGGU CUGAUGAG GCCGUUAGGC CGAA AUCUUUUG | 2439 | |
| 1476 | AAAAGAUU A CCUGCAGC | 251 | GCUGCAGG CUGAUGAG GCCGUUAGGC CGAA AAUCUUUU | 2440 | |
| 1489 | CAGCAGCU U CAGGAGGG | 252 | CCCUCCUG CUGAUGAG GCCGUUAGGC CGAA AGCUGCUG | 2441 | |
| 1490 | AGCAGCUU C AGGAGGGA | 253 | UCCCUCCU CUGAUGAG GCCGUUAGGC CGAA AAGCUGCU | 2442 | |
| 1502 | AGGGACGU C CAUCUGCA | 254 | UGCAGAUG CUGAUGAG GCCGUUAGGC CGAA ACGUCCCU | 2443 | |
| 1506 | ACGUCCAU C UGCAGCGG | 255 | CCGCUGCA CUGAUGAG GCCGUUAGGC CGAA AUGGACGU | 2444 | |
| 1518 | AGCGGGCU U CGAUCGGC | 256 | GCCGAUCG CUGAUGAG GCCGUUAGGC CGAA AGCCCGCU | 2445 | |
| 1519 | GCGGGCUU C GAUCGGCA | 257 | UGCCGAUC CUGAUGAG GCCGUUAGGC CGAA AAGCCCGC | 2446 | |
| 1523 | GCUUCGAU C GGCAUUUA | 258 | UAAAUGCC CUGAUGAG GCCGUUAGGC CGAA AUCGAAGC | 2447 | |
| 1529 | AUCGGCAU U UACUGUGA | 259 | UCACAGUA CUGAUGAG GCCGUUAGGC CGAA AUGCCGAU | 2448 | |
| 1530 | UCGGCAUU U ACUGUGAU | 260 | AUCACAGU CUGAUGAG GCCGUUAGGC CGAA AAUGCCGA | 2449 | |
| 1531 | CGGCAUUU A CUGUGAUU | 261 | AAUCACAG CUGAUGAG GCCGUUAGGC CGAA AAAUGCCG | 2450 | |
| 1539 | ACUGUGAU U AGGAAGAA | 262 | UUCUUCCU CUGAUGAG GCCGUUAGGC CGAA AUCACAGU | 2451 | |
| 1540 | CUGUGAUU A GGAAGAAA | 263 | UUUCUUCC CUGAUGAG GCCGUUAGGC CGAA AAUCACAG | 2452 | |
| 1550 | GAAGAAAU A UCCAACUG | 264 | CAGUUGGA CUGAUGAG GCCGUUAGGC CGAA AUUUCUUC | 2453 | |
| 1552 | AGAAAUAU C CAACUGAU | 265 | AUCAGUUG CUGAUGAG GCCGUUAGGC CGAA AUAUUUCU | 2454 | |
| 1565 | UGAUGGAU C UGAAAUUG | 266 | CAAUUUCA CUGAUGAG GCCGUUAGGC CGAA AUCCAUCA | 2455 | |
| 1572 | UCUGAAAU U GUGCUGCU | 267 | AGCAGCAC CUGAUGAG GCCGUUAGGC CGAA AUUUCAGA | 2456 | |
| 1603 | ACAACACU A UAAGUGGG | 268 | CCCACUUA CUGAUGAG GCCGUUAGGC CGAA AGUGUUGU | 2457 | |
| 1605 | AACACUAU A AGUGGGUG | 269 | CACCCACU CUGAUGAG GCCGUUAGGC CGAA AUAGUGUU | 2458 | |
| 1616 | UGGGUGCU U UAACGAGG | 270 | CCUCGUUA CUGAUGAG GCCGUUAGGC CGAA AGCACCCA | 2459 | |
| 1617 | GGGUGCUU U AACGAGGU | 271 | ACCUCGUU CUGAUGAG GCCGUUAGGC CGAA AAGCACCC | 2460 | |
| 1618 | GGUGCUUU A ACGAGGUC | 272 | GACCUCGU CUGAUGAG GCCGUUAGGC CGAA AAAGCACC | 2461 | |
| 1626 | AACGAGGU C AAACAAAG | 273 | CUUUGUUU CUGAUGAG GCCGUUAGGC CGAA ACCUCGUU | 2462 | |
| 1644 | GGUGCCAU C AUCCACAC | 274 | GUGUGGAU CUGAUGAG GCCGUUAGGC CGAA AUGGCACC | 2463 | |
| 1647 | GCCAUCAU C CACACAGU | 275 | ACUGUGUG CUGAUGAG GCCGUUAGGC CGAA AUGAUGGC | 2464 | |
| 1656 | CACACAGU C GCUUUGGG | 276 | CCCAAAGC CUGAUGAG GCCGUUAGGC CGAA ACUGUGUG | 2465 | 2465 |
| 1660 | CAGUCGCU U UGGGGCCC | 277 | GGGCCCCA CUGAUGAG GCCGUUAGGC CGAA AGCGACUG | 2466 | |
| 1661 | AGUCGCUU U GGGGCCCU | 278 | AGGGCCCC CUGAUGAG GCCGUUAGGC CGAA AAGCGACU | 2467 | |
| 1670 | GGGGCCCU C UGCAGCUC | 279 | GAGCUGCA CUGAUGAG GCCGUUAGGC CGAA AGGGCCCC | 2468 | |
| 1678 | CUGCAGCU C AAGAACUA | 280 | UAGUUCUU CUGAUGAG GCCGUUAGGC CGAA AGCUGCAG | 2469 | |
| 1686 | CAAGAACU A GAGGAGCU | 281 | AGCUCCUC CUGAUGAG GCCGUUAGGC CGAA AGUUCUUG | 2470 | |
| 1697 | GGAGCUGU C CAAAAUGA | 282 | UCAUUUUG CUGAUGAG GCCGUUAGGC CGAA ACAGCUCC | 2471 | |
| 1714 | CAGGAGGU U UACAGACA | 283 | UGUCUGUA CUGAUGAG GCCGUUAGGC CGAA ACCUCCUG | 2472 | |
| 1715 | AGGAGGUU U ACAGACAU | 284 | AUGUCUGU CUGAUGAG GCCGUUAGGC CGAA AACCUCCU | 2473 | |
| 1716 | GGAGGUUU A CAGACAUA | 285 | UAUGUCUG CUGAUGAG GCCGUUAGGC CGAA AAACCUCC | 2474 | |
| 1724 | ACAGACAU A UGCUUCAG | 286 | CUGAAGCA CUGAUGAG GCCGUUAGGC CGAA AUGUCUGU | 2475 | |
| 1729 | CAUAUGCU U CAGAUCAA | 287 | UUGAUCUG CUGAUGAG GCCGUUAGGC CGAA AGCAUAUG | 2476 | |
| 1730 | AUAUGCUU C AGAUCAAG | 288 | CUUGAUCU CUGAUGAG GCCGUUAGGC CGAA AAGCAUAU | 2477 | |
| 1735 | CUUCAGAU C AAGUUCAG | 289 | CUGAACUU CUGAUGAG GCCGUUAGGC CGAA AUCUGAAG | 2478 | |
| 1740 | GAUCAAGU U CAGAACAA | 290 | UUGUUCUG CUGAUGAG GCCGUUAGGC CGAA ACUUGAUC | 2479 | |
| 1741 | AUCAAGUU C AGAACAAU | 291 | AUUGUUCU CUGAUGAG GCCGUUAGGC CGAA AACUUGAU | 2480 | |
| 1755 | AAUGGCCU C AUUGAUGC | 292 | GCAUCAAU CUGAUGAG GCCGUUAGGC CGAA AGGCCAUU | 2481 | |
| 1758 | GGCCUCAU U GAUGCUUU | 293 | AAAGCAUC CUGAUGAG GCCGUUAGGC CGAA AUGAGGCC | 2482 | |
| 1765 | UUGAUGCU U UUGGGGCC | 294 | GGCCCCAA CUGAUGAG GCCGUUAGGC CGAA AGCAUCAA | 2483 | |
| 1766 | UGAUGCUU U UGGGGCCC | 295 | GGGCCCCA CUGAUGAG GCCGUUAGGC CGAA AAGCAUCA | 2484 | |
| 1767 | GAUGCUUU U GGGGCCCU | 296 | AGGGCCCC CUGAUGAG GCCGUUAGGC CGAA AAAGCAUC | 2485 | |
| 1776 | GGGGCCCU U UCAUCAGG | 297 | CCUGAUGA CUGAUGAG GCCGUUAGGC CGAA AGGGCCCC | 2486 | |
| 1777 | GGGCCCUU U CAUCAGGA | 298 | UCCUGAUG CUGAUGAG GCCGUUAGGC CGAA AAGGGCCC | 2487 | |
| 1778 | GGCCCUUU C AUCAGGAA | 299 | UUCCUGAU CUGAUGAG GCCGUUAGGC CGAA AAAGGGCC | 2488 | |
| 1781 | CCUUUCAU C AGGAAAUG | 300 | CAUUUCCU CUGAUGAG GCCGUUAGGC CGAA AUGAAAGG | 2489 | |
| 1797 | GGAGCUGU C UCUCAGCG | 301 | CGCUGAGA CUGAUGAG GCCGUUAGGC CGAA ACAGCUCC | 2490 | |
| 1799 | AGCUGUCU C UCAGCGCU | 302 | AGCGCUGA CUGAUGAG GCCGUUAGGC CGAA AGACAGCU | 2491 | |
| 1801 | CUGUCUCU C AGCGCUCC | 303 | GGAGCGCU CUGAUGAG GCCGUUAGGC CGAA AGAGACAG | 2492 | |
| 1808 | UCAGCGCU C CAUCCAGC | 304 | GCUGGAUG CUGAUGAG GCCGUUAGGC CGAA AGCGCUGA | 2493 | |
| 1812 | CGCUCCAU C CAGCUUGA | 305 | UCAAGCUG CUGAUGAG GCCGUUAGGC CGAA AUGGAGCG | 2494 | |
| 1818 | AUCCAGCU U GAGAGUAA | 306 | UUACUCUC CUGAUGAG GCCGUUAGGC CGAA AGCUGGAU | 2495 | |
| 1825 | UUGAGAGU A AGGGAUUA | 307 | UAAUCCCU CUGAUGAG GCCGUUAGGC CGAA ACUCUCAA | 2496 | |
| 1832 | UAAGGGAU U AACCCUCC | 308 | GGAGGGUU CUGAUGAG GCCGUUAGGC CGAA AUCCCUUA | 2497 | |
| 1833 | AAGGGAUU A ACCCUCCA | 309 | UGGAGGGU CUGAUGAG GCCGUUAGGC CGAA AAUCCCUU | 2498 | |
| 1839 | UUAACCCU C CAGAACAG | 310 | CUGUUCUG CUGAUGAG GCCGUUAGGC CGAA AGGGUUAA | 2499 | |
| 1872 | ACAGUGAU C GUGGACAG | 311 | CUGUCCAC CUGAUGAG GCCGUUAGGC CGAA AUCACUGU | 2500 | |
| 1900 | AGGACACU U UGUUUCUU | 312 | AAGAAACA CUGAUGAG GCCGUUAGGC CGAA AGUGUCCU | 2501 | |
| 1901 | GGACACUU U GUUUCUUA | 313 | UAAGAAAC CUGAUGAG GCCGUUAGGC CGAA AACUGUCC | 2502 | |
| 1904 | CACUUUGU U UCUUAUCA | 314 | UGAUAAGA CUGAUGAG GCCGUUAGGC CGAA ACAAAGUG | 2503 | |
| 1905 | ACUUUGUU U CUUAUCAC | 315 | GUGAUAAG CUGAUGAG GCCGUUAGGC CGAA AACAAAGU | 2504 | |
| 1906 | CUUUGUUU C UUAUCACC | 316 | GGUGAUAA CUGAUGAG GCCGUUAGGC CGAA AAACAAAG | 2505 | |
| 1908 | UUGUUUCU U AUCACCUG | 317 | CAGGUGAU CUGAUGAG GCCGUUAGGC CGAA AGAAACAA | 2506 | |
| 1909 | UGUUUCUU A UCACCUGG | 318 | CCAGGUGA CUGAUGAG GCCGUUAGGC CGAA AAGAAACA | 2507 | |
| 1911 | UUUCUUAU C ACCUGGAC | 319 | GUCCAGGU CUGAUGAG GCCGUUAGGC CGAA AUAAGAAA | 2508 | |
| 1930 | CGCAGCCU C CCCAAAUC | 320 | GAUUUGGG CUGAUGAG GCCGUUAGGC CGAA AGGCUGCG | 2509 | |
| 1938 | CCCCAAAU C CUUCUCUG | 321 | CAGAGAAG CUGAUGAG GCCGUUAGGC CGAA AUUUGGGG | 2510 | |
| 1941 | CAAAUCCU U CUCUGGGA | 322 | UCCCAGAG CUGAUGAG GCCGUUAGGC CGAA AGGAUUUG | 2511 | |
| 1942 | AAAUCCUU C UCUGGGAU | 323 | AUCCCAGA CUGAUGAG GCCGUUAGGC CGAA AAGGAUUU | 2512 | |
| 1944 | AUCCUUCU C UGGGAUCC | 324 | GGAUCCCA CUGAUGAG GCCGUUAGGC CGAA AGAAGGAU | 2513 | |
| 1951 | UCUGGGAU C CCAGUGGA | 325 | UCCACUGG CUGAUGAG GCCGUUAGGC CGAA AUCCCAGA | 2514 | |
| 1976 | AGGUGGCU U UGUAGUGG | 326 | CCACUACA CUGAUGAG GCCGUUAGGC CGAA AGCCACCU | 2515 | |
| 1977 | GGUGGCUU U GUAGUGGA | 327 | UCCACUAC CUGAUGAG GCCGUUAGGC CGAA AAGCCACC | 2516 | |
| 1980 | GGCUUUGU A GUGGACAA | 328 | UUGUCCAC CUGAUGAG GCCGUUAGGC CGAA ACAAAGCC | 2517 | |
| 2006 | AAUGGCCU A CCUCCAAA | 329 | UUUGGAGG CUGAUGAG GCCGUUAGGC CGAA AGGCCAUU | 2518 | |
| 2010 | GCCUACCU C CAAAUCCC | 330 | GGGAUUUG CUGAUGAG GCCGUUAGGC CGAA AGGUAGGC | 2519 | |
| 2016 | CUCCAAAU C CCAGGCAU | 331 | AUGCCUGG CUGAUGAG GCCGUUAGGC CGAA AUUUGGAG | 2520 | |
| 2025 | CCAGGCAU U GCUAAGGU | 332 | ACCUUAGC CUGAUGAG GCCGUUAGGC CGAA AUGCCUGG | 2521 | |
| 2029 | GCAUUGCU A AGGUUGGC | 333 | GCCAACCU CUGAUGAG GCCGUUAGGC CGAA AGCAAUGC | 2522 | |
| 2034 | GCUAAGGU U GGCACUUG | 334 | CAAGUGCC CUGAUGAG GCCGUUAGGC CGAA ACCUUAGC | 2523 | |
| 2041 | UUGGCACU U GGAAAUAC | 335 | GUAUUUCC CUGAUGAG GCCGUUAGGC CGAA AGUGCCAA | 2524 | |
| 2048 | UUGGAAAU A CAGUCUGC | 336 | GCAGACUG CUGAUGAG GCCGUUAGGC CGAA AUUUCCAA | 2525 | |
| 2053 | AAUACAGU C UGCAAGCA | 337 | UGCUUGCA CUGAUGAG GCCGUUAGGC CGAA ACUGUAUU | 2526 | |
| 2066 | AGCAAGCU C ACAAACCU | 338 | AGGUUUGU CUGAUGAG GCCGUUAGGC CGAA AGCUUGCU | 2527 | |
| 2075 | ACAAACCU U GACCCUGA | 339 | UCAGGGUC CUGAUGAG GCCGUUAGGC CGAA AGGUUUGU | 2528 | |
| 2088 | CUGACUGU C ACGUCCCG | 340 | CGGGACGU CUGAUGAG GCCGUUAGGC CGAA ACAGUCAG | 2529 | |
| 2093 | UGUCACGU C CCGUGCGU | 341 | ACGCACGG CUGAUGAG GCCGUUAGGC CGAA ACGUGACA | 2530 | |
| 2102 | CCGUGCGU C CAAUGCUA | 342 | UAGCAUUG CUGAUGAG GCCGUUAGGC CGAA ACGCACGG | 2531 | |
| 2110 | CCAAUGCU A CCCUGCCU | 343 | AGGCAGGG CUGAUGAG GCCGUUAGGC CGAA AGCAUUGG | 2532 | |
| 2119 | CCCUGCCU C CAAUUACA | 344 | UGUAAUUG CUGAUGAG GCCGUUAGGC CGAA AGGCAGGG | 2533 | |
| 2124 | CCUCCAAU U ACAGUGAC | 345 | GUCACUGU CUGAUGAG GCCGUUAGGC CGAA AUUGGAGG | 2534 | |
| 2125 | CUCCAAUU A CAGUGACU | 346 | AGUCACUG CUGAUGAG GCCGUUAGGC CGAA AAUUGGAG | 2535 | |
| 2134 | CAGUGACU U CCAAAACG | 347 | CGUUUUGG CUGAUGAG GCCGUUAGGC CGAA AGUCACUG | 2536 | |
| 2135 | AGUGACUU C CAAAACGA | 348 | UCGUUUUG CUGAUGAG GCCGUUAGGC CGAA AAGUCACU | 2537 | |
| 2162 | CAGCAAAU U CCCCAGCC | 349 | GGCUGGGG CUGAUGAG GCCGUUAGGC CGAA AUUUGCUG | 2538 | |
| 2163 | AGCAAAUU C CCCAGCCC | 350 | GGGCUGGG CUGAUGAG GCCGUUAGGC CGAA AAUUUGCU | 2539 | |
| 2173 | CCAGCCCU C UGGUAGUU | 351 | AACUACCA CUGAUGAG GCCGUUAGGC CGAA AGGGCUGG | 2540 | |
| 2178 | CCUCUGGU A GUUUAUGC | 352 | GCAUAAAC CUGAUGAG GCCGUUAGGC CGAA ACCAGAGG | 2541 | |
| 2181 | CUGGUAGU U UAUGCAAA | 353 | UUUGCAUA CUGAUGAG GCCGUUAGGC CGAA ACUACCAG | 2542 | |
| 2182 | UGGUAGUU U AUGCAAAU | 354 | AUUUGCAU CUGAUGAG GCCGUUAGGC CGAA AACUACCA | 2543 | |
| 2183 | GGUAGUUU A UGCAAAUA | 355 | UAUUUGCA CUGAUGAG GCCGUUAGGC CGAA AAACUACC | 2544 | |
| 2191 | AUGCAAAU A UUCGCCAA | 356 | UUGGCGAA CUGAUGAG GCCGUUAGGC CGAA AUUUGCAU | 2545 | |
| 2193 | GCAAAUAU U CGCCAAGG | 357 | CCUUGGCG CUGAUGAG GCCGUUAGGC CGAA AUAUUUGC | 2546 | |
| 2194 | CAAAUAUU C GCCAAGGA | 358 | UCCUUGGC CUGAUGAG GCCGUUAGGC CGAA AAUAUUUG | 2547 | |
| 2207 | AGGAGCCU C CCCAAUUC | 359 | GAAUUGGG CUGAUGAG GCCGUUAGGC CGAA AGGCUCCU | 2548 | |
| 2214 | UCCCCAAU U CUCAGGGC | 360 | GCCCUGAG CUGAUGAG GCCGUUAGGC CGAA AUUGGGGA | 2549 | |
| 2215 | CCCCAAUU C UCAGGGCC | 361 | GGCCCUGA CUGAUGAG GCCGUUAGGC CGAA AAUUGGGG | 2550 | |
| 2217 | CCAAUUCU C AGGGCCAG | 362 | CUGGCCCU CUGAUGAG GCCGUUAGGC CGAA AGAAUUGG | 2551 | |
| 2229 | GCCAGUGU C ACAGCCCU | 363 | AGGGCUGU CUGAUGAG GCCGUUAGGC CGAA ACACUGGC | 2552 | |
| 2241 | GCCCUGAU U GAAUCAGU | 364 | ACUGAUUC CUGAUGAG GCCGUUAGGC CGAA AUCAGGGC | 2553 | |
| 2246 | GAUUGAAU C AGUGAAUG | 365 | CAUUCACU CUGAUGAG GCCGUUAGGC CGAA AUUCAAUC | 2554 | |
| 2265 | AAAACAGU U ACCUUGGA | 366 | UCCAAGGU CUGAUGAG GCCGUUAGGC CGAA ACUGUUUU | 2555 | |
| 2266 | AAACAGUU A CCUUGGAA | 367 | UUCCAAGG CUGAUGAG GCCGUUAGGC CGAA AACUGUUU | 2556 | |
| 2270 | AGUUACCU U GGAACUAC | 368 | GUAGUUCC CUGAUGAG GCCGUUAGGC CGAA AGGUAACU | 2557 | |
| 2277 | UUGGAACU A CUGGAUAZ | 369 | UUAUCCAG CUGAUGAG GCCGUUAGGC CGAA AGUUCCAA | 2558 | |
| 2284 | UACUGGAU A AUGGAGCA | 370 | UGCUCCAU CUGAUGAG GCCGUUAGGC CGAA AUCCAGUA | 2559 | |
| 2305 | CUGAUGCU A CUAAGGAU | 371 | AUCCUUAG CUGAUGAG GCCGUUAGGC CGAA AGCAUCAG | 2560 | |
| 2308 | AUGCUACU A AGGAUGAC | 372 | GUCAUCCU CUGAUGAG GCCGUUAGGC CGAA AGUAGCAU | 2561 | |
| 2322 | GACGGUGU C UACUCAAG | 373 | CUUGAGUA CUGAUGAG GCCGUUAGGC CGAA ACACCGUC | 2562 | |
| 2324 | CGGUGUCU A CUCAAGGU | 374 | ACCUUGAG CUGAUGAG GCCGUUAGGC CGAA AGACACCG | 2563 | |
| 2327 | UGUCUACU C AAGGUAUU | 375 | AAUACCUU CUGAUGAG GCCGUUAGGC CGAA AGUAGACA | 2564 | |
| 2333 | CUCAAGGU A UUUCACAA | 376 | UUGUGAAA CUGAUGAG GCCGUUAGGC CGAA ACCUUGAG | 2565 | |
| 2335 | CAAGGUAU U UCACAACU | 377 | AGUUGUGA CUGAUGAG GCCGUUAGGC CGAA AUACCUUG | 2566 | |
| 2336 | AAGGUAUU U CACAACUU | 378 | AAGUUGUG CUGAUGAG GCCGUUAGGC CGAA AAUACCUU | 2567 | |
| 2337 | AGGUAUUU C ACAACUUA | 379 | UAAGUUGU CUGAUGAG GCCGUUAGGC CGAA AAAUACCU | 2568 | |
| 2344 | UCACAACU U AUGACACG | 380 | CGUGUCAU CUGAUGAG GCCGUUAGGC CGAA AGUUGUGA | 2569 | |
| 2345 | CACAACUU A UGACACGA | 381 | UCGUGUCA CUGAUGAG GCCGUUAGGC CGAA AAGUUGUG | 2570 | |
| 2359 | CGAAUGGU A GAUACAGU | 382 | ACUGUAUC CUGAUGAG GCCGUUAGGC CGAA ACCAUUCG | 2571 | |
| 2363 | UGGUAGAU A CAGUGUAA | 383 | UUACACUG CUGAUGAG GCCGUUAGGC CGAA AUCUACCA | 2572 | |
| 2370 | UACAGUGU A AAAGUGCG | 384 | CGCACUUU CUGAUGAG GCCGUUAGGC CGAA ACACUGUA | 2573 | |
| 2383 | UGCGGGCU C UGGGAGGA | 385 | UCCUCCCA CUGAUGAG GCCGUUAGGC CGAA AGCCCGCA | 2574 | |
| 2394 | GGAGGAGU U AACGCAGC | 386 | GCUGCGUU CUGAUGAG GCCGUUAGGC CGAA ACUCCUCC | 2575 | |
| 2395 | GAGGAGUU A ACGCAGCC | 387 | GGCUGCGU CUGAUGAG GCCGUUAGGC CGAA AACUCCUC | 2576 | |
| 2418 | AGAGUGAU A CCCCAGCA | 388 | UGCUGGGG CUGAUGAG GCCGUUAGGC CGAA AUCACUCU | 2577 | |
| 2441 | AGCACUGU A CAUACCUG | 389 | CAGGUAUG CUGAUGAG GCCGUUAGGC CGAA ACAGUGCU | 2578 | |
| 2445 | CUGUACAU A CCUGGCUG | 390 | CAGCCAGG CUGAUGAG GCCGUUAGGC CGAA AUGUACAG | 2579 | |
| 2457 | GGCUGGAU U GAGAAUGA | 391 | UCAUUCUC CUGAUGAG GCCGUUAGGC CGAA AUCCAGCC | 2580 | |
| 2472 | GAUGAAAU A CAAUGGAA | 392 | UUCCAUUG CUGAUGAG GCCGUUAGGC CGAA AUUUCAUC | 2581 | |
| 2482 | AAUGGAAU C CACCAAGA | 393 | UCUUGGUG CUGAUGAG GCCGUUAGGC CGAA AUUCCAUU | 2582 | |
| 2499 | CCUGAAAU U AAUAAGGA | 394 | UCCUUAUU CUGAUGAG GCCGUUAGGC CGAA AUUUCAGG | 2583 | |
| 2500 | CUGAAAUU A AUAAGGAU | 395 | AUCCUUAU CUGAUGAG GCCGUUAGGC CGAA AAUUUCAG | 2584 | |
| 2503 | AAAUUAAU A AGGAUGAU | 396 | AUCAUCCU CUGAUGAG GCCGUUAGGC CGAA AUUAAUUU | 2585 | |
| 2514 | GAUGAUGU U CAACACAA | 397 | UUGUGUUG CUGAUGAG GCCGUUAGGC CGAA ACAUCAUC | 2586 | |
| 2515 | AUGAUGUU C AACACAAG | 398 | CUUGUGUU CUGAUGAG GCCGUUAGGC CGAA AACAUCAU | 2587 | |
| 2533 | AAGUGUGU U UCAGCAGA | 399 | UCUGCUGA CUGAUGAG GCCGUUAGGC CGAA ACACACUU | 2588 | |
| 2534 | AGUGUGUU U CAGCAGAA | 400 | UUCUGCUG CUGAUGAG GCCGUUAGGC CGAA AACACACU | 2589 | |
| 2535 | GUGUGUUU C AGCAGAAC | 401 | GUUCUGCU CUGAUGAG GCCGUUAGGC CGAA AAACACAC | 2590 | |
| 2546 | CAGAACAU C CUCGGGAG | 402 | CUCCCGAG CUGAUGAG GCCGUUAGGC CGAA AUGUUCUG | 2591 | |
| 2549 | AACAUCCU C GGGAGGCU | 403 | AGCCUCCC CUGAUGAG GCCGUUAGGC CGAA AGGAUGUU | 2592 | |
| 2558 | GGGAGGCU C AUUUGUGG | 404 | CCACAAAU CUGAUGAG GCCGUUAGGC CGAA AGCCUCCC | 2593 | |
| 2561 | AGGCUCAU U UGUGGCUU | 405 | AAGCCACA CUGAUGAG GCCGUUAGGC CGAA AUGAGCCU | 2594 | |
| 2562 | GGCUCAUU U GUGGCUUC | 406 | GAAGCCAC CUGAUGAG GCCGUUAGGC CGAA AAUGAGCC | 2595 | |
| 2569 | UUGUGGCU U CUGAUGUC | 407 | GACAUCAG CUGAUGAG GCCGUUAGGC CGAA AGCCACAA | 2596 | |
| 2570 | UGUGGCUU C UGAUGUCC | 408 | GGACAUCA CUGAUGAG GCCGUUAGGC CGAA AAGCCACA | 2597 | |
| 2577 | UCUGAUGU C CCAAAUGC | 409 | GCAUUUGG CUGAUGAG GCCGUUAGGC CGAA ACAUCAGA | 2598 | |
| 2587 | CAAAUGCU C CCAUACCU | 410 | AGGUAUGG CUGAUGAG GCCGUUAGGC CGAA AGCAUUUG | 2599 | |
| 2592 | GCUCCCAU A CCUGAUCU | 411 | AGAUCAGG CUGAUGAG GCCGUUAGGC CGAA AUGGGAGC | 2600 | |
| 2599 | UACCUGAU C UCUUCCCA | 412 | UGGGAAGA CUGAUGAG GCCGUUAGGC CGAA AUCAGGUA | 2601 | |
| 2601 | CCUGAUCU C UUCCCACC | 413 | GGUGGGAA CUGAUGAG GCCGUUAGGC CGAA AGAUCAGG | 2602 | |
| 2603 | UGAUCUCU U CCCACCUG | 414 | CAGGUGGG CUGAUGAG GCCGUUAGGC CGAA AGAGAUCA | 2603 | |
| 2604 | GAUCUCUU C CCACCUGG | 415 | CCAGGUGG CUGAUGAG GCCGUUAGGC CGAA AAGAGAUC | 2604 | |
| 2619 | GGCCAAAU C ACCGACCU | 416 | AGGUCGGU CUGAUGAG GCCGUUAGGC CGAA AUUUGGCC | 2605 | |
| 2640 | GCGGAAAU U CACGGGGG | 417 | CCCCCGUG CUGAUGAG GCCGUUAGGC CGAA AUUUCCGC | 2606 | |
| 2641 | CGGAAAUU C ACGGGGGC | 418 | GCCCCCGU CUGAUGAG GCCGUUAGGC CGAA AAUUUCCG | 2607 | |
| 2653 | GGGGCAGU C UCAUUAAU | 419 | AUUAAUGA CUGAUGAG GCCGUUAGGC CGAA ACUGCCCC | 2608 | |
| 2655 | GGCAGUCU C AUUAAUCU | 420 | AGAUUAAU CUGAUGAG GCCGUUAGGC CGAA AGACUGCC | 2609 | |
| 2658 | AGUCUCAU U AAUCUGAC | 421 | GUCAGAUU CUGAUGAG GCCGUUAGGC CGAA AUGAGACU | 2610 | |
| 2659 | GUCUCAUU A AUCUGACU | 422 | AGUCAGAU CUGAUGAG GCCGUUAGGC CGAA AAUGAGAC | 2611 | |
| 2662 | UCAUUAAU C UGACUUGG | 423 | CCAAGUCA CUGAUGAG GCCGUUAGGC CGAA AUUAAUGA | 2612 | |
| 2668 | AUCUGACU U GGACAGCU | 424 | AGCUGUCC CUGAUGAG GCCGUUAGGC CGAA AGUCAGAU | 2613 | |
| 2677 | GGACAGCU C CUGGGGAU | 425 | AUCCCCAG CUGAUGAG GCCGUUAGGC CGAA AGCUGUCC | 2614 | |
| 2689 | GGGAUGAU U AUGACCAU | 426 | AUGGUCAU CUGAUGAG GCCGUUAGGC CGAA AUCAUCCC | 2615 | |
| 2690 | GGAUGAUU A UGACCAUG | 427 | CAUGGUCA CUGAUGAG GCCGUUAGGC CGAA AAUCAUCC | 2616 | |
| 2707 | GAACAGCU C ACAAGUAU | 428 | AUACUUGU CUGAUGAG GCCGUUAGGC CGAA AGCUGUUC | 2617 | |
| 2714 | UCACAAGU A UAUCAUUC | 429 | GAAUGAUA CUGAUGAG GCCGUUAGGC CGAA ACUUGUGA | 2618 | |
| 2716 | ACAAGUAU A UCAUUCGA | 430 | UCGAAUGA CUGAUGAG GCCGUUAGGC CGAA AUACUUGU | 2619 | |
| 2718 | AAGUAUAU C AUUCGAAU | 431 | AUUCGAAU CUGAUGAG GCCGUUAGGC CGAA AUAUACUU | 2620 | |
| 2721 | UAUAUCAU U CGAAUAAG | 432 | CUUAUUCG CUGAUGAG GCCGUUAGGC CGAA AUGAUAUA | 2621 | |
| 2722 | AUAUCAUU C GAAUAAGU | 433 | ACUUAUUC CUGAUGAG GCCGUUAGGC CGAA AAUGAUAU | 2622 | |
| 2727 | AUUCGAAU A AGUACAAG | 434 | CUUGUACU CUGAUGAG GCCGUUAGGC CGAA AUUCGAAU | 2623 | |
| 2731 | GAAUAAGU A CAAGUAUU | 435 | AAUACUUG CUGAUGAG GCCGUUAGGC CGAA ACUUAUUC | 2624 | |
| 2737 | GUACAAGU A UUCUUGAU | 436 | AUCAAGAA CUGAUGAG GCCGUUAGGC CGAA ACUUGUAC | 2625 | |
| 2739 | ACAAGUAU U CUUGAUCU | 437 | AGAUCAAG CUGAUGAG GCCGUUAGGC CGAA AUACUUGU | 2626 | |
| 2740 | CAAGUAUU C UUGAUCUC | 438 | GAGAUCAA CUGAUGAG GCCGUUAGGC CGAA AAUACUUG | 2627 | |
| 2742 | AGUAUUCU U GAUCUCAG | 439 | CUGAGAUC CUGAUGAG GCCGUUAGGC CGAA AGAAUACU | 2628 | |
| 2746 | UUCUUGAU C UCAGAGAC | 440 | GUCUCUGA CUGAUGAG GCCGUUAGGC CGAA AUCAAGAA | 2629 | |
| 2748 | CUUGAUCU C AGAGACAA | 441 | UUGUCUCU CUGAUGAG GCCGUUAGGC CGAA AGAUCAAG | 2630 | |
| 2759 | AGACAAGU U CAAUGAAU | 442 | AUUCAUUG CUGAUGAG GCCGUUAGGC CGAA ACUUGUCU | 2631 | |
| 2760 | GACAAGUU C AAUGAAUC | 443 | GAUUCAUU CUGAUGAG GCCGUUAGGC CGAA AACUUGUC | 2632 | |
| 2768 | CAAUGAAU C UCUUCAAG | 444 | CUUGAAGA CUGAUGAG GCCGUUAGGC CGAA AUUCAUUG | 2633 | |
| 2770 | AUGAAUCU C UUCAAGUG | 445 | CACUUGAA CUGAUGAG GCCGUUAGGC CGAA AGAUUCAU | 2634 | |
| 2772 | GAAUCUCU U CAAGUGAA | 446 | UUCACUUG CUGAUGAG GCCGUUAGGC CGAA AGAGAUUC | 2635 | |
| 2773 | AAUCUCUU C AAGUGAAU | 447 | AUUCACUU CUGAUGAG GCCGUUAGGC CGAA AAGAGAUU | 2636 | |
| 2782 | AAGUGAAU A CUACUGCU | 448 | AGCAGUAG CUGAUGAG GCCGUUAGGC CGAA AUUCACUU | 2637 | |
| 2785 | UGAAUACU A CUGCUCUC | 449 | GAGAGCAG CUGAUGAG GCCGUUAGGC CGAA AGUAUUCA | 2638 | |
| 2791 | CUACUGCU C UCAUCCCA | 450 | UGGGAUGA CUGAUGAG GCCGUUAGGC CGAA AGCAGUAG | 2639 | |
| 2793 | ACUGCUCU C AUCCCAAA | 451 | UUUGGGAU CUGAUGAG GCCGUUAGGC CGAA AGAGCAGU | 2640 | |
| 2796 | GCUCUCAU C CCAAAGGA | 452 | UCCUUUGG CUGAUGAG GCCGUUAGGC CGAA AUGAGAGC | 2641 | |
| 2813 | AGCCAACU C UGAGGAAG | 453 | CUUCCUCA CUGAUGAG GCCGUUAGGC CGAA AGUUGGCU | 2642 | |
| 2823 | GAGGAAGU C UUUUUGUU | 454 | AACAAAAA CUGAUGAG GCCGUUAGGC CGAA ACUUCCUC | 2643 | |
| 2825 | GGAAGUCU U UUUGUUUA | 455 | UAAACAAA CUGAUGAG GCCGUUAGGC CGAA AGACUUCC | 2644 | |
| 2826 | GAAGUCUU U UUGUUUAA | 456 | UUAAACAA CUGAUGAG GCCGUUAGGC CGAA AAGACUUC | 2645 | |
| 2827 | AAGUCUUU U UGUUUAAA | 457 | UUUAAACA CUGAUGAG GCCGUUAGGC CGAA AAAGACUU | 2646 | |
| 2828 | AGUCUUUU U GUUUAAAC | 458 | GUUUAAAC CUGAUGAG GCCGUUAGGC CGAA AAAAGACU | 2647 | |
| 2831 | CUUUUUGU U UAAACCAG | 459 | CUGGUUUA CUGAUGAG GCCGUUAGGC CGAA ACAAAAAG | 2648 | |
| 2832 | UUUUUGUU U AAACCAGA | 460 | UCUGGUUU CUGAUGAG GCCGUUAGGC CGAA AACAAAAA | 2649 | |
| 2833 | UUUUGUUU A AACCAGAA | 461 | UUCUGGUU CUGAUGAG GCCGUUAGGC CGAA AAACAAAA | 2650 | |
| 2847 | GAAAACAU U ACUUUUGA | 462 | UCAAAAGU CUGAUGAG GCCGUUAGGC CGAA AUGUUUUC | 2651 | |
| 2848 | AAAACAUU A CUUUUGAA | 463 | UUCAAAAG CUGAUGAG GCCGUUAGGC CGAA AAUGUUUU | 2652 | |
| 2851 | ACAUUACU U UUGAAAAU | 464 | AUUUUCAA CUGAUGAG GCCGUUAGGC CGAA AGUAAUGU | 2653 | |
| 2852 | CAUUACUU U UGAAAAUG | 465 | CAUUUUCA CUGAUGAG GCCGUUAGGC CGAA AAGUAAUG | 2654 | |
| 2853 | AUUACUUU U GAAAAUGG | 466 | CCAUUUUC CUGAUGAG GCCGUUAGGC CGAA AAAGUAAU | 2655 | |
| 2869 | GCACAGAU C UUUUCAUU | 467 | AAUGAAAA CUGAUGAG GCCGUUAGGC CGAA AUCUGUGC | 2656 | |
| 2871 | ACAGAUCU U UUCAUUGC | 468 | GCAAUGAA CUGAUGAG GCCGUUAGGC CGAA AGAUCUGU | 2657 | |
| 2872 | CAGAUCUU U UCAUUGCU | 469 | AGCAAUGA CUGAUGAG GCCGUUAGGC CGAA AAGAUCUG | 2658 | |
| 2873 | AGAUCUUU U CAUUGCUA | 470 | UAGCAAUG CUGAUGAG GCCGUUAGGC CGAA AAAGAUCU | 2659 | |
| 2874 | GAUCUUUU C AUUGCUAU | 471 | AUAGCAAU CUGAUGAG GCCGUUAGGC CGAA AAAAGAUC | 2660 | |
| 2877 | CUUUUCAU U GCUAUUCA | 472 | UGAAUAGC CUGAUGAG GCCGUUAGGC CGAA AUGAAAAG | 2661 | |
| 2881 | UCAUUGCU A UUCAGGCU | 473 | AGCCUGAA CUGAUGAG GCCGUUAGGC CGAA AGCAAUGA | 2662 | |
| 2883 | AUUGCUAU U CAGGCUGU | 474 | ACAGCCUG CUGAUGAG GCCGUUAGGC CGAA AUAGCAAU | 2663 | |
| 2884 | UUGCUAUU C AGGCUGUU | 475 | AACAGCCU CUGAUGAG GCCGUUAGGC CGAA AAUAGCAA | 2664 | |
| 2892 | CAGGCUGU U GAUAAGGU | 476 | ACCUUAUC CUGAUGAG GCCGUUAGGC CGAA ACAGCCUG | 2665 | |
| 2896 | CUGUUGAU A AGGUCGAU | 477 | AUCGACCU CUGAUGAG GCCGUUAGGC CGAA AUCAACAG | 2666 | |
| 2901 | GAUAAGGU C GAUCUGAA | 478 | UUCAGAUC CUGAUGAG GCCGUUAGGC CGAA ACCUUAUC | 2667 | |
| 2905 | AGGUCGAU C UGAAAUCA | 479 | UGAUUUCA CUGAUGAG GCCGUUAGGC CGAA AUCGACCU | 2668 | |
| 2912 | UCUGAAAU C AGAAAUAU | 480 | AUAUUUCU CUGAUGAG GCCGUUAGGC CGAA AUUUCAGA | 2669 | |
| 2919 | UCAGAAAU A UCCAACAU | 481 | AUGUUGGA CUGAUGAG GCCGUUAGGC CGAA AUUUCUGA | 2670 | |
| 2921 | AGAAAUAU C CAACAUUG | 482 | CAAUGUUG CUGAUGAG GCCGUUAGGC CGAA AUAUUUCU | 2671 | |
| 2928 | UCCAACAU U GCACGAGU | 483 | ACUCGUGC CUGAUGAG GCCGUUAGGC CGAA AUGUUGGA | 2672 | |
| 2937 | GCACGAGU A UCUUUGUU | 484 | AACAAAGA CUGAUGAG GCCGUUAGGC CGAA ACUCGUGC | 2673 | |
| 2939 | ACGAGUAU C UUUGUUUA | 485 | UAAACAAA CUGAUGAG GCCGUUAGGC CGAA AUACUCGU | 2674 | |
| 2941 | GAGUAUCU U UGUUUAUU | 486 | AAUAAACA CUGAUGAG GCCGUUAGGC CGAA AGAUACUC | 2675 | |
| 2942 | AGUAUCUU U GUUUAUUC | 487 | GAAUAAAC CUGAUGAG GCCGUUAGGC CGAA AAGAUACU | 2676 | |
| 2945 | AUCUUUGU U UAUUCCUC | 488 | GAGGAAUA CUGAUGAG GCCGUUAGGC CGAA ACAAAGAU | 2677 | |
| 2946 | UCUUUGUU U AUUCCUCC | 489 | GGAGGAAU CUGAUGAG GCCGUUAGGC CGAA AACAAAGA | 2678 | |
| 2947 | CUUUGUUU A UUCCUCCA | 490 | UGGAGGAA CUGAUGAG GCCGUUAGGC CGAA AAACAAAG | 2679 | |
| 2949 | UUGUUUAU U CCUCCACA | 491 | UGUGGAGG CUGAUGAG GCCGUUAGGC CGAA AUAAACAA | 2680 | |
| 2950 | UGUUUAUU C CUCCACAG | 492 | CUGUGGAG CUGAUGAG GCCGUUAGGC CGAA AAUAAACA | 2681 | |
| 2953 | UUAUUCCU C CACAGACU | 493 | AGUCUGUG CUGAUGAG GCCGUUAGGC CGAA AGGAAUAA | 2682 | |
| 2962 | CACAGACU C CGCCAGAG | 494 | CUCUGGCG CUGAUGAG GCCGUUAGGC CGAA AGUCUGUG | 2683 | |
| 2977 | AGACACCU A GUCCUGAU | 495 | AUCAGGAC CUGAUGAG GCCGUUAGGC CGAA AGGUGUCU | 2684 | |
| 2980 | CACCUAGU C CUGAUGAA | 496 | UUCAUCAG CUGAUGAG GCCGUUAGGC CGAA ACUAGGUG | 2685 | |
| 2993 | UGAAACGU C UGCUCCUU | 497 | AAGGAGCA CUGAUGAG GCCGUUAGGC CGAA ACGUUUCA | 2686 | |
| 2998 | CGUCUGCU C CUUGUCCU | 498 | AGGACAAG CUGAUGAG GCCGUUAGGC CGAA AGCAGACG | 2687 | |
| 3001 | CUGCUCCU U GUCCUAAU | 499 | AUUAGGAC CUGAUGAG GCCGUUAGGC CGAA ACAAGGAG | 2688 | |
| 3004 | CUCCUUGU C CUAAUAUU | 500 | AAUAUUAG CUGAUGAG GCCGUUAGGC CGAA ACAAGGAG | 2689 | 2689 |
| 3007 | CUUGUCCU A AUAUUCAU | 501 | AUGAAUAU CUGAUGAG GCCGUUAGGC CGAA AGGACAAG | 2690 | |
| 3010 | GUCCUAAU A UUCAUAUC | 502 | GAUAUGAA CUGAUGAG GCCGUUAGGC CGAA AUUAGGAC | 2691 | |
| 3012 | CCUAAUAU U CAUAUCAA | 503 | UUGAUAUG CUGAUGAG GCCGUUAGGC CGAA AUAUUAGG | 2692 | |
| 3013 | CUAAUAUU C AUAUCAAC | 504 | GUUGAUAU CUGAUGAG GCCGUUAGGC CGAA AZUAUUAG | 2693 | |
| 3016 | AUAUUCAU A UCAACAGC | 505 | GCUGUUGA CUGAUGAG GCCGUUAGGC CGAA AUGAAUAU | 2694 | |
| 3018 | AUUCAUAU C AACAGCAC | 506 | GUGCUGUU CUGAUGAG GCCGUUAGGC CGAA AUAUGAAU | 2695 | |
| 3030 | AGCACCAU U CCUGGCAU | 507 | AUGCCAGG CUGAUGAG GCCGUUAGGC CGAA AUGGUGCU | 2696 | |
| 3031 | GCACCAUU C CUGGCAUU | 508 | AAUGCCAG CUGAUGAG GCCGUUAGGC CGAA AAUGGUGC | 2697 | |
| 3039 | CCUGGCAU U CACAUUUU | 509 | AAAAUGUG CUGAUGAG GCCGUUAGGC CGAA AUGCCAGG | 2698 | |
| 3040 | CUGGCAUU C ACAUUUUA | 510 | UAAAAUGU CUGAUGAG GCCGUUAGGC CGAA AAUGCCAG | 2699 | |
| 3045 | AUUCACAU U UUAAAAAU | 511 | AUUUUUAA CUGAUGAG GCCGUUAGGC CGAA AUGUGAAU | 2700 | |
| 3046 | UUCACAUU U UAAAAAUU | 512 | AAUUUUUA CUGAUGAG GCCGUUAGGC CGAA AAUGUGAA | 2701 | |
| 3047 | UCACAUUU U AAAAAUUA | 513 | UAAUUUUU CUGAUGAG GCCGUUAGGC CGAA AAAUGUGA | 2702 | |
| 3048 | CACAUUUU A AAAAUUAU | 514 | AUAAUUUU CUGAUGAG GCCGUUAGGC CGAA AAAAUGUG | 2703 | |
| 3054 | UUAAAAAU U AUGUGGAA | 515 | UUCCACAU CUGAUGAG GCCGUUAGGC CGAA AUUUUUAA | 2704 | |
| 3055 | UAAAAAUU A UGUGGAAG | 516 | CUUCCACA CUGAUGAG GCCGUUAGGC CGAA AAUUUUUA | 2705 | |
| 3069 | AAGUGGAU A GGAGAACU | 517 | AGUUCUCC CUGAUGAG GCCGUUAGGC CGAA AUCCACUU | 2706 | |
| 3086 | GCAGCUGU C AAUAGCCU | 518 | AGGCUAUU CUGAUGAG GCCGUUAGGC CGAA ACAGCUGC | 2707 | |
| 3090 | CUGUCAAU A GCCUAGGG | 519 | CCCUAGGC CUGAUGAG GCCGUUAGGC CGAA AUUGACAG | 2708 | |
| 3095 | AAUAGCCU A CGGCUGAA | 520 | UUCAGCCC CUGAUCAG GCCGUUAGGC CGAA AGGCUAUU | 2709 | |
| 3105 | GGCUGAAU U UUUGUCAG | 521 | CUGACAAA CUGAUGAG GCCGUUAGGC CGAA AUUCAGCC | 2710 | |
| 3106 | GCUGAAUU U UUGUCAGA | 522 | UCUGACAA CUGAUGAG GCCGUUAGGC CGAA AAUUCAGC | 2711 | |
| 3107 | CUGAAUUU U UGUCAGAU | 523 | AUCUGACA CUGAUGAG GCCGUUAGGC CGAA AAAUUCAG | 2712 | |
| 3108 | UGAAUUUU U GUCAGAUA | 524 | UAUCUGAC CUGAUGAG GCCGUUAGGC CGAA AAAAUUCA | 2713 | |
| 3111 | AUUUUUGU C AGAUAAAU | 525 | AUUUAUCU CUGAUGAG GCCGUUAGGC CGAA ACAAAAAU | 2714 | |
| 3116 | UGUCAGAU A AAUAAAAU | 526 | AUUUUAUU CUGAUGAG GCCGUUAGGC CGAA AUCUGACA | 2715 | |
| 3120 | AGAUAAAU A AAAUAAAU | 527 | AUUUAUUU CUGAUGAG GCCGUUAGGC CGAA AUUUAUCU | 2716 | |
| 3125 | AAUAAAAU A AAUCAUUC | 528 | GAAUGAUU CUGAUGAG GCCGUUAGGC CGAA AUUUUAUU | 2717 | |
| 3129 | AAAUAAAU C AUUCAUCC | 529 | GGAUGAAU CUGAUGAG GCCGUUAGGC CGAA AUUUAUUU | 2718 | |
| 3132 | UAAAUCAU U CAUCCUUU | 530 | AAAGGAUG CUGAUGAG GCCGUUAGGC CGAA AUGAUUUA | 2719 | |
| 3133 | AAAUCAUU C AUCCUUUU | 531 | AAAAGGAU CUGAUGAG GCCGUUAGGC CGAA AAUGAUUU | 2720 | |
| 3136 | UCAUUCAU C CUUUUUUU | 532 | AAAAAAAG CUGAUGAG GCCGUUAGGC CGAA AUGAAUGA | 2721 | |
| 3139 | UUCAUCCU U UUUUUGAU | 533 | AUCAAAAA CUGAUGAG GCCGUUAGGC CGAA AGGAUGAA | 2722 | |
| 3140 | UCAUCCUU U UUUUGAUU | 534 | AAUCAAAA CUGAUGAG GCCGUUAGGC CGAA AAGGAUGA | 2723 | |
| 3141 | CAUCCUUU U UUUGAUUA | 535 | UAAUCAAA CUGAUGAG GCCGUUAGGC CGAA AAAGGAUG | 2724 | |
| 3142 | AUCCUUUU U UUGAUUAU | 536 | AUAAUCAA CUGAUGAG GCCGUUAGGC CGAA AAAAGGAU | 2725 | |
| 3143 | UCCUUUUU U UGAUUAUA | 537 | UAUAAUCA CUGAUGAG GCCGUUAGGC CGAA AAAAAGGA | 2726 | |
| 3144 | CCUUUUUU U GAUUAUAA | 538 | UUAUAAUC CUGAUGAG GCCGUUAGGC CGAA AAAAAAGG | 2727 | |
| 3148 | UUUUUGAU U AUAAAAUU | 539 | AAUUUUAU CUGAUGAG GCCGUUAGGC CGAA AUCAAAAA | 2728 | |
| 3149 | UUUUGAUU A UAAAAUUU | 540 | AAAUUUUA CUGAUGAG GCCGUUAGGC CGAA AAUCAAAA | 2729 | |
| 3151 | UUGAUUAU A AAAUUUUC | 541 | GAAAAUUU CUGAUGAG GCCGUUAGGC CGAA AUAAUCAA | 2730 | |
| 3156 | UAUAAAAU U UUCUAAAA | 542 | UUUUAGAA CUGAUGAG GCCGUUAGGC CGAA AUUUUAUA | 2731 | |
| 3157 | AUAAAAUU U UCUAAAAU | 543 | AUUUUAGA CUGAUGAG GCCGUUAGGC CGAA AAUUUUAU | 2732 | |
| 3158 | UAAAAUUU U CUAAAAUG | 544 | CAUUUUAG CUGAUGAG GCCGUUAGGC CGAA AAAUUUUA | 2733 | |
| 3159 | AAAAUUUU C UAAAAUGU | 545 | ACAUUUUA CUGAUGAG GCCGUUAGGC CGAA AAAAUUUU | 2734 | |
| 3161 | AAUUUUCU A AAAUGUAU | 546 | AUACAUUU CUGAUGAG GCCGUUAGGC CGAA AGAAAAUU | 2735 | |
| 3168 | UAAAAUGU A UUUUAGAC | 547 | GUCUAAAA CUGAUGAG GCCGUUAGGC CGAA ACAUUUUA | 2736 | |
| 3170 | AAAUGUAU U UUAGACUU | 548 | AAGUCUAA CUGAUGAG GCCGUUAGGC CGAA AUACAUUU | 2737 | |
| 3260 | AAAUGUAU U UUAGACUU | 548 | AAGUCUAA CUGAUGAG GCCGUUAGGC CGAA AUACAUUU | 2737 | |
| 3171 | AAUGUAUU U UAGACUUC | 549 | GAAGUCUA CUGAUGAG GCCGUUAGGC CGAA AAUACAUU | 2738 | |
| 3261 | AAUGUAUU U UAGACUUC | 549 | GAAGUCUA CUGAUGAG GCCGUUAGGC CGAA AAUACAUU | 2738 | |
| 3172 | AUGUAUUU U AGACUUCC | 550 | GGAAGUCU CUGAUGAG GCCGUUAGGC CGAA AAAUACAU | 2739 | |
| 3262 | AUGUAUUU U AGACUUCC | 550 | GGAAGUCU CUGAUGAG GCCGUUAGGC CGAA AAAUACAU | 2739 | |
| 3173 | UGUAUUUU A GACUUCCU | 551 | AGGAAGUC CUGAUGAG GCCGUUAGGC CGAA AAAAUACA | 2740 | |
| 3263 | UGUAUUUU A GACUUCCU | 551 | AGGAAGUC CUGAUGAG GCCGUUAGGC CGAA AAAAUACA | 2740 | |
| 3178 | UUUAGACU U CCUGUAGG | 552 | CCUACAGG CUGAUGAG GCCGUUAGGC CGAA AGUCUAAA | 2741 | |
| 3268 | UUUAGACU U CCUGUAGG | 552 | CCUACAGG CUGAUGAG GCCGUUAGGC CGAA AGUCUAAA | 2741 | |
| 3179 | UUAGACUU C CUGUAGGG | 553 | CCCUACAG CUGAUGAG GCCGUUAGGC CGAA AAGUCUAA | 2742 | |
| 3269 | UUAGACUU C CUGUAGGG | 553 | CCCUACAG CUGAUGAG GCCGUUAGGC CGAA AAGUCUAA | 2742 | |
| 3184 | CUUCCUGU A GGGGGCGA | 554 | UCGCCCCC CUGAUGAG GCCGUUAGGC CGAA ACAGGAAG | 2743 | |
| 3274 | CUUCCUGU A GGGGGCGA | 554 | UCGCCCCC CUGAUGAG GCCGUUAGGC CGAA ACAGGAAG | 2743 | |
| 3194 | GGGGCGAU A UACUAAAU | 555 | AUUUAGUA CUGAUGAG GCCGUUAGGC CGAA AUCGCCCC | 2744 | |
| 3247 | GGGGCGAU A UACUAAAU | 555 | AUUUAGUA CUGAUGAG GCCGUUAGGC CGAA AUCGCCCC | 2744 | |
| 3196 | GGCGAUAU A CUAAAUGU | 556 | ACAUUUAG CUGAUGAG GCCGUUAGGC CGAA AUAUCGCC | 2745 | |
| 3249 | GGCGAUAU A CUAAAUGU | 556 | ACAUUUAG CUGAUGAG GCCGUUAGGC CGAA AUAUCGCC | 2745 | |
| 3199 | GAUAUACU A AAUGUAUA | 557 | UAUACAUU CUGAUGAG GCCGUUAGGC CGAA AGUAUAUC | 2746 | |
| 3205 | CUAAAUGU A UAUAGUAC | 558 | GUACUAUA CUGAUGAG GCCGUUAGGC CGAA ACAUUUAG | 2747 | |
| 3207 | AAAUGUAU A UAGUACAU | 559 | AUGUACUA CUGAUGAG GCCGUUAGGC CGAA AUACAUUU | 2748 | |
| 3209 | AUGUAUAU A GUACAUUU | 560 | AAAUGUAC CUGAUGAG GCCGUUAGGC CGAA AUAUACAU | 2749 | |
| 3212 | UAUAUAGU A CAUUUAUA | 561 | UAUAAAUG CUGAUGAG GCCGUUAGGC CGAA ACUAUAUA | 2750 | |
| 3216 | UAGUACAU U UAUACUAA | 562 | UUAGUAUA CUGAUGAG GCCGUUAGGC CGAA AUGUACUA | 2751 | |
| 3217 | AGUACAUU U AUACUAAA | 563 | UUUAGUAU CUGAUGAG GCCGUUAGGC CGAA AAUGUACU | 2752 | |
| 3218 | GUACAUUU A UACUAAAU | 564 | AUUUAGUA CUGAUGAG GCCGUUAGGC CGAA AAAUGUAC | 2753 | |
| 3220 | ACAUUUAU A CUAAAUGU | 565 | ACAUUUAG CUGAUGAG GCCGUUAGGC CGAA AUAAAUGU | 2754 | |
| 3223 | UUUAUACU A AAUGUAUU | 566 | AAUACAUU CUGAUGAG GCCGUUAGGC CGAA AGUAUAAA | 2755 | |
| 3229 | CUAAAUGU A UUCCUGUA | 567 | UACAGGAA CUGAUGAG GCCGUUAGGC CGAA ACAUUUAG | 2756 | |
| 3231 | AAAUGUAU U CCUGUAGG | 568 | CCUACAGG CUGAUGAG GCCGUUAGGC CGAA AUACAUUU | 2757 | |
| 3232 | AAUGUAUU C CUGUAGGG | 569 | CCCUACAG CUGAUGAG GCCGUUAGGC CGAA AAUACAUU | 2758 | |
| 3237 | AUUCCUGU A GGGGGCGA | 570 | UCGCCCCC CUGAUGAG GCCGUUAGGC CGAA ACAGGAAU | 2759 | |
| 3252 | GAUAUACU A AAUGUAUU | 571 | AAUACAUU CUGAUGAG GCCGUUAGGC CGAA AGUAUAUC | 2760 | |
| 3258 | CUAAAUGU A UUUUAGAC | 572 | GUCUAAAA CUGAUGAG GCCGUUAGGC CGAA ACAUUUAG | 2761 | |
| 3284 | GGGGCGAU A AAAUAAAA | 573 | UUUUAUUU CUGAUGAG GCCGUUAGGC CGAA AUCGCCCC | 2762 | |
| 3289 | GAUAAAAU A AAAUGCUA | 574 | UAGCAUUU CUGAUGAG GCCGUUAGGC CGAA AUUUUAUC | 2763 | |
| 3297 | AAAAUGCU A AACAACUG | 575 | CAGUUGUU CUGAUGAG GCCGUUAGGC CGAA AGCAUUUU | 2764 | |

Input Sequence = NM_001285. Cut Site = UH/.
Arm Length = 8. Core Sequence = CUGAUGAG GCCGUUAGGC CGAA
Underlined region can be any X sequence or linker, as described herein.
NM_001285 (Homo sapiens chloride channel, calcium activated, 1 (CLCA1) mRNA, 3311 bp)

TABLE IV
Human CLCA1 Inozyme and Target Sequence 249.021

| | | | | Rz |
|---|---|---|---|---|
| | | Seq ID | | Seq ID |
| Pos | Substrate | No. | Inozyme | No. |
| 10 | GCUAAUGC U UUUGGUAC | 576 | GUACCAAA CUGAUGAG GCCGUUAGGC CGAA ICAUUAGC | 2765 |
| 19 | UUUGGUAC A AAUGGAUG | 577 | CAUCCAUU CUGAUGAG GCCGUUAGGC CGAA IUACCAAA | 2766 |
| 50 | AUAUUUUC U UGUUUAAG | 578 | CUUAAACA CUGAUGAG GCCGUUAGGC CGAA IAAAAUAU | 2767 |
| 65 | AGGGGAGC A UGAAGAGG | 579 | CCUCUUCA CUGAUGAG GCCGUUAGGC CGAA ICUCCCCU | 2768 |
| 89 | GUUAUGUC A AGCAUCUG | 580 | CAGAUGCU CUGAUGAG GCCGUUAGGC CGAA IACAUAAC | 2769 |
| 93 | UGUCAAGC A UCUGGCAC | 581 | GUGCCAGA CUGAUGAG GCCGUUAGGC CGAA ICUUGACA | 2770 |
| 96 | CAAGCAUC U GGCACAGC | 582 | GCUGUGCC CUGAUGAG GCCGUUAGGC CGAA IAUGCUUG | 2771 |
| 100 | CAUCUGGC A CAGCUGAA | 583 | UUCAGCUG CUGAUGAG GCCGUUAGGC CGAA ICCAGAUG | 2772 |
| 102 | UCUGGCAC A GCUGAAGG | 584 | CCUUCAGC CUGAUGAG GCCGUUAGGC CGAA IUGCCAGA | 2773 |
| 105 | GGCACAGC U GAAGGCAG | 585 | CUGCCUUC CUGAUGAG GCCGUUAGGC CGAA ICUGUGCC | 2774 |
| 112 | CUGAAGGC A GAUGGAAA | 586 | UUUCCAUC CUGAUGAG GCCGUUAGGC CGAA ICCUUCAG | 2775 |
| 128 | AUAUUUAC A AGUACGCA | 587 | UGCGUACU CUGAUGAG GCCGUUAGGC CGAA IUAAAUAU | 2776 |
| 136 | AAGUACGC A AUUUGAGA | 588 | UCUCAAAU CUGAUGAG GCCGUUAGGC CGAA ICGUACUU | 2777 |
| 146 | UUUGAGAC U AAGAUAUU | 589 | AAUAUCUU CUGAUGAG GCCGUUAGGC CGAA IUCUCAAA | 2778 |
| 161 | UUGUUAUC A UUCUCCUA | 590 | UAGGAGAA CUGAUGAG GCCGUUAGGC CGAA IAUAACAA | 2779 |
| 165 | UAUCAUUC U CCUAUUGA | 591 | UCAAUAGG CUGAUGAG GCCGUUAGGC CGAA IAAUGAUA | 2780 |
| 167 | UCAUUCUC C UAUUGAAG | 592 | CUUCAAUA CUGAUGAG GCCGUUAGGC CGAA IAGAAUGA | 2781 |
| 168 | CAUUCUCC U AUUGAAGA | 593 | UCUUCAAU CUGAUGAG GCCGUUAGGC CGAA IGAGAAUG | 2782 |
| 178 | UUGAAGAC A AGAGCAAU | 594 | AUUGCUCU CUGAUGAG GCCGUUAGGC CGAA IUCUUCAA | 2783 |
| 184 | ACAAGAGC A AUAGUAAA | 595 | UUUACUAU CUGAUGAG GCCGUUAGGC CGAA ICUCUUGU | 2784 |
| 195 | AGUAAAAC A CAUCAGGU | 596 | ACCUGAUG CUGAUGAG GCCGUUAGGC CGAA IUUUUACU | 2785 |
| 197 | UAAAACAC A UCAGGUCA | 597 | UGACCUGA CUGAUGAG GCCGUUAGGC CGAA IUGUUUUA | 2786 |
| 200 | AACACAUC A GGUCAGGG | 598 | CCCUGACC CUGAUGAG GCCGUUAGGC CGAA IAUGUGUU | 2787 |
| 205 | AUCAGGUC A GGGGGUUA | 599 | UAACCCCC CUGAUGAG GCCGUUAGGC CGAA IACCUGAU | 2788 |
| 219 | UUAAAGAC C UGUGAUAA | 600 | UUAUCACA CUGAUGAG GCCGUUAGGC CGAA IUCUUUAA | 2789 |
| 220 | UAAAGACC U GUGAUAAA | 601 | UUUAUCAC CUGAUGAG GCCGUUAGGC CGAA IGUCUUUA | 2790 |
| 230 | UGAUAAAC C ACUUCCGA | 602 | UCGGAAGU CUGAUGAG GCCGUUAGGC CGAA IUUUAUCA | 2791 |
| 231 | GAUAAACC A CUUCCGAU | 603 | AUCGGAAG CUGAUGAG GCCGUUAGGC CGAA IGUUUAUC | 2792 |
| 233 | UAAACCAC U UCCGAUAA | 604 | UUAUCGGA CUGAUGAG GCCGUUAGGC CGAA IUGGUUUA | 2793 |
| 236 | ACCACUUC C GAUAAGUU | 605 | AACUUAUC CUGAUGAG GCCGUUAGGC CGAA IAAGUGGU | 2794 |
| 258 | CGUGUGUC U AUAUUUUC | 606 | GAAAAUAU CUGAUGAG GCCGUUAGGC CGAA IACACACG | 2795 |
| 267 | AUAUUUUC A UAUCUGUA | 607 | UACAGAUA CUGAUGAG GCCGUUAGGC CGAA IAAAAUAU | 2796 |
| 272 | UUCAUAUC U GUAUAUAU | 608 | AUAUAUAC CUGAUGAG GCCGUUAGGC CGAA IAUAUGAA | 2797 |
| 299 | AGAAAGAC A CCUUCGUA | 609 | UACGAAGG CUGAUGAG GCCGUUAGGC CGAA IUCUUUCU | 2798 |
| 301 | AAAGACAC C UUCGUAAC | 610 | GUUACGAA CUGAUGAG GCCGUUAGGC CGAA IUGUCUUU | 2799 |
| 302 | AAGACACC U UCGUAACC | 611 | GGUUACGA CUGAUGAG GCCGUUAGGC CGAA IGUGUCUU | 2800 |
| 310 | UUCGUAAC C CGCAUUUU | 612 | AAAAUGCG CUGAUGAG GCCGUUAGGC CGAA IUUACGAA | 2801 |
| 311 | UCGUAACC C GCAUUUUC | 613 | GAAAAUGC CUGAUGAG GCCGUUAGGC CGAA IGUUACGA | 2802 |
| 314 | UAACCCGC A UUUUCCAA | 614 | UUGGAAAA CUGAUGAG GCCGUUAGGC CGAA ICGGGUUA | 2803 |
| 320 | GCAUUUUC C AAAGAGAG | 615 | CUCUCUUU CUGAUGAG GCCGUUAGGC CGAA IAAAAUGC | 2804 |
| 321 | CAUUUUCC A AAGAGAGG | 616 | CCUCUCUU CUGAUGAG GCCGUUAGGC CGAA IGAAAAUG | 2805 |
| 334 | GAGGAAUC A CAGGGAGA | 617 | UCUCCCUG CUGAUGAG GCCGUUAGGC CGAA IAUUCCUC | 2806 |
| 336 | GGAAUCAC A GGGAGAUG | 618 | CAUCUCCC CUGAUGAG GCCGUUAGGC CGAA IUGAUUCC | 2807 |
| 348 | AGAUGUAC A GCAAUGGG | 619 | CCCAUUGC CUGAUGAG GCCGUUAGGC CGAA IUACAUCU | 2808 |
| 351 | UGUACAGC A AUGGGGCC | 620 | GGCCCCAU CUGAUGAG GCCGUUAGGC CGAA ICUGUACA | 2809 |
| 359 | AAUGGGGC C AUUUAAGA | 621 | UCUUAAAU CUGAUGAG GCCGUUAGGC CGAA ICCCCAUU | 2810 |
| 360 | AUGGGGCC A UUUAAGAG | 622 | CUCUUAAA CUGAUGAG GCCGUUAGGC CGAA IGCCCCAU | 2811 |
| 372 | AAGAGUUC U GUGUUCAU | 623 | AUGAACAC CUGAUGAG GCCGUUAGGC CGAA IAACUCUU | 2812 |
| 379 | CUGUGUUC A UCUUGAUU | 624 | AAUCAAGA CUGAUGAG GCCGUUAGGC CGAA IAACACAG | 2813 |
| 382 | UGUUCAUC U UGAUUCUU | 625 | AAGAAUCA CUGAUGAG GCCGUUAGGC CGAA IAUGAACA | 2814 |
| 389 | CUUGAUUC U UCACCUUC | 626 | GAAGGUGA CUGAUGAG GCCGUUAGGC CGAA IAAUCAAG | 2815 |
| 392 | GAUUCUUC A CCUUCUAG | 627 | CUAGAAGG CUGAUGAG GCCGUUAGGC CGAA IAAGAAUC | 2816 |
| 394 | UUCUUCAC C UUCUAGAA | 628 | UUCUAGAA CUGAUGAG GCCGUUAGGC CGAA IUGAAGAA | 2817 |
| 395 | UCUUCACC U UCUAGAAG | 629 | CUUCUAGA CUGAUGAG GCCGUUAGGC CGAA IGUGAAGA | 2818 |
| 398 | UCACCUUC U AGAAGGGG | 630 | CCCCUUCU CUGAUGAG GCCGUUAGGC CGAA IAAGGUGA | 2819 |
| 408 | GAAGGGGC C CUGAGUAA | 631 | UUACUCAG CUGAUGAG GCCGUUAGGC CGAA ICCCCUUC | 2820 |
| 409 | AAGGGGCC C UGAGUAAU | 632 | AUUACUCA CUGAUGAG GCCGUUAGGC CGAA IGCCCCUU | 2821 |
| 410 | AGGGGCCC U GAGUAAUU | 633 | AAUUACUC CUGAUGAG GCCGUUAGGC CGAA IGGCCCCU | 2822 |
| 420 | AGUAAUUC A CUCAUUCA | 634 | UGAAUGAG CUGAUGAG GCCGUUAGGC CGAA IAAUUACU | 2823 |
| 422 | UAAUUCAC U CAUUCAGC | 635 | GCUGAAUG CUGAUGAG GCCGUUAGGC CGAA IUGAAUUA | 2824 |
| 424 | AUUCACUC A UUCAGCUG | 636 | CAGCUGAA CUGAUGAG GCCGUUAGGC CGAA IAGUGAAU | 2825 |
| 428 | ACUCAUUC A GCUGAACA | 637 | UGUUCAGC CUGAUGAG GCCGUUAGGC CGAA IAAUGAGU | 2826 |
| 431 | CAUUCAGC U GAACAACA | 638 | UGUUGUUC CUGAUGAG GCCGUUAGGC CGAA ICUGAAUG | 2827 |
| 436 | AGCUGAAC A ACAAUGGC | 639 | GCCAUUGU CUGAUGAG GCCGUUAGGC CGAA IUUCAGCU | 2828 |
| 439 | UGAACAAC A AUGGCUAU | 640 | AUAGCCAU CUGAUGAG GCCGUUAGGC CGAA IUUGUUCA | 2829 |
| 445 | ACAAUGGC U AUGAAGGC | 641 | GCCUUCAU CUGAUGAG GCCGUUAGGC CGAA ICCAUUGU | 2830 |
| 454 | AUGAAGGC A UUGUCGUU | 642 | AACGACAA CUGAUGAG GCCGUUAGGC CGAA ICCUUCAU | 2831 |
| 465 | GUCGUUGC A AUCGACCC | 643 | GGGUCGAU CUGAUGAG GCCGUUAGGC CGAA ICAACGAC | 2832 |
| 472 | CAAUCGAC C CCAAUGUG | 644 | CACAUUGG CUGAUGAG GCCGUUAGGC CGAA IUCGAUUG | 2833 |
| 473 | AAUCGACC C CAAUGUGC | 645 | GCACAUUG CUGAUGAG GCCGUUAGGC CGAA IGUCGAUU | 2834 |
| 474 | AUCGACCC C AAUGUGCC | 646 | GGCACAUU CUGAUGAG GCCGUUAGGC CGAA IGGUCGAU | 2835 |
| 475 | UCGACCCC A AUGUGCCA | 647 | UGGCACAU CUGAUGAG GCCGUUAGGC CGAA IGGGUCGA | 2836 |
| 482 | CAAUGUGC C AGAAGAUG | 648 | CAUCUUCU CUGAUGAG GCCGUUAGGC CGAA ICACAUUG | 2837 |
| 483 | AAUGUGCC A GAAGAUGA | 649 | UCAUCUUC CUGAUGAG GCCGUUAGGC CGAA IGCACAUU | 2838 |
| 495 | GAUGAAAC A CUCAUUCA | 650 | UGAAUGAG CUGAUGAG GCCGUUAGGC CGAA IUUUCAUC | 2839 |
| 497 | UGAAACAC U CAUUCAAC | 651 | GUUGAAUG CUGAUGAG GCCGUUAGGC CGAA IUGUUUCA | 2840 |
| 499 | AAACACUC A UUCAACAA | 652 | UUGUUGAA CUGAUGAG GCCGUUAGGC CGAA IAGUGUUU | 2841 |
| 503 | ACUCAUUC A ACAAAUAA | 653 | UUAUUUGU CUGAUGAG GCCGUUAGGC CGAA IAAUGAGU | 2842 |
| 506 | CAUUCAAC A AAUAAAGG | 654 | CCUUUAUU CUGAUGAG GCCGUUAGGC CGAA IUUGAAUG | 2843 |
| 517 | UAAAGGAC A UGGUGACC | 655 | GGUCACCA CUGAUGAG GCCGUUAGGC CGAA IUCCUUUA | 2844 |
| 525 | AUGGUGAC C CAGGCAUC | 656 | GAUGCCUG CUGAUGAG GCCGUUAGGC CGAA IUCACCAU | 2845 |
| 526 | UGGUGACC C AGGCAUCU | 657 | AGAUGCCU CUGAUGAG GCCGUUAGGC CGAA IGUCACCA | 2846 |
| 527 | GGUGACCC A GGCAUCUC | 658 | GAGAUGCC CUGAUGAG GCCGUUAGGC CGAA IGGUCACC | 2847 |
| 531 | ACCCAGGC A UCUCUGUA | 659 | UACAGAGA CUGAUGAG GCCGUUAGGC CGAA ICCUGGGU | 2848 |
| 534 | CAGGCAUC U CUGUAUCU | 660 | AGAUACAG CUGAUGAG GCCGUUAGGC CGAA IAUGCCUG | 2849 |
| 536 | GGCAUCUC U GUAUCUGU | 661 | ACAGAUAC CUGAUGAG GCCGUUAGGC CGAA IAGAUGCC | 2850 |
| 542 | UCUGUAUC U GUUUGAAG | 662 | CUUCAAAC CUGAUGAG GCCGUUAGGC CGAA IAUACAGA | 2851 |
| 552 | UUUGAAGC U ACAGGAAA | 663 | UUUCCUGU CUGAUGAG GCCGUUAGGC CGAA ICUUCAAA | 2852 |
| 555 | GAAGCUAC A GGAAAGCG | 664 | CGCUUUCC CUGAUGAG GCCGUUAGGC CGAA IUAGCUUC | 2853 |
| 574 | UUUAUUUC A AAAAUGUU | 665 | AACAUUUU CUGAUGAG GCCGUUAGGC CGAA IAAAUAAA | 2854 |
| 585 | AAUGUUGC C AUUUUGAU | 666 | AUCAAAAU CUGAUGAG GCCGUUAGGC CGAA ICAACAUU | 2855 |
| 586 | AUGUUGCC A UUUUGAUU | 667 | AAUCAAAA CUGAUGAG GCCGUUAGGC CGAA IGCAACAU | 2856 |
| 596 | UUUGAUUC C UGAAACAU | 668 | AUGUUUCA CUGAUGAG GCCGUUAGGC CGAA IAAUCAAA | 2857 |
| 597 | UUGAUUCC U GAAACAUG | 669 | CAUGUUUC CUGAUGAG GCCGUUAGGC CGAA IGAAUCAA | 2858 |
| 603 | CCUGAAAC A UGGAAGAC | 670 | GUCUUCCA CUGAUGAG GCCGUUAGGC CGAA IUUUCAGG | 2859 |
| 612 | UGGAAGAC A AAGGCUGA | 671 | UCAGCCUU CUGAUGAG GCCGUUAGGC CGAA IUCUUCCA | 2860 |
| 618 | ACAAAGGC U GACUAUGU | 672 | ACAUAGUC CUGAUGAG GCCGUUAGGC CGAA ICCUUUGU | 2861 |
| 622 | AGGCUGAC U AUGUGAGA | 673 | UCUCACAU CUGAUGAG GCCGUUAGGC CGAA IUCAGCCU | 2862 |
| 632 | UGUGAGAC C AAAACUUG | 674 | CAAGUUUU CUGAUGAG GCCGUUAGGC CGAA IUCUCACA | 2863 |
| 633 | GUGAGACC A AAACUUGA | 675 | UCAAGUUU CUGAUGAG GCCGUUAGGC CGAA IGUCUCAC | 2864 |
| 638 | ACCAAAAC U UGAGACCU | 676 | AGGUCUCA CUGAUGAG GCCGUUAGGC CGAA IUUUUGGU | 2865 |
| 645 | CUUGAGAC C UACAAAAA | 677 | UUUUUGUA CUGAUGAG GCCGUUAGGC CGAA IUCUCAAG | 2866 |
| 646 | UUGAGACC U ACAAAAAU | 678 | AUUUUUGU CUGAUGAG GCCGUUAGGC CGAA IGUCUCAA | 2867 |
| 649 | AGACCUAC A AAAAUGCU | 679 | AGCAUUUU CUGAUGAG GCCGUUAGGC CGAA IUAGGUCU | 2868 |
| 657 | AAAAAUGC U GAUGUUCU | 680 | AGAACAUC CUGAUGAG GCCGUUAGGC CGAA ICAUUUUU | 2869 |
| 665 | UGAUGUUC U GGUUGCUG | 681 | CAGCAACC CUGAUGAG GCCGUUAGGC CGAA IAACAUCA | 2870 |
| 672 | CUGGUUGC U GAGUCUAC | 682 | GUAGACUC CUGAUGAG GCCGUUAGGC CGAA ICAACCAG | 2871 |
| 678 | GCUGAGUC U ACUCCUCC | 683 | GGAGGAGU CUGAUGAG GCCGUUAGGC CGAA IACUCAGC | 2872 |
| 681 | GAGUCUAC U CCUCCAGG | 684 | CCUGGAGG CUGAUGAG GCCGUUAGGC CGAA IUAGACUC | 2873 |
| 683 | GUCUACUC C UCCAGGUA | 685 | UACCUGGA CUGAUGAG GCCGUUAGGC CGAA IAGUAGAC | 2874 |
| 684 | UCUACUCC U CCAGGUAA | 686 | UUACCUGG CUGAUGAG GCCGUUAGGC CGAA IGAGUAGA | 2875 |
| 686 | UACUCCUC C AGGUAAUG | 687 | CAUUACCU CUGAUGAG GCCGUUAGGC CGAA IAGGAGUA | 2876 |
| 687 | ACUCCUCC A GGUAAUGA | 688 | UCAUUACC CUGAUGAG GCCGUUAGGC CGAA IGAGGAGU | 2877 |
| 701 | UGAUGAAC C CUACACUG | 689 | CAGUGUAG CUGAUGAG GCCGUUAGGC CGAA IUUCAUCA | 2878 |
| 702 | GAUGAACC C UACACUGA | 690 | UCAGUGUA CUGAUGAG GCCGUUAGGC CGAA IGUUCAUC | 2879 |
| 703 | AUGAACCC U ACACUGAG | 691 | CUCAGUGU CUGAUGAG GCCGUUAGGC CGAA IGGUUCAU | 2880 |
| 706 | AACCCUAC A CUGAGCAG | 692 | CUGCUCAG CUGAUGAG GCCGUUAGGC CGAA IUAGGGUU | 2881 |
| 708 | CCCUACAC U GAGCAGAU | 693 | AUCUGCUC CUGAUGAG GCCGUUAGGC CGAA IUGUAGGG | 2882 |
| 713 | CACUGAGC A GAUGGGCA | 694 | UGCCCAUC CUGAUGAG GCCGUUAGGC CGAA ICUCAGUG | 2883 |
| 721 | AGAUGGGC A ACUGUGGA | 695 | UCCACAGU CUGAUGAG GCCGUUAGGC CGAA ICCCAUCU | 2884 |
| 724 | UGGGCAAC U GUGGAGAG | 696 | CUCUCCAC CUGAUGAG GCCGUUAGGC CGAA IUUGCCCA | 2885 |
| 748 | AAAGGAUC C ACCUCACU | 697 | AGUGAGGU CUGAUGAG GCCGUUAGGC CGAA IAUCCUUU | 2886 |
| 749 | AAGGAUCC A CCUCACUC | 698 | GAGUGAGG CUGAUGAG GCCGUUAGGC CGAA IGAUCCUU | 2887 |
| 751 | GGAUCCAC C UCACUCCU | 699 | AGGAGUGA CUGAUGAG GCCGUUAGGC CGAA IUGGAUCC | 2888 |
| 752 | GAUCCACC U CACUCCUG | 700 | CAGGAGUG CUGAUGAG GCCGUUAGGC CGAA IGUGGAUC | 2889 |
| 754 | UCCACCUC A CUCCUGAU | 701 | AUCAGGAG CUGAUGAG GCCGUUAGGC CGAA IAGGUGGA | 2890 |
| 756 | CACCUCAC U CCUGAUUU | 702 | AAAUCAGG CUGAUGAG GCCGUUAGGC CGAA IUGAGGUG | 2891 |
| 758 | CCUCACUC C UGAUUUCA | 703 | UGAAAUCA CUGAUGAG GCCGUUAGGC CGAA IAGUGAGG | 2892 |
| 759 | CUCACUCC U GAUUUCAU | 704 | AUGAAAUC CUGAUGAG GCCGUUAGGC CGAA IGAGUGAG | 2893 |
| 766 | CUGAUUUC A UUGCAGGA | 705 | UCCUGCAA CUGAUGAG GCCGUUAGGC CGAA IAAAUCAG | 2894 |
| 771 | UUCAUUGC A GGAAAAAA | 706 | UUUUUUCC CUGAUGAG GCCGUUAGGC CGAA ICAAUGAA | 2895 |
| 786 | AAGUUAGC U GAAUAUGG | 707 | CCAUAUUC CUGAUGAG GCCGUUAGGC CGAA ICUAACUU | 2896 |
| 797 | AUAUGGAC C ACAAGGUA | 708 | UACCUUGU CUGAUGAG GCCGUUAGGC CGAA IUCCAUAU | 2897 |
| 798 | UAUGGACC A CAAGGUAA | 709 | UUACCUUG CUGAUGAG GCCGUUAGGC CGAA IGUCCAUA | 2898 |
| 800 | UGGACCAC A AGGUAAGG | 710 | CCUUACCU CUGAUGAG GCCGUUAGGC CGAA IUGGUCCA | 2899 |
| 810 | GGUAAGGC A UUUGUCCA | 711 | UGGACAAA CUGAUGAG GCCGUUAGGC CGAA ICCUUACC | 2900 |
| 817 | CAUUUGUC C AUGAGUGG | 712 | CCACUCAU CUGAUGAG GCCGUUAGGC CGAA IACAAAUG | 2901 |
| 818 | AUUUGUCC A UGAGUGGG | 713 | CCCACUCA CUGAUGAG GCCGUUAGGC CGAA IGACAAAU | 2902 |
| 828 | GAGUGGGC U CAUCUACG | 714 | CGUAGAUG CUGAUGAG GCCGUUAGGC CGAA ICCCACUC | 2903 |
| 830 | GUGGGCUC A UCUACGAU | 715 | AUCGUAGA CUGAUGAG GCCGUUAGGC CGAA IAGCCCAC | 2904 |
| 833 | GGCUCAUC U ACGAUGGG | 716 | CCCAUCGU CUGAUGAG GCCGUUAGGC CGAA IAUGAGCC | 2905 |
| 859 | ACGAGUAC A AUAAUGAU | 717 | AUCAUUAU CUGAUGAG GCCGUUAGGC CGAA IUACUCGU | 2906 |
| 877 | AGAAAUUC U ACUUAUCC | 718 | GGAUAAGU CUGAUGAG GCCGUUAGGC CGAA IAAUUUCU | 2907 |
| 880 | AAUUCUAC U UAUCCAAU | 719 | AUUGGAUA CUGAUGAG GCCGUUAGGC CGAA IUAGAAUU | 2908 |
| 885 | UACUUAUC C AAUGGAAG | 720 | CUUCCAUU CUGAUGAG GCCGUUAGGC CGAA IAUAAGUA | 2909 |
| 886 | ACUUAUCC A AUGGAAGA | 721 | UCUUCCAU CUGAUGAG GCCGUUAGGC CGAA IGAUAAGU | 2910 |
| 899 | AAGAAUAC A AGCAGUAA | 722 | UUACUGCU CUGAUGAG GCCGUUAGGC CGAA IUAUUCUU | 2911 |
| 903 | AUACAAGC A GUAAGAUG | 723 | CAUCUUAC CUGAUGAG GCCGUUAGGC CGAA ICUUGUAU | 2912 |
| 915 | AGAUGUUC A GCAGGUAU | 724 | AUACCUGC CUGAUGAG GCCGUUAGGC CGAA IAACAUCU | 2913 |
| 918 | UGUUCAGC A GGUAUUAC | 725 | GUAAUACC CUGAUGAG GCCGUUAGGC CGAA ICUGAACA | 2914 |
| 927 | GGUAUUAC U GGUACAAA | 726 | UUUGUACC CUGAUGAG GCCGUUAGGC CGAA IUAAUACC | 2915 |
| 933 | ACUGGUAC A AAUGUAGU | 727 | ACUACAUU CUGAUGAG GCCGUUAGGC CGAA IUACCAGU | 2916 |
| 953 | GAAGUGUC A GGGAGGCA | 728 | UGCCUCCC CUGAUGAG GCCGUUAGGC CGAA IACACUUC | 2917 |
| 961 | AGGGAGGC A GCUGUUAC | 729 | GUAACAGC CUGAUGAG GCCGUUAGGC CGAA ICCUCCCU | 2918 |
| 964 | GAGGCAGC U GUUACACC | 730 | GGUGUAAC CUGAUGAG GCCGUUAGGC CGAA ICUGCCUC | 2919 |
| 970 | GCUGUUAC A CCAAAAGA | 731 | UCUUUUGG CUGAUGAG GCCGUUAGGC CGAA IUAACAGC | 2920 |
| 972 | UGUUACAC C AAAAGAUG | 732 | CAUCUUUU CUGAUGAG GCCGUUAGGC CGAA IUGUAACA | 2921 |
| 973 | GUUACACC A AAAGAUGC | 733 | GCAUCUUU CUGAUGAG GCCGUUAGGC CGAA IGUGUAAC | 2922 |
| 982 | AAAGAUGC A CAUUCAAU | 734 | AUUGAAUG CUGAUGAG GCCGUUAGGC CGAA ICAUCUUU | 2923 |
| 984 | AGAUGCAC A UUCAAUAA | 735 | UUAUUGAA CUGAUGAG GCCGUUAGGC CGAA IUGCAUCU | 2924 |
| 988 | GCACAUUC A AUAAAGUU | 736 | AACUUUAU CUGAUGAG GCCGUUAGGC CGAA IAAUGUGC | 2925 |
| 999 | AAAGUUAC A GGACUCUA | 737 | UAGAGUCC CUGAUGAG GCCGUUAGGC CGAA IUAACUUU | 2926 |
| 1004 | UACAGGAC U CUAUGAAA | 738 | UUUCAUAG CUGAUGAG GCCGUUAGGC CGAA IUCCUGUA | 2927 |
| 1006 | CAGGACUC U AUGAAAAA | 739 | UUUUUCAU CUGAUGAG GCCGUUAGGC CGAA IAGUCCUG | 2928 |
| 1031 | GUUUGUUC U CCAAUCCC | 740 | GGGAUUGG CUGAUGAG GCCGUUAGGC CGAA IAACAAAC | 2929 |
| 1033 | UUGUUCUC C AAUCCCGC | 741 | GCGGGAUU CUGAUGAG GCCGUUAGGC CGAA IAGAACAA | 2930 |
| 1034 | UGUUCUCC A AUCCCGCC | 742 | GGCGGGAU CUGAUGAG GCCGUUAGGC CGAA IGAGAACA | 2931 |
| 1038 | CUCCAAUC C CGCCAGAC | 743 | GUCUGGCG CUGAUGAG GCCGUUAGGC CGAA IAUUGGAG | 2932 |
| 1039 | UCCAAUCC C GCCAGACG | 744 | CGUCUGGC CUGAUGAG GCCGUUAGGC CGAA IGAUUGGA | 2933 |
| 1042 | AAUCCCGC C AGACGGAG | 745 | CUCCGUCU CUGAUGAG GCCGUUAGGC CGAA ICGGGAUU | 2934 |
| 1043 | AUCCCGCC A GACGGAGA | 746 | UCUCCGUC CUGAUGAG GCCGUUAGGC CGAA IGCGGGAU | 2935 |
| 1056 | GAGAAGGC U UCUAUAAU | 747 | AUUAUAGA CUGAUGAG GCCGUUAGGC CGAA ICCUUCUC | 2936 |
| 1059 | AAGGCUUC U AUAAUGUU | 748 | AACAUUAU CUGAUGAG GCCGUUAGGC CGAA IAAGCCUU | 2937 |
| 1071 | AUGUUUGC A CAACAUGU | 749 | ACAUGUUG CUGAUGAG GCCGUUAGGC CGAA ICAAACAU | 2938 |
| 1073 | GUUUGCAC A ACAUGUUG | 750 | CAACAUGU CUGAUGAG GCCGUUAGGC CGAA IUGCAAAC | 2939 |
| 1076 | UGCACAAC A UGUUGAUU | 751 | AAUCAACA CUGAUGAG GCCGUUAGGC CGAA IUUGUGCA | 2940 |
| 1086 | GUUGAUUC U AUAGUUGA | 752 | UCAACUAU CUGAUGAG GCCGUUAGGC CGAA IAAUCAAC | 2941 |
| 1099 | UUGAAUUC U GUACAGAA | 753 | UUCUGUAC CUGAUGAG GCCGUUAGGC CGAA IAAUUCAA | 2942 |
| 1104 | UUCUGUAC A GAACAAAA | 754 | UUUUGUUC CUGAUGAG GCCGUUAGGC CGAA IUACAGAA | 2943 |
| 1109 | UACAGAAC A AAACCACA | 755 | UGUGGUUU CUGAUGAG GCCGUUAGGC CGAA IUUCUGUA | 2944 |
| 1114 | AACAAAAC C ACAACAAA | 756 | UUUGUUGU CUGAUGAG GCCGUUAGGC CGAA IUUUUGUU | 2945 |
| 1115 | ACAAAACC A CAACAAAG | 757 | CUUUGUUG CUGAUGAG GCCGUUAGGC CGAA IGUUUUGU | 2946 |
| 1117 | AAAACCAC A ACAAAGAA | 758 | UUCUUUGU CUGAUGAG GCCGUUAGGC CGAA IUGGUUUU | 2947 |
| 1120 | ACCACAAC A AAGAAGCU | 759 | AGCUUCUU CUGAUGAG GCCGUUAGGC CGAA IUUGUGGU | 2948 |
| 1128 | AAAGAAGC U CCAAACAA | 760 | UUGUUUGG CUGAUGAG GCCGUUAGGC CGAA ICUUCUUU | 2949 |
| 1130 | AGAAGCUC C AAACAAGC | 761 | GCUUGUUU CUGAUGAG GCCGUUAGGC CGAA IAGCUUCU | 2950 |
| 1131 | GAAGCUCC A AACAAGCA | 762 | UGCUUGUU CUGAUGAG GCCGUUAGGC CGAA IGAGCUUC | 2951 |
| 1135 | CUCCAAAC A AGCAAAAU | 763 | AUUUUGCU CUGAUGAG GCCGUUAGGC CGAA IUUUGGAG | 2952 |
| 1139 | AAACAAGC A AAAUCAAA | 764 | UUUGAUUU CUGAUGAG GCCGUUAGGC CGAA ICUUGUUU | 2953 |
| 1145 | GCAAAAUC A AAAAUGCA | 765 | UGCAUUUU CUGAUGAG GCCGUUAGGC CGAA IAUUUUGC | 2954 |
| 1153 | AAAAAUGC A AUCUCCGA | 766 | UCGGAGAU CUGAUGAG GCCGUUAGGC CGAA ICAUUUUU | 2955 |
| 1157 | AUGCAAUC U CCGAAGCA | 767 | UGCUUCGG CUGAUGAG GCCGUUAGGC CGAA IAUUGCAU | 2956 |
| 1159 | GCAAUCUC C GAAGCACA | 768 | UGUGCUUC CUGAUGAG GCCGUUAGGC CGAA IAGAUUGC | 2957 |
| 1165 | UCCGAAGC A CAUGGGAA | 769 | UUCCCAUG CUGAUGAG GCCGUUAGGC CGAA ICUUCGGA | 2958 |
| 1167 | CGAAGCAC A UGGGAAGU | 770 | ACUUCCCA CUGAUGAG GCCGUUAGGC CGAA IUGCUUCG | 2959 |
| 1180 | AAGUGAUC C GUGAUUCU | 771 | AGAAUCAC CUGAUGAG GCCGUUAGGC CGAA IAUCACUU | 2960 |
| 1188 | CGUGAUUC U GAGGACUU | 772 | AAGUCCUC CUGAUGAG GCCGUUAGGC CGAA IAAUCACG | 2961 |
| 1195 | CUGAGGAC U UUAAGAAA | 773 | UUUCUUAA CUGAUGAG GCCGUUAGGC CGAA IUCCUCAG | 2962 |
| 1206 | AAGAAAAC C ACUCCUAU | 774 | AUAGGAGU CUGAUGAG GCCGUUAGGC CGAA IUUUUCUU | 2963 |
| 1207 | AGAAAACC A CUCCUAUG | 775 | CAUAGGAG CUGAUGAG GCCGUUAGGC CGAA IGUUUUCU | 2964 |
| 1209 | AAAACCAC U CCUAUGAC | 776 | GUCAUAGG CUGAUGAG GCCGUUAGGC CGAA IUGGUUUU | 2965 |
| 1211 | AACCACUC C UAUGACAA | 777 | UUGUCAUA CUGAUGAG GCCGUUAGGC CGAA IAGUGGUU | 2966 |
| 1212 | ACCACUCC U AUGACAAC | 778 | GUUGUCAU CUGAUGAG GCCGUUAGGC CGAA IGAGUGGU | 2967 |
| 1218 | CCUAUGAC A ACACAGCC | 779 | GGCUGUGU CUGAUGAG GCCGUUAGGC CGAA IUCAUAGG | 2968 |
| 1221 | AUGACAAC A CAGCCACC | 780 | GGUGGCUG CUGAUGAG GCCGUUAGGC CGAA IUUGUCAU | 2969 |
| 1223 | GACAACAC A GCCACCAA | 781 | UUGGUGGC CUGAUGAG GCCGUUAGGC CGAA IUGUUGUC | 2970 |
| 1226 | AACACAGC C ACCAAAUC | 782 | GAUUUGGU CUGAUGAG GCCGUUAGGC CGAA ICUGUGUU | 2971 |
| 1227 | ACACAGCC A CCAAAUCC | 783 | GGAUUUGG CUGAUGAG GCCGUUAGGC CGAA IGCUGUGU | 2972 |
| 1229 | ACAGCCAC C AAAUCCCA | 784 | UGGGAUUU CUGAUGAG GCCGUUAGGC CGAA IUGGCUGU | 2973 |
| 1230 | CAGCCACC A AAUCCCAC | 785 | GUGGGAUU CUGAUGAG GCCGUUAGGC CGAA IGUGGCUG | 2974 |
| 1235 | ACCAAAUC C CACCUUCU | 786 | AGAAGGUG CUGAUGAG GCCGUUAGGC CGAA IAUUUGGU | 2975 |
| 1236 | CCAAAUCC C ACCUUCUC | 787 | GAGAAGGU CUGAUGAG GCCGUUAGGC CGAA IGAUUUGG | 2976 |
| 1237 | CAAAUCCC A CCUUCUCA | 788 | UGAGAAGG CUGAUGAG GCCGUUAGGC CGAA IGGAUUUG | 2977 |
| 1239 | AAUCCCAC C UUCUCAUU | 789 | AAUGAGAA CUGAUGAG GCCGUUAGGC CGAA IUGGGAUU | 2978 |
| 1240 | AUCCCACC U UCUCAUUG | 790 | CAAUGAGA CUGAUGAG GCCGUUAGGC CGAA IGUGGGAU | 2979 |
| 1243 | CCACCUUC U CAUUGCUG | 791 | CAGCAAUG CUGAUGAG GCCGUUAGGC CGAA IAAGGUGG | 2980 |
| 1245 | ACCUUCUC A UUGCUGCA | 792 | UGCAGCAA CUGAUGAG GCCGUUAGGC CGAA IAGAAGGU | 2981 |
| 1250 | CUCAUUGC U GCAGAUUG | 793 | CAAUCUGC CUGAUGAG GCCGUUAGGC CGAA ICAAUGAG | 2982 |
| 1253 | AUUGCUGC A GAUUGGAC | 794 | GUCCAAUC CUGAUGAG GCCGUUAGGC CGAA ICAGCAAU | 2983 |
| 1262 | GAUUGGAC A AAGAAUUG | 795 | CAAUUCUU CUGAUGAG GCCGUUAGGC CGAA IUCCAAUC | 2984 |
| 1282 | GUUUAGUC C UUGACAAA | 796 | UUUGUCAA CUGAUGAG GCCGUUAGGC CGAA IACUAAAC | 2985 |
| 1283 | UUUAGUCC U UGACAAAU | 797 | AUUUGUCA CUGAUGAG GCCGUUAGGC CGAA IGACUAAA | 2986 |
| 1288 | UCCUUGAC A AAUCUGGA | 798 | UCCAGAUU CUGAUGAG GCCGUUAGGC CGAA IUCAAGGA | 2987 |
| 1293 | GACAAAUC U GGAAGCAU | 799 | AUGCUUCC CUGAUGAG GCCGUUAGGC CGAA IAUUUGUC | 2988 |
| 1300 | CUGGAAGC A UGGCGACU | 800 | AGUCGCCA CUGAUGAG GCCGUUAGGC CGAA ICUUCCAG | 2989 |
| 1308 | AUGGCGAC U GGUAACCG | 801 | CGGUUACC CUGAUGAG GCCGUUAGGC CGAA IUCGCCAU | 2990 |
| 1315 | CUGGUAAC C GCCUCAAU | 802 | AUUGAGGC CUGAUGAG GCCGUUAGGC CGAA IUUACCAG | 2991 |
| 1318 | GUAACCGC C UCAAUCGA | 803 | UCGAUUGA CUGAUGAG GCCGUUAGGC CGAA ICGGUUAC | 2992 |
| 1319 | UAACCGCC U CAAUCGAC | 804 | GUCGAUUG CUGAUGAG GCCGUUAGGC CGAA IGCGGUUA | 2993 |
| 1321 | ACCGCCUC A AUCGACUG | 805 | CAGUCGAU CUGAUGAG GCCGUUAGGC CGAA IAGGCGGU | 2994 |
| 1328 | CAAUCGAC U GAAUCAAG | 806 | CUUGAUUC CUGAUGAG GCCGUUAGGC CGAA IUCGAUUG | 2995 |
| 1334 | ACUGAAUC A AGCAGGCC | 807 | GGCCUGCU CUGAUGAG GCCGUUAGGC CGAA IAUUCAGU | 2996 |
| 1338 | AAUCAAGC A GGCCAGCU | 808 | AGCUGGCC CUGAUGAG GCCGUUAGGC CGAA ICUUGAUU | 2997 |
| 1342 | AAGCAGGC C AGCUUUUC | 809 | GAAAAGCU CUGAUGAG GCCGUUAGGC CGAA ICCUGCUU | 2998 |
| 1343 | AGCAGGCC A GCUUUUCC | 810 | GGAAAAGC CUGAUGAG GCCGUUAGGC CGAA IGCCUGCU | 2999 |
| 1346 | AGGCCAGC U UUUCCUGC | 811 | GCAGGAAA CUGAUGAG GCCGUUAGGC CGAA ICUGGCCU | 3000 |
| 1351 | AGCUUUUC C UGCUGCAG | 812 | CUGCAGCA CUGAUGAG GCCGUUAGGC CGAA IAAAAGCU | 3001 |
| 1352 | GCUUUUCC U GCUGCAGA | 813 | UCUGCAGC CUGAUGAG GCCGUUAGGC CGAA IGAAAAGC | 3002 |
| 1355 | UUUCCUGC U GCAGACAG | 814 | CUGUCUGC CUGAUGAG GCCGUUAGGC CGAA ICAGGAAA | 3003 |
| 1358 | CCUGCUGC A GACAGUUG | 815 | CAACUGUC CUGAUGAG GCCGUUAGGC CGAA ICAGCAGG | 3004 |
| 1362 | CUGCAGAC A GUUGAGCU | 816 | AGCUCAAC CUGAUGAG GCCGUUAGGC CGAA IUCUGCAG | 3005 |
| 1370 | AGUUGAGC U GGGGUCCU | 817 | AGGACCCC CUGAUGAG GCCGUUAGGC CGAA ICUCAACU | 3006 |
| 1377 | CUGGGGUC C UGGGUUGG | 818 | CCAACCCA CUGAUGAG GCCGUUAGGC CGAA IACCCCAG | 3007 |
| 1378 | UGGGGUCC U GGGUUGGG | 819 | CCCAACCC CUGAUGAG GCCGUUAGGC CGAA IGACCCCA | 3008 |
| 1395 | AUGGUGAC A UUUGACAG | 820 | CUGUCAAA CUGAUGAG GCCGUUAGGC CGAA IUCACCAU | 3009 |
| 1402 | CAUUUGAC A GUGCUGCC | 821 | GGCAGCAC CUGAUGAG GCCGUUAGGC CGAA IUCAAAUG | 3010 |
| 1407 | GACAGUGC U GCCCAUGU | 822 | ACAUGGGC CUGAUGAG GCCGUUAGGC CGAA ICACUGUC | 3011 |
| 1410 | AGUGCUGC C CAUGUACA | 823 | UGUACAUG CUGAUGAG GCCGUUAGGC CGAA ICAGCACU | 3012 |
| 1411 | GUGCUGCC C AUGUACAA | 824 | UUGUACAU CUGAUGAG GCCGUUAGGC CGAA IGCAGCAC | 3013 |
| 1412 | UGCUGCCC A UGUACAAA | 825 | UUUGUACA CUGAUGAG GCCGUUAGGC CGAA IGGCAGCA | 3014 |
| 1418 | CCAUGUAC A AAGUGAAC | 826 | GUUCACUU CUGAUGAG GCCGUUAGGC CGAA IUACAUGG | 3015 |
| 1427 | AAGUGAAC U CAUACAGA | 827 | UCUGUAUG CUGAUGAG GCCGUUAGGC CGAA IUUCACUU | 3016 |
| 1429 | GUGAACUC A UACAGAUA | 828 | UAUCUGUA CUGAUGAG GCCGUUAGGC CGAA IAGUUCAC | 3017 |
| 1433 | ACUCAUAC A GAUAAACA | 829 | UGUUUAUC CUGAUGAG GCCGUUAGGC CGAA IUAUGAGU | 3018 |
| 1441 | AGAUAAAC A GUGGCAGU | 830 | ACUGCCAC CUGAUGAG GCCGUUAGGC CGAA IUUUAUCU | 3019 |
| 1447 | ACAGUGGC A GUGACAGG | 831 | CCUGUCAC CUGAUGAG GCCGUUAGGC CGAA ICCACUGU | 3020 |
| 1453 | GCAGUGAC A GGGACACA | 832 | UGUGUCCC CUGAUGAG GCCGUUAGGC CGAA IUCACUGC | 3021 |
| 1459 | ACAGGGAC A CACUCGCC | 833 | GGCGAGUG CUGAUGAG GCCGUUAGGC CGAA IUCCCUGU | 3022 |
| 1461 | AGGGACAC A CUCGCCAA | 834 | UUGGCGAG CUGAUGAG GCCGUUAGGC CGAA IUGUCCCU | 3023 |
| 1463 | GGACACAC U CGCCAAAA | 835 | UUUUGGCG CUGAUGAG GCCGUUAGGC CGAA IUGUGUCC | 3024 |
| 1467 | ACACUCGC C AAAAGAUU | 836 | AAUCUUUU CUGAUGAG GCCGUUAGGC CGAA ICGAGUGU | 3025 |
| 1468 | CACUCGCC A AAAGAUUA | 837 | UAAUCUUU CUGAUGAG GCCGUUAGGC CGAA IGCGAGUG | 3026 |
| 1478 | AAGAUUAC C UGCAGCAG | 838 | CUGCUGCA CUGAUGAG GCCGUUAGGC CGAA IUAAUCUU | 3027 |
| 1479 | AGAUUACC U GCAGCAGC | 839 | GCUGCUGC CUGAUGAG GCCGUUAGGC CGAA IGUAAUCU | 3028 |
| 1482 | UUACCUGC A GCAGCUUC | 840 | GAAGCUGC CUGAUGAG GCCGUUAGGC CGAA ICAGGUAA | 3029 |
| 1485 | CCUGCAGC A GCUUCAGG | 841 | CCUGAAGC CUGAUGAG GCCGUUAGGC CGAA ICUGCAGG | 3030 |
| 1488 | GCAGCAGC U UCAGGAGG | 842 | CCUCCUGA CUGAUGAG GCCGUUAGGC CGAA ICUGCUGC | 3031 |
| 1491 | GCAGCUUC A GGAGGGAC | 843 | GUCCCUCC CUGAUGAG GCCGUUAGGC CGAA IAAGCUGC | 3032 |
| 1503 | GGGACGUC C AUCUGCAG | 844 | CUGCAGAU CUGAUGAG GCCGUUAGGC CGAA IACGUCCC | 3033 |
| 1504 | GGACGUCC A UCUGCAGC | 845 | GCUGCAGA CUGAUGAG GCCGUUAGGC CGAA IGACGUCC | 3034 |
| 1507 | CGUCCAUC U GCAGCGGG | 846 | CCCGCUGC CUGAUGAG GCCGUUAGGC CGAA IAUGGACG | 3035 |
| 1510 | CCAUCUGC A GCGGGCUU | 847 | AAGCCCGC CUGAUGAG GCCGUUAGGC CGAA ICAGAUGG | 3036 |
| 1517 | CAGCGGGC U UCGAUCGG | 848 | CCGAUCGA CUGAUGAG GCCGUUAGGC CGAA ICCCGCUG | 3037 |
| 1527 | CGAUCGGC A UUUACUGU | 849 | ACAGUAAA CUGAUGAG GCCGUUAGGC CGAA ICCGAUCG | 3038 |
| 1533 | GCAUUUAC U GUGAUUAG | 850 | CUAAUCAC CUGAUGAG GCCGUUAGGC CGAA IUAAAUGC | 3039 |
| 1553 | GAAAUAUC C AACUGAUG | 851 | CAUCAGUU CUGAUGAG GCCGUUAGGC CGAA IAUAUUUC | 3040 |
| 1554 | AAAUAUCC A ACUGAUGG | 852 | CCAUCAGU CUGAUGAG GCCGUUAGGC CGAA IGAUAUUU | 3041 |
| 1557 | UAUCCAAC U GAUGGAUC | 853 | GAUCCAUC CUGAUGAG GCCGUUAGGC CGAA IUUGGAUA | 3042 |
| 1566 | GAUGGAUC U GAAAUUGU | 854 | ACAAUUUC CUGAUGAG GCCGUUAGGC CGAA IAUCCAUC | 3043 |
| 1577 | AAUUGUGC U GCUGACGG | 855 | CCGUCAGC CUGAUGAG GCCGUUAGGC CGAA ICACAAUU | 3044 |
| 1580 | UGUGCUGC U GACGGAUG | 856 | CAUCCGUC CUGAUGAG GCCGUUAGGC CGAA ICAGCACA | 3045 |
| 1597 | GGGAAGAC A ACACUAUA | 857 | UAUAGUGU CUGAUGAG GCCGUUAGGC CGAA IUCUUCCC | 3046 |
| 1600 | AAGACAAC A CUAUAAGU | 858 | ACUUAUAG CUGAUGAG GCCGUUAGGC CGAA IUUGUCUU | 3047 |
| 1602 | GACAACAC U AUAAGUGG | 859 | CCACUUAU CUGAUGAG GCCGUUAGGC CGAA IUGUUGUC | 3048 |
| 1615 | GUGGGUGC U UUAACGAG | 860 | CUCGUUAA CUGAUGAG GCCGUUAGGC CGAA ICACCCAC | 3049 |
| 1627 | ACGAGGUC A AACAAAGU | 861 | ACUUUGUU CUGAUGAG GCCGUUAGGC CGAA IACCUCGU | 3050 |
| 1631 | GGUCAAAC A AAGUGGUG | 862 | CACCACUU CUGAUGAG GCCGUUAGGC CGAA IUUUGACC | 3051 |
| 1641 | AGUGGUGC C AUCAUCCA | 863 | UGGAUGAU CUGAUGAG GCCGUUAGGC CGAA ICACCACU | 3052 |
| 1642 | GUGGUGCC A UCAUCCAC | 864 | GUGGAUGA CUGAUGAG GCCGUUAGGC CGAA IGCACCAC | 3053 |
| 1645 | GUGCCAUC A UCCACACA | 865 | UGUGUGGA CUGAUGAG GCCGUUAGGC CGAA IAUGGCAC | 3054 |
| 1648 | CCAUCAUC C ACACAGUC | 866 | GACUGUGU CUGAUGAG GCCGUUAGGC CGAA IAUGAUGG | 3055 |
| 1649 | CAUCAUCC A CACAGUCG | 867 | CGACUGUG CUGAUGAG GCCGUUAGGC CGAA IGAUGAUG | 3056 |
| 1651 | UCAUCCAC A CAGUCGCU | 868 | AGCGACUG CUGAUGAG GCCGUUAGGC CGAA IUGGAUGA | 3057 |
| 1653 | AUCCACAC A GUCGCUUU | 869 | AAAGCGAC CUGAUGAG GCCGUUAGGC CGAA IUGUGGAU | 3058 |
| 1659 | ACAGUCGC U UUGGGGCC | 870 | GGCCCCAA CUGAUGAG GCCGUUAGGC CGAA ICGACUGU | 3059 |
| 1667 | UUUGGGGC C CUCUGCAG | 871 | CUGCAGAG CUGAUGAG GCCGUUAGGC CGAA ICCCCAAA | 3060 |
| 1668 | UUGGGGCC C UCUGCAGC | 872 | GCUGCAGA CUGAUGAG GCCGUUAGGC CGAA IGCCCCAA | 3061 |
| 1669 | UGGGGCCC U CUGCAGCU | 873 | AGCUGCAG CUGAUGAG GCCGUUAGGC CGAA IGGCCCCA | 3062 |
| 1671 | GGGCCCUC U GCAGCUCA | 874 | UGAGCUGC CUGAUGAG GCCGUUAGGC CGAA IAGGGCCC | 3063 |
| 1674 | CCCUCUGC A GCUCAAGA | 875 | UCUUGAGC CUGAUGAG GCCGUUAGGC CGAA ICAGAGGG | 3064 |
| 1677 | UCUGCAGC U CAAGAACU | 876 | AGUUCUUG CUGAUGAG GCCGUUAGGC CGAA ICUGCAGA | 3065 |
| 1679 | UGCAGCUC A AGAACUAG | 877 | CUAGUUCU CUGAUGAG GCCGUUAGGC CGAA IAGCUGCA | 3066 |
| 1685 | UCAAGAAC U AGAGGAGC | 878 | GCUCCUCU CUGAUGAG GCCGUUAGGC CGAA IUUCUUGA | 3067 |
| 1694 | AGAGGAGC U GUCCAAAA | 879 | UUUUGGAC CUGAUGAG GCCGUUAGGC CGAA ICUCCUCU | 3068 |
| 1698 | GAGCUGUC C AAAAUGAC | 880 | GUCAUUUU CUGAUGAG GCCGUUAGGC CGAA IACAGCUC | 3069 |
| 1699 | AGCUGUCC A AAAUGACA | 881 | UGUCAUUU CUGAUGAG GCCGUUAGGC CGAA IGACAGCU | 3070 |
| 1707 | AAAAUGAC A GGAGGUUU | 882 | AAACCUCC CUGAUGAG GCCGUUAGGC CGAA IUCAUUUU | 3071 |
| 1718 | AGGUUUAC A GACAUAUG | 883 | CAUAUGUC CUGAUGAG GCCGUUAGGC CGAA IUAAACCU | 3072 |
| 1722 | UUACAGAC A UAUGCUUC | 884 | GAAGCAUA CUGAUGAG GCCGUUAGGC CGAA IUCUGUAA | 3073 |
| 1728 | ACAUAUGC U UCAGAUCA | 885 | UGAUCUGA CUGAUGAG GCCGUUAGGC CGAA ICAUAUGU | 3074 |
| 1731 | UAUGCUUC A GAUCAAGU | 886 | ACUUGAUC CUGAUGAG GCCGUUAGGC CGAA IAAGCAUA | 3075 |
| 1736 | UUCAGAUC A AGUUCAGA | 887 | UCUGAACU CUGAUGAG GCCGUUAGGC CGAA IAUCUGAA | 3076 |
| 1742 | UCAAGUUC A GAACAAUG | 888 | CAUUGUUC CUGAUGAG GCCGUUAGGC CGAA IAACUUGA | 3077 |
| 1747 | UUCAGAAC A AUGGCCUC | 889 | GAGGCCAU CUGAUGAG GCCGUUAGGC CGAA IUUCUGAA | 3078 |
| 1753 | ACAAUGGC C UCAUUGAU | 890 | AUCAAUGA CUGAUGAG GCCGUUAGGC CGAA ICCAUUGU | 3079 |
| 1754 | CAAUGGCC U CAUUGAUG | 891 | CAUCAAUG CUGAUGAG GCCGUUAGGC CGAA IGCCAUUG | 3080 |
| 1756 | AUGGCCUC A UUGAUGCU | 892 | AGCAUCAA CUGAUGAG GCCGUUAGGC CGAA IAGGCCAU | 3081 |
| 1764 | AUUGAUGC U UUUGGGGC | 893 | GCCCCAAA CUGAUGAG GCCGUUAGGC CGAA ICAUCAAU | 3082 |
| 1773 | UUUGGGGC C CUUUCAUC | 894 | GAUGAAAG CUGAUGAG GCCGUUAGGC CGAA ICCCCAAA | 3083 |
| 1774 | UUGGGGCC C UUUCAUCA | 895 | UGAUGAAA CUGAUGAG GCCGUUAGGC CGAA IGCCCCAA | 3084 |
| 1775 | UGGGGCCC U UUCAUCAG | 896 | CUGAUGAA CUGAUGAG GCCGUUAGGC CGAA IGGCCCCA | 3085 |
| 1779 | GCCCUUUC A UCAGGAAA | 897 | UUUCCUGA CUGAUGAG GCCGUUAGGC CGAA IAAAGGGC | 3086 |
| 1782 | CUUUCAUC A GGAAAUGG | 898 | CCAUUUCC CUGAUGAG GCCGUUAGGC CGAA IAUGAAAG | 3087 |
| 1794 | AAUGGAGC U GUCUCUCA | 899 | UGAGAGAC CUGAUGAG GCCGUUAGGC CGAA ICUCCAUU | 3088 |
| 1798 | GAGCUGUC U CUCAGCGC | 900 | GCGCUGAG CUGAUGAG GCCGUUAGGC CGAA IACAGCUC | 3089 |
| 1800 | GCUGUCUC U CAGCGCUC | 901 | GAGCGCUG CUGAUGAG GCCGUUAGGC CGAA IAGACAGC | 3090 |
| 1802 | UGUCUCUC A GCGCUCCA | 902 | UGGAGCGC CUGAUGAG GCCGUUAGGC CGAA IAGAGACA | 3091 |
| 1807 | CUCAGCGC U CCAUCCAG | 903 | CUGGAUGG CUGAUGAG GCCGUUAGGC CGAA ICGCUGAG | 3092 |
| 1809 | CAGCGCUC C AUCCAGCU | 904 | AGCUGGAU CUGAUGAG GCCGUUAGGC CGAA IAGCGCUG | 3093 |
| 1810 | AGCGCUCC A UCCAGCUU | 905 | AAGCUGGA CUGAUGAG GCCGUUAGGC CGAA IGAGCGCU | 3094 |
| 1813 | GCUCCAUC C AGCUUGAG | 906 | CUCAAGCU CUGAUGAG GCCGUUAGGC CGAA IAUGGAGC | 3095 |
| 1814 | CUCCAUCC A GCUUGAGA | 907 | UCUCAAGC CUGAUGAG GCCGUUAGGC CGAA IGAUGGAG | 3096 |
| 1817 | CAUCCAGC U UGAGAGUA | 908 | UACUCUCA CUGAUGAG GCCGUUAGGC CGAA ICUGGAUG | 3097 |
| 1836 | GGAUUAAC C CUCCAGAA | 909 | UUCUGGAG CUGAUGAG GCCGUUAGGC CGAA IUUAAUCC | 3098 |
| 1837 | GAUUAACC C UCCAGAAC | 910 | GUUCUGGA CUGAUGAG GCCGUUAGGC CGAA IGUUAAUC | 3099 |
| 1838 | AUUAACCC U CCAGAACA | 911 | UGUUCUGG CUGAUGAG GCCGUUAGGC CGAA IGGUUAAU | 3100 |
| 1840 | UAACCCUC C AGAACAGC | 912 | GCUGUUCU CUGAUGAG GCCGUUAGGC CGAA IAGGGUUA | 3101 |
| 1841 | AACCCUCC A GAACAGCC | 913 | GGCUGUUC CUGAUGAG GCCGUUAGGC CGAA IGAGGGUU | 3102 |
| 1846 | UCCAGAAC A GCCAGUGG | 914 | CCACUGGC CUGAUGAG GCCGUUAGGC CGAA IUUCUGGA | 3103 |
| 1849 | AGAACAGC C AGUGGAUG | 915 | CAUCCACU CUGAUGAG GCCGUUAGGC CGAA ICUGUUCU | 3104 |
| 1850 | GAACAGCC A GUGGAUGA | 916 | UCAUCCAC CUGAUGAG GCCGUUAGGC CGAA IGCUGUUC | 3105 |
| 1864 | UGAAUGGC A CAGUGAUC | 917 | GAUCACUG CUGAUGAG GCCGUUAGGC CGAA ICCAUUCA | 3106 |
| 1866 | AAUGGCAC A GUGAUCGU | 918 | ACGAUCAC CUGAUGAG GCCGUUAGGC CGAA IUGCCAUU | 3107 |
| 1879 | UCGUGGAC A GCACCGUG | 919 | CACGGUGC CUGAUGAG GCCGUUAGGC CGAA IUCCACGA | 3108 |
| 1882 | UGGACAGC A CCGUGGGA | 920 | UCCCACGG CUGAUGAG GCCGUUAGGC CGAA ICUGUCCA | 3109 |
| 1884 | GACAGCAC C GUGGGAAA | 921 | UUUCCCAC CUGAUGAG GCCGUUAGGC CGAA IUGCUGUC | 3110 |
| 1897 | GAAAGGAC A CUUUGUUU | 922 | AAACAAAG CUGAUGAG GCCGUUAGGC CGAA IUCCUUUC | 3111 |
| 1899 | AAGGACAC U UUGUUUCU | 923 | AGAAACAA CUGAUGAG GCCGUUAGGC CGAA IUGUCCUU | 3112 |
| 1907 | UUUGUUUC U UAUCACCU | 924 | AGGUGAUA CUGAUGAG GCCGUUAGGC CGAA IAAACAAA | 3113 |
| 1912 | UUCUUAUC A CCUGGACA | 925 | UGUCCAGG CUGAUGAG GCCGUUAGGC CGAA IAUAAGAA | 3114 |
| 1914 | CUUAUCAC C UGGACAAC | 926 | GUUGUCCA CUGAUGAG GCCGUUAGGC CGAA IUGAUAAG | 3115 |
| 1915 | UUAUCACC U GGACAACG | 927 | CGUUGUCC CUGAUGAG GCCGUUAGGC CGAA IGUGAUAA | 3116 |
| 1920 | ACCUGGAC A ACGCAGCC | 928 | GGCUGCGU CUGAUGAG GCCGUUAGGC CGAA IUCCAGGU | 3117 |
| 1925 | GACAACGC A GCCUCCCC | 929 | GGGGAGGC CUGAUGAG GCCGUUAGGC CGAA ICGUUGUC | 3118 |
| 1928 | AACGCAGC C UCCCCAAA | 930 | UUUGGGGA CUGAUGAG GCCGUUAGGC CGAA ICUGCGUU | 3119 |
| 1929 | ACGCAGCC U CCCCAAAU | 931 | AUUUGGGG CUGAUGAG GCCGUUAGGC CGAA IGCUGCGU | 3120 |
| 1931 | GCAGCCUC C CCAAAUCC | 932 | GGAUUUGG CUGAUGAG GCCGUUAGGC CGAA IAGGCUGC | 3121 |
| 1932 | CAGCCUCC C CAAAUCCU | 933 | AGGAUUUG CUGAUGAG GCCGUUAGGC CGAA IGAGGCUG | 3122 |
| 1933 | AGCCUCCC C AAAUCCUU | 934 | AAGGAUUU CUGAUGAG GCCGUUAGGC CGAA IGGAGGCU | 3123 |
| 1934 | GCCUCCCC A AAUCCUUC | 935 | GAAGGAUU CUGAUGAG GCCGUUAGGC CGAA IGGGAGGC | 3124 |
| 1939 | CCCAAAUC C UUCUCUGG | 936 | CCAGAGAA CUGAUGAG GCCGUUAGGC CGAA IAUUUGGG | 3125 |
| 1940 | CCAAAUCC U UCUCUGGG | 937 | CCCAGAGA CUGAUGAG GCCGUUAGGC CGAA IGAUUUGG | 3126 |
| 1943 | AAUCCUUC U CUGGGAUC | 938 | GAUCCCAG CUGAUGAG GCCGUUAGGC CGAA IAAGGAUU | 3127 |
| 1945 | UCCUUCUC U GGGAUCCC | 939 | GGGAUCCC CUGAUGAG GCCGUUAGGC CGAA IAGAAGGA | 3128 |
| 1952 | CUGGGAUC C CAGUGGAC | 940 | GUCCACUG CUGAUGAG GCCGUUAGGC CGAA IAUCCCAG | 3129 |
| 1953 | UGGGAUCC C AGUGGACA | 941 | UGUCCACU CUGAUGAG GCCGUUAGGC CGAA IGAUCCCA | 3130 |
| 1954 | GGGAUCCC A GUGGACAG | 942 | CUGUCCAC CUGAUGAG GCCGUUAGGC CGAA IGGAUCCC | 3131 |
| 1961 | CAGUGGAC A GAAGCAAG | 943 | CUUGCUUC CUGAUGAG GCCGUUAGGC CGAA IUCCACUG | 3132 |
| 1967 | ACAGAAGC A AGGUGGCU | 944 | AGCCACCU CUGAUGAG GCCGUUAGGC CGAA ICUUCUGU | 3133 |
| 1975 | AAGGUGGC U UUGUAGUG | 945 | CACUACAA CUGAUGAG GCCGUUAGGC CGAA ICCACCUU | 3134 |
| 1987 | UAGUGGAC A AAAACACC | 946 | GGUGUUUU CUGAUGAG GCCGUUAGGC CGAA IUCCACUA | 3135 |
| 1993 | ACAAAAAC A CCAAAAUG | 947 | CAUUUUGG CUGAUGAG GCCGUUAGGC CGAA IUUUUUGU | 3136 |
| 1995 | AAAAACAC C AAAAUGGC | 948 | GCCAUUUU CUGAUGAG GCCGUUAGGC CGAA IUGUUUUU | 3137 |
| 1996 | AAAACACC A AAAUGGCC | 949 | GGCCAUUU CUGAUGAG GCCGUUAGGC CGAA IGUGUUUU | 3138 |
| 2004 | AAAAUGGC C UACCUCCA | 950 | UGGAGGUA CUGAUGAG GCCGUUAGGC CGAA ICCAUUUU | 3139 |
| 2005 | AAAUGGCC U ACCUCCAA | 951 | UUGGAGGU CUGAUGAG GCCGUUAGGC CGAA IGCCAUUU | 3140 |
| 2008 | UGGCCUAC C UCCAAAUC | 952 | GAUUUGGA CUGAUGAG GCCGUUAGGC CGAA IUAGGCCA | 3141 |
| 2009 | GGCCUACC U CCAAAUCC | 953 | GGAUUUGG CUGAUGAG GCCGUUAGGC CGAA IGUAGGCC | 3142 |
| 2011 | CCUACCUC C AAAUCCCA | 954 | UGGGAUUU CUGAUGAG GCCGUUAGGC CGAA IAGGUAGG | 3143 |
| 2012 | CUACCUCC A AAUCCCAG | 955 | CUGGGAUU CUGAUGAG GCCGUUAGGC CGAA IGAGGUAG | 3144 |
| 2017 | UCCAAAUC C CAGGCAUU | 956 | AAUGCCUG CUGAUGAG GCCGUUAGGC CGAA IAUUUGGA | 3145 |
| 2018 | CCAAAUCC C AGGCAUUG | 957 | CAAUGCCU CUGAUGAG GCCGUUAGGC CGAA IGAUUUGG | 3146 |
| 2019 | CAAAUCCC A GGCAUUGC | 958 | GCAAUGCC CUGAUGAG GCCGUUAGGC CGAA IGGAUUUG | 3147 |
| 2023 | UCCCAGGC A UUGCUAAG | 959 | CUUAGCAA CUGAUGAG GCCGUUAGGC CGAA ICCUGGGA | 3148 |
| 2028 | GGCAUUGC U AAGGUUGG | 960 | CCAACCUU CUGAUGAG GCCGUUAGGC CGAA ICAAUGCC | 3149 |
| 2038 | AGGUUGGC A CUUGGAAA | 961 | UUUCCAAG CUGAUGAG GCCGUUAGGC CGAA ICCAACCU | 3150 |
| 2040 | GUUGGCAC U UGGAAAUA | 962 | UAUUUCCA CUGAUGAG GCCGUUAGGC CGAA IUGCCAAC | 3151 |
| 2050 | GGAAAUAC A GUCUGCAA | 963 | UUGCAGAC CUGAUGAG GCCGUUAGGC CGAA IUAUUUCC | 3152 |
| 2054 | AUACAGUC U GCAAGCAA | 964 | UUGCUUGC CUGAUGAG GCCGUUAGGC CGAA IACUGUAU | 3153 |
| 2057 | CAGUCUGC A AGCAAGCU | 965 | AGCUUGCU CUGAUGAG GCCGUUAGGC CGAA ICAGACUG | 3154 |
| 2061 | CUGCAAGC A AGCUCACA | 966 | UGUGAGCU CUGAUGAG GCCGUUAGGC CGAA ICUUGCAG | 3155 |
| 2065 | AAGCAAGC U CACAAACC | 967 | GGUUUGUG CUGAUGAG GCCGUUAGGC CGAA ICUUGCUU | 3156 |
| 2067 | GCAAGCUC A CAAACCUU | 968 | AAGGUUUG CUGAUGAG GCCGUUAGGC CGAA IAGCUUGC | 3157 |
| 2069 | AAGCUCAC A AACCUUGA | 969 | UCAAGGUU CUGAUGAG GCCGUUAGGC CGAA IUGAGCUU | 3158 |
| 2073 | UCACAAAC C UUGACCCU | 970 | AGGGUCAA CUGAUGAG GCCGUUAGGC CGAA IUUUGUGA | 3159 |
| 2074 | CACAAACC U UGACCCUG | 971 | CAGGGUCA CUGAUGAG GCCGUUAGGC CGAA IGUUUGUG | 3160 |
| 2079 | ACCUUGAC C CUGACUGU | 972 | ACAGUCAG CUGAUGAG GCCGUUAGGC CGAA IUCAAGGU | 3161 |
| 2080 | CCUUGACC C UGACUGUC | 973 | GACAGUCA CUGAUGAG GCCGUUAGGC CGAA IGUCAAGG | 3162 |
| 2081 | CUUGACCC U GACUGUCA | 974 | UGACAGUC CUGAUGAG GCCGUUAGGC CGAA IGGUCAAG | 3163 |
| 2085 | ACCCUGAC U GUCACGUC | 975 | GACGUGAC CUGAUGAG GCCGUUAGGC CGAA IUCAGGGU | 3164 |
| 2089 | UGACUGUC A CGUCCCGU | 976 | ACGGGACG CUGAUGAG GCCGUUAGGC CGAA IACAGUCA | 3165 |
| 2094 | GUCACGUC C CGUGCGUC | 977 | GACGCACG CUGAUGAG GCCGUUAGGC CGAA IACGUGAC | 3166 |
| 2095 | UCACGUCC C GUGCGUCC | 978 | GGACGCAC CUGAUGAG GCCGUUAGGC CGAA IGACGUGA | 3167 |
| 2103 | CGUGCGUC C AAUGCUAC | 979 | GUAGCAUU CUGAUGAG GCCGUUAGGC CGAA IACGCACG | 3168 |
| 2104 | GUGCGUCC A AUGCUACC | 980 | GGUAGCAU CUGAUGAG GCCGUUAGGC CGAA IGACGCAC | 3169 |
| 2109 | UCCAAUGC U ACCCUGCC | 981 | GGCAGGGU CUGAUGAG GCCGUUAGGC CGAA ICAUUGGA | 3170 |
| 2112 | AAUGCUAC C CUGCCUCC | 982 | GGAGGCAG CUGAUGAG GCCGUUAGGC CGAA IUAGCAUU | 3171 |
| 2113 | AUGCUACC C UGCCUCCA | 983 | UGGAGGCA CUGAUGAG GCCGUUAGGC CGAA IGUAGCAU | 3172 |
| 2114 | UGCUACCC U GCCUCCAA | 984 | UUGGAGGC CUGAUGAG GCCGUUAGGC CGAA IGGUAGCA | 3173 |
| 2117 | UACCCUGC C UCCAAUUA | 985 | UAAUUGGA CUGAUGAG GCCGUUAGGC CGAA ICAGGGUA | 3174 |
| 2118 | ACCCUGCC U CCAAUUAC | 986 | GUAAUUGG CUGAUGAG GCCGUUAGGC CGAA IGCAGGGU | 3175 |
| 2120 | CCUGCCUC C AAUUACAG | 987 | CUGUAAUU CUGAUGAG GCCGUUAGGC CGAA IAGGCAGG | 3176 |
| 2121 | CUGCCUCC A AUUACAGU | 988 | ACUGUAAU CUGAUGAG GCCGUUAGGC CGAA IGAGGCAG | 3177 |
| 2127 | CCAAUUAC A GUGACUUC | 989 | GAAGUCAC CUGAUGAG GCCGUUAGGC CGAA IUAAUUGG | 3178 |
| 2133 | ACAGUGAC U UCCAAAAC | 990 | GUUUUGGA CUGAUGAG GCCGUUAGGC CGAA IUCACUGU | 3179 |
| 2136 | GUGACUUC C AAAACGAA | 991 | UUCGUUUU CUGAUGAG GCCGUUAGGC CGAA IAAGUCAC | 3180 |
| 2137 | UGACUUCC A AAACGAAC | 992 | GUUCGUUU CUGAUGAG GCCGUUAGGC CGAA IGAAGUCA | 3181 |
| 2146 | AAACGAAC A AGGACACC | 993 | GGUGUCCU CUGAUGAG GCCGUUAGGC CGAA IUUCGUUU | 3182 |
| 2152 | ACAAGGAC A CCAGCAAA | 994 | UUUGCUGG CUGAUGAG GCCGUUAGGC CGAA IUCCUUGU | 3183 |
| 2154 | AAGGACAC C AGCAAAUU | 995 | AAUUUGCU CUGAUGAG GCCGUUAGGC CGAA IUGUCCUU | 3184 |
| 2155 | AGGACACC A GCAAAUUC | 996 | GAAUUUGC CUGAUGAG GCCGUUAGGC CGAA IGUGUCCU | 3185 |
| 2158 | ACACCAGC A AAUUCCCC | 997 | GGGGAAUU CUGAUGAG GCCGUUAGGC CGAA ICUGGUGU | 3186 |
| 2164 | GCAAAUUC C CCAGCCCU | 998 | AGGGCUGG CUGAUGAG GCCGUUAGGC CGAA IAAUUUGC | 3187 |
| 2165 | CAAAUUCC C CAGCCCUC | 999 | GAGGGCUG CUGAUGAG GCCGUUAGGC CGAA IGAAUUUG | 3188 |
| 2166 | AAAUUCCC C AGCCCUCU | 1000 | AGAGGGCU CUGAUGAG GCCGUUAGGC CGAA IGGAAUUU | 3189 |
| 2167 | AAUUCCCC A GCCCUCUG | 1001 | CAGAGGGC CUGAUGAG GCCGUUAGGC CGAA IGGGAAUU | 3190 |
| 2170 | UCCCCAGC C CUCUGGUA | 1002 | UACCAGAG CUGAUGAG GCCGUUAGGC CGAA ICUGGGGA | 3191 |
| 2171 | CCCCAGCC C UCUGGUAG | 1003 | CUACCAGA CUGAUGAG GCCGUUAGGC CGAA IGCUGGGG | 3192 |
| 2172 | CCCAGCCC U CUGGUAGU | 1004 | ACUACCAG CUGAUGAG GCCGUUAGGC CGAA IGGCUGGG | 3193 |
| 2174 | CAGCCCUC U GGUAGUUU | 1005 | AAACUACC CUGAUGAG GCCGUUAGGC CGAA IAGGGCUG | 3194 |
| 2187 | GUUUAUGC A AAUAUUCG | 1006 | CGAAUAUU CUGAUGAG GCCGUUAGGC CGAA ICAUAAAC | 3195 |
| 2197 | AUAUUCGC C AAGGAGCC | 1007 | GGCUCCUU CUGAUGAG GCCGUUAGGC CGAA ICGAAUAU | 3196 |
| 2198 | UAUUCGCC A AGGAGCCU | 1008 | AGGCUCCU CUGAUGAG GCCGUUAGGC CGAA IGCGAAUA | 3197 |
| 2205 | CAAGGAGC C UCCCCAAU | 1009 | AUUGGGGA CUGAUGAG GCCGUUAGGC CGAA ICUCCUUG | 3198 |
| 2206 | AAGGAGCC U CCCCAAUU | 1010 | AAUUGGGG CUGAUGAG GCCGUUAGGC CGAA IGCUCCUU | 3199 |
| 2208 | GGAGCCUC C CCAAUUCU | 1011 | AGAAUUGG CUGAUGAG GCCGUUAGGC CGAA IAGGCUCC | 3200 |
| 2209 | GAGCCUCC C CAAUUCUC | 1012 | GAGAAUUG CUGAUGAG GCCGUUAGGC CGAA IGAGGCUC | 3201 |
| 2210 | AGCCUCCC C AAUUCUCA | 1013 | UGAGAAUU CUGAUGAG GCCGUUAGGC CGAA IGGAGGCU | 3202 |
| 2211 | GCCUCCCC A AUUCUCAG | 1014 | CUGAGAAU CUGAUGAG GCCGUUAGGC CGAA IGGGAGGC | 3203 |
| 2216 | CCCAAUUC U CAGGGCCA | 1015 | UGGCCCUG CUGAUGAG GCCGUUAGGC CGAA IAAUUGGG | 3204 |
| 2218 | CAAUUCUC A GGGCCAGU | 1016 | ACUGGCCC CUGAUGAG GCCGUUAGGC CGAA IAGAAUUG | 3205 |
| 2223 | CUCAGGGC C AGUGUCAC | 1017 | GUGACACU CUGAUGAG GCCGUUAGGC CGAA ICCCUGAG | 3206 |
| 2224 | UCAGGGCC A GUGUCACA | 1018 | UGUGACAC CUGAUGAG GCCGUUAGGC CGAA IGCCCUGA | 3207 |
| 2230 | CCAGUGUC A CAGCCCUG | 1019 | CAGGGCUG CUGAUGAG GCCGUUAGGC CGAA IACACUGG | 3208 |
| 2232 | AGUGUCAC A GCCCUGAU | 1020 | AUCAGGGC CUGAUGAG GCCGUUAGGC CGAA IUGACACU | 3209 |
| 2235 | GUCACAGC C CUGAUUGA | 1021 | UCAAUCAG CUGAUGAG GCCGUUAGGC CGAA ICUGUGAC | 3210 |
| 2236 | UCACAGCC C UGAUUGAA | 1022 | UUCAAUCA CUGAUGAG GCCGUUAGGC CGAA IGCUGUGA | 3211 |
| 2237 | CACAGCCC U GAUUGAAU | 1023 | AUUCAAUC CUGAUGAG GCCGUUAGGC CGAA IGGCUGUG | 3212 |
| 2247 | AUUGAAUC A GUGAAUGG | 1024 | CCAUUCAC CUGAUGAG GCCGUUAGGC CGAA IAUUCAAU | 3213 |
| 2262 | GGAAAAAC A GUUACCUU | 1025 | AAGGUAAC CUGAUGAG GCCGUUAGGC CGAA IUUUUUCC | 3214 |
| 2268 | ACAGUUAC C UUGGAACU | 1026 | AGUUCCAA CUGAUGAG GCCGUUAGGC CGAA IUAACUGU | 3215 |
| 2269 | CAGUUACC U UGGAACUA | 1027 | UAGUUCCA CUGAUGAG GCCGUUAGGC CGAA IGUAACUG | 3216 |
| 2276 | CUUGGAAC U ACUGGAUA | 1028 | UAUCCAGU CUGAUGAG GCCGUUAGGC CGAA IUUCCAAG | 3217 |
| 2279 | GGAACUAC U GGAUAAUG | 1029 | CAUUAUCC CUGAUGAG GCCGUUAGGC CGAA IUAGUUCC | 3218 |
| 2292 | AAUGGAGC A GGUGCUGA | 1030 | UCAGCACC CUGAUGAG GCCGUUAGGC CGAA ICUCCAUU | 3219 |
| 2298 | GCAGGUGC U GAUGCUAC | 1031 | GUAGCAUC CUGAUGAG GCCGUUAGGC CGAA ICACCUGC | 3220 |
| 2304 | GCUGAUGC U ACUAAGGA | 1032 | UCCUUAGU CUGAUGAG GCCGUUAGGC CGAA ICAUCAGC | 3221 |
| 2307 | GAUGCUAC U AAGGAUGA | 1033 | UCAUCCUU CUGAUGAG GCCGUUAGGC CGAA IUAGCAUC | 3222 |
| 2323 | ACGGUGUC U ACUCAAGG | 1034 | CCUUGAGU CUGAUGAG GCCGUUAGGC CGAA IACACCGU | 3223 |
| 2326 | GUGUCUAC U CAAGGUAU | 1035 | AUACCUUG CUGAUGAG GCCGUUAGGC CGAA IUAGACAC | 3224 |
| 2328 | GUCUACUC A AGGUAUUU | 1036 | AAAUACCU CUGAUGAG GCCGUUAGGC CGAA IAGUAGAC | 3225 |
| 2338 | GGUAUUUC A CAACUUAU | 1037 | AUAAGUUG CUGAUGAG GCCGUUAGGC CGAA IAAAUACC | 3226 |
| 2340 | UAUUUCAC A ACUUAUGA | 1038 | UCAUAAGU CUGAUGAG GCCGUUAGGC CGAA IUGAAAUA | 3227 |
| 2343 | UUCACAAC U UAUGACAC | 1039 | GUGUCAUA CUGAUGAG GCCGUUAGGC CGAA IUUGUGAA | 3228 |
| 2350 | CUUAUGAC A CGAAUGGU | 1040 | ACCAUUCG CUGAUGAG GCCGUUAGGC CGAA IUCAUAAG | 3229 |
| 2365 | GUAGAUAC A GUGUAAAA | 1041 | UUUUACAC CUGAUGAG GCCGUUAGGC CGAA IUAUCUAC | 3230 |
| 2382 | GUGCGGGC U CUGGGAGG | 1042 | CCUCCCAG CUGAUGAG GCCGUUAGGC CGAA ICCCGCAC | 3231 |
| 2384 | GCGGGCUC U GGGAGGAG | 1043 | CUCCUCCC CUGAUGAG GCCGUUAGGC CGAA IAGCCCGC | 3232 |
| 2400 | GUUAACGC A GCCAGACG | 1044 | CGUCUGGC CUGAUGAG GCCGUUAGGC CGAA ICGUUAAC | 3233 |
| 2403 | AACGCAGC C AGACGGAG | 1045 | CUCCGUCU CUGAUGAG GCCGUUAGGC CGAA ICUGCGUU | 3234 |
| 2404 | ACGCAGCC A GACGGAGA | 1046 | UCUCCGUC CUGAUGAG GCCGUUAGGC CGAA IGCUGCGU | 3235 |
| 2420 | AGUGAUAC C CCAGCAGA | 1047 | UCUGCUGG CUGAUGAG GCCGUUAGGC CGAA IUAUCACU | 3236 |
| 2421 | GUGAUACC C CAGCAGAG | 1048 | CUCUGCUG CUGAUGAG GCCGUUAGGC CGAA IGUAUCAC | 3237 |
| 2422 | UGAUACCC C AGCAGAGU | 1049 | ACUCUGCU CUGAUGAG GCCGUUAGGC CGAA IGGUAUCA | 3238 |
| 2423 | GAUACCCC A GCAGAGUG | 1050 | CACUCUGC CUGAUGAG GCCGUUAGGC CGAA IGGGUAUC | 3239 |
| 2426 | ACCCCAGC A GAGUGGAG | 1051 | CUCCACUC CUGAUGAG GCCGUUAGGC CGAA ICUGGGGU | 3240 |
| 2436 | AGUGGAGC A CUGUACAU | 1052 | AUGUACAG CUGAUGAG GCCGUUAGGC CGAA ICUCCACU | 3241 |
| 2438 | UGGAGCAC U GUACAUAC | 1053 | GUAUGUAC CUGAUGAG GCCGUUAGGC CGAA IUGCUCCA | 3242 |
| 2443 | CACUGUAC A UACCUGGC | 1054 | GCCAGGUA CUGAUGAG GCCGUUAGGC CGAA IUACAGUG | 3243 |
| 2447 | GUACAUAC C UGGCUGGA | 1055 | UCCAGCCA CUGAUGAG GCCGUUAGGC CGAA IUAUGUAC | 3244 |
| 2448 | UACAUACC U GGCUGGAU | 1056 | AUCCAGCC CUGAUGAG GCCGUUAGGC CGAA IGUAUGUA | 3245 |
| 2452 | UACCUGGC U GGAUUGAG | 1057 | CUCAAUCC CUGAUGAG GCCGUUAGGC CGAA ICCAGGUA | 3246 |
| 2474 | UGAAAUAC A AUGGAAUC | 1058 | GAUUCCAU CUGAUGAG GCCGUUAGGC CGAA IUAUUUCA | 3247 |
| 2483 | AUGGAAUC C ACCAAGAC | 1059 | GUCUUGGU CUGAUGAG GCCGUUAGGC CGAA IAUUCCAU | 3248 |
| 2484 | UGGAAUCC A CCAAGACC | 1060 | GGUCUUGG CUGAUGAG GCCGUUAGGC CGAA IGAUUCCA | 3249 |
| 2486 | GAAUCCAC C AAGACCUG | 1061 | CAGGUCUU CUGAUGAG GCCGUUAGGC CGAA IUGGAUUC | 3250 |
| 2487 | AAUCCACC A AGACCUGA | 1062 | UCAGGUCU CUGAUGAG GCCGUUAGGC CGAA IGUGGAUU | 3251 |
| 2492 | ACCAAGAC C UGAAAUUA | 1063 | UAAUUUCA CUGAUGAG GCCGUUAGGC CGAA IUCUUGGU | 3252 |
| 2493 | CCAAGACC U GAAAUUAA | 1064 | UUAAUUUC CUGAUGAG GCCGUUAGGC CGAA IGUCUUGG | 3253 |
| 2516 | UGAUGUUC A ACACAAGC | 1065 | GCUUGUGU CUGAUGAG GCCGUUAGGC CGAA IAACAUCA | 3254 |
| 2519 | UGUUCAAC A CAAGCAAG | 1066 | CUUGCUUG CUGAUGAG GCCGUUAGGC CGAA IUUGAACA | 3255 |
| 2521 | UUCAACAC A AGCAAGUG | 1067 | CACUUGCU CUGAUGAG GCCGUUAGGC CGAA IUGUUGAA | 3256 |
| 2525 | ACACAAGC A AGUGUGUU | 1068 | AACACACU CUGAUGAG GCCGUUAGGC CGAA ICUUGUGU | 3257 |
| 2536 | UGUGUUUC A GCAGAACA | 1069 | UGUUCUGC CUGAUGAG GCCGUUAGGC CGAA IAAACACA | 3258 |
| 2539 | GUUUCAGC A GAACAUCC | 1070 | GGAUGUUC CUGAUGAG GCCGUUAGGC CGAA ICUGAAAC | 3259 |
| 2544 | AGCAGAAC A UCCUCGGG | 1071 | CCCGAGGA CUGAUGAG GCCGUUAGGC CGAA IUUCUGCU | 3260 |
| 2547 | AGAACAUC C UCGGGAGG | 1072 | CCUCCCGA CUGAUGAG GCCGUUAGGC CGAA IAUGUUCU | 3261 |
| 2548 | GAACAUCC U CGGGAGGC | 1073 | GCCUCCCG CUGAUGAG GCCGUUAGGC CGAA IGAUGUUC | 3262 |
| 2557 | CGGGAGGC U CAUUUGUG | 1074 | CACAAAUG CUGAUGAG GCCGUUAGGC CGAA ICCUCCCG | 3263 |
| 2559 | GGAGGCUC A UUUGUGGC | 1075 | GCCACAAA CUGAUGAG GCCGUUAGGC CGAA IAGCCUCC | 3264 |
| 2568 | UUUGUGGC U UCUGAUGU | 1076 | ACAUCAGA CUGAUGAG GCCGUUAGGC CGAA ICCACAAA | 3265 |
| 2571 | GUGGCUUC U GAUGUCCC | 1077 | GGGACAUC CUGAUGAG GCCGUUAGGC CGAA IAAGCCAC | 3266 |
| 2578 | CUGAUGUC C CAAAUGCU | 1078 | AGCAUUUG CUGAUGAG GCCGUUAGGC CGAA IACAUCAG | 3267 |
| 2579 | UGAUGUCC C AAAUGCUC | 1079 | GAGCAUUU CUGAUGAG GCCGUUAGGC CGAA IGACAUCA | 3268 |
| 2580 | GAUGUCCC A AAUGCUCC | 1080 | GGAGCAUU CUGAUGAG GCCGUUAGGC CGAA IGGACAUC | 3269 |
| 2586 | CCAAAUGC U CCCAUACC | 1081 | GGUAUGGG CUGAUGAG GCCGUUAGGC CGAA ICAUUUGG | 3270 |
| 2588 | AAAUGCUC C CAUACCUG | 1082 | CAGGUAUG CUGAUGAG GCCGUUAGGC CGAA IAGCAUUU | 3271 |
| 2589 | AAUGCUCC C AUACCUGA | 1083 | UCAGGUAU CUGAUGAG GCCGUUAGGC CGAA IGAGCAUU | 3272 |
| 2590 | AUGCUCCC A UACCUGAU | 1084 | AUCAGGUA CUGAUGAG GCCGUUAGGC CGAA IGGAGCAU | 3273 |
| 2594 | UCCCAUAC C UGAUCUCU | 1085 | AGAGAUCA CUGAUGAG GCCGUUAGGC CGAA IUAUGGGA | 3274 |
| 2595 | CCCAUACC U GAUCUCUU | 1086 | AAGAGAUC CUGAUGAG GCCGUUAGGC CGAA IGUAUGGG | 3275 |
| 2600 | ACCUGAUC U CUUCCCAC | 1087 | GUGGGAAG CUGAUGAG GCCGUUAGGC CGAA IAUCAGGU | 3276 |
| 2602 | CUGAUCUC U UCCCACCU | 1088 | AGGUGGGA CUGAUGAG GCCGUUAGGC CGAA IAGAUCAG | 3277 |
| 2605 | AUCUCUUC C CACCUGGC | 1089 | GCCAGGUG CUGAUGAG GCCGUUAGGC CGAA IAAGAGAU | 3278 |
| 2606 | UCUCUUCC C ACCUGGCC | 1090 | GGCCAGGU CUGAUGAG GCCGUUAGGC CGAA IGAAGAGA | 3279 |
| 2607 | CUCUUCCC A CCUGGCCA | 1091 | UGGCCAGG CUGAUGAG GCCGUUAGGC CGAA IGGAAGAG | 3280 |
| 2609 | CUUCCCAC C UGGCCAAA | 1092 | UUUGGCCA CUGAUGAG GCCGUUAGGC CGAA IUGGGAAG | 3281 |
| 2610 | UUCCCACC U GGCCAAAU | 1093 | AUUUGGCC CUGAUGAG GCCGUUAGGC CGAA IGUGGGAA | 3282 |
| 2614 | CACCUGGC C AAAUCACC | 1094 | GGUGAUUU CUGAUGAG GCCGUUAGGC CGAA ICCAGGUG | 3283 |
| 2615 | ACCUGGCC A AAUCACCG | 1095 | CGGUGAUU CUGAUGAG GCCGUUAGGC CGAA IGCCAGGU | 3284 |
| 2620 | GCCAAAUC A CCGACCUG | 1096 | CAGGUCGG CUGAUGAG GCCGUUAGGC CGAA IAUUUGGC | 3285 |
| 2622 | CAAAUCAC C GACCUGAA | 1097 | UUCAGGUC CUGAUGAG GCCGUUAGGC CGAA IUGAUUUG | 3286 |
| 2626 | UCACCGAC C UGAAGGCG | 1098 | CGCCUUCA CUGAUGAG GCCGUUAGGC CGAA IUCGGUGA | 3287 |
| 2627 | CACCGACC U GAAGGCGG | 1099 | CCGCCUUC CUGAUGAG GCCGUUAGGC CGAA IGUCGGUG | 3288 |
| 2642 | GGAAAUUC A CGGGGGCA | 1100 | UGCCCCCG CUGAUGAG GCCGUUAGGC CGAA IAAUUUCC | 3289 |
| 2650 | ACGGGGGC A GUCUCAUU | 1101 | AAUGAGAC CUGAUGAG GCCGUUAGGC CGAA ICCCCCGU | 3290 |
| 2654 | GGGCAGUC U CAUUAAUC | 1102 | GAUUAAUG CUGAUGAG GCCGUUAGGC CGAA IACUGCCC | 3291 |
| 2656 | GCAGUCUC A UUAAUCUG | 1103 | CAGAUUAA CUGAUGAG GCCGUUAGGC CGAA IAGACUGC | 3292 |
| 2663 | CAUUAAUC U GACUUGGA | 1104 | UCCAAGUC CUGAUGAG GCCGUUAGGC CGAA IAUUAAUG | 3293 |
| 2667 | AAUCUGAC U UGGACAGC | 1105 | GCUGUCCA CUGAUGAG GCCGUUAGGC CGAA IUCAGAUU | 3294 |
| 2673 | ACUUGGAC A GCUCCUGG | 1106 | CCAGGAGC CUGAUGAG GCCGUUAGGC CGAA IUCCAAGU | 3295 |
| 2676 | UGGACAGC U CCUGGGGA | 1107 | UCCCCAGG CUGAUGAG GCCGUUAGGC CGAA ICUGUCCA | 3296 |
| 2678 | GACAGCUC C UGGGGAUG | 1108 | CAUCCCCA CUGAUGAG GCCGUUAGGC CGAA IAGCUGUC | 3297 |
| 2679 | ACAGCUCC U GGGGAUGA | 1109 | UCAUCCCC CUGAUGAG GCCGUUAGGC CGAA IGAGCUGU | 3298 |
| 2695 | AUUAUGAC C AUGGAACA | 1110 | UGUUCCAU CUGAUGAG GCCGUUAGGC CGAA IUCAUAAU | 3299 |
| 2696 | UUAUGACC A UGGAACAG | 1111 | CUGUUCCA CUGAUGAG GCCGUUAGGC CGAA IGUCAUAZ | 3300 |
| 2703 | CAUGGAAC A GCUCACAA | 1112 | UUGUGAGC CUGAUGAG GCCGUUAGGC CGAA IUUCCAUG | 3301 |
| 2706 | GGAACAGC U CACAAGUA | 1113 | UACUUGUG CUGAUGAG GCCGUUAGGC CGAA ICUGUUCC | 3302 |
| 2708 | AACAGCUC A CAAGUAUA | 1114 | UAUACUUG CUGAUGAG GCCGUUAGGC CGAA IAGCUGUU | 3303 |
| 2710 | CAGCUCAC A AGUAUAUC | 1115 | GAUAUACU CUGAUGAG GCCGUUAGGC CGAA IUGAGCUG | 3304 |
| 2719 | AGUAUAUC A UUCGAAUA | 1116 | UAUUCGAA CUGAUGAG GCCGUUAGGC CGAA IAUAUACU | 3305 |
| 2733 | AUAAGUAC A AGUAUUCU | 1117 | AGAAUACU CUGAUGAG GCCGUUAGGC CGAA IUACUUAU | 3306 |
| 2741 | AAGUAUUC U UGAUCUCA | 1118 | UGAGAUCA CUGAUGAG GCCGUUAGGC CGAA IAAUACUU | 3307 |
| 2747 | UCUUGAUC U CAGAGACA | 1119 | UGUCUCUG CUGAUGAG GCCGUUAGGC CGAA IAUCAAGA | 3308 |
| 2749 | UUGAUCUC A GAGACAAG | 1120 | CUUGUCUC CUGAUGAG GCCGUUAGGC CGAA IAGAUCAA | 3309 |
| 2755 | UCAGAGAC A AGUUCAAU | 1121 | AUUGAACU CUGAUGAG GCCGUUAGGC CGAA IUCUCUGA | 3310 |
| 2761 | ACAAGUUC A AUGAAUCU | 1122 | AGAUUCAU CUGAUGAG GCCGUUAGGC CGAA IAACUUGU | 3311 |
| 2769 | AAUGAAUC U CUUCAAGU | 1123 | ACUUGAAG CUGAUGAG GCCGUUAGGC CGAA IAUUCAUU | 3312 |
| 2771 | UGAAUCUC U UCAAGUGA | 1124 | UCACUUGA CUGAUGAG GCCGUUAGGC CGAA IAGAUUCA | 3313 |
| 2774 | AUCUCUUC A AGUGAAUA | 1125 | UAUUCACU CUGAUGAG GCCGUUAGGC CGAA IAAGAGAU | 3314 |
| 2784 | GUGAAUAC U ACUGCUCU | 1126 | AGAGCAGU CUGAUGAG GCCGUUAGGC CGAA IUAUUCAC | 3315 |
| 2787 | AAUACUAC U GCUCUCAU | 1127 | AUGAGAGC CUGAUGAG GCCGUUAGGC CGAA IUAGUAUU | 3316 |
| 2790 | ACUACUGC U CUCAUCCC | 1128 | GGGAUGAG CUGAUGAG GCCGUUAGGC CGAA ICAGUAGU | 3317 |
| 2792 | UACUGCUC U CAUCCCAA | 1129 | UUGGGAUG CUGAUGAG GCCGUUAGGC CGAA IAGCAGUA | 3318 |
| 2794 | CUGCUCUC A UCCCAAAG | 1130 | CUUUGGGA CUGAUGAG GCCGUUAGGC CGAA IAGAGCAG | 3319 |
| 2797 | CUCUCAUC C CAAAGGAA | 1131 | UUCCUUUG CUGAUGAG GCCGUUAGGC CGAA IAUGAGAG | 3320 |
| 2798 | UCUCAUCC C AAAGGAAG | 1132 | CUUCCUUU CUGAUGAG GCCGUUAGGC CGAA IGAUGAGA | 3321 |
| 2799 | CUCAUCCC A AAGGAAGC | 1133 | GCUUCCUU CUGAUGAG GCCGUUAGGC CGAA IGGAUGAG | 3322 |
| 2808 | AAGGAAGC C AACUCUGA | 1134 | UCAGAGUU CUGAUGAG GCCGUUAGGC CGAA ICUUCCUU | 3323 |
| 2809 | AGGAAGCC A ACUCUGAG | 1135 | CUCAGAGU CUGAUGAG GCCGUUAGGC CGAA IGCUUCCU | 3324 |
| 2812 | AAGCCAAC U CUGAGGAA | 1136 | UUCCUCAG CUGAUGAG GCCGUUAGGC CGAA IUUGGCUU | 3325 |
| 2814 | GCCAACUC U GAGGAAGU | 1137 | ACUUCCUC CUGAUGAG GCCGUUAGGC CGAA IAGUUGGC | 3326 |
| 2824 | AGGAAGUC U UUUUGUUU | 1138 | AAACAAAA CUGAUGAG GCCGUUAGGC CGAA IACUUCCU | 3327 |
| 2837 | GUUUAAAC C AGAAAACA | 1139 | UGUUUUCU CUGAUGAG GCCGUUAGGC CGAA IUUUAAAC | 3328 |
| 2838 | UUUAAACC A GAAAACAU | 1140 | AUGUUUUC CUGAUGAG GCCGUUAGGC CGAA IGUUUAAA | 3329 |
| 2845 | CAGAAAAC A UUACUUUU | 1141 | AAAAGUAA CUGAUGAG GCCGUUAGGC CGAA IUUUUCUG | 3330 |
| 2850 | AACAUUAC U UUUGAAAA | 1142 | UUUUCAAA CUGAUGAG GCCGUUAGGC CGAA IUAAUGUU | 3331 |
| 2863 | AAAAUGGC A CAGAUCUU | 1143 | AAGAUCUG CUGAUGAG GCCGUUAGGC CGAA ICCAUUUU | 3332 |
| 2865 | AAUGGCAC A GAUCUUUU | 1144 | AAAAGAUC CUGAUGAG GCCGUUAGGC CGAA IUGCCAUU | 3333 |
| 2870 | CACAGAUC U UUUCAUUG | 1145 | CAAUGAAA CUGAUGAG GCCGUUAGGC CGAA IAUCUGUG | 3334 |
| 2875 | AUCUUUUC A UUGCUAUU | 1146 | AAUAGCAA CUGAUGAG GCCGUUAGGC CGAA IAAAAGAU | 3335 |
| 2880 | UUCAUUGC U AUUCAGGC | 1147 | GCCUGAZU CUGAUGAG GCCGUUAGGC CGAA ICAAUGAA | 3336 |
| 2885 | UGCUAUUC A GGCUGUUG | 1148 | CAACAGCC CUGAUGAG GCCGUUAGGC CGAA IAAUAGCA | 3337 |
| 2889 | AUUCAGGC U GUUGAUAA | 1149 | UUAUCAAC CUGAUGAG GCCGUUAGGC CGAA ICCUGAAU | 3338 |
| 2906 | GGUCGAUC U GAAAUCAG | 1150 | CUGAUUUC CUGAUGAG GCCGUUAGGC CGAA IAUCGACC | 3339 |
| 2913 | CUGAAAUC A GAAAUAUC | 1151 | GAUAUUUC CUGAUGAG GCCGUUAGGC CGAA IAUUUCAG | 3340 |
| 2922 | GAAAUAUC C AACAUUGC | 1152 | GCAAUGUU CUGAUGAG GCCGUUAGGC CGAA IAUAUUUC | 3341 |
| 2923 | AAAUAUCC A ACAUUGCA | 1153 | UGCAAUGU CUGAUGAG GCCGUUAGGC CGAA IGAUAUUU | 3342 |
| 2926 | UAUCCAAC A UUGCACGA | 1154 | UCGUGCAA CUGAUGAG GCCGUUAGGC CGAA IUUGGAUA | 3343 |
| 2931 | AACAUUGC A CGAGUAUC | 1155 | GAUACUCG CUGAUGAG GCCGUUAGGC CGAA ICAAUGUU | 3344 |
| 2940 | CGAGUAUC U UUGUUUAU | 1156 | AUAAACAA CUGAUGAG GCCGUUAGGC CGAA IAUACUCG | 3345 |
| 2951 | GUUUAUUC C UCCACAGA | 1157 | UCUGUGGA CUGAUGAG GCCGUUAGGC CGAA IAAUAAAC | 3346 |
| 2952 | UUUAUUCC U CCACAGAC | 1158 | GUCUGUGG CUGAUGAG GCCGUUAGGC CGAA IGAAUAAA | 3347 |
| 2954 | UAUUCCUC C ACAGACUC | 1159 | GAGUCUGU CUGAUGAG GCCGUUAGGC CGAA IAGGAAUA | 3348 |
| 2955 | AUUCCUCC A CAGACUCC | 1160 | OGAGUCUG CUGAUGAG GCCGUUAGGC CGAA IGAGGAAU | 3349 |
| 2957 | UCCUCCAC A GACUCCGC | 1161 | GCGGAGUC CUGAUGAG GCCGUUAGGC CGAA IUGGAGGA | 3350 |
| 2961 | CCACAGAC U CCGCCAGA | 1162 | UCUGGCGG CUGAUGAG GCCGUUAGGC CGAA IUCUGUGG | 3351 |
| 2963 | ACAGACUC C GCCAGAGA | 1163 | UCUCUGGC CUGAUGAG GCCGUUAGGC CGAA IAGUCUGU | 3352 |
| 2966 | GACUCCGC C AGAGACAC | 1164 | GUGUCUCU CUGAUGAG GCCGUUAGGC CGAA ICGGAGUC | 3353 |
| 2967 | ACUCCGCC A GAGACACC | 1165 | GGUGUCUC CUGAUGAG GCCGUUAGGC CGAA IGCGGAGU | 3354 |
| 2973 | CCAGAGAC A CCUAGUCC | 1166 | GGACUAGG CUGAUGAG GCCGUUAGGC CGAA IUCUCUGG | 3355 |
| 2975 | AGAGACAC C UAGUCCUG | 1167 | CAGGACUA CUGAUGAG GCCGUUAGGC CGAA IUGUCUCU | 3356 |
| 2976 | GAGACACC U AGUCCUGA | 1168 | UCAGGACU CUGAUGAG GCCGUUAGGC CGAA IGUGUCUC | 3357 |
| 2981 | ACCUAGUC C UGAUGAAA | 1169 | UUUCAUCA CUGAUGAG GCCGUUAGGC CGAA IACUAGGU | 3358 |
| 2982 | CCUAGUCC U GAUGAAAC | 1170 | GUUUCAUC CUGAUGAG GCCGUUAGGC CGAA IGACUAGG | 3359 |
| 2994 | GAAACGUC U GCUCCUUG | 1171 | CAAGGAGC CUGAUGAG GCCGUUAGGC CGAA IACGUUUC | 3360 |
| 2997 | ACGUCUGC U CCUUGUCC | 1172 | GGACAAGG CUGAUGAG GCCGUUAGGC CGAA ICAGACGU | 3361 |
| 2999 | GUCUGCUC C UUGUCCUA | 1173 | UAGGACAA CUGAUGAG GCCGUUAGGC CGAA IAGCAGAC | 3362 |
| 3000 | UCUGCUCC U UGUCCUAA | 1174 | UUAGGACA CUGAUGAG GCCGUUAGGC CGAA IGAGCAGA | 3363 |
| 3005 | UCCUUGUC C UAAUAUUC | 1175 | GAAUAUUA CUGAUGAG GCCGUUAGGC CGAA IACAAGGA | 3364 |
| 3006 | CCUUGUCC U AAUAUUCA | 1176 | UGAAUAUU CUGAUGAG GCCGUUAGGC CGAA IGACAAGG | 3365 |
| 3014 | UAAUAUUC A UAUCAACA | 1177 | UGUUGAUA CUGAUGAG GCCGUUAGGC CGAA IAAUAUUA | 3366 |
| 3019 | UUCAUAUC A ACAGCACC | 1178 | GGUGCUGU CUGAUGAG GCCGUUAGGC CGAA IAUAUGAA | 3367 |
| 3022 | AUAUCAAC A GCACCAUU | 1179 | AAUGGUGC CUGAUGAG GCCGUUAGGC CGAA IUUGAUAU | 3368 |
| 3025 | UCAACAGC A CCAUUCCU | 1180 | AGGAAUGG CUGAUGAG GCCGUUAGGC CGAA ICUGUUGA | 3369 |
| 3027 | AACAGCAC C AUUCCUGG | 1181 | CCAGGAAU CUGAUGAG GCCGUUAGGC CGAA IUGCUGUU | 3370 |
| 3028 | ACAGCACC A UUCCUGGC | 1182 | GCCAGGAA CUGAUGAG GCCGUUAGGC CGAA IGUGCUGU | 3371 |
| 3032 | CACCAUUC C UGGCAUUC | 1183 | GAAUGCCA CUGAUGAG GCCGUUAGGC CGAA IAAUGGUG | 3372 |
| 3033 | ACCAUUCC U GGCAUUCA | 1184 | UGAAUGCC CUGAUGAG GCCGUUAGGC CGAA IGAAUGGU | 3373 |
| 3037 | UUCCUGGC A UUCACAUU | 1185 | AAUGUGAA CUGAUGAG GCCGUUAGGC CGAA ICCAGGAA | 3374 |
| 3041 | UGGCAUUC A CAUUUUAA | 1186 | UUAAAAUG CUGAUGAG GCCGUUAGGC CGAA IAAUGCCA | 3375 |
| 3043 | GCAUUCAC A UUUUAAAA | 1187 | UUUUAAAA CUGAUGAG GCCGUUAGGC CGAA IUGAAUGC | 3376 |
| 3077 | AGGAGAAC U GCAGCUGU | 1188 | ACAGCUGC CUGAUGAG GCCGUUAGGC CGAA IUUCUCCU | 3377 |
| 3080 | AGAACUGC A GCUGUCAA | 1189 | UUGACAGC CUGAUGAG GCCGUUAGGC CGAA ICAGUUCU | 3378 |
| 3083 | ACUGCAGC U GUCAAUAG | 1190 | CUAUUGAC CUGAUGAG GCCGUUAGGC CGAA ICUGCAGU | 3379 |
| 3087 | CAGCUGUC A AUAGCCUA | 1191 | UAGGCUAU CUGAUGAG GCCGUUAGGC CGAA IACAGCUG | 3380 |
| 3093 | UCAAUAGC C UAGGGCUG | 1192 | CAGCCCUA CUGAUGAG GCCGUUAGGC CGAA ICUAUUGA | 3381 |
| 3094 | CAAUAGCC U AGGGCUGA | 1193 | UCAGCCCU CUGAUGAG GCCGUUAGGC CGAA IGCUAUUG | 3382 |
| 3100 | CCUAGGGC U GAAUUUUU | 1194 | AAAAAUUC CUGAUGAG GCCGUUAGGC CGAA ICCCUAGG | 3383 |
| 3112 | UUUUUGUC A GAUAAAUA | 1195 | UAUUUAUC CUGAUGAG GCCGUUAGGC CGAA IACAAAAA | 3384 |
| 3130 | AAUAAAUC A UUCAUCCU | 1196 | AGGAUGAA CUGAUGAG GCCGUUAGGC CGAA IAUUUAUU | 3385 |
| 3134 | AAUCAUUC A UCCUUUUU | 1197 | AAAAAGGA CUGAUGAG GCCGUUAGGC CGAA IAAUGAUU | 3386 |
| 3137 | CAUUCAUC C UUUUUUUG | 1198 | CAAAAAAA CUGAUGAG GCCGUUAGGC CGAA IAUGAAUG | 3387 |
| 3138 | AUUCAUCC U UUUUUUGA | 1199 | UCAAAAAA CUGAUGAG GCCGUUAGGC CGAA IGAUGAAU | 3388 |
| 3160 | AAAUUUUC U AAAAUGUA | 1200 | UACAUUUU CUGAUGAG GCCGUUAGGC CGAA IAAAAUUU | 3389 |
| 3177 | UUUUAGAC U UCCUGUAG | 1201 | CUACAGGA CUGAUGAG GCCGUUAGGC CGAA IUCUAAAA | 3390 |
| 3267 | UUUUAGAC U UCCUGUAG | 1201 | CUACAGGA CUGAUGAG GCCGUUAGGC CGAA IUCUAAAA | 3390 |
| 3180 | UAGACUUC C UGUAGGGG | 1202 | CCCCUACA CUGAUGAG GCCGUUAGGC CGAA IAAGUCUA | 3391 |
| 3270 | UAGACUUC C UGUAGGGG | 1202 | CCCCUACA CUGAUGAG GCCGUUAGGC CGAA IAAGUCUA | 3391 |
| 3181 | AGACUUCC U GUAGGGGG | 1203 | CCCCCUAC CUGAUGAG GCCGUUAGGC CGAA IGAAGUCU | 3392 |
| 3271 | AGACUUCC U GUAGGGGG | 1203 | CCCCCUAC CUGAUGAG GCCGUUAGGC CGAA IGAAGUCU | 3392 |
| 3198 | CGAUAUAC U AAAUGUAU | 1204 | AUACAUUU CUGAUGAG GCCGUUAGGC CGAA IUAUAUCG | 3393 |
| 3251 | CGAUAUAC U AAAUGUAU | 1204 | AUACAUUU CUGAUGAG GCCGUUAGGC CGAA IUAUAUCG | 3393 |
| 3214 | UAUAGUAC A UUUAUACU | 1205 | AGUAUAAA CUGAUGAG GCCGUUAGGC CGAA IUACUAUA | 3394 |
| 3222 | AUUUAUAC U AAAUGUAU | 1206 | AUACAUUU CUGAUGAG GCCGUUAGGC CGAA IUAUAAAU | 3395 |
| 3233 | AUGUAUUC C UGUAGGGG | 1207 | CCCCUACA CUGAUGAG GCCGUUAGGC CGAA IAAUACAU | 3396 |
| 3234 | UGUAUUCC U GUAGGGGG | 1208 | CCCCCUAC CUGAUGAG GCCGUUAGGC CGAA IGAAUACA | 3397 |
| 3296 | UAAAAUGC U AAACAACU | 1209 | AGUUGUUU CUGAUGAG GCCGUUAGGC CGAA ICAUUUUA | 3398 |
| 3301 | UGCUAAAC A ACUGGGUA | 1210 | UACCCAGU CUGAUGAG GCCGUUAGGC CGAA IUUUAGCA | 3399 |

Input Sequence = NM_001285. Cut Site = CH/.
Arm Length = 8. Core Sequence = CUGAUGAG GCCGUUAGGC CGAA
Underlined region can be any X sequence or linker, as described herein.
NM_001285 (Homo sapiens chloride channel, calcium activated, 1 (CLCA1) mRNA, 3311 bp)

TABLE V
Human CLCA1 G-cleaver Ribozyme and Target Sequence

| | | Seq ID | | Rz Seq |
|---|---|---|---|---|
| Pos | Substrate | No. | Ribozyme | ID No. |
| 40 | AUAUAAUU G AAUAUUUU | 1211 | AAAAUAUU UGAUG GCAUGCACUAUGC GCG AAUUAUAU | 3400 |
| 67 | GGGAGCAU G AAGAGGUG | 1212 | CACCUCUU UGAUG GCAUGCACUAUGC GCG AUGCUCCC | 3401 |
| 78 | GAGGUGUU G AGGUUAUG | 1213 | CAUAACCU UGAUG GCAUGCACUAUGC GCG AACACCUC | 3402 |
| 106 | GCACAGCU G AAGGCAGA | 1214 | UCUGCCUU UGAUG GCAUGCACUAUGC GCG AGCUGUGC | 3403 |
| 134 | ACAAGUAC G CAAUUUGA | 1215 | UCAAAUUG UGAUG GCAUGCACUAUGC GCG GUACUUGU | 3404 |
| 141 | CGCAAUUU G AGACUAAG | 1216 | CUUAGUCU UGAUG GCAUGCACUAUGC GCG AAAUUGCG | 3405 |
| 172 | CUCCUAUU G AAGACAAG | 1217 | CUUGUCUU UGAUG GCAUGCACUAUGC GCG AAUAGGAG | 3406 |
| 223 | AGACCUGU G AUAAACCA | 1218 | UGGUUUAU UGAUG GCAUGCACUAUGC GCG ACAGGUCU | 3407 |
| 237 | CCACUUCC G AUAAGUUG | 1219 | CAACUUAU UGAUG GCAUGCACUAUGC GCG GGAAGUGG | 3408 |
| 312 | CGUAACCC G CAUUUUCC | 1220 | GGAAAAUG UGAUG GCAUGCACUAUGC GCG GGGUUACG | 3409 |
| 384 | UUCAUCUU G AUUCUUCA | 1221 | UGAAGAAU UGAUG GCAUGCACUAUGC GCG AAGAUGAA | 3410 |
| 411 | GGGGCCCU G AGUAAUUC | 1222 | GAAUUACU UGAUG GCAUGCACUAUGC GCG AGGGCCCC | 3411 |
| 432 | AUUCAGCU G AACAACAA | 1223 | UUGUUGUU UGAUG GCAUGCACUAUGC GCG AGCUGAAU | 3412 |
| 448 | AUGGCUAU G AAGGCAUU | 1224 | AAUGCCUU UGAUG GCAUGCACUAUGC GCG AUAGCCAU | 3413 |
| 463 | UUGUCGUU G CAAUCGAC | 1225 | GUCGAUUG UGAUG GCAUGCACUAUGC GCG AACGACAA | 3414 |
| 469 | UUGCAAUC G ACCCCAAU | 1226 | AUUGGGGU UGAUG GCAUGCACUAUGC GCG GAUUGCAA | 3415 |
| 480 | CCCAAUGU G CCAGAAGA | 1227 | UCUUCUGG UGAUG GCAUGCACUAUGC GCG ACAUUGGG | 3416 |
| 490 | CAGAAGAU G AAACACUC | 1228 | GAGUGUUU UGAUG GCAUGCACUAUGC GCG AUCUUCUG | 3417 |
| 522 | GACAUGGU G ACCCAGGC | 1229 | GCCUGGGU UGAUG GCAUGCACUAUGC GCG ACCAUGUC | 3418 |
| 547 | AUCUGUUU G AAGCUACA | 1230 | UGUAGCUU UGAUG GCAUGCACUAUGC GCG AAACAGAU | 3419 |
| 563 | AGGAAAGC G AUUUUAUU | 1231 | AAUAAAAU UGAUG GCAUGCACUAUGC GCG GCUUUCCU | 3420 |
| 583 | AAAAUGUU G CCAUUUUG | 1232 | CAAAAUGG UGAUG GCAUGCACUAUGC GCG AACAUUUU | 3421 |
| 591 | GCCAUUUU G AUUCCUGA | 1233 | UCAGGAAU UGAUG GCAUGCACUAUGC GCG AAAAUGGC | 3422 |
| 598 | UGAUUCCU G AAACAUGG | 1234 | CCAUGUUU UGAUG GCAUGCACUAUGC GCG AGGAAUCA | 3423 |
| 619 | CAAAGGCU G ACUAUGUG | 1235 | CACAUAGU UGAUG GCAUGCACUAUGC GCG AGCCUUUG | 3424 |
| 627 | GACUAUGU G AGACCAAA | 1236 | UUUGGUCU UGAUG GCAUGCACUAUGC GCG ACAUAGUC | 3425 |
| 640 | CAAAACUU G AGACCUAC | 1237 | GUAGGUCU UGAUG GCAUGCACUAUGC GCG AAGUUUUG | 3426 |
| 655 | ACAAAAAU G CUGAUGUU | 1238 | AACAUCAG UGAUG GCAUGCACUAUGC GCG AUUUUUGU | 3427 |
| 658 | AAAAUGCU G AUGUUCUG | 1239 | CAGAACAU UGAUG GCAUGCACUAUGC GCG AGCAUUUU | 3428 |
| 670 | UUCUGGUU G CUGAGUCU | 1240 | AGACUCAG UGAUG GCAUGCACUAUGC GCG AACCAGAA | 3429 |
| 673 | UGGUUGCU G AGUCUACU | 1241 | AGUAGACU UGAUG GCAUGCACUAUGC GCG AGCAACCA | 3430 |
| 694 | CAGGUAAU G AUGAACCC | 1242 | GGGUUCAU UGAUG GCAUGCACUAUGC GCG AUUACCUG | 3431 |
| 697 | GUAAUGAU G AACCCUAC | 1243 | GUAGGGUU UGAUG GCAUGCACUAUGC GCG AUCAUUAC | 3432 |
| 709 | CCUACACU G AGCAGAUG | 1244 | CAUCUGCU UGAUG GCAUGCACUAUGC GCG AGUGUAGG | 3433 |
| 739 | AGAAGGGU G AAAGGAUC | 1245 | GAUCCUUU UGAUG GCAUGCACUAUGC GCG ACCCUUCU | 3434 |
| 760 | UCACUCCU G AUUUCAUU | 1246 | AAUGAAAU UGAUG GCAUGCACUAUGC GCG AGGAGUGA | 3435 |
| 769 | AUUUCAUU G CAGGAAAA | 1247 | UUUUCCUG UGAUG GCAUGCACUAUGC GCG AAUGAAAU | 3436 |
| 787 | AGUUAGCU G AAUAUGGA | 1248 | UCCAUAUU UGAUG GCAUGCACUAUGC GCG AGCUAACU | 3437 |
| 820 | UUGUCCAU G AGUGGGCU | 1249 | AGCCCACU UGAUG GCAUGCACUAUGC GCG AUGGACAA | 3438 |
| 836 | UCAUCUAC G AUGGGGAG | 1250 | CUCCCCAU UGAUG GCAUGCACUAUGC GCG GUAGAUGA | 3439 |
| 850 | GAGUAUUU G ACGAGUAC | 1251 | GUACUCGU UGAUG GCAUGCACUAUGC GCG AAAUACUC | 3440 |
| 853 | UAUUUGAC G AGUACAAU | 1252 | AUUGUACU UGAUG GCAUGCACUAUGC GCG GUCAAAUA | 3441 |
| 865 | ACAAUAAU G AUGAGAAA | 1253 | UUUCUCAU UGAUG GCAUGCACUAUGC GCG AUUAUUGU | 3442 |
| 868 | AUAAUGAU G AGAAAUUC | 1254 | GAAUUUCU UGAUG GCAUGCACUAUGC GCG AUCAUUAU | 3443 |
| 980 | CAAAAGAU G CACAUUCA | 1255 | UGAAUGUG UGAUG GCAUGCACUAUGC GCG AUCUUUUG | 3444 |
| 1009 | GACUCUAU G AAAAAGGA | 1256 | UCCUUUUU UGAUG GCAUGCACUAUGC GCG AUAGAGUC | 3445 |
| 1021 | AAGGAUGU G AGUUUGUU | 1257 | AACAAACU UGAUG GCAUGCACUAUGC GCG ACAUCCUU | 3446 |
| 1040 | CCAAUCCC G CCAGACGG | 1258 | CCGUCUGG UGAUG GCAUGCACUAUGC GCG GGGAUUGG | 3447 |
| 1069 | UAAUGUUU G CACAACAU | 1259 | AUGUUGUG UGAUG GCAUGCACUAUGC GCG AAACAUUA | 3448 |
| 1081 | AACAUGUU G AUUCUAUA | 1260 | UAUAGAAU UGAUG GCAUGCACUAUGC GCG AACAUGUU | 3449 |
| 1093 | CUAUAGUU G AAUUCUGU | 1261 | ACAGAAUU UGAUG GCAUGCACUAUGC GCG AACUAUAG | 3450 |
| 1151 | UCAAAAAU G CAAUCUCC | 1262 | GGAGAUUG UGAUG GCAUGCACUAUGC GCG AUUUUUGA | 3451 |
| 1160 | CAAUCUCC G AAGCACAU | 1263 | AUGUGCUU UGAUG GCAUGCACUAUGC GCG GGAGAUUG | 3452 |
| 1176 | UGGGAAGU G AUCCGUGA | 1264 | UCACGGAU UGAUG GCAUGCACUAUGC GCG ACUUCCCA | 3453 |
| 1183 | UGAUCCGU G AUUCUGAG | 1265 | CUCAGAAU UGAUG GCAUGCACUAUGC GCG ACGGAUCA | 3454 |
| 1189 | GUGAUUCU G AGGACUUU | 1266 | AAAGUCCU UGAUG GCAUGCACUAUGC GCG AGAAUCAC | 3455 |
| 1215 | ACUCCUAU G ACAACACA | 1267 | UGUGUUGU UGAUG GCAUGCACUAUGC GCG AUAGGAGU | 3456 |
| 1248 | UUCUCAUU G CUGCAGAU | 1268 | AUCUGCAG UGAUG GCAUGCACUAUGC GCG AAUGAGAA | 3457 |
| 1251 | UCAUUGCU G CAGAUUGG | 1269 | CCAAUCUG UGAUG GCAUGCACUAUGC GCG AGCAAUGA | 3458 |
| 1285 | UAGUCCUU G ACAAAUCU | 1270 | AGAUUUGU UGAUG GCAUGCACUAUGC GCG AAGGACUA | 3459 |
| 1305 | AGCAUGGC G ACUGGUAA | 1271 | UUACCAGU UGAUG GCAUGCACUAUGC GCG GCCAUGCU | 3460 |
| 1316 | UGGUAACC G CCUCAAUC | 1272 | GAUUGAGG UGAUG GCAUGCACUAUGC GCG GGUUACCA | 3461 |
| 1325 | CCUCAAUC G ACUGAAUC | 1273 | GAUUCAGU UGAUG GCAUGCACUAUGC GCG GAUUGAGG | 3462 |
| 1329 | AAUCGACU G AAUCAAGC | 1274 | GCUUGAUU UGAUG GCAUGCACUAUGC GCG AGUCGAUU | 3463 |
| 1353 | CUUUUCCU G CUGCAGAC | 1275 | GUCUGCAG UGAUG GCAUGCACUAUGC GCG AGGAAAAG | 3464 |
| 1356 | UUCCUGCU G CAGACAGU | 1276 | ACUGUCUG UGAUG GCAUGCACUAUGC GCG AGCAGGAA | 3465 |
| 1366 | AGACAGUU G AGCUGGGG | 1277 | CCCCAGCU UGAUG GCAUGCACUAUGC GCG AACUGUCU | 3466 |
| 1392 | GGGAUGGU G ACAUUUGA | 1278 | UCAAAUGU UGAUG GCAUGCACUAUGC GCG ACCAUCCC | 3467 |
| 1399 | UGACAUUU G ACAGUGCU | 1279 | AGCACUGU UGAUG GCAUGCACUAUGC GCG AAAUGUCA | 3468 |
| 1405 | UUGACAGU G CUGCCCAU | 1280 | AUGGGCAG UGAUG GCAUGCACUAUGC GCG ACUGUCAA | 3469 |
| 1408 | ACAGUGCU G CCCAUGUA | 1281 | UACAUGGG UGAUG GCAUGCACUAUGC GCG AGCACUGU | 3470 |
| 1423 | UACAAAGU G AACUCAUA | 1282 | UAUGAGUU UGAUG GCAUGCACUAUGC GCG ACUUUGUA | 3471 |
| 1450 | GUGGCAGU G ACAGGGAC | 1283 | GUCCCUGU UGAUG GCAUGCACUAUGC GCG ACUGCCAC | 3472 |
| 1465 | ACACACUC G CCAAAAGA | 1284 | UCUUUUGG UGAUG GCAUGCACUAUGC GCG GAGUGUGU | 3473 |
| 1480 | GAUUACCU G CAGCAGCU | 1285 | AGCUGCUG UGAUG GCAUGCACUAUGC GCG AGGUAAUC | 3474 |
| 1508 | GUCCAUCU G CAGCGGGC | 1286 | GCCCGCUG UGAUG GCAUGCACUAUGC GCG AGAUGGAC | 3475 |
| 1520 | CGGGCUUC G AUCGGCAU | 1287 | AUGCCGAU UGAUG GCAUGCACUAUGC GCG GAAGCCCG | 3476 |
| 1536 | UUUACUGU G AUUAGGAA | 1288 | UUCCUAAU UGAUG GCAUGCACUAUGC GCG ACAGUAAA | 3477 |
| 1558 | AUCCAACU G AUGGAUCU | 1289 | AGAUCCAU UGAUG GCAUGCACUAUGC GCG AGUUGGAU | 3478 |
| 1567 | AUGGAUCU G AAAUUGUG | 1290 | CACAAUUU UGAUG GCAUGCACUAUGC GCG AGAUCCAU | 3479 |
| 1575 | GAAAUUGU G CUGCUGAC | 1291 | GUCAGCAG UGAUG GCAUGCACUAUGC GCG ACAAUUUC | 3480 |
| 1578 | AUUGUGCU G CUGACGGA | 1292 | UCCGUCAG UGAUG GCAUGCACUAUGC GCG AGCACAAU | 3481 |
| 1581 | GUGCUGCU G ACGGAUGG | 1293 | CCAUCCGU UGAUG GCAUGCACUAUGC GCG AGCAGCAC | 3482 |
| 1613 | AAGUGGGU G CUUUAACG | 1294 | CGUUAAAG UGAUG GCAUGCACUAUGC GCG ACCCACUU | 3483 |
| 1621 | GCUUUAAC G AGGUCAAA | 1295 | UUUGACCU UGAUG GCAUGCACUAUGC GCG GUUAAAGC | 3484 |
| 1639 | AAAGUGGU G CCAUCAUC | 1296 | GAUGAUGG UGAUG GCAUGCACUAUGC GCG ACCACUUU | 3485 |
| 1657 | ACACAGUC G CUUUGGGG | 1297 | CCCCAAAG UGAUG GCAUGCACUAUGC GCG GACUGUGU | 3486 |
| 1672 | GGCCCUCU G CAGCUCAA | 1298 | UUGAGCUG UGAUG GCAUGCACUAUGC GCG AGAGGGCC | 3487 |
| 1704 | UCCAAAAU G ACAGGAGG | 1299 | CCUCCUGU UGAUG GCAUGCACUAUGC GCG AUUUUGGA | 3488 |
| 1726 | AGACAUAU G CUUCAGAU | 1300 | AUCUGAAG UGAUG GCAUGCACUAUGC GCG AUAUGUCU | 3489 |
| 1759 | GCCUCAUU G AUGCUUUU | 1301 | AAAAGCAU UGAUG GCAUGCACUAUGC GCG AAUGAGGC | 3490 |
| 1762 | UCAUUGAU G CUUUUGGG | 1302 | CCCAAAAG UGAUG GCAUGCACUAUGC GCG AUCAAUGA | 3491 |
| 1805 | CUCUCAGC G CUCCAUCC | 1303 | GGAUGGAG UGAUG GCAUGCACUAUGC GCG GCUGAGAG | 3492 |
| 1819 | UCCAGCUU G AGAGUAAG | 1304 | CUUACUCU UGAUG GCAUGCACUAUGC GCG AAGCUGGA | 3493 |
| 1857 | CAGUGGAU G AAUGGCAC | 1305 | GUGCCAUU UGAUG GCAUGCACUAUGC GCG AUCCACUG | 3494 |
| 1869 | GGCACAGU G AUCGUGGA | 1306 | UCCACGAU UGAUG GCAUGCACUAUGC GCG ACUGUGCC | 3495 |
| 1923 | UGGACAAC G CAGCCUCC | 1307 | GGAGGCUG UGAUG GCAUGCACUAUGC GCG GUUGUCCA | 3496 |
| 2026 | CAGGCAUU G CUAAGGUU | 1308 | AACCUUAG UGAUG GCAUGCACUAUGC GCG AAUGCCUG | 3497 |
| 2055 | UACAGUCU G CAAGCAAG | 1309 | CUUGCUUG UGAUG GCAUGCACUAUGC GCG AGACUGUA | 3498 |
| 2076 | CAAACCUU G ACCCUGAC | 1310 | GUCAGGGU UGAUG GCAUGCACUAUGC GCG AAGGUUUG | 3499 |
| 2082 | UUGACCCU G ACUGUCAC | 1311 | GUGACAGU UGAUG GCAUGCACUAUGC GCG AGGGUCAA | 3500 |
| 2098 | CGUCCCGU G CGUCCAAU | 1312 | AUUGGACG UGAUG GCAUGCACUAUGC GCG ACGGGACG | 3501 |
| 2107 | CGUCCAAU G CUACCCUG | 1313 | CAGGGUAG UGAUG GCAUGCACUAUGC GCG AUUGGACG | 3502 |
| 2115 | GCUACCCU G CCUCCAAU | 1314 | AUUGGAGG UGAUG GCAUGCACUAUGC GCG AGGGUAGC | 3503 |
| 2130 | AUUACAGU G ACUUCCAA | 1315 | UUGGAAGU UGAUG GCAUGCACUAUGC GCG ACUGUAAU | 3504 |
| 2142 | UCCAAAAC G AACAAGGA | 1316 | UCCUUGUU UGAUG GCAUGCACUAUGC GCG GUUUUGGA | 3505 |
| 2185 | UAGUUUAU G CAAAUAUU | 1317 | AAUAUUUG UGAUG GCAUGCACUAUGC GCG AUAAACUA | 3506 |
| 2195 | AAAUAUUC G CCAAGGAG | 1318 | CUCCUUGG UGAUG GCAUGCACUAUGC GCG GAAUAUUU | 3507 |
| 2238 | ACAGCCCU G AUUGAAUC | 1319 | GAUUCAAU UGAUG GCAUGCACUAUGC GCG AGGGCUGU | 3508 |
| 2242 | CCCUGAUU G AAUCAGUG | 1320 | CACUGAUU UGAUG GCAUGCACUAUGC GCG AAUCAGGG | 3509 |
| 2250 | GAAUCAGU G AAUGGAAA | 1321 | UUUCCAUU UGAUG GCAUGCACUAUGC GCG ACUGAUUC | 3510 |
| 2296 | GAGCAGGU G CUGAUGCU | 1322 | AGCAUCAG UGAUG GCAUGCACUAUGC GCG ACCUGCUC | 3511 |
| 2299 | CAGGUGCU G AUGCUACU | 1323 | AGUAGCAU UGAUG GCAUGCACUAUGC GCG AGCACCUG | 3512 |
| 2302 | GUGCUGAU G CUACUAAG | 1324 | CUUAGUAG UGAUG GCAUGCACUAUGC GCG AUCAGCAC | 3513 |
| 2314 | CUAAGGAU G ACGGUGUC | 1325 | GACACCGU UGAUG GCAUGCACUAUGC GCG AUCCUUAG | 3514 |
| 2347 | CAACUUAU G ACACGAAU | 1326 | AUUCGUGU UGAUG GCAUGCACUAUGC GCG AUAAGUUG | 3515 |
| 2352 | UAUGACAC G AAUGGUAG | 1327 | CUACCAUU UGAUG GCAUGCACUAUGC GCG GUGUCAUA | 3516 |
| 2376 | GUAAAAGU G CGGGCUCU | 1328 | AGAGCCCG UGAUG GCAUGCACUAUGC GCG ACUUUUAC | 3517 |
| 2398 | GAGUUAAC G CAGCCAGA | 1329 | UCUGGCUG UGAUG GCAUGCACUAUGC GCG GUUAACUC | 3518 |
| 2415 | CGGAGAGU G AUACCCCA | 1330 | UGGGGUAU UGAUG GCAUGCACUAUGC GCG ACUCUCCG | 3519 |
| 2458 | GCUGGAUU G AGAAUGAU | 1331 | AUCAUUCU UGAUG GCAUGCACUAUGC GCG AAUCCAGC | 3520 |
| 2464 | UUGAGAAU G AUGAAAUA | 1332 | UAUUUCAU UGAUG GCAUGCACUAUGC GCG AUUCUCAA | 3521 |
| 2467 | AGAAUGAU G AAAUACAA | 1333 | UUGUAUUU UGAUG GCAUGCACUAUGC GCG AUCAUUCU | 3522 |
| 2494 | CAAGACCU G AAAUUAAU | 1334 | AUUAAUUU UGAUG GCAUGCACUAUGC GCG AGGUCUUG | 3523 |
| 2509 | AUAAGGAU G AUGUUCAA | 1335 | UUGAACAU UGAUG GCAUGCACUAUGC GCG AUCCUUAU | 3524 |
| 2572 | UGGCUUCU G AUGUCCCA | 1336 | UGGGACAU UGAUG GCAUGCACUAUGC GCG AGAAGCCA | 3525 |
| 2584 | UCCCAAAU G CUCCCAUA | 1337 | UAUGGGAG UGAUG GCAUGCACUAUGC GCG AUUUGGGA | 3526 |
| 2596 | CCAUACCU G AUCUCUUC | 1338 | GAAGAGAU UGAUG GCAUGCACUAUGC GCG AGGUAUGG | 3527 |
| 2623 | AAAUCACC G ACCUGAAG | 1339 | CUUCAGGU UGAUG GCAUGCACUAUGC GCG GGUGAUUU | 3528 |
| 2628 | ACCGACCU G AAGGCGGA | 1340 | UCCGCCUU UGAUG GCAUGCACUAUGC GCG AGGUCGGU | 3529 |
| 2664 | AUUAAUCU G ACUUGGAC | 1341 | GUCCAAGU UGAUG GCAUGCACUAUGC GCG AGAUUAAU | 3530 |
| 2686 | CUGGGGAU G AUUAUGAC | 1342 | GUCAUAAU UGAUG GCAUGCACUAUGC GCG AUCCCCAG | 3531 |
| 2692 | AUGAUUAU G ACCAUGGA | 1343 | UCCAUGGU UGAUG GCAUGCACUAUGC GCG AUAAUCAU | 3532 |
| 2723 | UAUCAUUC G AAUAAGUA | 1344 | UACUUAUU UGAUG GCAUGCACUAUGC GCG GAAUGAUA | 3533 |
| 2743 | GUAUUCUU G AUCUCAGA | 1345 | UCUGAGAU UGAUG GCAUGCACUAUGC GCG AAGAAUAC | 3534 |
| 2764 | AGUUCAAU G AAUCUCUU | 1346 | AAGAGAUU UGAUG GCAUGCACUAUGC GCG AUUGAACU | 3535 |
| 2778 | CUUCAAGU G AAUACUAC | 1347 | GUAGUAUU UGAUG GCAUGCACUAUGC GCG ACUUGAAG | 3536 |
| 2788 | AUACUACU G CUCUCAUC | 1348 | GAUGAGAG UGAUG GCAUGCACUAUGC GCG AGUAGUAU | 3537 |
| 2815 | CCAACUCU G AGGAAGUC | 1349 | GACUUCCU UGAUG GCAUGCACUAUGC GCG AGAGUUGG | 3538 |
| 2854 | UUACUUUU G AAAAUGGC | 1350 | GCCAUUUU UGAUG GCAUGCACUAUGC GCG AAAAGUAA | 3539 |
| 2878 | UUUUCAUU G CUAUUCAG | 1351 | CUGAAUAG UGAUG GCAUGCACUAUGC GCG AAUGAAAA | 3540 |
| 2893 | AGGCUGUU G AUAAGGUC | 1352 | GACCUUAU UGAUG GCAUGCACUAUGC GCG AACAGCCU | 3541 |
| 2902 | AUAAGGUC G AUCUGAAA | 1353 | UUUCAGAU UGAUG GCAUGCACUAUGC GCG GACCUUAU | 3542 |
| 2907 | GUCGAUCU G AAAUCAGA | 1354 | UCUGAUUU UGAUG GCAUGCACUAUGC GCG AGAUCGAC | 3543 |
| 2929 | CCAACAUU G CACGAGUA | 1355 | UACUCGUG UGAUG GCAUGCACUAUGC GCG AAUGUUGG | 3544 |
| 2933 | CAUUGCAC G AGUAUCUU | 1356 | AAGAUACU UGAUG GCAUGCACUAUGC GCG GUGCAAUG | 3545 |
| 2964 | CAGACUCC G CCAGAGAC | 1357 | GUCUCUGG UGAUG GCAUGCACUAUGC GCG GGAGUCUG | 3546 |
| 2983 | CUAGUCCU G AUGAAACG | 1358 | CGUUUCAU UGAUG GCAUGCACUAUGC GCG AGGACUAG | 3547 |
| 2986 | GUCCUGAU G AAACGUCU | 1359 | AGACGUUU UGAUG GCAUGCACUAUGC GCG AUCAGGAC | 3548 |
| 2995 | AAACGUCU G CUCCUUGU | 1360 | ACAAGGAG UGAUG GCAUGCACUAUGC GCG AGACGUUU | 3549 |
| 3078 | GGAGAACU G CAGCUGUC | 1361 | GACAGCUG UGAUG GCAUGCACUAUGC GCG AGUUCUCC | 3550 |
| 3101 | CUAGGGCU G AAUUUUUG | 1362 | CAAAAAUU UGAUG GCAUGCACUAUGC GCG AGCCCUAG | 3551 |
| 3145 | CUUUUUUU G AUUAUAAA | 1363 | UUUAUAAU UGAUG GCAUGCACUAUGC GCG AAAAAAAG | 3552 |
| 3191 | UAGGGGGC G AUAUACUA | 1364 | UAGUAUAU UGAUG GCAUGCACUAUGC GCG GCCCCCUA | 3553 |
| 3244 | UAGGGGGC G AUAUACUA | 1364 | UAGUAUAU UGAUG GCAUGCACUAUGC GCG GCCCCCUA | 3553 |
| 3281 | UAGGGGGC G AUAAAAUA | 1365 | UAUUUUAU UGAUG GCAUGCACUAUGC GCG GCCCCCUA | 3554 |
| 3294 | AAUAAAAU G CUAAACAA | 1366 | UUGUUUAG UGAUG GCAUGCACUAUGC GCG AUUUUAUU | 3555 |
| 27 | AAAUGGAU G UGGAAUAU | 1367 | AUAUUCCA UGAUG GCAUGCACUAUGC GCG AUCCAUUU | 3556 |
| 52 | AUUUUCUU G UUUAAGGG | 1368 | CCCUUAAA UGAUG GCAUGCACUAUGC GCG AAGAAAAU | 3557 |
| 75 | GAAGAGGU G UUGAGGUU | 1369 | AACCUCAA UGAUG GCAUGCACUAUGC GCG ACCUCUUC | 3558 |
| 86 | GAGGUUAU G UCAAGCAU | 1370 | AUGCUUGA UGAUG GCAUGCACUAUGC GCG AUAACCUC | 3559 |
| 155 | AAGAUAUU G UUAUCAUU | 1371 | AAUGAUAA UGAUG GCAUGCACUAUGC GCG AAUAUCUU | 3560 |
| 221 | AAAGACCU G UGAUAAAC | 1372 | GUUUAUCA UGAUG GCAUGCACUAUGC GCG AGGUCUUU | 3561 |
| 253 | GGAAACGU G UGUGUAUA | 1373 | UAUAGACA UGAUG GCAUGCACUAUGC GCG ACGUUUCC | 3562 |
| 255 | AAACGUGU G UCUAUAUU | 1374 | AAUAUAGA UGAUG GCAUGCACUAUGC GCG ACACGUUU | 3563 |
| 273 | UCAUAUCU G UAUAUAUA | 1375 | UAUAUAUA UGAUG GCAUGCACUAUGC GCG AGAUAUGA | 3564 |
| 344 | AGGGAGAU G UACAGCAA | 1376 | UUGCUGUA UGAUG GCAUGCACUAUGC GCG AUCUCCCU | 3565 |
| 373 | AGAGUUCU G UGUUCAUC | 1377 | GAUGAACA UGAUG GCAUGCACUAUGC GCG AGAACUCU | 3566 |
| 375 | AGUUCUGU G UUCAUCUU | 1378 | AAGAUGAA UGAUG GCAUGCACUAUGC GCG ACAGAACU | 3567 |
| 457 | AAGGCAUU G UCGUUGCA | 1379 | UGCAACGA UGAUG GCAUGCACUAUGC GCG AAUGCCUU | 3568 |
| 478 | ACCCCAAU G UGCCAGAA | 1380 | UUCUGGCA UGAUG GCAUGCACUAUGC GCG AUUGGGGU | 3569 |
| 537 | GCAUCUCU G UAUCUGUU | 1381 | AACAGAUA UGAUG GCAUGCACUAUGC GCG AGAGAUGC | 3570 |
| 543 | CUGUAUCU G UUUGAAGC | 1382 | GCUUCAAA UGAUG GCAUGCACUAUGC GCG AGAUACAG | 3571 |
| 580 | UCAAAAAU G UUGCCAUU | 1383 | AAUGGCAA UGAUG GCAUGCACUAUGC GCG AUUUUUGA | 3572 |
| 625 | CUGACUAU G UGAGACCA | 1384 | UGGUCUCA UGAUG GCAUGCACUAUGC GCG AUAGUCAG | 3573 |
| 661 | AUGCUGAU G UUCUGGUU | 1385 | AACCAGAA UGAUG GCAUGCACUAUGC GCG AUCAGCAU | 3574 |
| 725 | GGGCAACU G UGGAGAGA | 1386 | UCUCUCCA UGAUG GCAUGCACUAUGC GCG AGUUGCCC | 3575 |
| 814 | AGGCAUUU G UCCAUGAG | 1387 | CUCAUGGA UGAUG GCAUGCACUAUGC GCG AAAUGCCU | 3576 |
| 911 | AGUAAGAU G UUCAGCAG | 1388 | CUGCUGAA UGAUG GCAUGCACUAUGC GCG AUCUUACU | 3577 |
| 937 | GUACAAAU G UAGUAAAG | 1389 | CUUUACUA UGAUG GCAUGCACUAUGC GCG AUUUGUAC | 3578 |
| 950 | AAAGAAGU G UCAGGGAG | 1390 | CUCCCUGA UGAUG GCAUGCACUAUGC GCG ACUUCUUU | 3579 |
| 965 | AGGCAGCU G UUACACCA | 1391 | UGGUGUAA UGAUG GCAUGCACUAUGC GCG AGCUGCCU | 3580 |
| 1019 | AAAAGGAU G UGAGUUUG | 1392 | CAAACUCA UGAUG GCAUGCACUAUGC GCG AUCCUUUU | 3581 |
| 1027 | GUGAGUUU G UUCUCCAA | 1393 | UUGGAGAA UGAUG GCAUGCACUAUGC GCG AAACUCAC | 3582 |
| 1065 | UCUAUAAU G UUUGCACA | 1394 | UGUGCAAA UGAUG GCAUGCACUAUGC GCG AUUAUAGA | 3583 |
| 1078 | CACAACAU G UUGAUUCU | 1395 | AGAAUCAA UGAUG GCAUGCACUAUGC GCG AUGUUGUG | 3584 |
| 1100 | UGAAUUCU G UACAGAAC | 1396 | GUUCUGUA UGAUG GCAUGCACUAUGC GCG AGAAUUCA | 3585 |
| 1270 | AAAGAAUU G UGUGUUUA | 1397 | UAAACACA UGAUG GCAUGCACUAUGC GCG AAUUCUUU | 3586 |
| 1272 | AGAAUUGU G UGUUUAGU | 1398 | ACUAAACA UGAUG GCAUGCACUAUGC GCG ACAAUUCU | 3587 |
| 1274 | AAUUGUGU G UUUAGUCC | 1399 | GGACUAAA UGAUG GCAUGCACUAUGC GCG ACACAAUU | 3588 |
| 1414 | CUGCCCAU G UACAAAGU | 1400 | ACUUUGUA UGAUG GCAUGCACUAUGC GCG AUGOGGAG | 3589 |
| 1534 | CAUUUACU G UGAUUAGG | 1401 | CCUAAUCA UGAUG GCAUGCACUAUGC GCG AGUAAAUG | 3590 |
| 1573 | CUGAAAUU G UGCUGCUG | 1402 | CAGCAGCA UGAUG GCAUGCACUAUGC GCG AAUUUCAG | 3591 |
| 1695 | GAGGAGCU G UCCAAAAU | 1403 | AUUUUGGA UGAUG GCAUGCACUAUGC GCG AGCUCCUC | 3592 |
| 1795 | AUGGAGCU G UCUCUCAG | 1404 | CUGAGAGA UGAUG GCAUGCACUAUGC GCG AGCUCCAU | 3593 |
| 1902 | GACACUUU G UUUCUUAU | 1405 | AUAAGAAA UGAUG GCAUGCACUAUGC GCG AAAGUGUC | 3594 |
| 1978 | GUGGCUUU G UAGUGGAC | 1406 | GUCCACUA UGAUG GCAUGCACUAUGC GCG AAAGCCAC | 3595 |
| 2086 | CCCUGACU G UCACGUCC | 1407 | GGACGUGA UGAUG GCAUGCACUAUGC GCG AGUCAGGG | 3596 |
| 2227 | GGGCCAGU G UCACAGCC | 1408 | GGCUGUGA UGAUG GCAUGCACUAUGC GCG ACUGGCCC | 3597 |
| 2320 | AUGACGGU G UCUACUCA | 1409 | UGAGUAGA UGAUG GCAUGCACUAUGC GCG ACCGUCAU | 3598 |
| 2368 | GAUACAGU G UAAAAGUG | 1410 | CACUUUUA UGAUG GCAUGCACUAUGC GCG ACUGUAUC | 3599 |
| 2439 | GGAGCACU G UACAUACC | 1411 | GGUAUGUA UGAUG GCAUGCACUAUGC GCG AGUGCUCC | 3600 |
| 2512 | AGGAUGAU G UUCAACAC | 1412 | GUGUUGAA UGAUG GCAUGCACUAUGC GCG AUCAUCCU | 3601 |
| 2529 | AAGCAAGU G UGUUUCAG | 1413 | CUGAAACA UGAUG GCAUGCACUAUGC GCG ACUUGCUU | 3602 |
| 2531 | GCAAGUGU G UUCAGCA | 1414 | UGCUGAAA UGAUG GCAUGCACUAUGC GCG ACACUUGC | 3603 |
| 2563 | GCUCAUUU G UGGCUUCU | 1415 | AGAAGCCA UGAUG GCAUGCACUAUGC GCG AAAUGAGC | 3604 |
| 2575 | CUUCUGAU G UCCCAAAU | 1416 | AUUUGGGA UGAUG GCAUGCACUAUGC GCG AUCAGAAG | 3605 |
| 2829 | GUCUUUUU G UUUAAACC | 1417 | GGUUUAAA UGAUG GCAUGCACUAUGC GCG AAAAAGAC | 3606 |
| 2890 | UUCAGGCU G UUGAUAAG | 1418 | CUUAUCAA UGAUG GCAUGCACUAUGC GCG AGCCUGAA | 3607 |
| 2943 | GUAUCUUU G UUUAUUCC | 1419 | GGAAUAAA UGAUG GCAUGCACUAUGC GCG AAAGAUAC | 3608 |
| 3002 | UGCUCCUU G UCCUAAUA | 1420 | UAUUAGGA UGAUG GCAUGCACUAUGC GCG AAGGAGCA | 3609 |
| 3057 | AAAAUUAU G UGGAAGUG | 1421 | CACUUCCA UGAUG GCAUGCACUAUGC GCG AUAAUUUU | 3610 |
| 3084 | CUGCAGCU G UCAAUAGC | 1422 | GCUAUUGA UGAUG GCAUGCACUAUGC GCG AGCUGCAG | 3611 |
| 3109 | GAAUUUUU G UCAGAUAA | 1423 | UUAUCUGA UGAUG GCAUGCACUAUGC GCG AAAAAUUC | 3612 |
| 3166 | UCUAAAAU G UAUUUUAG | 1424 | CUAAAAUA UGAUG GCAUGCACUAUGC GCG AUUUUAGA | 3613 |
| 3182 | GACUUCCU G UAGGGGGC | 1425 | GCCCCCUA UGAUG GCAUGCACUAUGC GCG AGGAAGUC | 3614 |
| 3272 | GACUUCCU G UAGGGGGC | 1425 | GCCCCCUA UGAUG GCAUGCACUAUGC GCG AGGAAGUC | 3614 |
| 3203 | UACUAAAU G UAUAUAGU | 1426 | ACUAUAUA UGAUG GCAUGCACUAUGC GCG AUUUAGUA | 3615 |
| 3227 | UACUAAAU G UAUUCCUG | 1427 | CAGGAAUA UGAUG GCAUGCACUAUGC GCG AUUUAGUA | 3616 |
| 3235 | GUAUUCCU G UAGGGGGC | 1428 | GCCCCCUA UGAUG GCAUGCACUAUGC GCG AGGAAUAC | 3617 |
| 3256 | UACUAAAU G UAUUUUAG | 1429 | CUAAAAUA UGAUG GCAUGCACUAUGC GCG AUUUAGUA | 3618 |

Input Sequence = NM_001285. Cut Site = YG/M or UG/U.
Arm Length = 8. Core Sequence = UGAUG GCAUGCACUAUGC GCG
NM_001285 (Homo sapiens chloride channel, calcium activated, 1 (CLCA 1) mRNA, 3311 bp)

TABLE VI
Human CLCA1 Zinzyme and Target Sequence 249.021

| | | Seq | | Rz Seq |
|---|---|---|---|---|
| Pos | Substrate | ID | Zinzyme | ID |
| 134 | ACAAGUAC G CAAUUUGA | 1215 | UCAAAUUG GCCGAAAGGCGAGUGAGGUCU GUACUUGU | 3619 |
| 312 | CGUAACCC G CAUUUUCC | 1220 | GGAAAAUG GCCGAAAGGCGAGUGAGGUCU GGGUUACG | 3620 |
| 463 | UUGUCGUU G CAAUCGAC | 1225 | GUCGAUUG GCCGAAAGGCGAGUGAGGUCU AACGACAA | 3621 |
| 480 | CCCAAUGU G CCAGAAGA | 1227 | UCUUCUGG GCCGAAAGGCGAGUGAGGUCU ACAUUGGG | 3622 |
| 583 | AAAAUGUU G CCAUUUUG | 1232 | CAAAAUGG GCCGAAAGGCGAGUGAGGUCU AACAUUUU | 3623 |
| 655 | ACAAAAAU G CUGAUGUU | 1238 | AACAUCAG GCCGAAAGGCGAGUGAGGUCU AUUUUUGU | 3624 |
| 670 | UUCUGGUU G CUGAGUCU | 1240 | AGACUCAG GCCGAAAGGCGAGUGAGGUCU AACCAGAA | 3625 |
| 769 | AUUUCAUU G CAGGAAAA | 1247 | UUUUCCUG GCCGAAAGGCGAGUGAGGUCU AAUGAAAU | 3626 |
| 980 | CAAAAGAU G CACAUUCA | 1255 | UGAAUGUG GCCGAAAGGCGAGUGAGGUCU AUCUUUUG | 3627 |
| 1040 | CCAAUCCC G CCAGACGG | 1258 | CCGUCUGG GCCGAAAGGCGAGUGAGGUCU GGGAUUGG | 3628 |
| 1069 | UAAUGUUU G CACAACAU | 1259 | AUGUUGUG GCCGAAAGGCGAGUGAGGUCU AAACAUUA | 3629 |
| 1151 | UCAAAAAU G CAAUCUCC | 1262 | GGAGAUUG GCCGAAAGGCGAGUGAGGUCU AUUUUUGA | 3630 |
| 1248 | UUCUCAUU G CUGCAGAU | 1268 | AUCUGCAG GCCGAAAGGCGAGUGAGGUCU AAUGAGAA | 3631 |
| 1251 | UCAUUGCU G CAGAUUGG | 1269 | CCAAUCUG GCCGAAAGGCGAGUGAGGUCU AGCAAUGA | 3632 |
| 1316 | UGGUAACC G CCUCAAUC | 1272 | GAUUGAGG GCCGAAAGGCGAGUGAGGUCU GGUUACCA | 3633 |
| 1353 | CUUUUCCU G CUGCAGAC | 1275 | GUCUGCAG GCCGAAAGGCGAGUGAGGUCU AGGAAAAG | 3634 |
| 1356 | UUCCUGCU G CAGACAGU | 1276 | ACUGUCUG GCCGAAAGGCGAGUGAGGUCU AGCAGGAA | 3635 |
| 1405 | UUGACAGU G CUGCCCAU | 1280 | AUGGGCAG GCCGAAAGGCGAGUGAGGUCU ACUGUCAA | 3636 |
| 1408 | ACAGUGCU G CCCAUGUA | 1281 | UACAUGGG GCCGAAAGGCGAGUGAGGUCU AGCACUGU | 3637 |
| 1465 | ACACACUC G CCAAAAGA | 1284 | UCUUUUGG GCCGAAAGGCGAGUGAGGUCU GAGUGUGU | 3638 |
| 1480 | GAUUACCU G CAGCAGCU | 1285 | AGCUGCUG GCCGAAAGGCGAGUGAGGUCU AGGUAAUC | 3639 |
| 1508 | GUCCAUCU G CAGCGGGC | 1286 | GCCCGCUG GCCGAAAGGCGAGUGAGGUCU AGAUGGAC | 3640 |
| 1575 | GAAAUUGU G CUGCUGAC | 1291 | GUCAGCAG GCCGAAAGGCGAGUGAGGUCU ACAAUUUC | 3641 |
| 1578 | AUUGUGCU G CUGACGGA | 1292 | UCCGUCAG GCCGAAAGGCGAGUGAGGUCU AGCACAAU | 3642 |
| 1613 | AAGUGGGU G CUUUAACG | 1294 | CGUUAAAG GCCGAAAGGCGAGUGAGGUCU ACCCACUU | 3643 |
| 1639 | AAAGUGGU G CCAUCAUC | 1296 | GAUGAUGG GCCGAAAGGCGAGUGAGGUCU ACCACUUU | 3644 |
| 1657 | ACACAGUC G CUUUGGGG | 1297 | CCCCAAAG GCCGAAAGGCGAGUGAGGUCU GACUGUGU | 3645 |
| 1672 | GGCCCUCU G CAGCUCAA | 1298 | UUGAGCUG GCCGAAAGGCGAGUGAGGUCU AGAGGGCC | 3646 |
| 1726 | AGACAUAU G CUUCAGAU | 1300 | AUCUGAAG GCCGAAAGGCGAGUGAGGUCU AUAUGUCU | 3647 |
| 1762 | UCAUUGAU G CUUUUGGG | 1302 | CCCAAAAG GCCGAAAGGCGAGUGAGGUCU AUCAAUGA | 3648 |
| 1805 | CUCUCAGC G CUCCAUCC | 1303 | GGAUGGAG GCCGAAAGGCGAGUGAGGUCU GCUGAGAG | 3649 |
| 1923 | UGGACAAC G CAGCCUCC | 1307 | GGAGGCUG GCCGAAAGGCGAGUGAGGUCU GUUGUCCA | 3650 |
| 2026 | CAGGCAUU G CUAAGGUU | 1308 | AACCUUAG GCCGAAAGGCGAGUGAGGUCU AAUGCCUG | 3651 |
| 2055 | UACAGUCU G CAAGCAAG | 1309 | CUUGCUUG GCCGAAAGGCGAGUGAGGUCU AGACUGUA | 3652 |
| 2098 | CGUCCCGU G CGUCCAAU | 1312 | AUUGGACG GCCGAAAGGCGAGUGAGGUCU ACGGGACG | 3653 |
| 2107 | CGUCCAAU G CUACCCUG | 1313 | CAGGGUAG GCCGAAAGGCGAGUGAGGUCU AUUGGACG | 3654 |
| 2115 | GCUACCCU G CCUCCAAU | 1314 | AUGGAGG GCCGAAAGGCGAGUGAGGUCU AGGGUAGC | 3655 |
| 2185 | UAGUUUAU G CAAAUAUU | 1317 | AAUAUUUG GCCGAAAGGCGAGUGAGGUCU AUAAACUA | 3656 |
| 2195 | AAAUAUUC G CCAAGGAG | 1318 | CUCCUUGG GCCGAAAGGCGAGUGAGGUCU GAAUAUUU | 3657 |
| 2296 | GAGCAGGU G CUGAUGCU | 1322 | AGCAUCAG GCCGAAAGGCGAGUGAGGUCU ACCUGCUC | 3658 |
| 2302 | GUGCUGAU G CUACUAAG | 1324 | CUUAGUAG GCCGAAAGGCGAGUGAGGUCU AUCAGCAC | 3659 |
| 2376 | GUAAAAGU G CGGGCUCU | 1328 | AGAGCCCG GCCGAAAGGCGAGUGAGGUCU ACUUUUAC | 3660 |
| 2398 | GAGUUAAC G CAGCCAGA | 1329 | UCUGGCUG GCCGAAAGGCGAGUGAGGUCU GUUAACUC | 3661 |
| 2584 | UCCCAAAU G CUCCCAUA | 1337 | UAUGGGAG GCCGAAAGGCGAGUGAGGUCU AUUUGGGA | 3662 |
| 2788 | AUACUACU G CUCUCAUC | 1348 | GAUGAGAG GCCGAAAGGCGAGUGAGGUCU AGUAGUAU | 3663 |
| 2878 | UUUUCAUU G CUAUUCAG | 1351 | CUGAAUAG GCCGAAAGGCGAGUGAGGUCU AAUGAAAA | 3664 |
| 2929 | CCAACAUU G CACGAGUA | 1355 | UACUCGUG GCCGAAAGGCGAGUGAGGUCU AAUGUUGG | 3665 |
| 2964 | CAGACUCC G CCAGAGAC | 1357 | GUCUCUGG GCCGAAAGGCGAGUGAGGUCU GGAGUCUG | 3666 |
| 2995 | AAACGUCU G CUCCUUGU | 1360 | ACAAGGAG GCCGAAAGGCGAGUGAGGUCU AGACGUUU | 3667 |
| 3078 | GGAGAACU G CAGCUGUC | 1361 | GACAGCUG GCCGAAAGGCGAGUGAGGUCU AGUUCUCC | 3668 |
| 3294 | AAUAAAAU G CUAAACAA | 1366 | UUGUUUAG GCCGAAAGGCGAGUGAGGUCU AUUUUAUU | 3669 |
| 27 | AAAUGGAU G UGGAAUAU | 1367 | AUAUUCCA GCCGAAAGGCGAGUGAGGUCU AUCCAUUU | 3670 |
| 52 | AUUUUCUU G UUUAAGGG | 1368 | CCCUUAAA GCCGAAAGGCGAGUGAGGUCU AAGAAAAU | 3671 |
| 75 | GAAGAGGU G UUGAGGUU | 1369 | AACCUCAA GCCGAAAGGCGAGUGAGGUCU ACCUCUUC | 3672 |
| 86 | GAGGUUAU G UCAAGCAU | 1370 | AUGCUUGA GCCGAAAGGCGAGUGAGGUCU AUAACCUC | 3673 |
| 155 | AAGAUAUU G UUAUCAUU | 1371 | AAUGAUAA GCCGAAAGGCGAGUGAGGUCU AAUAUCUU | 3674 |
| 221 | AAAGACCU G UGAUAAAC | 1372 | GUUUAUCA GCCGAAAGGCGAGUGAGGUCU AGGUCUUU | 3675 |
| 253 | GGAAACGU G UGUCUAUA | 1373 | UAUAGACA GCCGAAAGGCGAGUGAGGUCU ACGUUUCC | 3676 |
| 255 | AAACGUGU G UCUAUAUU | 1374 | AAUAUAGA GCCGAAAGGCGAGUGAGGUCU ACACGUUU | 3677 |
| 273 | UCAUAUCU G UAUAUAUA | 1375 | UAUAUAUA GCCGAAAGGCGAGUGAGGUCU AGAUAUGA | 3678 |
| 344 | AGGGAGAU G UACAGCAA | 1376 | UUGCUGUA GCCGAAAGGCGAGUGAGGUCU AUCUCCCU | 3679 |
| 373 | AGAGUUCU G UGUUCAUC | 1377 | GAUGAACA GCCGAAAGGCGAGUGAGGUCU AGAACUCU | 3680 |
| 375 | AGUUCUGU G UUCAUCUU | 1378 | AAGAUGAA GCCGAAAGGCGAGUGAGGUCU ACAGAACU | 3681 |
| 457 | AAGGCAUU G UCGUUGCA | 1379 | UGCAACGA GCCGAAAGGCGAGUGAGGUCU AAUGCCUU | 3682 |
| 478 | ACCCCAAU G UGCCAGAA | 1380 | UUCUGGCA GCCGAAAGGCGAGUGAGGUCU AUUGGGGU | 3683 |
| 537 | GCAUCUCU G UAUCUGUU | 1381 | AACAGAUA GCCGAAAGGCGAGUGAGGUCU AGAGAUGC | 3684 |
| 543 | CUGUAUCU G UUUGAAGC | 1382 | GCUUCAAA GCCGAAAGGCGAGUGAGGUCU AGAUACAG | 3685 |
| 580 | UCAAAAAU G UUGCCAUU | 1383 | AAUGGCAA GCCGAAAGGCGAGUGAGGUCU AUUUUUGA | 3686 |
| 625 | CUGACUAU G UGAGACCA | 1384 | UGGUCUCA GCCGAAAGGCGAGUGAGGUCU AUAGUCAG | 3687 |
| 661 | AUGCUGAU G UUCUGGUU | 1385 | AACCAGAA GCCGAAAGGCGAGUGAGGUCU AUCAGCAU | 3688 |
| 725 | GGGCAACU G UGGAGAGA | 1386 | UCUCUCCA GCCGAAAGGCGAGUGAGGUCU AGUUGCCC | 3689 |
| 814 | AGGCAUUU G UCCAUGAG | 1387 | CUCAUGGA GCCGAAAGGCGAGUGAGGUCU AAAUGCCU | 3690 |
| 911 | AGUAAGAU G UUCAGCAG | 1388 | CUGCUGAA GCCGAAAGGCGAGUGAGGUCU AUCUUACU | 3691 |
| 937 | GUACAAAU G UAGUAAAG | 1389 | CUUUACUA GCCGAAAGGCGAGUGAGGUCU AUUUUAC | 3692 |
| 950 | AAAGAAGU G UCAGGGAG | 1390 | CUCCCUGA GCCGAAAGGCGAGUGAGGUCU ACUUCUUU | 3693 |
| 965 | AGGCAGCU G UUACACCA | 1391 | UGGUGUAA GCCGAAAGGCGAGUGAGGUCU AGCUGCCU | 3694 |
| 1019 | AAAAGGAU G UGAGUUUG | 1392 | CAAACUCA GCCGAAAGGCGAGUGAGGUCU AUCCUUUU | 3695 |
| 1027 | GUGAGUUU G UUCUCCAA | 1393 | UUGGAGAA GCCGAAAGGCGAGUGAGGUCU AAACUCAC | 3696 |
| 1065 | UCUAUAAU G UUUGCACA | 1394 | UGUGCAAA GCCGAAAGGCGAGUGAGGUCU AUUAUAGA | 3697 |
| 1078 | CACAACAU G UUGAUUCU | 1395 | AGAAUCAA GCCGAAAGGCGAGUGAGGUCU AUGUUGUG | 3698 |
| 1100 | UGAAUUCU G UACAGAAC | 1396 | GUUCUGUA GCCGAAAGGCGAGUGAGGUCU AGAAUUCA | 3699 |
| 1270 | AAAGAAUU G UGUGUUUA | 1397 | UAAACACA GCCGAAAGGCGAGUGAGGUCU AAUUCUUU | 3700 |
| 1272 | AGAAUUGU G UGUUUAGU | 1398 | ACUAAACA GCCGAAAGGCGAGUGAGGUCU ACAAUUCU | 3701 |
| 1274 | AAUUGUGU G UUUAGUCC | 1399 | GGACUAAA GCCGAAAGGCGAGUGAGGUCU ACACAAUU | 3702 |
| 1414 | CUGCCCAU G UACAAAGU | 1400 | ACUUUGUA GCCGAAAGGCGAGUGAGGUCU AUGGGCAG | 3703 |
| 1534 | CAUUUACU G UGAUUAGG | 1401 | CCUAAUCA GCCGAAAGGCGAGUGAGGUCU AGUAAAUG | 3704 |
| 1573 | CUGAAAUU G UGCUGCUG | 1402 | CAGGAGGA GCCGAAAGGCGAGUGAGGUCU AAUUUCAG | 3705 |
| 1695 | GAGGAGCU G UCCAAAAU | 1403 | AUUUUGGA GCCGAAAGGCGAGUGAGGUCU AGCUCCUC | 3706 |
| 1795 | AUGGAGCU G UCUCUCAG | 1404 | CUGAGAGA GCCGAAAGGCGAGUGAGGUCU AGCUCCAU | 3707 |
| 1902 | GACACUUU G UUUCUUAU | 1405 | AUAAGAAA GCCGAAAGGCGAGUGAGGUCU AAAGUGUC | 3708 |
| 1978 | GUGGCUUU G UAGUGGAC | 1406 | GUCCACUA GCCGAAAGGCGAGUGAGGUCU AAAGCCAC | 3709 |
| 2086 | CCCUGACU G UCACGUCC | 1407 | GGACGUGA GCCGAAAGGCGAGUGAGGUCU AGUCAGGG | 3710 |
| 2227 | GGGCCAGU G UCACAGCC | 1408 | GGCUGUGA GCCGAAAGGCGAGUGAGGUCU ACUGGCCC | 3711 |
| 2320 | AUGACGGU G UCUACUCA | 1409 | UGAGUAGA GCCGAAAGGCGAGUGAGGUCU ACCGUCAU | 3712 |
| 2368 | GAUACAGU G UAAAGUG | 1410 | CACUUUUA GCCGAAAGGCGAGUGAGGUCU ACUGUAUC | 3713 |
| 2439 | GGAGCACU G UACAUACC | 1411 | GGUAUGUA GCCGAAAGGCGAGUGAGGUCU AGUGCUCC | 3714 |
| 2512 | AGGAUGAU G UUCAACAC | 1412 | GUGUUGAA GCCGAAAGGCGAGUGAGGUCU AUCAUCCU | 3715 |
| 2529 | AAGCAAGU G UGUUUCAG | 1413 | CUGAAACA GCCGAAAGGCGAGUGAGGUCU ACUUGCUU | 3716 |
| 2531 | GCAAGUGU G UUUCAGCA | 1414 | UGCUGAAA GCCGAAAGGCGAGUGAGGUCU ACACUUGC | 3717 |
| 2563 | GCUCAUUU G UGGCUUCU | 1415 | AGAAGCCA GCCGAAAGGCGAGUGAGGUCU AAAUGAGC | 3718 |
| 2575 | CUUCUGAU G UCCCAAAU | 1416 | AUUUGGGA GCCGAAAGGCGAGUGAGGUCU AUCAGAAG | 3719 |
| 2829 | GUCUUUUU G UUUAUACC | 1417 | GGUUUAAA GCCGAAAGGCGAGUGAGGUCU AAAAAGAC | 3720 |
| 2890 | UUCAGGCU G UUGAUAAG | 1418 | CUUAUCAA GCCGAAAGGCGAGUGAGGUCU AGCCUGAA | 3721 |
| 2943 | GUAUCUUU G UUUAUUCC | 1419 | GGAAUAAA GCCGAAAGGCGAGUGAGGUCU AAAGAUAC | 3722 |
| 3002 | UGCUCCUU G UCCUAAUA | 1420 | UAUUAGGA GCCGAAAGGCGAGUGAGGUCU AAGGAGCA | 3723 |
| 3057 | AAAAUUAU G UGGAAGUG | 1421 | CACUUCCA GCCGAAAGGCGAGUGAGGUCU AUAAUUUU | 3724 |
| 3084 | CUGCAGCU G UCAAUAGC | 1422 | GCUAUUGA GCCGAAAGGCGAGUGAGGUCU AGCUGCAG | 3725 |
| 3109 | GAAUUUUU G UCAGAUAA | 1423 | UUAUCUGA GCCGAAAGGCGAGUGAGGUCU AAAAAUUC | 3726 |
| 3166 | UCUAAAAU G UAUUUUAG | 1424 | CUAAAAUA GCCGAAAGGCGAGUGAGGUCU AUUUUAGA | 3727 |
| 3182 | GACUUCCU G UAGGGGGC | 1425 | GCCCCCUA GCCGAAAGGCGAGUGAGGUCU AGGAAGUC | 3728 |
| 3272 | GACUUCCU G UAGGGGGC | 1425 | GCCCCCUA GCCGAAAGGCGAGUGAGGUCU AGGAAGUC | 3728 |
| 3203 | UACUAAAU G UAUAUAGU | 1426 | ACUAUAUA GCCGAAAGGCGAGUGAGGUCU AUUUAGUA | 3729 |
| 3227 | UACUAAAU G UAUUCCUG | 1427 | CAGGAAUA GCCGAAAGGCGAGUGAGGUCU AUUUAGUA | 3730 |
| 3235 | GUAUUCCU G UAGGGGGC | 1428 | GCCCCCUA GCCGAAAGGCGAGUGAGGUCU AGGAAUAC | 3731 |
| 3256 | UACUAAAU G UAUUUUAG | 1429 | CUAAAAUA GCCGAAAGGCGAGUGAGGUCU AUUUAGUA | 3732 |
| 15 | UGCUUUUG G UACAAAUG | 1430 | CAUUUGUA GCCGAAAGGCGAGUGAGGUCU CAAAAGCA | 3733 |
| 63 | UAAGGGGA G CAUGAAGA | 1431 | UCUUCAUG GCCGAAAGGCGAGUGAGGUCU UCCCCUUA | 3734 |
| 73 | AUGAAGAG G UGUUGAGG | 1432 | CCUCAACA GCCGAAAGGCGAGUGAGGUCU CUCUUCAU | 3735 |
| 81 | GUGUUGAG G UUAUGUCA | 1433 | UGACAUAA GCCGAAAGGCGAGUGAGGUCU CUCAACAC | 3736 |
| 91 | UAUGUCAA G CAUCUGGC | 1434 | GCCAGAUG GCCGAAAGGCGAGUGAGGUCU UUGACAUA | 3737 |
| 98 | AGCAUCUG G CACAGCUG | 1435 | CAGCUGUG GCCGAAAGGCGAGUGAGGUCU CAGAUGCU | 3738 |
| 103 | CUGGCACA G CUGAAGGC | 1436 | GCCUUCAG GCCGAAAGGCGAGUGAGGUCU UGUGCCAG | 3739 |
| 110 | AGCUGAAG G CAGAUGGA | 1437 | UCCAUCUG GCCGAAAGGCGAGUGAGGUCU CUUCAGCU | 3740 |
| 130 | AUUUACAA G UACGCAAU | 1438 | AUUGCGUA GCCGAAAGGCGAGUGAGGUCU UUGUAAAU | 3741 |
| 182 | AGACAAGA G CAAUAGUA | 1439 | UACUAUUG GCCGAAAGGCGAGUGAGGUCU UCUUGUCU | 3742 |
| 188 | GAGCAAUA G UAAAACAC | 1440 | GUGUUUUA GCCGAAAGGCGAGUGAGGUCU UAUUGCUC | 3743 |
| 202 | CACAUCAG G UCAGGGGG | 1441 | CCCCCUGA GCCGAAAGGCGAGUGAGGUCU CUGAUGUG | 3744 |
| 210 | GUCAGGGG G UUAAAGAC | 1442 | GUCUUUAA GCCGAAAGGCGAGUGAGGUCU CCCCUGAC | 3745 |
| 242 | UCCGAUAA G UUGGAAAC | 1443 | GUUUCCAA GCCGAAAGGCGAGUGAGGUCU UUAUCGGA | 3746 |
| 251 | UUGGAAAC G UGUGUCUA | 1444 | UAGACACA GCCGAAAGGCGAGUGAGGUCU GUUUCCAA | 3747 |
| 287 | AUAUAAUG G UAAAGAAA | 1445 | UUUCUUUA GCCGAAAGGCGAGUGAGGUCU CAUUAUAU | 3748 |
| 305 | ACACCUUC G UAACCCGC | 1446 | GCGGGUUA GCCGAAAGGCGAGUGAGGUCU GAAGGUGU | 3749 |
| 349 | GAUGUACA G CAAUGGGG | 1447 | CCCCAUUG GCCGAAAGGCGAGUGAGGUCU UGUACAUC | 3750 |
| 357 | GCAAUGGG G CCAUUUAA | 1448 | UUAAAUGG GCCGAAAGGCGAGUGAGGUCU CCCAUUGC | 3751 |
| 368 | AUUUAAGA G UUCUGUGU | 1449 | ACACAGAA GCCGAAAGGCGAGUGAGGUCU UCUUAAAU | 3752 |
| 406 | UAGAAGGG G CCCUGAGU | 1450 | ACUCAGGG GCCGAAAGGCGAGUGAGGUCU CCCUUCUA | 3753 |
| 413 | GGCCCUGA G UAAUUCAC | 1451 | GUGAAUUA GCCGAAAGGCGAGUGAGGUCU UCAGGGCC | 3754 |
| 429 | CUCAUUCA G CUGAACAA | 1452 | UUGUUCAG GCCGAAAGGCGAGUGAGGUCU UGAAUGAG | 3755 |
| 443 | CAACAAUG G CUAUGAAG | 1453 | CUUCAUAG GCCGAAAGGCGAGUGAGGUCU CAUUGUUG | 3756 |
| 452 | CUAUGAAG G CAUUGUCG | 1454 | CGACAAUG GCCGAAAGGCGAGUGAGGUCU CUUCAUAG | 3757 |
| 460 | GCAUUGUC G UUGCAAUC | 1455 | GAUUGCAA GCCGAAAGGCGAGUGAGGUCU GACAAUGC | 3758 |
| 520 | AGGACAUG G UGACCCAG | 1456 | CUGGGUCA GCCGAAAGGCGAGUGAGGUCU CAUGUCCU | 3759 |
| 529 | UGACCCAG G CAUCUCUG | 1457 | CAGAGAUG GCCGAAAGGCGAGUGAGGUCU CUGGGUCA | 3760 |
| 550 | UGUUUGAA G CUACAGGA | 1458 | UCCUGUAG GCCGAAAGGCGAGUGAGGUCU UUCAAACA | 3761 |
| 561 | ACAGGAAA G CGAUUUUA | 1459 | UAAAAUCG GCCGAAAGGCGAGUGAGGUCU UUUCCUGU | 3762 |
| 616 | AGACAAAG G CUGACUAU | 1460 | AUAGUCAG GCCGAAAGGCGAGUGAGGUCU CUUUGUCU | 3763 |
| 667 | AUGUUCUG G UUGCUGAG | 1461 | CUCAGCAA GCCGAAAGGCGAGUGAGGUCU CAGAACAU | 3764 |
| 675 | GUUGCUGA G UCUACUCC | 1462 | GGAGUAGA GCCGAAAGGCGAGUGAGGUCU UCAGCAAC | 3765 |
| 689 | UCCUCCAG G UAAUGAUG | 1463 | CAUCAUUA GCCGAAAGGCGAGUGAGGUCU CUGGAGGA | 3766 |
| 711 | UACACUGA G CAGAUGGG | 1464 | CCCAUCUG GCCGAAAGGCGAGUGAGGUCU UCAGUGUA | 3767 |
| 719 | GCAGAUGG G CAACUGUG | 1465 | CACAGUUG GCCGAAAGGCGAGUGAGGUCU CCAUCUGC | 3768 |
| 737 | AGAGAAGG G UGAAAGGA | 1466 | UCCUUUCA GCCGAAAGGCGAGUGAGGUCU CCUUCUCU | 3769 |
| 780 | GGAAAAAA G UUAGCUGA | 1467 | UCAGCUAA GCCGAAAGGCGAGUGAGGUCU UUUUUUCC | 3770 |
| 784 | AAAAGUUA G CUGAAUAU | 1468 | AUAUUCAG GCCGAAAGGCGAGUGAGGUCU UAACUUUU | 3771 |
| 803 | ACCACAAG G UAAGGCAU | 1469 | AUGCCUUA GCCGAAAGGCGAGUGAGGUCU CUUGUGGU | 3772 |
| 808 | AAGGUAAG G CAUUUGUC | 1470 | GACAAAUG GCCGAAAGGCGAGUGAGGUCU CUUACCUU | 3773 |
| 822 | GUCCAUGA G UGGGCUCA | 1471 | UGAGCCCA GCCGAAAGGCGAGUGAGGUCU UCAUGGAC | 3774 |
| 826 | AUGAGUGG G CUCAUCUA | 1472 | UAGAUGAG GCCGAAAGGCGAGUGAGGUCU CCACUCAU | 3775 |
| 844 | GAUGGGGA G UAUUUGAC | 1473 | GUCAAAUA GCCGAAAGGCGAGUGAGGUCU UCCCCAUC | 3776 |
| 855 | UUUGACGA G UACAAUAA | 1474 | UUAUUGUA GCCGAAAGGCGAGUGAGGUCU UCGUCAAA | 3777 |
| 901 | GAAUACAA G CAGUAAGA | 1475 | UCUUACUG GCCGAAAGGCGAGUGAGGUCU UUGUAUUC | 3778 |
| 904 | UACAAGCA G UAAGAUGU | 1476 | ACAUCUUA GCCGAAAGGCGAGUGAGGUCU UGCUUGUA | 3779 |
| 916 | GAUGUUCA G CAGGUAUU | 1477 | AAUACCUG GCCGAAAGGCGAGUGAGGUCU UGAACAUC | 3780 |
| 920 | UUCAGCAG G UAUUACUG | 1478 | CAGUAAUA GCCGAAAGGCGAGUGAGGUCU CUGCUGAA | 3781 |
| 929 | UAUUACUG G UACAAAUG | 1479 | CAUUUGUA GCCGAAAGGCGAGUGAGGUCU CAGUAAUA | 3782 |
| 940 | CAAAUGUA G UAAAGAAG | 1480 | CUUCUUUA GCCGAAAGGCGAGUGAGGUCU UACAUUUG | 3783 |
| 948 | GUAAAGAA G UGUCAGGG | 1481 | CCCUGACA GCCGAAAGGCGAGUGAGGUCU UUCUUUAC | 3784 |
| 959 | UCAGGGAG G CAGCUGUU | 1482 | AACAGCUG GCCGAAAGGCGAGUGAGGUCU CUCCCUGA | 3785 |
| 962 | GGGAGGCA G CUGUUACA | 1483 | UGUAACAG GCCGAAAGGCGAGUGAGGUCU UGCCUCCC | 3786 |
| 994 | UCAAUAAA G UUACAGGA | 1484 | UCCUGUAA GCCGAAAGGCGAGUGAGGUCU UUUAUUGA | 3787 |
| 1023 | GGAUGUGA G UUUGUUCU | 1485 | AGAACAAA GCCGAAAGGCGAGUGAGGUCU UCACAUCC | 3788 |
| 1054 | CGGAGAAG G CUUCUAUA | 1486 | UAUAGAAG GCCGAAAGGCGAGUGAGGUCU CUUCUCCG | 3789 |
| 1090 | AUUCUAUA G UUGAAUUC | 1487 | GAAUUCAA GCCGAAAGGCGAGUGAGGUCU UAUAGAAU | 3790 |
| 1126 | ACAAAGAA G CUCCAAAC | 1488 | GUUUGGAG GCCGAAAGGCGAGUGAGGUCU UUCUUUGU | 3791 |
| 1137 | CCAAACAA G CAAAAUCA | 1489 | UGAUUUUG GCCGAAAGGCGAGUGAGGUCU UUGUUUGG | 3792 |
| 1163 | UCUCCGAA G CACAUGGG | 1490 | CCCAUGUG GCCGAAAGGCGAGUGAGGUCU UUCGGAGA | 3793 |
| 1174 | CAUGGGAA G UGAUCCGU | 1491 | ACGGAUCA GCCGAAAGGCGAGUGAGGUCU UUCCCAUG | 3794 |
| 1181 | AGUGAUCC G UGAUUCUG | 1492 | CAGAAUCA GCCGAAAGGCGAGUGAGGUCU GGAUCACU | 3795 |
| 1224 | ACAACACA G CCACCAAA | 1493 | UUUGGUGG GCCGAAAGGCGAGUGAGGUCU UGUGUUGU | 3796 |
| 1279 | UGUGUUUA G UCCUUGAC | 1494 | GUCAAGGA GCCGAAAGGCGAGUGAGGUCU UAAACACA | 3797 |
| 1298 | AUCUGGAA G CAUGGCGA | 1495 | UCGCCAUG GCCGAAAGGCGAGUGAGGUCU UUCCAGAU | 3798 |
| 1303 | GAAGCAUG G CGACUGGU | 1496 | ACCAGUCG GCCGAAAGGCGAGUGAGGUCU CAUGCUUC | 3799 |
| 1310 | GGCGACUG G UAACCGCC | 1497 | GGCGGUUA GCCGAAAGGCGAGUGAGGUCU CAGUCGCC | 3800 |
| 1336 | UGAAUCAA G CAGGCCAG | 1498 | CUGGCCUG GCCGAAAGGCGAGUGAGGUCU UUGAUUCA | 3801 |
| 1340 | UCAAGCAG G CCAGCUUU | 1499 | AAAGCUGG GCCGAAAGGCGAGUGAGGUCU CUGCUUGA | 3802 |
| 1344 | GCAGGCCA G CUUUUCCU | 1500 | AGGAAAAG GCCGAAAGGCGAGUGAGGUCU UGGCCUGC | 3803 |
| 1363 | UGCAGACAG UUGAGCUG | 1501 | CAGCUCAA GCCGAAAGGCGAGUGAGGUCU UGUCUGCA | 3804 |
| 1368 | ACAGUUGA G CUGGGGUC | 1502 | GACCCCAG GCCGAAAGGCGAGUGAGGUCU UCAACUGU | 3805 |
| 1374 | GAGCUGGG G UCCUGGGU | 1503 | ACCCAGGA GCCGAAAGGCGAGUGAGGUCU CCCAGCUC | 3806 |
| 1381 | GGUCCUGG G UUGGGAUG | 1504 | CAUCCCAA GCCGAAAGGCGAGUGAGGUCU CCAGGACC | 3807 |
| 1390 | UUGGGAUG G UGACAUUU | 1505 | AAAUGUCA GCCGAAAGGCGAGUGAGGUCU CAUCCCAA | 3808 |
| 1403 | AUUUGACA G UGCUGCCC | 1506 | GGGCAGCA GCCGAAAGGCGAGUGAGGUCU UGUCAAAU | 3809 |
| 1421 | UGUACAAA G UGAACUCA | 1507 | UGAGUUCA GCCGAAAGGCGAGUGAGGUCU UUUGUACA | 3810 |
| 1442 | GAUAAACA G UGGCAGUG | 1508 | CACUGCCA GCCGAAAGGCGAGUGAGGUCU UGUUUAUC | 3811 |
| 1445 | AAACAGUG G CAGUGACA | 1509 | UGUCACUG GCCGAAAGGCGAGUGAGGUCU CACUGUUU | 3812 |
| 1448 | CAGUGGCA G UGACAGGG | 1510 | CCCUGUCA GCCGAAAGGCGAGUGAGGUCU UGCCACUG | 3813 |
| 1483 | UACCUGCA G CAGCUUCA | 1511 | UGAAGCUG GCCGAAAGGCGAGUGAGGUCU UGCAGGUA | 3814 |
| 1486 | CUGCAGCA G CUUCAGGA | 1512 | UCCUGAAG GCCGAAAGGCGAGUGAGGUCU UGCUGCAG | 3815 |
| 1500 | GGAGGGAC G UCCAUCUG | 1513 | CAGAUGGA GCCGAAAGGCGAGUGAGGUCU GUCCCUCC | 3816 |
| 1511 | CAUCUGCA G CGGGCUUC | 1514 | GAAGCCCG GCCGAAAGGCGAGUGAGGUCU UGCAGAUG | 3817 |
| 1515 | UGCAGCGG G CUUCGAUC | 1515 | GAUCGAAG GCCGAAAGGCGAGUGAGGUCU CCGCUGCA | 3818 |
| 1525 | UUCGAUCG G CAUUUACU | 1516 | AGUAAAUG GCCGAAAGGCGAGUGAGGUCU CGAUCGAA | 3819 |
| 1607 | CACUAUAA G UGGGUGCU | 1517 | AGCACCCA GCCGAAAGGCGAGUGAGGUCU UUAUAGUG | 3820 |
| 1611 | AUAAGUGG G UGCUUUAA | 1518 | UUAAAGCA GCCGAAAGGCGAGUGAGGUCU CCACUUAU | 3821 |
| 1624 | UUAACGAG G UCAAACAA | 1519 | UUGUUUGA GCCGAAAGGCGAGUGAGGUCU CUCGUUAA | 3822 |
| 1634 | CAAACAAA G UGGUGCCA | 1520 | UGGCACCA GCCGAAAGGCGAGUGAGGUCU UUUGUUUG | 3823 |
| 1637 | ACAAAGUG G UGCCAUCA | 1521 | UGAUGGCA GCCGAAAGGCGAGUGAGGUCU CACUUUGU | 3824 |
| 1654 | UCCACACA G UCGCUUUG | 1522 | CAAAGCGA GCCGAAAGGCGAGUGAGGUCU UGUGUGGA | 3825 |
| 1665 | GCUUUGGG G CCCUCUGC | 1523 | GCAGAGGG GCCGAAAGGCGAGUGAGGUCU CCCAAAGC | 3826 |
| 1675 | CCUCUGCA G CUCAAGAA | 1524 | UUCUUGAG GCCGAAAGGCGAGUGAGGUCU UGCAGAGG | 3827 |
| 1692 | CUAGAGGA G CUGUCCAA | 1525 | UUGGACAG GCCGAAAGGCGAGUGAGGUCU UCCUCUAG | 3828 |
| 1712 | GACAGGAG G UUUACAGA | 1526 | UCUGUAAA GCCGAAAGGCGAGUGAGGUCU CUCCUGUC | 3829 |
| 1738 | CAGAUCAA G UUCAGAAC | 1527 | GUUCUGAA GCCGAAAGGCGAGUGAGGUCU UUGAUCUG | 3830 |
| 1751 | GAACAAUG G CCUCAUUG | 1528 | CAAUGAGG GCCGAAAGGCGAGUGAGGUCU CAUUGUUC | 3831 |
| 1771 | CUUUUGGG G CCCUUUCA | 1529 | UGAAAGGG GCCGAAAGGCGAGUGAGGUCU CCCAAAAG | 3832 |
| 1792 | GAAAUGGA G CUGUCUCU | 1530 | AGAGACAG GCCGAAAGGCGAGUGAGGUCU UCCAUUUC | 3833 |
| 1803 | GUCUCUCA G CGCUCCAU | 1531 | AUGGAGCG GCCGAAAGGCGAGUGAGGUCU UGAGAGAC | 3834 |
| 1815 | UCCAUCCA G CUUGAGAG | 1532 | CUCUCAAG GCCGAAAGGCGAGUGAGGUCU UGGAUGGA | 3835 |
| 1823 | GCUUGAGA G UAAGGGAU | 1533 | AUCCCUUA GCCGAAAGGCGAGUGAGGUCU UCUCAAGC | 3836 |
| 1847 | CCAGAACA G CCAGUGGA | 1534 | UCCACUGG GCCGAAAGGCGAGUGAGGUCU UGUUCUGG | 3837 |
| 1851 | AACAGCCA G UGGAUGAA | 1535 | UUCAUCCA GCCGAAAGGCGAGUGAGGUCU UGGCUGUU | 3838 |
| 1862 | GAUGAAUG G CACAGUGA | 1536 | UCACUGUG GCCGAAAGGCGAGUGAGGUCU CAUUCAUC | 3839 |
| 1867 | AUGGCACA G UGAUCGUG | 1537 | CACGAUCA GCCGAAAGGCGAGUGAGGUCU UGUGCCAU | 3840 |
| 1873 | CAGUGAUC G UGGACAGC | 1538 | GCUGUCCA GCCGAAAGGCGAGUGAGGUCU GAUCACUG | 3841 |
| 1880 | CGUGGACA G CACCGUGG | 1539 | CCACGGUG GCCGAAAGGCGAGUGAGGUCU UGUCCACG | 3842 |
| 1885 | ACAGCACC G UGGGAAAG | 1540 | CUUUCCCA GCCGAAAGGCGAGUGAGGUCU GGUGCUGU | 3843 |
| 1926 | ACAACGCA G CCUCCCCA | 1541 | UGGGGAGG GCCGAAAGGCGAGUGAGGUCU UGCGUUGU | 3844 |
| 1955 | GGAUCCCA G UGGACAGA | 1542 | UCUGUCCA GCCGAAAGGCGAGUGAGGUCU UGGGAUCC | 3845 |
| 1965 | GGACAGAA G CAAGGUGG | 1543 | CCACCUUG GCCGAAAGGCGAGUGAGGUCU UUCUGUCC | 3846 |
| 1970 | GAAGCAAG G UGGCUUUG | 1544 | CAAAGCCA GCCGAAAGGCGAGUGAGGUCU CUUGCUUC | 3847 |
| 1973 | GCAAGGUG G CUUUGUAG | 1545 | CUACAAAG GCCGAAAGGCGAGUGAGGUCU CACCUUGC | 3848 |
| 1981 | GCUUUGUA G UGGACAAA | 1546 | UUUGUCCA GCCGAAAGGCGAGUGAGGUCU UACAAAGC | 3849 |
| 2002 | CCAAAAUG G CCUACCUC | 1547 | GAGGUAGG GCCGAAAGGCGAGUGAGGUCU CAUUUUGG | 3850 |
| 2021 | AAUCCCAG G CAUUGCUA | 1548 | UAGCAAUG GCCGAAAGGCGAGUGAGGUCU CUGGGAUU | 3851 |
| 2032 | UUGCUAAG G UUGGCACU | 1549 | AGUGCCAA GCCGAAAGGCGAGUGAGGUCU CUUAGCAA | 3852 |
| 2036 | UAAGGUUG G CACUUGGA | 1550 | UCCAAGUG GCCGAAAGGCGAGUGAGGUCU CAACCUUA | 3853 |
| 2051 | GAAAUACA G UCUGCAAG | 1551 | CUUGCAGA GCCGAAAGGCGAGUGAGGUCU UGUAUUUC | 3854 |
| 2059 | GUCUGCAA G CAAGCUCA | 1552 | UGAGCUUG GCCGAAAGGCGAGUGAGGUCU UUGCAGAC | 3855 |
| 2063 | GCAAGCAA G CUCACAAA | 1553 | UUUGUGAG GCCGAAAGGCGAGUGAGGUCU UUGCUUGC | 3856 |
| 2091 | ACUGUCAC G UCCCGUGC | 1554 | GCACGGGA GCCGAAAGGCGAGUGAGGUCU GUGACAGU | 3857 |
| 2096 | CACGUCCC G UGCGUCCA | 1555 | UGGACGCA GCCGAAAGGCGAGUGAGGUCU GGGACGUG | 3858 |
| 2100 | UCCCGUGC G UCCAAUGC | 1556 | GCAUUGGA GCCGAAAGGCGAGUGAGGUCU GCACGGGA | 3859 |
| 2128 | CAAUUACA G UGACUUCC | 1557 | GGAAGUCA GCCGAAAGGCGAGUGAGGUCU UGUAAUUG | 3860 |
| 2156 | GGACACCA G CAAAUUCC | 1558 | GGAAUUUG GCCGAAAGGCGAGUGAGGUCU UGGUGUCC | 3861 |
| 2168 | AUUCCCCA G CCCUCUGG | 1559 | CCAGAGGG GCCGAAAGGCGAGUGAGGUCU UGGGGAAU | 3862 |
| 2176 | GCCCUCUG G UAGUUUAU | 1560 | AUAAACUA GCCGAAAGGCGAGUGAGGUCU CAGAGGGC | 3863 |
| 2179 | CUCUGGUA G UUUAUGCA | 1561 | UGCAUAAA GCCGAAAGGCGAGUGAGGUCU UACCAGAG | 3864 |
| 2203 | GCCAAGGA G CCUCCCCA | 1562 | UGGGGAGG GCCGAAAGGCGAGUGAGGUCU UCCUUGGC | 3865 |
| 2221 | UUCUCAGG G CCAGUGUC | 1563 | GACACUGG GCCGAAAGGCGAGUGAGGUCU CCUGAGAA | 3866 |
| 2225 | CAGGGCCA G UGUCACAG | 1564 | CUGUGACA GCCGAAAGGCGAGUGAGGUCU UGGCCCUG | 3867 |
| 2233 | GUGUCACA G CCCUGAUU | 1565 | AAUCAGGG GCCGAAAGGCGAGUGAGGUCU UGUGACAC | 3868 |
| 2248 | UUGAAUCA G UGAAUGGA | 1566 | UCCAUUCA GCCGAAAGGCGAGUGAGGUCU UGAUUCAA | 3869 |
| 2263 | GAAAAACA G UUACCUUG | 1567 | CAAGGUAA GCCGAAAGGCGAGUGAGGUCU UGUUUUUC | 3870 |
| 2290 | AUAAUGGA G CAGGUGCU | 1568 | AGCACCUG GCCGAAAGGCGAGUGAGGUCU UCCAUUAU | 3871 |
| 2294 | UGGAGCAG G UGCUGAUG | 1569 | CAUCAGCA GCCGAAAGGCGAGUGAGGUCU CUGCUCCA | 3872 |
| 2318 | GGAUGACG G UGUCUACU | 1570 | AGUAGACA GCCGAAAGGCGAGUGAGGUCU CGUCAUCC | 3873 |
| 2331 | UACUCAAG G UAUUUCAC | 1571 | GUGAAAUA GCCGAAAGGCGAGUGAGGUCU CUUGAGUA | 3874 |
| 2357 | CACGAAUG G UAGAUACA | 1572 | UGUAUCUA GCCGAAAGGCGAGUGAGGUCU CAUUCGUG | 3875 |
| 2366 | UAGAUACA G UGUAAAAG | 1573 | CUUUUACA GCCGAAAGGCGAGUGAGGUCU UGUAUCUA | 3876 |
| 2374 | GUGUAAAA G UGCGGGCU | 1574 | AGCCCGCA GCCGAAAGGCGAGUGAGGUCU UUUUACAC | 3877 |
| 2380 | AAGUGCGG G CUCUGGGA | 1575 | UCCCAGAG GCCGAAAGGCGAGUGAGGUCU CCGCACUU | 3878 |
| 2392 | UGGGAGGA G UUAACGCA | 1576 | UGCGUUAA GCCGAAAGGCGAGUGAGGUCU UCCUCCCA | 3879 |
| 2401 | UUAACGCA G CCAGACGG | 1577 | CCGUCUGG GCCGAAAGGCGAGUGAGGUCU UGCGUUAA | 3880 |
| 2413 | GACGGAGA G UGAUACCC | 1578 | GGGUAUCA GCCGAAAGGCGAGUGAGGUCU UCUCCGUC | 3881 |
| 2424 | AUACCCCA G CAGAGUGG | 1579 | CCACUCUG GCCGAAAGGCGAGUGAGGUCU UGGGGUAU | 3882 |
| 2429 | CCAGCAGA G UGGAGCAC | 1580 | GUGCUCCA GCCGAAAGGCGAGUGAGGUCU UCUGCUGG | 3883 |
| 2434 | AGAGUGGA G CACUGUAC | 1581 | GUACAGUG GCCGAAAGGCGAGUGAGGUCU UCCACUCU | 3884 |
| 2450 | CAUACCUG G CUGGAUUG | 1582 | CAAUCCAG GCCGAAAGGCGAGUGAGGUCU CAGGUAUG | 3885 |
| 2523 | CAACACAA G CAAGUGUG | 1583 | CACACUUG GCCGAAAGGCGAGUGAGGUCU UUGUGUUG | 3886 |
| 2527 | ACAAGCAA G UGUGUUEU | 1584 | GAAACACA GCCGAAAGGCGAGUGAGGUCU UUGCUUGU | 3887 |
| 2537 | GUGUUUCA G CAGAACAU | 1585 | AUGUUCUG GCCGAAAGGCGAGUGAGGUCU UGAAACAC | 3888 |
| 2555 | CUCGGGAG G CUCAUUUG | 1586 | CAAAUGAG GCCGAAAGGCGAGUGAGGUCU CUCCCGAG | 3889 |
| 2566 | CAUUUGUG G CUUCUGAU | 1587 | AUCAGAAG GCCGAAAGGCGAGUGAGGUCU CACAAAUG | 3890 |
| 2612 | CCCACCUG G CCAAAUCA | 1588 | UGAUUUGG GCCGAAAGGCGAGUGAGGUCU CAGGUGGG | 3891 |
| 2632 | ACCUGAAG G CGGAAAUU | 1589 | AAUUUCCG GCCGAAAGGCGAGUGAGGUCU CUUCAGGU | 3892 |
| 2648 | UCACGGGG G CAGUCUCA | 1590 | UGAGACUG GCCGAAAGGCGAGUGAGGUCU CCCCGUGA | 3893 |
| 2651 | CGGGGGCA G UCUCAUUA | 1591 | UAAUGAGA GCCGAAAGGCGAGUGAGGUCU UGCCCCCG | 3894 |
| 2674 | CUUGGACA G CUCCUGGG | 1592 | CCCAGGAG GCCGAAAGGCGAGUGAGGUCU UGUCCAAG | 3895 |
| 2704 | AUGGAACA G CUCACAAG | 1593 | CUUGUGAG GCCGAAAGGCGAGUGAGGUCU UGUUCCAU | 3896 |
| 2712 | GCUCACAA G UAUAUCAU | 1594 | AUGAUAUA GCCGAAAGGCGAGUGAGGUCU UUGUGAGC | 3897 |
| 2729 | UCGAAUAA G UACAAGUA | 1595 | UACUUGUA GCCGAAAGGCGAGUGAGGUCU UUAUUCGA | 3898 |
| 2735 | AAGUACAA G UAUUCUUG | 1596 | CAAGAAUA GCCGAAAGGCGAGUGAGGUCU UUGUACUU | 3899 |
| 2757 | AGAGACAA G UUCAAUGA | 1597 | UCAUUGAA GCCGAAAGGCGAGUGAGGUCU UUGUCUCU | 3900 |
| 2776 | CUCUUCAA G UGAAUACU | 1598 | AGUAUUCA GCCGAAAGGCGAGUGAGGUCU UUGAAGAG | 3901 |
| 2806 | CAAAGGAA G CCAACUCU | 1599 | AGAGUUGG GCCGAAAGGCGAGUGAGGUCU UUCCUUUG | 3902 |
| 2821 | CUGAGGAA G UCUUUUUG | 1600 | CAAAAAGA GCCGAAAGGCGAGUGAGGUCU UUCCUCAG | 3903 |
| 2861 | UGAAAAUG G CACAGAUC | 1601 | GAUCUGUG GCCGAAAGGCGAGUGAGGUCU CAUUUUCA | 3904 |
| 2887 | CUAUUCAG G CUGUUGAU | 1602 | AUCAACAG GCCGAAAGGCGAGUGAGGUCU CUGAAUAG | 3905 |
| 2899 | UUGAUAAG G UCGAUCUG | 1603 | CAGAUCGA GCCGAAAGGCGAGUGAGGUCU CUUAUCAA | 3906 |
| 2935 | UUGCACGA G UAUCUUUG | 1604 | CAAAGAUA GCCGAAAGGCGAGUGAGGUCU UCGUGCAA | 3907 |
| 2978 | GACACCUA G UCCUGAUG | 1605 | CAUCAGGA GCCGAAAGGCGAGUGAGGUCU UAGGUGUC | 3908 |
| 2991 | GAUGAAAC G UCUGCUCC | 1606 | GGAGCAGA GCCGAAAGGCGAGUGAGGUCU GUUUCAUC | 3909 |
| 3023 | UAUCAACA G CACCAUUC | 1607 | GAAUGGUG GCCGAAAGGCGAGUGAGGUCU UGUUGAUA | 3910 |
| 3035 | CAUUCCUG G CAUUCACA | 1068 | UGUGAAUG GCCGGAAAGGCGAGUGAGGUCU CAGGAAUG | 3911 |
| 3063 | AUGUGGAA G UGGAUAGG | 1609 | CCUAUCCA GCCGAAAGGCGGAGUGAGGUCU UUCCACAU | 3912 |
| 3081 | GAACUGCA G CUGUCAAU | 1610 | AUUGACAG GCCGAAAGGCGAGUGAGGUCU UGCAGUUC | 3913 |
| 3091 | UGUCAAUA G CCUAGGGC | 1611 | GCCCUAGG GCCGAAAGGCGAGUGAGGUCU UAUUGACA | 3914 |
| 3098 | AGCCUAGG G CUGAAUUU | 1612 | AAAUUCAG GCCGAAAGGCGAGUGAGGUCU CCUAGGCU | 3915 |
| 3189 | UGUAGGGG G CGAUAUAC | 1613 | GUAAUCG GCCGAAAGGCGAGUGAGGUCU CCCCUACA | 3916 |
| 3242 | UGUAGGGG G CGAUAUAC | 1613 | GUAUAUCG GCCGAAAGGCGAGUGAGGUCU CCCCUACA | 3916 |
| 3210 | UGUAUAUA G UACAUUUA | 1614 | UAAAUGUA GCCGAAAGGCGAGUGAGGUCU UAUAUACA | 3917 |
| 3279 | UGUAGGGG G CGAUAAAA | 1615 | UUUUAUCG GCCGAAAGGCGAGUGAGGUCU CCCCUACA | 3918 |

Input Sequence = NM_001285. Cut Site = G/Y
Arm Length = 8. Core Sequence = GCcgaaagGCGaGuCaaGGuCu
NM_001285 (Homo sapiens chloride channel, calcium activated, 1 (CLCA1) mRNA, 3311bp)

TABLE VII
Human CLCA1 DNAzyme and Target Sequence 249.021

| | | Seq | | Rz |
|---|---|---|---|---|
| | | ID | | Seq ID |
| Pos | Substrate | No | Dnazyme | No |
| 17 | CUUUUGGU A CAAAUGGA | 4 | TCCATTTG GGCTAGCTACAACGA ACCAAAAG | 3919 |
| 34 | UGUGGAAU A UAAUUGAA | 5 | TTCAATTA GGCTAGCTACAACGA ATTCCACA | 3920 |
| 44 | AAUUGAAU A UUUUCUUG | 8 | CAAGAAAA GGCTAGCTACAACGA ATTCAATT | 3921 |
| 84 | UUGAGGUU A UGUCAAGC | 19 | GCTTGACA GGCTAGCTACAACGA AACCTCAA | 3922 |
| 122 | AUGGAAAU A UUUACAAG | 22 | CTTGTAAA GGCTAGCTACAACGA ATTTCCAT | 3923 |
| 126 | AAAUAUUU A CAAGUACG | 25 | CGTACTTG GGCTAGCTACAACGA AAATATTT | 3924 |
| 132 | UUACAAGU A CGCAAUUU | 26 | AAATTGCG GGCTAGCTACAACGA ACTTGTAA | 3925 |
| 152 | ACUAAGAU A UUGUUAUC | 30 | GATAACAA GGCTAGCTACAACGA ATCTTAGT | 3926 |
| 158 | AUAUUGUU A UCAUUCUC | 33 | GAGAATGA GGCTAGCTACAACGA AACAATAT | 3927 |
| 169 | AUUCUCCU A UUGAAGAC | 38 | GTCTTCAA GGCTAGCTACAACGA AGGAGAAT | 3928 |
| 259 | GUGUGUCU A UAUUUUCA | 52 | TGAAAATA GGCTAGCTACAACGA AGACACAC | 3929 |
| 261 | GUGUCUAU A UUUUCAUA | 53 | TATGAAAA GGCTAGCTACAACGA ATAGACAC | 3930 |
| 269 | AUUUUCAU A UCUGUAUA | 58 | TATACAGA GGCTAGCTACAACGA ATGAAAAT | 3931 |
| 275 | AUAUCUGU A UAUAUAUA | 60 | TATATATA GGCTAGCTACAACGA ACAGATAT | 3932 |
| 277 | AUCUGUAU A UAUAUAAU | 61 | ATTATATA GGCTAGCTACAACGA ATACAGAT | 3933 |
| 279 | CUGUAUAU A UAUAAUGG | 62 | CCATTATA GGCTAGCTACAACGA ATATACAG | 3934 |
| 281 | GUAUAUAU A UAAUGGUA | 63 | TACCATTA GGCTAGCTACAACGA ATATATAC | 3935 |
| 346 | GGAGAUGU A CAGCAAUG | 74 | CATTGCTG GGCTAGCTACAACGA ACATCTCC | 3936 |
| 446 | CAAUGGCU A UGAAGGCA | 97 | TGCCTTCA GGCTAGCTACAACGA AGCCATTG | 3937 |
| 539 | AUCUCUGU A UCUGUUUG | 108 | CAAACAGA GGCTAGCTACAACGA ACAGAGAT | 3938 |
| 553 | UUGAAGCU A CAGGAAAG | 112 | CTTTCCTG GGCTAGCTACAACGA AGCTTCAA | 3939 |
| 569 | GCGAUUUU A UUUCAAAA | 116 | TTTTGAAA GGCTAGCTACAACGA AAAATCGC | 3940 |
| 623 | GGCUGACU A UGUGAGAC | 126 | GTCTCACA GGCTAGCTACAACGA AGTCAGCC | 3941 |
| 647 | UGAGACCU A CAAAAAUG | 128 | CATTTTTG GGCTAGCTACAACGA AGGTCTCA | 3942 |
| 679 | CUGAGUCU A CUCCUCCA | 133 | TGGAGGAG GGCTAGCTACAACGA AGACTCAG | 3943 |
| 704 | UGAACCCU A CACUGAGC | 137 | GCTCAGTG GGCTAGCTACAACGA AGGGTTCA | 3944 |
| 791 | AGCUGAAU A UGGACCAC | 147 | GTGGTCCA GGCTAGCTACAACGA ATTCAGCT | 3945 |
| 834 | GCUCAUCU A CGAUGGGG | 154 | CCCCATCG GGCTAGCTACAACGA AGATGAGC | 3946 |
| 846 | UGGGGAGU A UUUGACGA | 155 | TCGTCAAA GGCTAGCTACAACGA ACTCCCCA | 3947 |
| 857 | UGACGAGU A CAAUAAUG | 158 | CATTATTG GGCTAGCTACAACGA ACTCGTCA | 3948 |
| 878 | GAAAUUCU A CUUAUCCA | 162 | TGGATAAG GGCTAGCTACAACGA AGAATTTC | 3949 |
| 882 | UUCUACUU A UCCAAUGG | 164 | CCATTGGA GGCTAGCTACAACGA AAGTAGAA | 3950 |
| 897 | GGAAGAAU A CAAGCAGU | 166 | ACTGCTTG GGCTAGCTACAACGA ATTCTTCC | 3951 |
| 922 | CAGCAGGU A UUACUGGU | 170 | ACCAGTAA GGCTAGCTACAACGA ACCTGCTG | 3952 |
| 925 | CAGGUAUU A CUGGUACA | 172 | TGTACCAG GGCTAGCTACAACGA AATACCTG | 3953 |
| 931 | UUACUGGU A CAAAUGUA | 173 | TACATTTG GGCTAGCTACAACGA ACCAGTAA | 3954 |
| 968 | CAGCUGUU A CACCAAAA | 178 | TTTTGGTG GGCTAGCTACAACGA AACAGCTG | 3955 |
| 997 | AUAAAGUU A CAGGACUC | 183 | GAGTCCTG GGCTAGCTACAACGA AACTTTAT | 3956 |
| 1007 | AGGACUCU A UGAAAAAG | 185 | CTTTTTCA GGCTAGCTACAACGA AGAGTCCT | 3957 |
| 1060 | AGGCUUCU A UAAUGUUU | 194 | AAACATTA GGCTAGCTACAACGA AGAAGCCT | 3958 |
| 1087 | UUGAUUCU A UAGUUGAA | 201 | TTCAACTA GGCTAGCTACAACGA AGAATCAA | 3959 |
| 1102 | AAUUCUGU A CAGAACAA | 206 | TTGTTCTG GGCTAGCTACAACGA ACAGAATT | 3960 |
| 1213 | CCACUCCU A UGACAACA | 218 | TGTTGTCA GGCTAGCTACAACGA AGGAGTGG | 3961 |
| 1416 | GCCCAUGU A CAAAGUGA | 245 | TCACTTTG GGCTAGCTACAACGA ACATGGGC | 3962 |
| 1431 | GAACUCAU A CAGAUAAA | 247 | TTTATCTG GGCTAGCTACAACGA ATGAGTTC | 3963 |
| 1476 | AAAAGAUU A CCUGCAGC | 251 | GCTGCAGG GGCTAGCTACAACGA AATCTTTT | 3964 |
| 1531 | CGGCAUUU A CUGUGAUU | 261 | AATCACAG GGCTAGCTACAACGA AAATGCCG | 3965 |
| 1550 | GAAGAAAU A UCCAACUG | 264 | CAGTTGGA GGCTAGCTACAACGA ATTTCTTC | 3966 |
| 1603 | ACAACACU A UAAGUGGG | 268 | CCCACTTA GGCTAGCTACAACGA AGTGTTGT | 3967 |
| 1716 | GGAGGUUU A CAGACAUA | 285 | TATGTCTG GGCTAGCTACAACGA AAACCTCC | 3968 |
| 1724 | ACAGACAU A UGCUUCAG | 286 | CTGAAGCA GGCTAGCTACAACGA ATGTCTGT | 3969 |
| 1909 | UGUUUCUU A UCACCUGG | 318 | CCAGGTGA GGCTAGCTACAACGA AAGAAACA | 3970 |
| 2006 | AAUGGCCU A CCUCCAAA | 329 | TTTGGAGG GGCTAGCTACAACGA AGGCCATT | 3971 |
| 2048 | UUGGAAAU A CAGUCUGC | 336 | GCAGACTG GGCTAGCTACAACGA ATTTCCAA | 3972 |
| 2110 | CCAAUGCU A CCCUGCCU | 343 | AGGCAGGG GGCTAGCTACAACGA AGCATTGG | 3973 |
| 2125 | CUCCAAUU A CAGUGACU | 346 | AGTCACTG GGCTAGCTACAACGA AATTGGAG | 3974 |
| 2183 | GGUAGUUU A UGCAAAUA | 355 | TATTTGCA GGCTAGCTACAACGA AAACTACC | 3975 |
| 2191 | AUGCAAAU A UUCGCCAA | 356 | TTGGCGAA GGCTAGCTACAACGA ATTTGCAT | 3976 |
| 2266 | AAACAGUU A CCUUGGAA | 367 | TTCCAAGG GGCTAGCTACAACGA AACTGTTT | 3977 |
| 2277 | UUGGAACU A CUGGAUAA | 369 | TTATCCAG GGCTAGCTACAACGA AGTTCCAA | 3978 |
| 2305 | CUGAUGCU A CUAAGGAU | 371 | ATCCTTAG GGCTAGCTACAACGA AGCATCAG | 3979 |
| 2324 | CGGUGUCU A CUCAAGGU | 374 | ACCTTGAG GGCTAGCTACAACGA AGACACCG | 3980 |
| 2333 | CUCAAGGU A UUUCACAA | 376 | TTGTGAAA GGCTAGCTACAACGA ACCTTGAG | 3981 |
| 2345 | CACAACUU A UGACACGA | 381 | TCGTGTCA GGCTAGCTACAACGA AAGTTGTG | 3982 |
| 2363 | UGGUAGAU A CAGUGUAA | 383 | TTACACTG GGCTAGCTACAACGA ATCTACCA | 3983 |
| 2418 | AGAGUGAU A CCCCAGCA | 388 | TGCTGGGG GGCTAGCTACAACGA ATCACTCT | 3984 |
| 2441 | AGCACUGU A CAUACCUG | 389 | CAGGTATG GGCTAGCTACAACGA ACAGTGCT | 3985 |
| 2445 | CUGUACAU A CCUGGCUG | 390 | CAGCCAGG GGCTAGCTACAACGA ATGTACAG | 3986 |
| 2472 | GAUGAAAU A CAAUGGAA | 392 | TTCCATTG GGCTAGCTACAACGA ATTTCATC | 3987 |
| 2592 | GCUCCCAU A CCUGAUCU | 411 | AGATCAGG GGCTAGCTACAACGA ATGGGAGC | 3988 |
| 2690 | GGAUGAUU A UGACCAUG | 427 | CATGGTCA GGCTAGCTACAACGA AATCATCC | 3989 |
| 2714 | UCACAAGU A UAUCAUUC | 429 | GAATGATA GGCTAGCTACAACGA ACTTGTGA | 3990 |
| 2716 | ACAAGUAU A UCAUUCGA | 430 | TCGAATGA GGCTAGCTACAACGA ATACTTGT | 3991 |
| 2731 | GAAUAAGU A CAAGUAUU | 435 | AATACTTG GGCTAGCTACAACGA ACTTATTC | 3992 |
| 2737 | GUACAAGU A UUCUUGAU | 436 | ATCAAGAA GGCTAGCTACAACGA ACTTGTAC | 3993 |
| 2782 | AAGUGAAU A CUACUGCU | 448 | AGCAGTAG GGCTAGCTACAACGA ATTCACTT | 3994 |
| 2785 | UGAAUACU A CUGCUCUC | 449 | GAGAGCAG GGCTAGCTACAACGA AGTATTCA | 3995 |
| 2848 | AAAACAUU A CUUUUGAA | 463 | TTCAAAAG GGCTAGCTACAACGA AATGTTTT | 3996 |
| 2881 | UCAUUGCU A UUCAGGCU | 473 | AGCCTGAA GGCTAGCTACAACGA AGCAATGA | 3997 |
| 2919 | UCAGAAAU A UCCAACAU | 481 | ATGTTGGA GGCTAGCTACAACGA ATTTCTGA | 3998 |
| 2937 | GCACGAGU A UCUUUGUU | 484 | AACAAAGA GGCTAGCTACAACGA ACTCGTGC | 3999 |
| 2947 | CUUUGUUU A UUCCUCCA | 490 | TGGAGGAA GGCTAGCTACAACGA AAACAAAG | 4000 |
| 3010 | GUCCUAAU A UUCAUAUC | 502 | GATATGAA GGCTAGCTACAACGA ATTAGGAC | 4001 |
| 3016 | AUAUUCAU A UCAACAGC | 505 | GCTGTTGA GGCTAGCTACAACGA ATGAATAT | 4002 |
| 3055 | UAAAAAUU A UGUGGAAG | 516 | CTTCCACA GGCTAGCTACAACGA AATTTTTA | 4003 |
| 3149 | UUUUGAUU A UAAAAUUU | 540 | AAATTTTA GGCTAGCTACAACGA AATCAAAA | 4004 |
| 3168 | UAAAAUGU A UUUUAGAC | 547 | GTCTAAAA GGCTAGCTACAACGA ACATTTTA | 4005 |
| 3194 | GGGGCGAU A UACUAAAU | 555 | ATTTAGTA GGCTAGCTACAACGA ATCGCCCC | 4006 |
| 3247 | GGGGCGAU A UACUAAAU | 555 | ATTTAGTA GGCTAGCTACAACGA ATCGCCCC | 4006 |
| 3196 | GGCGAUAU A CUAAAUGU | 556 | ACATTTAG GGCTAGCTACAACGA ATATCGCC | 4007 |
| 3249 | GGCGAUAU A CUAAAUGU | 556 | ACATTTAG GGCTAGCTACAACGA ATATCGCC | 4007 |
| 3205 | CUAAAUGU A UAUAGUAC | 558 | GTACTATA GGCTAGCTACAACGA ACATTTAG | 4008 |
| 3207 | AAAUGUAU A UAGUACAU | 559 | ATGTACTA GGCTAGCTACAACGA ATACATTT | 4009 |
| 3212 | UAUAUAGU A CAUUUAUA | 561 | TATAAATG GGCTAGCTACAACGA ACTATATA | 4010 |
| 3218 | GUACAUUU A UACUAAAU | 564 | ATTTAGTA GGCTAGCTACAACGA AAATGTAC | 4011 |
| 3220 | ACAUUUAU A CUAAAUGU | 565 | ACATTTAG GGCTAGCTACAACGA ATAAATGT | 4012 |
| 3229 | CUAAAUGU A UUCCUGUA | 567 | TACAGGAA GGCTAGCTACAACGA ACATTTAG | 4013 |
| 3258 | CUAAAUGU A UUUUAGAC | 572 | GTCTAAAA GGCTAGCTACAACGA ACATTTAG | 4014 |
| 65 | AGGGGAGC A UGAAGAGG | 579 | CCTCTTCA GGCTAGCTACAACGA GCTCCCCT | 4015 |
| 93 | UGUCAAGC A UCUGGCAC | 581 | GTGCCAGA GGCTAGCTACAACGA GCTTGACA | 4016 |
| 100 | CAUCUGGC A CAGCUGAA | 583 | TTCAGCTG GGCTAGCTACAACGA GCCAGATG | 4017 |
| 161 | UUGUUAUC A UUCUCCUA | 590 | TAGGAGAA GGCTAGCTACAACGA GATAACAA | 4018 |
| 195 | AGUAAAAC A CAUCAGGU | 596 | ACCTGATG GGCTAGCTACAACGA GTTTTACT | 4019 |
| 197 | UAAAACAC A UCAGGUCA | 597 | TGACCTGA GGCTAGCTACAACGA GTGTTTTA | 4020 |
| 231 | GAUAAACC A CUUCCGAU | 603 | ATCGGAAG GGCTAGCTACAACGA GGTTTATC | 4021 |
| 267 | AUAUUUUC A UAUCUGUA | 607 | TACAGATA GGCTAGCTACAACGA GAAAATAT | 4022 |
| 299 | AGAAAGAC A CCUUCGUA | 609 | TACGAAGG GGCTAGCTACAACGA GTCTTTCT | 4023 |
| 314 | UAACCCGC A UUUUCCAA | 614 | TTGGAAAA GGCTAGCTACAACGA GCGGGTTA | 4024 |
| 334 | GAGGAAUC A CAGGGAGA | 617 | TCTCCCTG GGCTAGCTACAACGA GATTCCTC | 4025 |
| 360 | AUGGGGCC A UUUAAGAG | 622 | CTCTTAAA GGCTAGCTACAACGA GGCCCCAT | 4026 |
| 379 | CUGUGUUC A UCUUGAUU | 624 | AATCAAGA GGCTAGCTACAACGA GAACACAG | 4027 |
| 392 | GAUUCUUC A CCUUCUAG | 627 | CTAGAAGG GGCTAGCTACAACGA GAAGAATC | 4028 |
| 420 | AGUAAUUC A CUCAUUCA | 634 | TGAATGAG GGCTAGCTACAACGA GAATTACT | 4029 |
| 424 | AUUCACUC A UUCAGCUG | 636 | CAGCTGAA GGCTAGCTACAACGA GAGTGAAT | 4030 |
| 454 | AUGAAGGC A UUGUCGUU | 642 | AACGACAA GGCTAGCTACAACGA GCCTTCAT | 4031 |
| 495 | GAUGAAAC A CUCAUUCA | 650 | TGAATGAG GGCTAGCTACAACGA GTTTCATC | 4032 |
| 499 | AAACACUC A UUCAACAA | 652 | TTGTTGAA GGCTAGCTACAACGA GAGTGTTT | 4033 |
| 517 | UAAAGGAC A UGGUGACC | 655 | GGTCACCA GGCTAGCTACAACGA GTCCTTTA | 4034 |
| 531 | ACCCAGGC A UCUCUGUA | 659 | TACAGAGA GGCTAGCTACAACGA GCCTGGGT | 4035 |
| 586 | AUGUUGCC A UUUUGAUU | 667 | AATCAAAA GGCTAGCTACAACGA GGCAACAT | 4036 |
| 603 | CCUGAAAC A UGGAAGAC | 670 | GTCTTCCA GGCTAGCTACAACGA GTTTCAGG | 4037 |
| 706 | AACCCUAC A CUGAGCAG | 692 | CTGCTCAG GGCTAGCTACAACGA GTAGGGTT | 4038 |
| 749 | AAGGAUCC A CCUCACUC | 698 | GAGTGAGG GGCTAGCTACAACGA GGATCCTT | 4039 |
| 754 | UCCACCUC A CUCCUGAU | 701 | ATCAGGAG GGCTAGCTACAACGA GAGGTGGA | 4040 |
| 766 | CUGAUUUC A UUGCAGGA | 705 | TCCTGCAA GGCTAGCTACAACGA GAAATCAG | 4041 |
| 798 | UAUGGACC A CAAGGUAA | 709 | TTACCTTG GGCTAGCTACAACGA GGTCCATA | 4042 |
| 810 | GGUAAGGC A UUUGUCCA | 711 | TGGACAAA GGCTAGCTACAACGA GCCTTACC | 4043 |
| 818 | AUUUGUCC A UGAGUGGG | 713 | CCCACTCA GGCTAGCTACAACGA GGACAAAT | 4044 |
| 830 | GUGCGCUC A UCUACGAU | 715 | ATCGTAGA GGCTAGCTACAACGA GAGCCCAC | 4045 |
| 970 | GCUGUUAC A CCAAAAGA | 731 | TCTTTTGG GGCTAGCTACAACGA GTAACAGC | 4046 |
| 982 | AAAGAUGC A CAUUCAAU | 734 | ATTGAATG GGCTAGCTACAACGA GCATCTTT | 4047 |
| 984 | AGAUGCAC A UUCAAUAA | 735 | TTATTGAA GGCTAGCTACAACGA GTGCATCT | 4048 |
| 1071 | AUGUUUGC A CAACAUGU | 749 | ACATGTTG GGCTAGCTACAACGA GCAAACAT | 4049 |
| 1076 | UGCACAAC A UGUUGAUU | 751 | AATCAACA GGCTAGCTACAACGA GTTGTGCA | 4050 |
| 1115 | ACAAAACC A CAACAAAG | 757 | CTTTGTTG GGCTAGCTACAACGA GGTTTTGT | 4051 |
| 1165 | UCCGAAGC A CAUGGGAA | 769 | TTCCCATG GGCTAGCTACAACGA GCTTCGGA | 4052 |
| 1167 | CGAAGCAC A UGGGAAGU | 770 | ACTTCCCA GGCTAGCTACAACGA GTGCTTCG | 4053 |
| 1207 | AGAAAACC A CUCCUAUG | 775 | CATAGGAG GGCTAGCTACAACGA GGTTTTCT | 4054 |
| 1221 | AUGACAAC A CAGCCACC | 780 | GGTGGCTG GGCTAGCTACAACGA GTTGTCAT | 4055 |
| 1227 | ACACAGCC A CCAAAUCC | 783 | GGATTTGG GGCTAGCTACAACGA GGCTGTGT | 4056 |
| 1237 | CAAAUCCC A CCUUCUCA | 788 | TGAGAAGG GGCTAGCTACAACGA GGGATTTG | 4057 |
| 1245 | ACCUUCUC A UUGCUGCA | 792 | TGCAGCAA GGCTAGCTACAACGA GAGAAGGT | 4058 |
| 1300 | CUGGAAGC A UGGCGACU | 800 | AGTCGCCA GGCTAGCTACAACGA GCTTCCAG | 4059 |
| 1395 | AUGGUGAC A UUUGACAG | 820 | CTGTCAAA GGCTAGCTACAACGA GTCACCAT | 4060 |
| 1412 | UGCUGCCC A UGUACAAA | 825 | TTTGTACA GGCTAGCTACAACGA GGGCAGCA | 4061 |
| 1429 | GUGAACUC A UACAGAUA | 828 | TATCTGTA GGCTAGCTACAACGA GAGTTCAC | 4062 |
| 1459 | ACAGGGAC A CACUCGCC | 833 | GGCGAGTG GGCTAGCTACAACGA GTCCCTGT | 4063 |
| 1461 | AGGGACAC A CUCGCCAA | 834 | TTGGCGAG GGCTAGCTACAACGA GTGTCCCT | 4064 |
| 1504 | GGACGUCC A UCUGCAGC | 845 | GCTGCAGA GGCTAGCTACAACGA GGACGTCC | 4065 |
| 1527 | CGAUCGGC A UUUACUGU | 849 | ACAGTAAA GGCTAGCTACAACGA GCCGATCG | 4066 |
| 1600 | AAGACAAC A CUAUAAGU | 858 | ACTTATAG GGCTAGCTACAACGA GTTGTCTT | 4067 |
| 1642 | GUGGUGCC A UCAUCCAC | 864 | GTGGATGA GGCTAGCTACAACGA GGCACCAC | 4068 |
| 1645 | GUGCCAUC A UCCACACA | 865 | TGTGTGGA GGCTAGCTACAACGA GATGGCAC | 4069 |
| 1649 | CAUCAUCC A CACAGUCG | 867 | CGACTGTG GGCTAGCTACAACGA GGATGATG | 4070 |
| 1651 | UCAUCCAC A CAGUCGCU | 868 | AGCGACTG GGCTAGCTACAACGA GTGGATGA | 4071 |
| 1722 | UUACAGAC A UAUGCUUC | 884 | GAAGCATA GGCTAGCTACAACGA GTCTGTAA | 4072 |
| 1756 | AUGGCCUC A UUGAUGCU | 892 | AGCATCAA GGCTAGCTACAACGA GAGGCCAT | 4073 |
| 1779 | GCCCUUUC A UCAGGAAA | 897 | TTTCCTGA GGCTAGCTACAACGA GAAAGGGC | 4074 |
| 1810 | AGCGCUCC A UCCAGCUU | 905 | AAGCTGGA GGCTAGCTACAACGA GGAGCGCT | 4075 |
| 1864 | UGAAUGGC A CAGUGAUC | 917 | GATCACTG GGCTAGCTACAACGA GCCATTCA | 4076 |
| 1882 | UGGACAGC A CCGUGGGA | 920 | TCCCACGG GGCTAGCTACAACGA GCTGTCCA | 4077 |
| 1897 | GAAAGGAC A CUUUGUUU | 922 | AAACAAAG GGCTAGCTACAACGA GTCCTTTC | 4078 |
| 1912 | UUCUUAUC A CCUGGACA | 925 | TGTCCAGG GGCTAGCTACAACGA GATAAGAA | 4079 |
| 1993 | ACAAAAAC A CCAAAAUG | 947 | CATTTTGG GGCTAGCTACAACGA GTTTTTGT | 4080 |
| 2023 | UCCCAGGC A UUGCUAAG | 959 | CTTAGCAA GGCTAGCTACAACGA GCCTGGGA | 4081 |
| 2038 | AGGUUGGC A CUUGGAAA | 961 | TTTCCAAG GGCTAGCTACAACGA GCCAACCT | 4082 |
| 2067 | GCAAGCUC A CAAACCUU | 968 | AAGGTTTG GGCTAGCTACAACGA GAGCTTGC | 4083 |
| 2089 | UGACUGUC A CGUCCCGU | 976 | ACGGGACG GGCTAGCTACAACGA GACAGTCA | 4084 |
| 2152 | ACAAGGAC A CCAGCAAA | 994 | TTTGCTGG GGCTAGCTACAACGA GTCCTTGT | 4085 |
| 2230 | CCAGUGUC A CAGCCCUG | 1019 | CAGGGCTG GGCTAGCTACAACGA GACACTGG | 4086 |
| 2338 | GGUAUUUC A CAACUUAU | 1037 | ATAAGTTG GGCTAGCTACAACGA GAAATACC | 4087 |
| 2350 | CUUAUGAC A CGAAUGGU | 1040 | ACCATTCG GGCTAGCTACAACGA GTCATAAG | 4088 |
| 2436 | AGUGGAGC A CUGUACAU | 1052 | ATGTACAG GGCTAGCTACAACGA GCTCCACT | 4089 |
| 2443 | CACUGUAC A UACCUGGC | 1054 | GCCAGGTA GGCTAGCTACAACGA GTACAGTG | 4090 |
| 2484 | UGGAAUCC A CCAAGACC | 1060 | GGTCTTGG GGCTAGCTACAACGA GGATTCCA | 4091 |
| 2519 | UGUUCAAC A CAAGCAAG | 1066 | CTTGCTTG GGCTAGCTACAACGA GTTGAACA | 4092 |
| 2544 | AGCAGAAC A UCCUCGGG | 1071 | CCCGAGGA GGCTAGCTACAACGA GTTCTGCT | 4093 |
| 2559 | GGAGGCUC A UUUGUGGC | 1075 | GCCACAAA GGCTAGCTACAACGA GAGCCTCC | 4094 |
| 2590 | AUGCUCCC A UACCUGAU | 1084 | ATCAGGTA GGCTAGCTACAACGA GGGAGCAT | 4095 |
| 2607 | CUCUUCCC A CCUGGCCA | 1091 | TGGCCAGG GGCTAGCTACAACGA GGGAAGAG | 4096 |
| 2620 | GCCAAAUC A CCGACCUG | 1096 | CAGGTCGG GGCTAGCTACAACGA GATTTGGC | 4097 |
| 2642 | GGAAAUUC A CGGGGGCA | 1100 | TGCCCCCG GGCTAGCTACAACGA GAATTTCC | 4098 |
| 2656 | GCAGUCUC A UUAAUCUG | 1103 | CAGATTAA GGCTAGCTACAACGA GAGACTGC | 4099 |
| 2696 | UUAUGACC A UGGAACAG | 1111 | CTGTTCCA GGCTAGCTACAACGA GGTCATAA | 4100 |
| 2708 | AACAGCUC A CAAGUAUA | 1114 | TATACTTG GGCTAGCTACAACGA GAGCTGTT | 4101 |
| 2719 | AGUAUAUC A UUCGAAUA | 1116 | TATTCGAA GGCTAGCTACAACGA GATATACT | 4102 |
| 2794 | CUGCUCUC A UCCCAAAG | 1130 | CTTTGGGA GGCTAGCTACAACGA GAGAGCAG | 4103 |
| 2845 | CAGAAAAC A UUACUUUU | 1141 | AAAAGTAA GGCTAGCTACAACGA GTTTTCTG | 4104 |
| 2863 | AAAAUGGC A CAGAUCUU | 1143 | AAGATCTG GGCTAGCTACAACGA GCCATTTT | 4105 |
| 2875 | AUCUUUUC A UUGCUAUU | 1146 | AATAGCAA GGCTAGCTACAACGA GAAAAGAT | 4106 |
| 2926 | UAUCCAAC A UUGCACCA | 1154 | TCGTGCAA GGCTAGCTACAACGA GTTGGATA | 4107 |
| 2931 | AACAUUGC A CGAGUAUC | 1155 | GATACTCG GGCTAGCTACAACGA GCAATGTT | 4108 |
| 2955 | AUUCCUCC A CAGACUCC | 1160 | GGAGTCTG GGCTAGCTACAACGA GGAGGAAT | 4109 |
| 2973 | CCAGAGAC A CCUAGUCC | 1166 | GGACTAGG GGCTAGCTACAACGA GTCTCTGG | 4110 |
| 3014 | UAAUAUUC A UAUCAACA | 1177 | TGTTGATA GGCTAGCTACAACGA GAATATTA | 4111 |
| 3025 | UCAACAGC A CCAUUCCU | 1180 | AGGAATGG GGCTAGCTACAACGA GCTGTTGA | 4112 |
| 3028 | ACAGCACC A UUCCUGGC | 1182 | GCCAGGAA GGCTAGCTACAACGA GGTGCTGT | 4113 |
| 3037 | UUCCUGGC A UUCACAUU | 1185 | AATGTGAA GGCTAGCTACAACGA GCCAGGAA | 4114 |
| 3041 | UGGCAUUC A CAUUUUAA | 1186 | TTAAAATG GGCTAGCTACAACGA GAATGCCA | 4115 |
| 3043 | GCAUUCAC A UUUUAAAA | 1187 | TTTTAAAA GGCTAGCTACAACGA GTGAATGC | 4116 |
| 3130 | AAUAAAUC A UUCAUCCU | 1196 | AGGATGAA GGCTAGCTACAACGA GATTTATT | 4117 |
| 3134 | AAUCAUUC A UCCUUUUU | 1197 | AAAAAGGA GGCTAGCTACAACGA GAATGATT | 4118 |
| 3214 | UAUAGUAC A UUUAUACU | 1205 | AGTATAAA GGCTAGCTACAACGA GTACTATA | 4119 |
| 134 | ACAAGUAC G CAAUUUGA | 1215 | TCAAATTG GGCTAGCTACAACGA GTACTTGT | 4120 |
| 312 | CGUAACCC G CAUUUUCC | 1220 | GGAAAATG GGCTAGCTACAACGA GGGTTACG | 4121 |
| 463 | UUGUCGUU G CAAUCGAC | 1225 | GTCGATTG GGCTAGCTACAACGA AACGACAA | 4122 |
| 480 | CCCAAUGU G CCAGAAGA | 1227 | TCTTCTGG GGCTAGCTACAACGA ACATTGGG | 4123 |
| 583 | AAAAUGUU G CCAUUUUG | 1232 | CAAAATGG GGCTAGCTACAACGA AACATTTT | 4124 |
| 655 | ACAAAAAU G CUGAUGUU | 1238 | AACATCAG GGCTAGCTACAACGA ATTTTTGT | 4125 |
| 670 | UUCUGGUU G CUGAGUCU | 1240 | AGACTCAG GGCTAGCTACAACGA AACCAGAA | 4126 |
| 769 | AUUUCAUU G CAGGAAAA | 1247 | TTTTCCTG GGCTAGCTACAACGA AATGAAAT | 4127 |
| 980 | CAAAAGAU G CACAUUCA | 1255 | TGAATGTG GGCTAGCTACAACGA ATCTTTTG | 4128 |
| 1040 | CCAAUCCC G CCAGACGG | 1258 | CCGTCTGG GGCTAGCTACAACGA GGGATTGG | 4129 |
| 1069 | UAAUGUUU G CACAACAU | 1259 | ATGTTGTG GGCTAGCTACAACGA AAACATTA | 4130 |
| 1151 | UCAAAAAU G CAAUCUCC | 1262 | GGAGATTG GGCTAGCTACAACGA ATTTTTGA | 4131 |
| 1248 | UUCUCAUU G CUGCAGAU | 1268 | ATCTGCAG GGCTAGCTACAACGA AATGAGAA | 4132 |
| 1251 | UCAUUGCU G CAGAUUGG | 1269 | CCAATCTG GGCTAGCTACAACGA AGCAATGA | 4133 |
| 1316 | UGGUAACC G CCUCAAUC | 1272 | GATTGAGG GGCTAGCTACAACGA GGTTACCA | 4134 |
| 1353 | CUUUUCCU G CUGCAGAC | 1275 | GTCTGCAG GGCTAGCTACAACGA AGGAAAAG | 4135 |
| 1356 | UUCCUGCU G CAGACAGU | 1276 | ACTGTCTG GGCTAGCTACAACGA AGCAGGAA | 4136 |
| 1405 | UUGACAGU G CUGCCCAU | 1280 | ATGGGCAG GGCTAGCTACAACGA ACTGTCAA | 4137 |
| 1408 | ACAGUGCU G CCCAUGUA | 1281 | TACATGGG GGCTAGCTACAACGA AGCACTGT | 4138 |
| 1465 | ACACACUC G CCAAAAGA | 1284 | TCTTTTGG GGCTAGCTACAACGA GAGTGTGT | 4139 |
| 1480 | GAUUACCU G CAGCAGCU | 1285 | AGCTGCTG GGCTAGCTACAACGA AGGTAATC | 4140 |
| 1508 | GUCCAUCU G CAGCGGGC | 1286 | GCCCGCTG GGCTAGCTACAACGA AGATGGAC | 4141 |
| 1575 | GAAAUUGU G CUGCUGAC | 1291 | GTCAGCAG GGCTAGCTACAACGA ACAATTTC | 4142 |
| 1578 | AUUGUGCU G CUGACGGA | 1292 | TCCGTCAG GGCTAGCTACAACGA AGCACAAT | 4143 |
| 1613 | AAGUGGGU G CUUUAACG | 1294 | CGTTAAAG GGCTAGCTACAACGA ACCCACTT | 4144 |
| 1639 | AAAGUGGU G CCAUCAUC | 1296 | GATGATGG GGCTAGCTACAACGA ACCACTTT | 4145 |
| 1657 | ACACAGUC G CUUUGGGG | 1297 | CCCCAAAG GGCTAGCTACAACGA GACTGTGT | 4146 |
| 1672 | GGCCCUCU G CAGCUCAA | 1298 | TTGAGCTG GGCTAGCTACAACGA AGAGGGCC | 4147 |
| 1726 | AGACAUAU G CUUCAGAU | 1300 | ATCTGAAG GGCTAGCTACAACGA ATATGTCT | 4148 |
| 1762 | UCAUUGAU G CUUUUGGG | 1302 | CCCAAAAG GGCTAGCTACAACGA ATCAATGA | 4149 |
| 1805 | CUCUCAGC G CUCCAUCC | 1303 | GGATGGAG GGCTAGCTACAACGA GCTGAGAG | 4150 |
| 1923 | UGGACAAC G CAGCCUCC | 1307 | GGAGGCTG CGCTAGCTACAACGA GTTGTCCA | 4151 |
| 2026 | CAGGCAUU G CUAAGGUU | 1308 | AACCTTAG GGCTAGCTACAACGA AATGCCTG | 4152 |
| 2055 | UACAGUCU G CAAGCAAG | 1309 | CTTGCTTG GGCTAGCTACAACGA AGACTGTA | 4153 |
| 2098 | CGUCCCGU G CGUCCAAU | 1312 | ATTGGACG GGCTAGCTACAACGA ACGGGACG | 4154 |
| 2107 | CGUCCAAU G CUACCCUG | 1313 | CAGGGTAG GGCTAGCTACAACGA ATTGGACG | 4155 |
| 2115 | GCUACCCU G CCUCCAAU | 1314 | ATTGGAGG GGCTAGCTACAACGA AGGGTAGC | 4156 |
| 2185 | UAGUUUAU G CAAAUAUU | 1317 | AATATTTG GGCTAGCTACAACGA ATAAACTA | 4157 |
| 2195 | AAAUAUUC G CCAAGGAG | 1318 | CTCCTTGG GGCTAGCTACAACGA GAATATTT | 4158 |
| 2296 | GAGCAGGU G CUGAUGCU | 1322 | AGCATCAG GGCTAGCTACAACGA ACCTGCTC | 4159 |
| 2302 | GUGCUGAU G CUACUAAG | 1324 | CTTAGTAG GGCTAGCTACAACGA ATCAGCAC | 4160 |
| 2376 | GUAAAAGU G CGGGCUCU | 1328 | AGAGCCCG GGCTAGCTACAACGA ACTTTTAC | 4161 |
| 2398 | GAGUUAAC G CAGCCAGA | 1329 | TCTGGCTG GGCTAGCTACAACGA GTTAACTC | 4162 |
| 2584 | UCCCAAAU G CUCCCAUA | 1337 | TATGGGAG GGCTAGCTACAACGA ATTTGGGA | 4163 |
| 2788 | AUACUACU G CUCUCAUC | 1348 | GATGAGAG GGCTAGCTACAACGA AGTAGTAT | 4164 |
| 2878 | UUUUCAUU G CUAUUCAG | 1351 | CTGAATAG GGCTAGCTACAACGA AATGAAAA | 4165 |
| 2929 | CCAACAUU G CACGAGUA | 1355 | TACTCGTG GGCTAGCTACAACGA AATGTTGG | 4166 |
| 2964 | CAGACUCC G CCAGAGAC | 1357 | GTCTCTGG GGCTAGCTACAACGA GGAGTCTG | 4167 |
| 2995 | AAACGUCU G CUCCUUGU | 1360 | ACAAGGAG GGCTAGCTACAACGA AGACGTTT | 4168 |
| 3078 | GGAGAACU G CAGCUGUC | 1361 | GACAGCTG GGCTAGCTACAACGA AGTTCTCC | 4169 |
| 3294 | AAUAAAAU G CUAAACAA | 1366 | TTGTTTAG GGCTAGCTACAACGA ATTTTATT | 4170 |
| 27 | AAAUGGAU G UGGAAUAU | 1367 | ATATTCCA GGCTAGCTACAACGA ATCCATTT | 4171 |
| 52 | AUUUUCUU G UUUAAGGG | 1368 | CCCTTAAA GGCTAGCTACAACGA AAGAAAAT | 4172 |
| 75 | GAAGAGGU G UUGAGGUU | 1369 | AACCTCAA GGCTAGCTACAACGA ACCTCTTC | 4173 |
| 86 | GAGGUUAU G UCAAGCAU | 1370 | ATGCTTGA GGCTAGCTACAACGA ATAACCTC | 4174 |
| 155 | AAGAUAUU G UUAUCAUU | 1371 | AATGATAA GGCTAGCTACAACGA AATATCTT | 4175 |
| 221 | AAAGACCU G UGAUAAAC | 1372 | GTTTATCA GGCTAGCTACAACGA AGGTCTTT | 4176 |
| 253 | GGAAACGU G UGUCUAUA | 1373 | TATAGACA GGCTAGCTACAACGA ACGTTTCC | 4177 |
| 255 | AAACGUGU G UCUAUAUU | 1374 | AATATAGA GGCTAGCTACAACGA ACACGTTT | 4178 |
| 273 | UCAUAUCU G UAUAUAUA | 1375 | TATATATA GGCTAGCTACAACGA AGATATGA | 4179 |
| 344 | AGGGAGAU G UACAGCAA | 1376 | TTGCTGTA GGCTAGCTACAACGA ATCTCCCT | 4180 |
| 373 | AGAGUUCU G UGUUCAUC | 1377 | GATGAACA GGCTAGCTACAACGA AGAACTCT | 4181 |
| 375 | AGUUCUGU G UUCAUCUU | 1378 | AAGATGAA GGCTAGCTACAACGA ACAGAACT | 4182 |
| 457 | AAGGCAUU G UCGUUGCA | 1379 | TGCAACGA GGCTAGCTACAACGA AATGCCTT | 4183 |
| 478 | ACCCCAAU G UGCCAGAA | 1380 | TTCTGGCA GGCTAGCTACAACGA ATTGGGGT | 4184 |
| 537 | GCAUCUCU G UAUCUGUU | 1381 | AACAGATA GGCTAGCTACAACGA AGAGATGC | 4185 |
| 543 | CUGUAUCU G UUUGAAGC | 1382 | GCTTCAAA GGCTAGCTACAACGA AGATACAG | 4186 |
| 580 | UCAAAAAU G UUGCCAUU | 1383 | AATGGCAA GGCTAGCTACAACGA ATTTTTGA | 4187 |
| 625 | CUGACUAU G UGAGACCA | 1384 | TGGTCTCA GGCTAGCTACAACGA ATAGTCAG | 4188 |
| 661 | AUGCUGAU G UUCUGGUU | 1385 | AACCAGAA GGCTAGCTACAACGA ATCAGCAT | 4189 |
| 725 | GGGCAACU G UGGAGAGA | 1386 | TCTCTCCA GGCTAGCTACAACGA AGTTGCCC | 4190 |
| 814 | AGGCAUUU G UCCAUGAG | 1387 | CTCATGGA GGCTAGCTACAACGA AAATGCCT | 4191 |
| 911 | AGUAAGAU G UUCACCAC | 1388 | CTGCTGAA GGCTAGCTACAACGA ATCTTACT | 4192 |
| 937 | GUACAAAU G UAGUAAAG | 1389 | CTTTACTA GGCTAGCTACAACGA ATTTGTAC | 4193 |
| 950 | AAAGAAGU G UCAGGGAG | 1390 | CTCCCTGA GGCTAGCTACAACGA ACTTCTTT | 4194 |
| 965 | AGGCAGCU G UUACACCA | 1391 | TGGTGTAA GGCTAGCTACAACGA AGCTGCCT | 4195 |
| 1019 | AAAAGGAU G UGAGUUUG | 1392 | CAAACTCA GGCTAGCTACAACGA ATCCTTTT | 4196 |
| 1027 | GUGAGUUU G UUCUCCAA | 1393 | TTGGAGAA GGCTAGCTACAACGA AAACTCAC | 4197 |
| 1065 | UCUAUAAU G UUUGCACA | 1394 | TGTGCAAA GGCTAGCTACAACGA ATTATAGA | 4198 |
| 1078 | CACAACAU G UUGAUUCU | 1395 | AGAATCAA GGCTAGCTACAACGA ATGTTGTG | 4199 |
| 1100 | UGAAUUCU G UACAGAAC | 1396 | GTTCTGTA GGCTAGCTACAACGA AGAATTCA | 4200 |
| 1270 | AAAGAAUU G UGUGUUUA | 1397 | TAAACACA GGCTAGCTACAACGA AATTCTTT | 4201 |
| 1272 | AGAAUUGU G UGUUUAGU | 1398 | ACTAAACA GGCTAGCTACAACGA ACAATTCT | 4202 |
| 1274 | AAUUGUGU G UUUAGUCC | 1399 | GGACTAAA GGCTAGCTACAACGA ACACAATT | 4203 |
| 1414 | CUGCCCAU G UACAAAGU | 1400 | ACTTTGTA GGCTAGCTACAACGA ATGGGCAC | 4204 |
| 1534 | CAUUUACU G UGAUUAGG | 1401 | CCTAATCA GGCTAGCTACAACGA AGTAAATG | 4205 |
| 1573 | CUCAAAUU G UGCUGCUG | 1402 | CAGCAGCA GGCTAGCTACAACGA AATTTCAG | 4206 |
| 1695 | CAGGAGCU G UCCAAAAU | 1403 | ATTTTGGA GGCTAGCTACAACGA AGCTCCTC | 4207 |
| 1795 | AUGGAGCU G UCUCUCAG | 1404 | CTGAGAGA GGCTAGCTACAACGA AGCTCCAT | 4208 |
| 1902 | GACACUUU G UUUCUUAU | 1405 | ATAAGAAA GGCTAGCTACAACGA AAAGTCTC | 4209 |
| 1978 | GUGGCUUU G UAGUGGAC | 1406 | GTCCACTA GGCTAGCTACAACGA AAAGCCAC | 4210 |
| 2086 | CCCUGACU G UCACGUCC | 1407 | GGACGTGA GGCTAGCTACAACGA AGTCAGGG | 4211 |
| 2227 | GGGCCAGU G UCACAGCC | 1408 | GGCTGTGA GGCTAGCTACAACGA ACTGGCCC | 4212 |
| 2320 | AUGACGGU G UCUACUCA | 1409 | TGAGTACA GGCTAGCTACAACGA ACCGTCAT | 4213 |
| 2368 | GAUACAGU G UAAAAGUG | 1410 | CACTTTTA GGCTAGCTACAACGA ACTCTATC | 4214 |
| 2439 | GGAGCACU G UACAUACC | 1411 | GGTATGTA GGCTAGCTACAACGA AGTGCTCC | 4215 |
| 2512 | AGGAUGAU G UUCAACAC | 1412 | GTGTTGAA GGCTAGCTACAACGA ATCATCCT | 4216 |
| 2529 | AAGCAAGU G UGUUUCAG | 1413 | CTGAAACA GGCTAGCTACAACGA ACTTGCTT | 4217 |
| 2531 | GCAAGUCU G UUUCAGCA | 1414 | TGCTGAAA GGCTAGCTACAACGA ACACTTGC | 4218 |
| 2563 | GCUCAUUU G UGGCUUCU | 1415 | AGAAGCCA GGCTAGCTACAACGA AAATGAGC | 4219 |
| 2575 | CUUCUGAU G UCCCAAAU | 1416 | ATTTGGGA GGCTAGCTACAACGA ATCAGAAG | 4220 |
| 2829 | GUCUUUUU G UUUAAACC | 1417 | GGTTTAAA GGCTAGCTACAACGA AAAAAGAC | 4221 |
| 2890 | UUCAGGCU G UUGAUAAG | 1418 | CTTATCAA GGCTAGCTACAACGA AGCCTGAA | 4222 |
| 2943 | GUAUCUUU G UUUAUUCC | 1419 | GGAATAAA GGCTAGCTACAACGA AAAGATAC | 4223 |
| 3002 | UGCUCCUU G UCCUAAUA | 1420 | TATTAGGA GGCTAGCTACAACGA AAGGAGCA | 4224 |
| 3057 | AAAAUUAU G UGGAAGUG | 1421 | CACTTCCA GGCTAGCTACAACGA ATAATTTT | 4225 |
| 3084 | CUGCACCU G UCAAUAGC | 1422 | GCTATTGA GGCTAGCTACAACGA AGCTGCAG | 4226 |
| 3109 | GAAUUUUU G UCAGAUAA | 1423 | TTATCTGA GGCTAGCTACAACGA AAAAATTC | 4227 |
| 3166 | UCUAAAAU G UAUUUUAG | 1424 | CTAAAATA GGCTAGCTACAACGA ATTTTAGA | 4228 |
| 3182 | GACUUCCU G UAGGGGGC | 1425 | GCCCCCTA GGCTAGCTACAACGA AGGAAGTC | 4229 |
| 3272 | GACUUCCU G UAGGGGGC | 1425 | GCCCCCTA GGCTAGCTACAACGA AGGAAGTC | 4229 |
| 3203 | UACUAAAU G UAUAUACU | 1426 | ACTATATA GGCTAGCTACAACGA ATTTAGTA | 4230 |
| 3227 | UACUAAAU G UAUUCCUG | 1427 | CAGGAATA GGCTAGCTACAACGA ATTTAGTA | 4231 |
| 3235 | GUAUUCCU G UAGGGGGC | 1428 | GCCCCCTA GGCTAGCTACAACGA AGGAATAC | 4232 |
| 3256 | UACUAAAU G UAUUUUAG | 1429 | CTAAAATA GGCTAGCTACAACGA ATTTAGGTA | 4233 |
| 15 | UGCUUUUG G UACAAAUG | 1430 | CATTTGTA GGCTAGCTACAACGA CAAAAGCA | 4234 |
| 63 | UAAGGGGA G CAUGAAGA | 1431 | TCTTCATG GGCTAGCTACAACGA TCCCCTTA | 4235 |
| 73 | AUGAAGAG G UCUUGAGG | 1432 | CCTCAACA GGCTAGCTACAACGA CTCTTCAT | 4236 |
| 81 | GUGUUGAG G UUAUGUCA | 1433 | TGACATAA GGCTAGCTACAACGA CTCAACAC | 4237 |
| 91 | UAUGUCAA G CAUCUGGC | 1434 | GCCAGATG GGCTAGCTACAACGA TTGACATA | 4238 |
| 98 | AGCAUCUG G CACAGCUG | 1435 | CAGCTGTG GGCTAGCTACAACGA CAGATGCT | 4239 |
| 103 | CUGGCACA G CUGAAGGC | 1436 | GCCTTCAG GGCTAGCTACAACGA TGTGCCAG | 4240 |
| 110 | AGCUGAAG G CAGAUGGA | 1437 | TCCATCTG GGCTAGCTACAACGA CTTCAGCT | 4241 |
| 130 | AUUUACAA G UACGCAAU | 1438 | ATTGCGTA GGCTAGCTACAACGA TTGTAAAT | 4242 |
| 182 | AGACAAGA G CAAUAGUA | 1439 | TACTATTG GGCTAGCTACAACGA TCTTGTCT | 4243 |
| 188 | GACCAAUA G UAAAACAC | 1440 | GTGTTTTA GGCTAGCTACAACGA TATTGCTC | 4244 |
| 202 | CACAUCAG G UCAGGGGG | 1441 | CCCCCTGA GGCTAGCTACAACGA CTGATCTC | 4245 |
| 210 | GUCAGGGG G UUAAAGAC | 1442 | GTCTTTAA GGCTAGCTACAACGA CCCCTGAC | 4246 |
| 242 | UCCGAUAA G UUGGAAAC | 1443 | GTTTCCAA GGCTAGCTACAACGA TTATCGGA | 4247 |
| 251 | UUGGAAAC G UGUGUCUA | 1444 | TAGACACA GGCTAGCTACAACGA GTTTCCAA | 4248 |
| 287 | AUAUAAUG G UAAAGAAA | 1445 | TTTCTTTA GGCTAGCTACAACGA CATTATAT | 4249 |
| 305 | ACACCUUC G UAACCCGC | 1446 | GCGGGTTA GGCTAGCTACAACGA GAAGGTGT | 4250 |
| 349 | GAUGUACA G CAAUGGGG | 1447 | CCCCATTG GGCTAGCTACAACGA TGTACATC | 4251 |
| 357 | GCAAUGGG G CCAUUUAA | 1448 | TTAAATGG GGCTAGCTACAACGA CCCATTGC | 4252 |
| 368 | AUUUAAGA G UUCUGUGU | 1449 | ACACAGAA GGCTAGCTACAACGA TCTTAAAT | 4253 |
| 406 | UAGAAGGG G CCCUGAGU | 1450 | ACTCAGGG GGCTAGCTACAACGA CCCTTCTA | 4254 |
| 413 | GGCCCUGA G UAAUUCAC | 1451 | GTGAATTA GGCTAGCTACAACGA TCAGGGCC | 4255 |
| 429 | CUCAUUCA G CUGAACAA | 1452 | TTGTTCAG GGCTAGCTACAACGA TGAATGAG | 4256 |
| 443 | CAACAAUG G CUAUGAAG | 1453 | CTTCATAG GGCTAGCTACAACGA CATTGTTG | 4257 |
| 452 | CUAUGAAG G CAUUGUCG | 1454 | CGACAATG GGCTAGCTACAACGA CTTCATAG | 4258 |
| 460 | GCAUUGUC G UUGCAAUC | 1455 | GATTGCAA GGCTAGCTACAACGA GACAATGC | 4259 |
| 520 | AGGACAUG G UGACCCAG | 1456 | CTGGGTCA GGCTAGCTACAACGA CATGTCCT | 4260 |
| 529 | UGACCCAG G CAUCUCUG | 1457 | CAGAGATG GGCTAGCTACAACGA CTGGGTCA | 4261 |
| 550 | UGUUUGAA G CUACAGGA | 1458 | TCCTGTAG GGCTAGCTACAACGA TTCAAACA | 4262 |
| 561 | ACAGGAAA G CGAUUUUA | 1459 | TAAAATCG GGCTAGCTACAACGA TTTCCTGT | 4263 |
| 616 | AGACAAAG G CUGACUAU | 1460 | ATAGTCAG GGCTAGCTACAACGA CTTTGTCT | 4264 |
| 667 | AUGUUCUG G UUGCUGAG | 1461 | CTCAGCAA GGCTAGCTACAACGA CAGAACAT | 4265 |
| 675 | GUUGCUGA G UCUACUCC | 1462 | GGAGTAGA GGCTAGCTACAACGA TCAGCAAC | 4266 |
| 689 | UCCUCCAG G UAAUGAUG | 1463 | CATCATTA GGCTAGCTACAACGA CTGGAGGA | 4267 |
| 711 | UACACUGA G CAGAUGGG | 1464 | CCCATCTG GGCTAGCTACAACGA TCAGTGTA | 4268 |
| 719 | GCAGAUGG G CAACUGUG | 1465 | CACAGTTG GGCTAGCTACAACGA CCATCTGC | 4269 |
| 737 | AGAGAAGG G UGAAAGGA | 1466 | TCCTTTCA GGCTACCTACAACGA CCTTCTCT | 4270 |
| 780 | GGAAAAAA G UUAGCUGA | 1467 | TCAGCTAA GGCTAGCTACAACGA TTTTTTCC | 4271 |
| 784 | AAAAGUUA G CUGAAUAU | 1468 | ATATTCAG GGCTAGCTACAACGA TAACTTTT | 4272 |
| 803 | ACCACAAG G UAAGGCAU | 1469 | ATGCCTTA GGCTAGCTACAACGA CTTGTGGT | 4273 |
| 808 | AAGGUAAG G CAUUUGUC | 1470 | GACAAATG GGCTAGCTACAACGA CTTACCTT | 4274 |
| 822 | GUCCAUGA G UGGGCUCA | 1471 | TGAGCCCA GGCTAGCTACAACGA TCATGGAC | 4275 |
| 826 | AUGAGUGG G CUCAUCUA | 1472 | TAGATGAG GGCTAGCTACAACGA CCACTCAT | 4276 |
| 844 | GAUGGGGA G UAUUUGAC | 1473 | GTCAAATA GGCTAGCTACAACGA TCCCCATC | 4277 |
| 855 | UUUGACGA G UACAAUAA | 1474 | TTATTGTA GGCTAGCTACAACGA TCGTCAAA | 4278 |
| 901 | GAAUACAA G CAGUAAGA | 1475 | TCTTACTG GGCTAGCTACAACGA TTGTATTC | 4279 |
| 904 | UACAAGCA G UAAGAUGU | 1476 | ACATCTTA GGCTAGCTACAACGA TGCTTGTA | 4280 |
| 916 | GAUGUUCA G CAGGUAUU | 1477 | AATACCTG GGCTAGCTACAACGA TGAACATC | 4281 |
| 920 | UUCAGCAG G UAUUACUG | 1478 | CAGTAATA GGCTAGCTACAACGA CTGCTGAA | 4282 |
| 929 | UAUUACUG G UACAAAUG | 1479 | CATTTGTA GGCTAGCTACAACGA CAGTAATA | 4283 |
| 940 | CAAAUGUA G UAAAGAAG | 1480 | CTTCTTTA GGCTAGCTACAACGA TACATTTG | 4284 |
| 948 | GUAAAGAA G UGUCAGGG | 1481 | CCCTGACA GGCTAGCTACAACGA TTCTTTAC | 4285 |
| 959 | UCAGGGAG G CAGCUGUU | 1482 | AACAGCTG GGCTAGCTACAACGA CTCCCTGA | 4286 |
| 962 | GGGAGGCA G CUGUUACA | 1483 | TCTAACAG GGCTAGCTACAACGA TGCCTCCC | 4287 |
| 994 | UCAAUAAA G UUACAGGA | 1484 | TCCTGTAA GGCTAGCTACAACGA TTTATTGA | 4288 |
| 1023 | GGAUGUGA G UUUGUUCU | 1485 | AGAACAAA GGCTAGCTACAACGA TCACATCC | 4289 |
| 1054 | CGGAGAAG G CUUCUAUA | 1486 | TATAGAAG GGCTAGCTACAACGA CTTCTCCG | 4290 |
| 1090 | AUUCUAUA G UUGAAUUC | 1487 | GAATTCAA GGCTAGCTACAACGA TATAGAAT | 4291 |
| 1126 | ACAAAGAA G CUCCAAAC | 1488 | CTTTCCAG GGCTAGCTACAACGA TTCTTTGT | 4292 |
| 1137 | CCAAACAA G CAAAAUCA | 1489 | TCATTTTC GGCTAGCTACAACGA TTGTTTCC | 4293 |
| 1163 | UCUCCCAA G CACAUGGG | 1490 | CCCATGTG GGCTAGCTACAACGA TTCGGAGA | 4294 |
| 1174 | CAUGGGAA G UGAUCCGU | 1491 | ACGGATCA GGCTAGCTACAACGA TTCCCATC | 4295 |
| 1181 | AGUGAUCC G UGAUUCUG | 1492 | CAGAATCA GGCTAGCTACAACGA GGATCACT | 4296 |
| 1224 | ACAACACA G CCACCAAA | 1493 | TTTGCTGG GGCTAGCTACAACGA TGTGTTGT | 4297 |
| 1279 | UGUGUUUA G UCCUUGAC | 1494 | GTCAAGGA GGCTAGCTACAACGA TAAACACA | 4298 |
| 1298 | AUCUCCAA G CAUGGCGA | 1495 | TCGCCATG GGCTAGCTACAACGA TTCCAGAT | 4299 |
| 1303 | GAAGCAUG G CGACUGGU | 1496 | ACCAGTCC GGCTAGCTACAACGA CATGCTTC | 4300 |
| 1310 | GGCGACUG G UAACCGCC | 1497 | GGCGGTTA GGCTAGCTACAACGA CAGTCGCC | 4301 |
| 1336 | UGAAUCAA G CAGGCCAG | 1498 | CTGGCCTC GGCTAGCTACAACGA TTGATTCA | 4302 |
| 1340 | UCAAGCAG G CCAGCUUU | 1499 | AAAGCTGG GGCTAGCTACAACGA CTGCTTGA | 4303 |
| 1344 | GCAGGCCA G CUUUUCCU | 1500 | AGGAAAAG GGCTAGCTACAACGA TGGCCTGC | 4304 |
| 1363 | UGCAGACA G UUGAGCUG | 1501 | CAGCTCAA GGCTAGCTACAACGA TGTCTGCA | 4305 |
| 1368 | ACAGUUGA G CUGGGGUC | 1502 | GACCCCAG GGCTAGCTACAACGA TCAACTGT | 4306 |
| 1374 | GAGCUGGG G UCCUGGGU | 1503 | ACCCAGGA GGCTAGCTACAACGA CCCAGCTC | 4307 |
| 1381 | GGUCCUGG G UUGGGAUG | 1504 | CATCCCAA GGCTAGCTACAACGA CCAGGACC | 4308 |
| 1390 | UUGGGAUG G UGACAUUU | 1505 | AAATGTCA GGCTAGCTACAACGA CATCCCAA | 4309 |
| 1403 | AUUUGACA G UGCUGCCC | 1506 | GGGCAGCA GGCTAGCTACAACGA TGTCAAAT | 4310 |
| 1421 | UGUACAAA G UGAACUCA | 1507 | TGAGTTCA GGCTAGCTACAACGA TTTGTACA | 4311 |
| 1442 | GAUAGACA G UGGCAGUG | 1508 | CACTGCCA GGCTAGCTACAACGA TGTTTATC | 4312 |
| 1445 | AAACAGUG G CAGUGACA | 1509 | TGTCACTG GGCTAGCTACAACGA CACTGTTT | 4313 |
| 1448 | CAGUGGCA G UGACAGGG | 1510 | CCCTGTCA GGCTAGCTACAACGA TGCCACTG | 4314 |
| 1483 | UACCUGCA G CAGCUUCA | 1511 | TGAAGCTG GGCTAGCTACAACGA TGCAGGTA | 4315 |
| 1486 | CUGCAGCA G CUUCAGGA | 1512 | TCCTGAAG GGCTAGCTACAACGA TGCTGCAG | 4316 |
| 1500 | GGAGGGAC G UCCAUCUG | 1513 | CAGATGGA GGCTAGCTACAACGA GTCCCTCC | 4317 |
| 1511 | CAUCUGCA G CGGGCUUC | 1514 | GAAGCCCG GGCTAGCTACAACGA TGCAGATG | 4318 |
| 1515 | UGCAGCGG G CUUCGAUC | 1515 | GATCGAAG GGCTAGCTACAACGA CCGCTGCA | 4319 |
| 1525 | UUCGAUCG G CAUUUACU | 1516 | AGTAAATG GGCTAGCTACAACGA CGATCGAA | 4320 |
| 1607 | CACUAUAA G UGGGUGCU | 1517 | AGCACCCA GGCTAGCTACAACGA TTATAGTG | 4321 |
| 1611 | AUAAGUGG G UGCUUUAA | 1518 | TTAAAGCA GGCTAGCTACAACGA CCACTTAT | 4322 |
| 1624 | UUAACGAG G UCAAACAA | 1519 | TTGTTTGA GGCTAGCTACAACGA CTCGTTAA | 4323 |
| 1634 | CAAACAAA G UGGUGCCA | 1520 | TGGCACCA GGCTAGCTACAACGA TTTGTTTG | 4324 |
| 1637 | ACAAAGUG G UGCCAUCA | 1521 | TGATGGCA GGCTAGCTACAACGA CACTTTGT | 4325 |
| 1654 | UCCACACA G UCGCUUUG | 1522 | CAAAGCGA GGCTAGCTACAACGA TGTGTGGA | 4326 |
| 1665 | GCUUUGGG G CCCUCUGC | 1523 | GCAGAGGG GGCTAGCTACAACGA CCCAAAGC | 4327 |
| 1675 | CCUCUGCA G CUCAAGAA | 1524 | TTCTTGAG GGCTAGCTACAACGA TGCAGAGG | 4328 |
| 1692 | CUAGAGGA G CUGUCCAA | 1525 | TTGGACAG GGCTAGCTACAACGA TCCTCTAG | 4329 |
| 1712 | GACAGGAG G UUUACAGA | 1526 | TCTGTAAA GGCTAGCTACAACGA CTCCTGTC | 4330 |
| 1738 | CAGAUCAA G UUCAGAAC | 1527 | GTTCTGAA GGCTAGCTACAACGA TTGATCTG | 4331 |
| 1751 | GAACAAUG G CCUCAUUG | 1528 | CAATGAGG GGCTAGCTACAACGA CATTGTTC | 4332 |
| 1771 | CUUUUGGG G CCCUUUCA | 1529 | TGAAAGGG GGCTAGCTACAACGA CCCAAAAG | 4333 |
| 1792 | GAAAUGGA G CUGUCUCU | 1530 | AGAGACAG GGCTAGCTACAACGA TCCATTTC | 4334 |
| 1803 | GUCUCUCA G CGCUCCAU | 1531 | ATGGAGCG GGCTAGCTACAACGA TGAGAGAC | 4335 |
| 1815 | UCCAUCCA G CUUGAGAG | 1532 | CTCTCAAG GGCTAGCTACAACGA TGGATGGA | 4336 |
| 1823 | GCUUGAGA G UAAGGGAU | 1533 | ATCCCTTA GGCTAGCTACAACGA TCTCAAGC | 4337 |
| 1847 | CCAGAACA G CCAGUGGA | 1534 | TCCACTGG GGCTAGCTACAACGA TGTTCTGG | 4338 |
| 1851 | AACAGCCA G UGGAUGAA | 1535 | TTCATCCA GGCTAGCTACAACGA TGGCTGTT | 4339 |
| 1862 | GAUGAAUG G CACAGUGA | 1536 | TCACTGTG GGCTAGCTACAACGA CATTCATC | 4340 |
| 1867 | AUGGCACA G UGAUCGUG | 1537 | CACGATCA GGCTAGCTACAACGA TGTGCCAT | 4341 |
| 1873 | CAGUGAUC G UGGACAGC | 1538 | GCTGTCCA GGCTAGCTACAACGA GATCACTG | 4342 |
| 1880 | CGUGGACA G CACCGUGG | 1539 | CCACGGTG GGCTAGCTACAACGA TGTCCACG | 4343 |
| 1885 | ACAGCACC G UGGGAAAG | 1540 | CTTTCCCA GGCTAGCTACAACGA GGTGCTGT | 4344 |
| 1926 | ACAACGCA G CCUCCCCA | 1541 | TGGGGAGG GGCTAGCTACAACGA TGCGTTGT | 4345 |
| 1955 | GGAUCCCA G UGGACAGA | 1542 | TCTGTCCA GGCTAGCTACAACGA TGGGATCC | 4346 |
| 1965 | GGACAGAA G CAAGGUGG | 1543 | CCACCTTG GGCTAGCTACAACGA TTCTGTCC | 4347 |
| 1970 | GAAGCAAG G UGGCUUUG | 1544 | CAAAGCCA GGCTAGCTACAACGA CTTGCTTC | 4348 |
| 1973 | GCAAGGUG G CUUUGUAG | 1545 | CTACAAAG GGCTAGCTACAACGA CACCTTGC | 4349 |
| 1981 | GCUUUGUA G UGGACAAA | 1546 | TTTGTCCA GGCTAGCTACAACGA TACAAAGC | 4350 |
| 2002 | CCAAAAUG G CCUACCUC | 1547 | GAGGTAGG GGCTAGCTACAACGA CATTTTGG | 4351 |
| 2021 | AAUCCCAG G CAUUGCUA | 1548 | TAGCAATG GGCTAGCTACAACGA CTGGGATT | 4352 |
| 2032 | UUGCUAAG G UUGGCACU | 1549 | AGTGCCAA GGCTAGCTACAACGA CTTAGCAA | 4353 |
| 2036 | UAAGGUUG G CACUUGGA | 1550 | TCCAAGTG GGCTAGCTACAACGA CAACCTTA | 4354 |
| 2051 | GAAAUACA G UCUGCAAG | 1551 | CTTGCAGA GGCTAGCTACAACGA TGTATTTC | 4355 |
| 2059 | GUCUGCAA G CAAGCUCA | 1552 | TGAGCTTG GGCTAGCTACAACGA TTGCAGAC | 4356 |
| 2063 | GCAAGCAA G CUCACAAA | 1553 | TTTGTGAG GGCTAGCTACAACGA TTGCTTGC | 4357 |
| 2091 | ACUGUCAC G UCCCGUGC | 1554 | GCACGGGA GGCTAGCTACAACGA GTGACAGT | 4358 |
| 2096 | CACGUCCC G UGCGUCCA | 1555 | TGGACGCA GGCTAGCTACAACGA GGGACGTG | 4359 |
| 2100 | UCCCGUGC G UCCAAUGC | 1556 | GCATTGGA GGCTAGCTACAACGA GCACGGGA | 4360 |
| 2128 | CAAUUACA G UGACUUCC | 1557 | GGAAGTCA GGCTAGCTACAACGA TGTAATTG | 4361 |
| 2156 | GGACACCA G CAAUUCC | 1558 | GGAATTTG GGCTAGCTACAACGA TGGTGTCC | 4362 |
| 2168 | AUUCCCCA G CCCUCUGG | 1559 | CCAGAGGG GGCTAGCTACAACGA TGGGGAAT | 4363 |
| 2176 | GCCCUCUG G UAGUUUAU | 1560 | ATAAACTA GGCTAGCTACAACGA CAGAGGGC | 4364 |
| 2179 | CUCUGGUA G UUUAUGCA | 1561 | TGCATAAA GGCTAGCTACAACGA TACCAGAG | 4365 |
| 2203 | GCCAAGGA G CCUCCCCA | 1562 | TGGGGAGG GGCTAGCTACAACGA TCCTTGGC | 4366 |
| 2221 | UUCUCAGG G CCAGUGUC | 1563 | GACACTGG GGCTAGCTACAACGA CCTGAGAA | 4367 |
| 2225 | CAGGGCCA G UGUCACAC | 1564 | CTGTGACA GGCTAGCTACAACGA TGGCCCTG | 4368 |
| 2233 | GUGUCACA G CCCUCAUU | 1565 | AATCAGGG GGCTAGCTACAACGA TGTGACAC | 4369 |
| 2248 | UUGAAUCA G UGAAUGGA | 1566 | TCCATTCA GGCTAGCTACAACGA TGATTCAA | 4370 |
| 2263 | GAAAAACA G UUACCUUG | 1567 | CAAGGTAA GGCTAGCTACAACGA TGTTTTTC | 4371 |
| 2290 | AUAAUGGA G CAGGUGCU | 1568 | AGCACCTG GGCTAGCTACAACGA TCCATTAT | 4372 |
| 2294 | UCGAGCAG G UCCUGAUG | 1569 | CATCAGCA GGCTAGCTACAACGA CTGCTCCA | 4373 |
| 2318 | GGAUGACG G UGUCUACU | 1570 | ACTAGACA CGCTAGCTACAACGA CGTCATCC | 4374 |
| 2331 | UACUCAAG G UAUUUCAC | 1571 | GTCAAATA GGCTAGCTACAACCA CTTCACTA | 4375 |
| 2357 | CACGAAUG G UAGAUACA | 1572 | TGTATCTA GGCTAGCTACAACGA CATTCGTG | 4376 |
| 2366 | UAGAUACA G UGUAAAAG | 1573 | CTTTTACA GGCTAGCTACAACGA TGTATCTA | 4377 |
| 2374 | GUGUAAAA G UGCGGGCU | 1574 | AGCCCGCA GGCTAGCTACAACGA TTTTACAC | 4378 |
| 2380 | AAGUGCGG G CUCUGGGA | 1575 | TCCCAGAG GGCTAGCTACAACGA CCGCACTT | 4379 |
| 2392 | UGGGAGGA G UUAACGCA | 1576 | TGCGTTAA GGCTAGCTACAACGA TCCTCCCA | 4380 |
| 2401 | UUAACGCA G CCAGACGG | 1577 | CCGTCTGG GGCTAGCTACAACGA TGCGTTAA | 4381 |
| 2413 | GACGGAGA G UGAUACCC | 1578 | GGGTATCA GGCTAGCTACAACGA TCTCCGTC | 4382 |
| 2424 | AUACCCCA G CAGAGUGG | 1579 | CCACTCTG GGCTAGCTACAACGA TGGGGTAT | 4383 |
| 2429 | CCAGCAGA G UGGAGCAC | 1580 | GTGCTCCA GGCTAGCTACAACGA TCTGCTGG | 4384 |
| 2434 | AGAGUGGA G CACUGUAC | 1581 | GTACAGTG GGCTAGCTACAACGA TCCACTCT | 4385 |
| 2450 | CAUACCUG G CUGGAUUG | 1582 | CAATCCAG GGCTAGCTACAACGA CAGGTATG | 4386 |
| 2523 | CAACACAA G CAAGUGUG | 1583 | CACACTTG CGCTAGCTACAACGA TTGTCTTG | 4387 |
| 2527 | ACAAGCAA G UGUGUUUC | 1584 | GAAACACA GGCTAGCTACAACGA TTGCTTCT | 4388 |
| 2537 | GUCUUUCA G CAGAACAU | 1585 | ATGTTCTG GCCTAGCTACAACGA TGAAACAC | 4389 |
| 2555 | CUCGGGAG G CUCAUUUG | 1586 | CAAATGAG GGCTAGCTACAACGA CTCCCGAG | 4390 |
| 2566 | CAUUUGUG G CUUCUGAU | 1587 | ATCAGAAG GGCTAGCTACAACGA CACAAATG | 4391 |
| 2612 | CCCACCUG G CCAAAUCA | 1588 | TGATTTGG GGCTAGCTACAACGA CAGGTCCC | 4392 |
| 2632 | ACCUGAAG G CGGAAAUU | 1589 | AATTTCCG GGCTAGCTACAACGA CTTCAGGT | 4393 |
| 2648 | UCACGGGG G CAGUCUCA | 1590 | TGACACTG GGCTAGCTACAACGA CCCCGTGA | 4394 |
| 2651 | CGGGGGCA G UCUCAUUA | 1591 | TAATGAGA GGCTAGCTACAACGA TGCCCCCG | 4395 |
| 2674 | CUUGGACA G CUCCUGGG | 1592 | CCCAGGAG GGCTAGCTACAACGA TGTCCAAG | 4396 |
| 2704 | AUGGAACA G CUCACAAG | 1593 | CTTGTGAG GGCTAGCTACAACGA TGTTCCAT | 4397 |
| 2712 | GCUCACAA G UAUAUCAU | 1594 | ATGATATA GCCTAGCTACAACGA TTGTCAGC | 4398 |
| 2729 | UCGAAUAA G UACAAGUA | 1595 | TACTTGTA GGCTAGCTACAACGA TTATTCCA | 4399 |
| 2735 | AAGUACAA G UAUUCUUG | 1596 | CAAGAATA GGCTAGCTACAACGA TTGTACTT | 4400 |
| 2757 | AGAGACAA G UUCAAUGA | 1597 | TCATTGAA GGCTAGCTACAACGA TTGTCTCT | 4401 |
| 2776 | CUCUUCAA G UGAAUACU | 1598 | AGTATTCA GGCTAGCTACAACGA TTGAACAG | 4402 |
| 2806 | CAAAGGAA G CCAACUCU | 1599 | AGAGTTGG GGCTAGCTACAACGA TTCCTTTG | 4403 |
| 2821 | CUGAGGAA G UCUUUUUG | 1600 | CAAAAAGA GGCTAGCTACAACGA TTCCTCAG | 4404 |
| 2861 | UGAAAAUG G CACAGAUC | 1601 | GATCTGTG GGCTAGCTACAACGA CATTTTCA | 4405 |
| 2887 | CUAUUCAG G CUGUUGAU | 1602 | ATCAACAG GGCTAGCTACAACGA CTGAATAG | 4406 |
| 2899 | UUGAUAAC G UCGAUCUG | 1603 | CAGATCGA GGCTAGCTACAACGA CTTATCAA | 4407 |
| 2935 | UUGCACGA G UAUCUUUG | 1604 | CAAAGATA GGCTAGCTACAACGA TCGTGCAA | 4408 |
| 2978 | GACACCUA G UCCUGAUG | 1605 | CATCAGGA GGCTAGCTACAACGA TAGGTGTC | 4409 |
| 2991 | GAUGAAAC G UCUGCUCC | 1606 | GGAGCAGA GGCTAGCTACAACGA GTTTCATC | 4410 |
| 3023 | UAUCAACA G CACCAUUC | 1607 | GAATGGTG GGCTAGCTACAACGA TGTTGATA | 4411 |
| 3035 | CAUUCCUG G CAUUCACA | 1608 | TGTGAATG GGCTAGCTACAACGA CAGGAATG | 4412 |
| 3063 | AUGUGGAA G UGGAUAGG | 1609 | CCTATCCA GGCTAGCTACAACGA TTCCACAT | 4413 |
| 3081 | GAACUGCA G CUGUCAAU | 1610 | ATTGACAG GGCTAGCTACAACGA TGCAGTTC | 4414 |
| 3091 | UGUCAAUA G CCUAGGGC | 1611 | GCCCTAGG GGCTAGCTACAACGA TATTGACA | 4415 |
| 3098 | AGCCUAGG G CUGAAUUU | 1612 | AUATTCAG GGCTAGCTACAACGA CCTAGGCT | 4416 |
| 3189 | UGUAGGGG G CGAUAUAC | 1613 | GTATATCG GGCTAGCTACAACGA CCCCTACA | 4417 |
| 3242 | UGUAGGGG G CGAUAUAC | 1613 | GTATATCG GGCTAGCTACAACGA CCCCTACA | 4417 |
| 3210 | UGUAUAUA G UACAUUUA | 1614 | TAAATGTA GGCTAGCTACAACGA TATATACA | 4418 |
| 3279 | UGUAGGGG G CGAUAAAA | 1615 | TTTTATCG GGCTAGCTACAACGA CCCCTACA | 4419 |
| 21 | UGGUACAA A UGGAUGUG | 1616 | CACATCCA GGCTAGCTACAACGA TTGTACCA | 4420 |
| 25 | ACAAAUGG A UGUGGAAU | 1617 | ATTCCACA GGCTAGCTACAACGA CCATTTGT | 4421 |
| 32 | GAUGUGGA A UAUAAUUG | 1618 | CAATTATA GGCTAGCTACAACGA TCCACATC | 4422 |
| 37 | GGAAUAUA A UUGAAUAU | 1619 | ATATTCAA GGCTAGCTACAACGA TATATTCC | 4423 |
| 42 | AUAAUUGA A UAUUUUCU | 1620 | AGAAAATA GGCTAGCTACAACGA TCAATTAT | 4424 |
| 114 | GAAGGCAG A UGGAAAUA | 1621 | TATTTCCA GGCTAGCTACAACGA CTGCCTTC | 4425 |
| 120 | AGAUGGAA A UAUUUACA | 1622 | TGTAAATA GGCTAGCTACAACGA TTCCATCT | 4426 |
| 137 | AGUACGCA A UUUGAGAC | 1623 | GTCTCAAA GGCTAGCTACAACGA TGCGTACT | 4427 |
| 144 | AAUUUGAG A CUAAGAUA | 1624 | TATCTTAG GGCTAGCTACAACGA CTCAAATT | 4428 |
| 150 | AGACUAAG A UAUUGUUA | 1625 | TAACAATA GGCTAGCTACAACGA CTTAGTCT | 4429 |
| 176 | UAUUGAAG A CAAGAGCA | 1626 | TGCTCTTG GGCTAGCTACAACGA CTTCAATA | 4430 |
| 185 | CAAGAGCA A UAGUAAAA | 1627 | TTTTACTA GGCTAGCTACAACGA TGCTCTTG | 4431 |
| 193 | AUAGUAAA A CACAUCAG | 1628 | CTGATGTG GGCTAGCTACAACGA TTTACTAT | 4432 |
| 217 | GGUUAAAG A CCUGUGAU | 1629 | ATCACAGG GGCTAGCTACAACGA CTTTAACC | 4433 |
| 224 | GACCUGUG A UAAACCAC | 1630 | GTGGTTTA GGCTAGCTACAACGA CACAGGTC | 4434 |
| 228 | UGUGAUAA A CCACUUCC | 1631 | GGAAGTGG GGCTAGCTACAACGA TTATCACA | 4435 |
| 238 | CACUUCCG A UAAGUUGG | 1632 | CCAACTTA GGCTAGCTACAACGA CGGAAGTG | 4436 |
| 249 | AGUUGGAA A CGUGUGUC | 1633 | GACACACG GGCTAGCTACAACGA TTCCAACT | 4437 |
| 284 | UAUAUAUA A UGGUAAAG | 1634 | CTTTACCA GGCTAGCTACAACGA TATATATA | 4438 |
| 297 | AAAGAAAG A CACCUUCG | 1635 | CGAAGGTG GGCTAGCTACAACGA CTTTCTTT | 4439 |
| 308 | CCUUCGUA A CCCGCAUU | 1636 | AATGCGGG GGCTAGCTACAACGA TACGAAGG | 4440 |
| 331 | AGAGAGGA A UCACAGGG | 1637 | CCCTGTGA GGCTAGCTACAACGA TCCTCTCT | 4441 |
| 342 | ACAGGGAG A UGUACAGC | 1638 | GCTGTACA GGCTAGCTACAACGA CTCCCTGT | 4442 |
| 352 | GUACAGCA A UGGGGCCA | 1639 | TGGCCCCA GGCTAGCTACAACGA TGCTGTAC | 4443 |
| 385 | UCAUCUUG A UUCUUCAC | 1640 | GTGAAGAA GGCTAGCTACAACGA CAAGATGA | 4444 |
| 416 | CCUGAGUA A UUCACUCA | 1641 | TGAGTGAA GGCTAGCTACAACGA TACTCAGG | 4445 |
| 434 | UCAGCUGA A CAACAAUG | 1642 | CATTGTTG GGCTAGCTACAACGA TCAGCTGA | 4446 |
| 437 | GCUGAACA A CAAUGGCU | 1643 | AGCCATTG GGCTAGCTACAACGA TGTTCAGC | 4447 |
| 440 | GAACAACA A UGGCUAUG | 1644 | CATAGCCA GGCTAGCTACAACGA TGTTGTTC | 4448 |
| 466 | UCGUUGCA A UCGACCCC | 1645 | GGGGTCGA GGCTAGCTACAACGA TGCAACGA | 4449 |
| 470 | UGCAAUCG A CCCCAAUG | 1646 | CATTGGGG GGCTAGCTACAACGA CGATTGCA | 4450 |
| 476 | CGACCCCA A UGUGCCAG | 1647 | CTGGCACA GGCTAGCTACAACGA TGGGGTCG | 4451 |
| 488 | GCCAGAAG A UGAAACAC | 1648 | GTGTTTCA GGCTAGCTACAACGA CTTCTGGC | 4452 |
| 493 | AAGAUGAA A CACUCAUU | 1649 | AATGAGTG GGCTAGCTACAACGA TTCATCTT | 4453 |
| 504 | CUCAUUCA A CAAAUAAA | 1650 | TTTATTTG GGCTAGCTACAACGA TGAATGAG | 4454 |
| 508 | UUCAACAA A UAAAGGAC | 1651 | GTCCTTTA GGCTAGCTACAACGA TTGTTGAA | 4455 |
| 515 | AAUAAAGG A CAUGGUGA | 1652 | TCACCATG GGCTAGCTACAACGA CCTTTATT | 4456 |
| 523 | ACAUGGUG A CCCAGGCA | 1653 | TGCCTGGG GGCTAGCTACAACGA CACCATGT | 4457 |
| 564 | GGAAAGCG A UUUUAUUU | 1654 | AAATAAAA GGCTAGCTACAACGA CGCTTTCC | 4458 |
| 578 | UUUCAAAA A UGUUGCCA | 1655 | TGGCAACA GGCTAGCTACAACGA TTTTGAAA | 4459 |
| 592 | CCAUUUUG A UUCCUGAA | 1656 | TTCAGGAA GGCTAGCTACAACGA CAAAATGG | 4460 |
| 601 | UUCCUGAA A CAUGGAAG | 1657 | CTTCCATG GGCTAGCTACAACGA TTCAGGAA | 4461 |
| 610 | CAUGGAAG A CAAAGGCU | 1658 | AGCCTTTG GGCTAGCTACAACGA CTTCCATG | 4462 |
| 620 | AAAGGCUG A CUAUGUGA | 1659 | TCACATAG GGCTAGCTACAACGA CAGCCTTT | 4463 |
| 630 | UAUGUGAG A CCAAAACU | 1660 | AGTTTTGG GGCTAGCTACAACGA CTCACATA | 4464 |
| 636 | AGACCAAA A CUUGAGAC | 1661 | GTCTCAAG GGCTAGCTACAACGA TTTGGTCT | 4465 |
| 643 | AACUUGAG A CCUACAAA | 1662 | TTTGTAGG GGCTAGCTACAACGA CTCAAGTT | 4466 |
| 653 | CUACAAAA A UGCUGAUG | 1663 | CATCAGCA GGCTAGCTACAACGA TTTTGTAG | 4467 |
| 659 | AAAUGCUG A UGUUCUGG | 1664 | CCAGAACA GGCTAGCTACAACGA CAGCATTT | 4468 |
| 692 | UCCAGGUA A UGAUGAAC | 1665 | GTTCATCA GGCTAGCTACAACGA TACCTGGA | 4469 |
| 695 | AGGUAAUG A UGAACCCU | 1666 | AGGGTTCA GGCTAGCTACAACGA CATTACCT | 4470 |
| 699 | AAUGAUGA A CCCUACAC | 1667 | GTGTAGGG GGCTAGCTACAACGA TCATCATT | 4471 |
| 715 | CUGAGCAG A UGGGCAAC | 1668 | GTTGCCCA GGCTAGCTACAACGA CTGCTCAG | 4472 |
| 722 | GAUGGGCA A CUGUGGAG | 1669 | CTCCACAG GGCTAGCTACAACGA TGCCCATC | 4473 |
| 745 | GUGAAAGG A UCCACCUC | 1670 | GAGGTGGA GGCTAGCTACAACGA CCTTTCAC | 4474 |
| 761 | CACUCCUG A UUUCAUUG | 1671 | CAATGAAA GGCTAGCTACAACGA CAGGAGTG | 4475 |
| 789 | UUAGCUGA A UAUGGACC | 1672 | GGTCCATA GGCTAGCTACAACGA TCAGCTAA | 4476 |
| 795 | GAAUAUGG A CCACAAGG | 1673 | CCTTGTGG GGCTAGCTACAACGA CCATATTC | 4477 |
| 837 | CAUCUACG A UGGGGAGU | 1674 | ACTCCCCA GGCTAGCTACAACGA CGTAGATG | 4478 |
| 851 | AGUAUUUG A CGAGUACA | 1675 | TGTACTCG GGCTAGCTACAACGA CAAATACT | 4479 |
| 860 | CGAGUACA A UAAUGAUG | 1676 | CATCATTA GGCTAGCTACAACGA TGTACTCG | 4480 |
| 863 | GUACAAUA A UGAUGAGA | 1677 | TCTCATCA GGCTAGCTACAACGA TATTGTAC | 4481 |
| 866 | CAAUAAUG A UGAGAAAU | 1678 | ATTTCTCA GGCTAGCTACAACGA CATTATTG | 4482 |
| 873 | GAUGAGAA A UUCUACUU | 1679 | AAGTAGAA GGCTAGCTACAACGA TTCTCATC | 4483 |
| 887 | CUUAUCCA A UGGAAGAA | 1680 | TTCTTCCA GGCTAGCTACAACGA TGGATAAG | 4484 |
| 895 | AUGGAAGA A UACAAGCA | 1681 | TGCTTGTA GGCTAGCTACAACGA TCTTCCAT | 4485 |
| 909 | GCAGUAAG A UGUUCAGC | 1682 | GCTGAACA GGCTAGCTACAACGA CTTACTGC | 4486 |
| 935 | UCGUACAA A UGUAGUAA | 1683 | TTACTACA GGCTAGCTACAACGA TTGTACCA | 4487 |
| 978 | ACCAAAAG A UGCACAUU | 1684 | AATGTGCA GGCTAGCTACAACGA CTTTTGGT | 4488 |
| 989 | CACAUUCA A UAAAGUUA | 1685 | TAACTTTA GGCTAGCTACAACGA TGAATGTG | 4489 |
| 1002 | GUUACAGG A CUCUAUGA | 1686 | TCATAGAG GGCTAGCTACAACGA CCTGTAAC | 4490 |
| 1017 | GAAAAAGG A UGUGAGUU | 1687 | AACTCACA GGCTAGCTACAACGA CCTTTTTC | 4491 |
| 1035 | GUUCUCCA A UCCCGCCA | 1688 | TGGCGGGA GGCTAGCTACAACGA TGGAGAAC | 4492 |
| 1045 | CCCGCCAG A CGGAGAAG | 1689 | CTTCTCCG GGCTAGCTACAACGA CTGGCGGG | 4493 |
| 1063 | CUUCUAUA A UGUUUGCA | 1690 | TGCAAACA GGCTAGCTACAACGA TATAGAAG | 4494 |
| 1074 | UUUGCACA A CAUGUUGA | 1691 | TCAACATG GGCTAGCTACAACGA TGTGCAAA | 4495 |
| 1082 | ACAUGUUG A UUCUAUAG | 1692 | CTATAGAA GGCTAGCTACAACGA CAACATGT | 4496 |
| 1095 | AUAGUUGA A UUCUGUAC | 1693 | GTACAGAA GGCTAGCTACAACGA TCAACTAT | 4497 |
| 1107 | UGUACAGA A CAAAACCA | 1694 | TGGTTTTG GGCTAGCTACAACGA TCTGTACA | 4498 |
| 1112 | AGAACAAA A CCACAACA | 1695 | TCTTGTGG GGCTAGCTACAACGA TTTGTTCT | 4499 |
| 1118 | AAACCACA A CAAAGAAG | 1696 | CTTCTTTG GGCTAGCTACAACGA TGTGGTTT | 4500 |
| 1133 | AGCUCCAA A CAAGCAAA | 1697 | TTTGCTTG GGCTAGCTACAACGA TTGGAGCT | 4501 |
| 1142 | CAAGCAAA A UCAAAAAU | 1698 | ATTTTTGA GGCTAGCTACAACGA TTTGCTTG | 4502 |
| 1149 | AAUCAAAA A UGCAAUCU | 1699 | AGATTGCA GGCTAGCTACAACGA TTTTGATT | 4503 |
| 1154 | AAAAUGCA A UCUCCGAA | 1700 | TTCGGAGA GGCTAGCTACAACGA TGCATTTT | 4504 |
| 1177 | GGGAAGUG A UCCGUGAU | 1701 | ATCACGGA GGCTAGCTACAACGA CACTTCCC | 4505 |
| 1184 | GAUCCGUG A UUCUGAGG | 1702 | CCTCAGAA GGCTAGCTACAACGA CACGGATC | 4506 |
| 1193 | UUCUGAGG A CUUUAAGA | 1703 | TCTTAAAG GGCTAGCTACAACGA CCTCAGAA | 4507 |
| 1204 | UUAAGAAA A CCACUCCU | 1704 | AGGAGTGG GGCTAGCTACAACGA TTTCTTAA | 4508 |
| 1216 | CUCCUAUG A CAACACAG | 1705 | CTGTGTTG GGCTAGCTACAACGA CATAGGAG | 4509 |
| 1219 | CUAUGACA A CACAGCCA | 1706 | TGGCTGTG GGCTAGCTACAACGA TGTCATAG | 4510 |
| 1232 | GCCACCAA A UCCCACCU | 1707 | AGGTGGGA GGCTAGCTACAACGA TTGGTGGC | 4511 |
| 1255 | UGCUGCAG A UUGGACAA | 1708 | TTGTCCAA GGCTAGCTACAACGA CTGCAGCA | 4512 |
| 1260 | CAGAUUGG A CAAAGAAU | 1709 | ATTCTTTG GGCTAGCTACAACGA CCAATCTG | 4513 |
| 1267 | GACAAAGA A UUGUGUGU | 1710 | ACACACAA GGCTAGCTACAACGA TCTTTGTC | 4514 |
| 1286 | AGUCCUUG A CAAAUCUG | 1711 | CAGATTTG GGCTAGCTACAACGA CAAGGACT | 4515 |
| 1290 | CUUGACAA A UCUGGAAG | 1712 | CTTCCAGA GGCTAGCTACAACGA TTGTCAAG | 4516 |
| 1306 | GCAUGGCG A CUGGUAAC | 1713 | GTTACCAG GGCTAGCTACAACGA CGCCATGC | 4517 |
| 1313 | GACUGGUA A CCGCCUCA | 1714 | TGAGGCGG GGCTAGCTACAACGA TACCAGTC | 4518 |
| 1322 | CCGCCUCA A UCGACUGA | 1715 | TCAGTCGA GGCTAGCTACAACGA TGAGGCGG | 4519 |
| 1326 | CUCAAUCG A CUGAAUCA | 1716 | TGATTCAG GGCTAGCTACAACGA CGATTGAG | 4520 |
| 1331 | UCGACUGA A UCAAGCAG | 1717 | CTGCTTGA GGCTAGCTACAACGA TCAGTCGA | 4521 |
| 1360 | UGCUGCAG A CAGUUGAG | 1718 | CTCAACTG GGCTAGCTACAACGA CTGCAGCA | 4522 |
| 1387 | GGGUUGGG A UGGUGACA | 1719 | TGTCACCA GGCTAGCTACAACGA CCCAACCC | 4523 |
| 1393 | GGAUGGUG A CAUUUGAC | 1720 | GTCAAATG GGCTAGCTACAACGA CACCATCC | 4524 |
| 1400 | GACAUUUG A CAGUGCUG | 1721 | CAGCACTG GGCTAGCTACAACGA CAAATGTC | 4525 |
| 1425 | CAAAGUGA A CUCAUACA | 1722 | TGTATGAG GGCTAGCTACAACGA TCACTTTG | 4526 |
| 1435 | UCAUACAG A UAAACAGU | 1723 | ACTGTTTA GGCTAGCTACAACGA CTGTATGA | 4527 |
| 1439 | ACAGAUAA A CAGUGGCA | 1724 | TGCCACTG GGCTAGCTACAACGA TTATCTGT | 4528 |
| 1451 | UGGCAGUG A CAGGGACA | 1725 | TGTCCCTG GGCTAGCTACAACGA CACTGCCA | 4529 |
| 1457 | UGACAGGG A CACACUCG | 1726 | CGAGTGTG GGCTAGCTACAACGA CCCTGTCA | 4530 |
| 1473 | GCCAAAAG A UUACCUGC | 1727 | GCAGGTAA GGCTAGCTACAACGA CTTTTGGC | 4531 |
| 1498 | CAGGAGGG A CGUCCAUC | 1728 | GATGGACG GGCTAGCTACAACGA CCCTCCTG | 4532 |
| 1521 | GGGCUUCG A UCGGCAUU | 1729 | AATGCCGA GGCTAGCTACAACGA CGAAGCCC | 4533 |
| 1537 | UUACUGUG A UUAGGAAG | 1730 | CTTCCTAA GGCTAGCTACAACGA CACAGTAA | 4534 |
| 1548 | AGGAAGAA A UAUCCAAC | 1731 | GTTGGATA GGCTAGCTACAACGA TTCTTCCT | 4535 |
| 1555 | AAUAUCCA A CUGAUGGA | 1732 | TCCATCAG GGCTAGCTACAACGA TGGATATT | 4536 |
| 1559 | UCCAACUG A UGGAUCUG | 1733 | CAGATCCA GGCTAGCTACAACGA CAGTTGGA | 4537 |
| 1563 | ACUGAUGG A UCUGAAAU | 1734 | ATTTCAGA GGCTAGCTACAACGA CCATCAGT | 4538 |
| 1570 | GAUCUGAA A UUGUGCUG | 1735 | CAGCACAA GGCTAGCTACAACGA TTCAGATC | 4539 |
| 1582 | UGCUGCUG A CGGAUGGG | 1736 | CCCATCCG GGCTAGCTACAACGA CAGCAGCA | 4540 |
| 1586 | GCUGACGG A UGGGGAAG | 1737 | CTTCCCCA GGCTAGCTACAACGA CCGTCAGC | 4541 |
| 1595 | UGGGGAAG A CAACACUA | 1738 | TAGTGTTG GGCTAGCTACAACGA CTTCCCCA | 4542 |
| 1598 | GGAAGACA A CACUAUAA | 1739 | TTATAGTG GGCTAGCTACAACGA TGTCTTCC | 4543 |
| 1619 | GUGCUUUA A CGAGGUCA | 1740 | TGACCTCG GGCTAGCTACAACGA TAAAGCAC | 4544 |
| 1629 | GAGGUCAA A CAAAGUGG | 1741 | CCACTTTG GGCTAGCTACAACGA TTGACCTC | 4545 |
| 1683 | GCUCAAGA A CUAGAGGA | 1742 | TCCTCTAG GGCTAGCTACAACGA TCTTGAGC | 4546 |
| 1702 | UGUCCAAA A UGACAGGA | 1743 | TCCTGTCA GGCTAGCTACAACGA TTTGGACA | 4547 |
| 1705 | CCAAAAUG A CAGGAGGU | 1744 | ACCTCCTG GGCTAGCTACAACGA CATTTTGG | 4548 |
| 1720 | GUUUACAG A CAUAUGCU | 1745 | AGCATATG GGCTAGCTACAACGA CTGTAAAC | 4549 |
| 1733 | UGCUUCAG A UCAAGUUC | 1746 | GAACTTGA GGCTAGCTACAACGA CTGAAGCA | 4550 |
| 1745 | AGUUCAGA A CAAUGGCC | 1747 | GGCCATTG GGCTAGCTACAACGA TCTGAACT | 4551 |
| 1748 | UCAGAACA A UGGCCUCA | 1748 | TGAGGCCA GGCTAGCTACAACGA TGTTCTGA | 4552 |
| 1760 | CCUCAUUG A UGCUUUUG | 1749 | CAAAAGCA GGCTAGCTACAACGA CAATGAGG | 4553 |
| 1787 | AUCAGGAA A UGGAGCUG | 1750 | CAGCTCCA GGCTAGCTACAACGA TTCCTGAT | 4554 |
| 1830 | AGUAAGGG A UUAACCCU | 1751 | AGGGTTAA GGCTAGCTACAACGA CCCTTACT | 4555 |
| 1834 | AGGGAUUA A CCCUCCAG | 1752 | CTGGAGGG GGCTAGCTACAACGA TAATCCCT | 4556 |
| 1844 | CCUCCAGA A CAGCCAGU | 1753 | ACTGGCTG GGCTAGCTACAACGA TCTGGAGG | 4557 |
| 1855 | GCCAGUGG A UGAAUGGC | 1754 | GCCATTCA GGCTAGCTACAACGA CCACTGGC | 4558 |
| 1859 | GUGGAUGA A UGGCACAG | 1755 | CTGTGCCA GGCTAGCTACAACGA TCATCCAC | 4559 |
| 1870 | GCACAGUG A UCGUGGAC | 1756 | GTCCACGA GGCTAGCTACAACGA CACTGTGC | 4560 |
| 1877 | GAUCGUGG A CAGCACCG | 1757 | CGGTGCTG GGCTAGCTACAACGA CCACGATC | 4561 |
| 1895 | GGGAAAGG A CACUUUGU | 1758 | ACAAAGTG GGCTAGCTACAACGA CCTTTCCC | 4562 |
| 1918 | UCACCUGG A CAACGCAG | 1759 | CTGCGTTG GGCTAGCTACAACGA CCAGGTGA | 4563 |
| 1921 | CCUGGACA A CGCAGCCU | 1760 | AGGCTGCG GGCTAGCTACAACGA TGTCCAGG | 4564 |
| 1936 | CUCCCCAA A UCCUUCUC | 1761 | GAGAAGGA GGCTAGCTACAACGA TTGGGGAG | 4565 |
| 1949 | UCUCUGGG A UCCCAGUG | 1762 | CACTGGGA GGCTAGCTACAACGA CCCAGAGA | 4566 |
| 1959 | CCCAGUGG A CAGAAGCA | 1763 | TGCTTCTG GGCTAGCTACAACGA CCACTGGG | 4567 |
| 1985 | UGUAGUGG A CAAAAACA | 1764 | TGTTTTTG GGCTAGCTACAACGA CCACTACA | 4568 |
| 1991 | GGACAAAA A CACCAAAA | 1765 | TTTTGGTG GGCTAGCTACAACGA TTTTGTCC | 4569 |
| 1999 | ACACCAAA A UGGCCUAC | 1766 | GTAGGCCA GGCTAGCTACAACGA TTTGGTGT | 4570 |
| 2014 | ACCUCCAA A UCCCAGGC | 1767 | GCCTGGGA GGCTAGCTACAACGA TTGGAGGT | 4571 |
| 2046 | ACUUGGAA A UACAGUCU | 1768 | AGACTGTA GGCTAGCTACAACGA TTCCAAGT | 4572 |
| 2071 | GCUCACAA A CCUUGACC | 1769 | GGTCAAGG GGCTAGCTACAACGA TTGTGAGC | 4573 |
| 2077 | AAACCUUG A CCCUGACU | 1770 | AGTCAGGG GGCTAGCTACAACGA CAAGGTTT | 4574 |
| 2083 | UGACCCUG A CUGUCACG | 1771 | CGTGACAG GGCTAGCTACAACGA CAGGGTCA | 4575 |
| 2105 | UGCGUCCA A UGCUACCC | 1772 | GGGTAGCA GGCTAGCTACAACGA TGGACGCA | 4576 |
| 2122 | UGCCUCCA A UUACAGUG | 1773 | CACTGTAA GGCTAGCTACAACGA TGGAGGCA | 4577 |
| 2131 | UUACAGUG A CUUCCAAA | 1774 | TTTGGAAG GGCTAGCTACAACGA CACTGTAA | 4578 |
| 2140 | CUUCCAAA A CGAACAAG | 1775 | CTTGTTCG GGCTAGCTACAACGA TTTGGAAG | 4579 |
| 2144 | CAAAACGA A CAAGGACA | 1776 | TGTCCTTG GGCTAGCTACAACGA TCGTTTTG | 4580 |
| 2150 | GAACAAGG A CACCAGCA | 1777 | TGCTGGTG GGCTAGCTACAACGA CCTTGTTC | 4581 |
| 2160 | ACCAGCAA A UUCCCCAG | 1778 | CTGGGGAA GGCTAGCTACAACGA TTGCTGGT | 4582 |
| 2189 | UUAUGCAA A UAUUCGCC | 1779 | GGCGAATA GGCTAGCTACAACGA TTGCATAA | 4583 |
| 2212 | CCUCCCCA A UUCUCAGG | 1780 | CCTGAGAA GGCTAGCTACAACGA TGGGGAGG | 4584 |
| 2239 | CAGCCCUG A UUGAAUCA | 1781 | TGATTCAA GGCTAGCTACAACGA CAGGGCTG | 4585 |
| 2244 | CUGAUUGA A UCAGUGAA | 1782 | TTCACTGA GGCTAGCTACAACGA TCAATCAG | 4586 |
| 2252 | AUCAGUGA A UGGAAAAA | 1783 | TTTTTCCA GGCTAGCTACAACGA TCACTGAT | 4587 |
| 2260 | AUGGAAAA A CAGUUACC | 1784 | GGTAACTG GGCTAGCTACAACGA TTTTCCAT | 4588 |
| 2274 | ACCUUGGA A CUACUGGA | 1785 | TCCAGTAG GGCTAGCTACAACGA TCCAAGGT | 4589 |
| 2282 | ACUACUGG A UAAUGGAG | 1786 | CTCCATTA GGCTAGCTACAACGA CCAGTAGT | 4590 |
| 2285 | ACUGGAUA A UGGAGCAG | 1787 | CTGCTCCA GGCTAGCTACAACGA TATCCAGT | 4591 |
| 2300 | AGGUGCUG A UGCUACUA | 1788 | TAGTAGCA GGCTAGCTACAACGA CAGCACCT | 4592 |
| 2312 | UACUAAGG A UGACGGUG | 1789 | CACCGTCA GGCTAGCTACAACGA CCTTAGTA | 4593 |
| 2315 | UAAGGAUG A CGGUGUCU | 1790 | AGACACCG GGCTAGCTACAACGA CATCCTTA | 4594 |
| 2341 | AUUUCACA A CUUAUGAC | 1791 | GTCATAAG GGCTAGCTACAACGA TGTGAAAT | 4595 |
| 2348 | AACUUAUG A CACGAAUG | 1792 | CATTCGTG GGCTAGCTACAACGA CATAAGTT | 4596 |
| 2354 | UGACACCA A UGGUAGAU | 1793 | ATCTACCA GGCTAGCTACAACGA TCGTGTCA | 4597 |
| 2361 | AAUGGUAG A UACAGUGU | 1794 | ACACTGTA GGCTAGCTACAACGA CTACCATT | 4598 |
| 2396 | AGGAGUUA A CGCAGCCA | 1795 | TGGCTGCG GGCTAGCTACAACGA TAACTCCT | 4599 |
| 2406 | GCAGCCAG A CGGAGAGU | 1796 | ACTCTCCG GGCTAGCTACAACGA CTGGCTGC | 4600 |
| 2416 | GGAGAGUG A UACCCCAG | 1797 | CTGGGGTA GGCTAGCTACAACGA CACTCTCC | 4601 |
| 2455 | CUGGCUGG A UUGAGAAU | 1798 | ATTCTCAA GGCTAGCTACAACGA CCAGCCAG | 4602 |
| 2462 | GAUUGAGA A UGAUGAAA | 1799 | TTTCATCA GGCTAGCTACAACGA TCTCAATC | 4603 |
| 2465 | UGAGAAUG A UGAAAUAC | 1800 | GTATTTCA GGCTAGCTACAACGA CATTCTCA | 4604 |
| 2470 | AUGAUGA A AUCAAUGG | 1801 | CCATTGTA GGCTAGCTACAACGA TTCATCAT | 4605 |
| 2475 | GAAAUACA A UGGAAUCC | 1802 | GGATTCCA GGCTAGCTACAACGA TGTATTTC | 4606 |
| 2480 | ACAAUGGA A UCCACCAA | 1803 | TTGGTGGA GGCTAGCTACAACGA TCCATTGT | 4607 |
| 2490 | CCACCAAG A CCUGAAAU | 1804 | ATTTCAGG GGCTAGCTACAACGA CTTGGTGG | 4608 |
| 2497 | GACCUGAA A UUAAUAAG | 1805 | CTTATTAA GGCTAGCTACAACGA TTCAGGTC | 4609 |
| 2501 | UGAAAUUA A UAAGGAUG | 1806 | CATCCTTA GGCTAGCTACAACGA TAATTTCA | 4610 |
| 2507 | UAAUAAGG A UGAUGUUC | 1807 | GAACATCA GGCTAGCTACAACGA CCTTATTA | 4611 |
| 2510 | UAAGGAUG A UGUUCAAC | 1808 | GTTGAACA GGCTAGCTACAACGA CATCCTTA | 4612 |
| 2517 | GAUGUUCA A CACAAGCA | 1809 | TGCTTGTG GGCTAGCTACAACGA TGAACATC | 4613 |
| 2542 | UCAGCAGA A CAUCCUCG | 1810 | CGAGGATG GGCTAGCTACAACGA TCTGCTGA | 4614 |
| 2573 | GGCUUCUG A UGUCCCAA | 1811 | TTGGGACA GGCTAGCTACAACGA CAGAAGCC | 4615 |
| 2582 | UGUCCCAA A UGCUCCCA | 1812 | TGGGAGCA GGCTAGCTACAACGA TTGGGACA | 4616 |
| 2597 | CAUACCUG A UCUCUUCC | 1813 | GGAAGAGA GGCTAGCTACAACGA CAGGTATG | 4617 |
| 2617 | CUGGCCAA A UCACCGAC | 1814 | GTCGGTGA GGCTAGCTACAACGA TTGGCCAG | 4618 |
| 2624 | AAUCACCG A CCUGAAGG | 1815 | CCTTCAGG GGCTAGCTACAACGA CGGTGATT | 4619 |
| 2638 | AGGCGGAA A UUCACGGG | 1816 | CCCGTGAA GGCTAGCTACAACGA TTCCGCCT | 4620 |
| 2660 | UCUCAUUA A UCUGACUU | 1817 | AAGTCAGA GGCTAGCTACAACGA TAATGAGA | 4621 |
| 2665 | UUAAUCUG A CUUGGACA | 1818 | TGTCCAAG GGCTAGCTACAACGA CAGATTAA | 4622 |
| 2671 | UGACUUGG A CAGCUCCU | 1819 | AGGAGCTG GGCTAGCTACAACGA CCAAGTCA | 4623 |
| 2684 | UCCUGGGG A UGAUUAUG | 1820 | CATAATCA GGCTAGCTACAACGA CCCCAGGA | 4624 |
| 2687 | UGGGGAUG A UUAUGACC | 1821 | GGTCATAA GGCTAGCTACAACGA CATCCCCA | 4625 |
| 2693 | UGAUUAUG A CCAUGGAA | 1822 | TTCCATGG GGCTAGCTACAACGA CATAATCA | 4626 |
| 2701 | ACCAUGGA A CAGCUCAC | 1823 | GTGAGCTG GGCTAGCTACAACGA TCCATGGT | 4627 |
| 2725 | UCAUUCGA A UAAGUACA | 1824 | TGTACTTA GGCTAGCTACAACGA TCGAATGA | 4628 |
| 2744 | UAUUCUUG A UCUCAGAG | 1825 | CTCTGAGA GGCTAGCTACAACGA CAAGAATA | 4629 |
| 2753 | UCUCAGAG A CAAGUUCA | 1826 | TGAACTTG GGCTAGCTACAACGA CTCTGAGA | 4630 |
| 2762 | CAAGUUCA A UGAAUCUC | 1827 | GAGATTCA GGCTAGCTACAACGA TGAACTTG | 4631 |
| 2766 | UUCAAUGA A UCUCUUCA | 1828 | TGAAGAGA GGCTAGCTACAACGA TCATTGAA | 4632 |
| 2780 | UCAAGUGA A UACUACUG | 1829 | CAGTAGTA GGCTAGCTACAACGA TCACTTGA | 4633 |
| 2810 | GGAAGCCA A CUCUGAGG | 1830 | CCTCAGAG GGCTAGCTACAACGA TGGCTTCC | 4634 |
| 2835 | UUGUUUAA A CCAGAAAA | 1831 | TTTTCTGG GGCTAGCTACAACGA TTAAACAA | 4635 |
| 2843 | ACCAGAAA A CAUUACUU | 1832 | AAGTAATG GGCTAGCTACAACGA TTTCTGGT | 4636 |
| 2858 | UUUUGAAA A UGGCACAG | 1833 | CTGTGCCA GGCTAGCTACAACGA TTTCAAAA | 4637 |
| 2867 | UGGCACAG A UCUUUUCA | 1834 | TGAAAAGA GGCTAGCTACAACGA CTGTGCCA | 4638 |
| 2894 | GGCUGUUG A UAAGGUCG | 1835 | CGACCTTA GGCTAGCTACAACGA CAACAGCC | 4639 |
| 2903 | UAAGGUCG A UCUGAAAU | 1836 | ATTTCAGA GGCTAGCTACAACGA CGACCTTA | 4640 |
| 2910 | GAUCUGAA A UCAGAAAU | 1837 | ATTTCTGA GGCTAGCTACAACGA TTCAGATC | 4641 |
| 2917 | AAUCAGAA A UAUCCAAC | 1838 | GTTGGATA GGCTAGCTACAACGA TTCTGATT | 4642 |
| 2924 | AAUAUCCA A CAUUGCAC | 1839 | GTGCAATG GGCTAGCTACAACGA TGGATATT | 4643 |
| 2959 | CUCCACAG A CUCCGCCA | 1840 | TGGCGGAG GGCTAGCTACAACGA CTGTGGAG | 4644 |
| 2971 | CGCCAGAG A CACCUAGU | 1841 | ACTAGGTG GGCTAGCTACAACGA CTCTGGCG | 4645 |
| 2984 | UAGUCCUG A UGAAACGU | 1842 | ACGTTTCA GGCTAGCTACAACGA CAGGACTA | 4646 |
| 2989 | CUGAUGAA A CGUCUGCU | 1843 | AGCAGACG GGCTAGCTACAACGA TTCATCAG | 4647 |
| 3008 | UUGUCCUA A UAUUCAUA | 1844 | TATGAATA GGCTAGCTACAACGA TAGGACAA | 4648 |
| 3020 | UCAUAUCA A CAGCACCA | 1845 | TGGTGCTG GGCTAGCTACAACGA TGATATGA | 4649 |
| 3052 | UUUUAAAA A UUAUGUGG | 1846 | CCACATAA GGCTAGCTACAACGA TTTTAAAA | 4650 |
| 3067 | GGAAGUGG A UAGGAGAA | 1847 | TTCTCCTA GGCTAGCTACAACGA CCACTTCC | 4651 |
| 3075 | AUAGGAGA A CUGCAGCU | 1848 | AGCTGCAG GGCTAGCTACAACGA TCTCCTAT | 4652 |
| 3088 | AGCUGUCA A UAGCCUAG | 1849 | CTAGGCTA GGCTAGCTACAACGA TGACAGCT | 4653 |
| 3103 | AGGGCUGA A UUUUUGUC | 1850 | GACAAAAA GGCTAGCTACAACGA TCAGCCCT | 4654 |
| 3114 | UUUGUCAG A UAAAUAAA | 1851 | TTTATTTA GGCTAGCTACAACGA CTGACAAA | 4655 |
| 3118 | UCAGAUAA A UAAAAUAA | 1852 | TTATTTTA GGCTAGCTACAACGA TTATCTGA | 4656 |
| 3123 | UAAAUAAA A UAAAUCAU | 1853 | ATGATTTA GGCTAGCTACAACGA TTTATTTA | 4657 |
| 3127 | UAAAAUAA A UCAUUCAU | 1854 | ATGAATGA GGCTAGCTACAACGA TTATTTTA | 4658 |
| 3146 | UUUUUUUG A UUAUAAAA | 1855 | TTTTATAA GGCTAGCTACAACGA CAAAAAAA | 4659 |
| 3154 | AUUAUAAA A UUUUCUAA | 1856 | TTAGAAAA GGCTAGCTACAACGA TTTATAAT | 4660 |
| 3164 | UUUCUAAA A UGUAUUUU | 1857 | AAAATACA GGCTAGCTACAACGA TTTAGAAA | 4661 |
| 3175 | UAUUUUAG A CUUCCUGU | 1858 | ACAGGAAG GGCTAGCTACAACGA CTAAAATA | 4662 |
| 3265 | UAUUUUAG A CUUCCUGU | 1858 | ACAGGAAG GGCTAGCTACAACGA CTAAAATA | 4662 |
| 3192 | AGGGGGCG A UAUACUAA | 1859 | TTAGTATA GGCTAGCTACAACGA CGCCCCCT | 4663 |
| 3245 | AGGGGGCG A UAUACUAA | 1859 | TTAGTATA GGCTAGCTACAACGA CGCCCCCT | 4663 |
| 3201 | UAUACUAA A UGUAUAUA | 1860 | TATATACA GGCTAGCTACAACGA TTAGTATA | 4664 |
| 3225 | UAUACUAA A UGUAUUCC | 1861 | GGAATACA GCCTAGCTACAACGA TTAGTATA | 4665 |
| 3254 | UAUACUAA A UGUAUUUU | 1862 | AAAATACA GGCTAGCTACAACGA TTAGTATA | 4666 |
| 3282 | AGGGGGCG A UAAAAUAA | 1863 | TTATTTTA GGCTAGCTACAACGA CGCCCCCT | 4667 |
| 3287 | GCGAUAAA A UAAAAUGC | 1864 | GCATTTTA GGCTAGCTACAACGA TTTATCGC | 4668 |
| 3292 | AAAAUAAA A UGCUAAAC | 1865 | GTTTAGCA GGCTAGCTACAACGA TTTATTTT | 4669 |
| 3299 | AAUGCUAA A CAACUGGG | 1866 | CCCAGTTG GGCTAGCTACAACGA TTAGCATT | 4670 |
| 3302 | GCUAAACA A CUGGGUAA | 1867 | TTACCCAG GGCTAGCTACAACGA TGTTTAGC | 4671 |

Input Sequence = NM_001285. Cut Site = R/Y
Arm Length = 8. Core Sequence = GGCTAGCTACAACGA
NM_001285 (Homo sapiens chloride channel, calcium activated, 1 (CLCA1) mRNA, 3311 bp)

TABLE VIII

| | | Human CLCA1 Amberzyme and Target Sequence 249.021 | | |
|---|---|---|---|---|
| | | | | Rz |
| | | Seq | | Seq |
| | | ID | | ID |
| Pos | Substrate | No. | Amberzyme | No. |
| 40 | AUAUAAUU G AAUAUUUU | 1211 | AAAAUAUU GGA GCCGUUAGGC UCCCUUCAAGGA GCCGUUAGGC UCCGGG AAUUAUAU | 4672 |
| 67 | GGGAGCAU G AAGAGGUG | 1212 | CACCUCUU GGA GCCGUUAGGC UCCCUUCAAGGA GCCGUUAGGC UCCGGG AUGCUCCC | 4673 |
| 78 | GAGGUGUU G AGGUUAUG | 1213 | CAUAACCU GGA GCCGUUAGGC UCCCUUCAAGGA GCCGUUAGGC UCCGGG AACACCUC | 4674 |
| 106 | GCACAGCU G AAGGCAGA | 1214 | UCUGCCUU GGA GCCGUUAGGC UCCCUUCAAGGA GCCGUUAGGC UCCGGG AGCUGUGC | 4675 |
| 134 | ACAAGUAC G CAAUUUGA | 1215 | UCAAAUUG GGA GCCGUUAGGC UCCCUUCAAGGA GCCGUUAGGC UCCGGG GUACUUGU | 4676 |
| 141 | CGCAAUUU G AGACUAAG | 1216 | CUUAGUCU GGA GCCGUUAGGC UCCCUUCAAGGA GCCGUUAGGC UCCGGG AAAUUGCG | 4677 |
| 172 | CUCCUAUU G AAGACAAG | 1217 | CUUGUCUU GGA GCCGUUAGGC UCCCUUCAAGGA GCCGUUAGGC UCCGGG AAUAGGAG | 4678 |
| 223 | AGACCUGU G AUAAACCA | 1218 | UGGUUUAU GGA GCCGUUAGGC UCCCUUCAAGGA GCCGUUAGGC UCCGGG ACAGGUCU | 4679 |
| 237 | CCACUUCC G AUAAGUUG | 1219 | CAACUUAU GGA GCCGUUAGGC UCCCUUCAAGGA GCCGUUAGGC UCCGGG GGAAGUGG | 4680 |
| 312 | CGUAACCC G CAUUUUCC | 1220 | GGAAAAUG GGA GCCGUUAGGC UCCCUUCAAGGA GCCGUUAGGC UCCGGG GGGUUACG | 4681 |
| 384 | UUCAUCUU G AUUCUUCA | 1221 | UGAAGAAU GGA GCCGUUAGGC UCCCUUCAAGGA GCCGUUAGGC UCCGGG AAGAUGAA | 4682 |
| 411 | GGGGCCCU G AGUAAUUC | 1222 | GAAUUACU GGA GCCGUUAGGC UCCCUUCAAGGA GCCGUUAGGC UCCGGG AGGGCCCC | 4683 |
| 432 | AUUCAGCU G AACAACAA | 1223 | UUGUUGUU GGA GCCGUUAGGC UCCCUUCAAGGA GCCGUUAGGC UCCGGG AGCUGAAU | 4684 |
| 448 | AUGGCUAU G AAGGCAUU | 1224 | AAUGCCUU GGA GCCGUUAGGC UCCCUUCAAGGA GCCGUUAGGC UCCGGG AUAGCCAU | 4685 |
| 463 | UUGUCGUU G CAAUCGAC | 1225 | GUCGAUUG GGA GCCGUUAGGC UCCCUUCAAGGA GCCGUUAGGC UCCGGG AACGACAA | 4686 |
| 469 | UUGCAAUC G ACCCCAAU | 1226 | AUUGGGGU GGA GCCGUUAGGC UCCCUUCAAGGA GCCGUUAGGC UCCGGG GAUUGCAA | 4687 |
| 480 | CCCAAUGU G CCAGAAGA | 1227 | UCUUCUGG GGA GCCGUUAGGC UCCCUUCAAGGA GCCGUUAGGC UCCGGG ACAUUGGG | 4688 |
| 490 | CAGAAGAU G AAACACUC | 1228 | GAGUGUUU GGA GCCGUUAGGC UCCCUUCAAGGA GCCGUUAGGC UCCGGG AUCUUCUG | 4689 |
| 522 | GACAUGGU G ACCCAGGC | 1229 | GCCUGGGU GGA GCCGUUAGGC UCCCUUCAAGGA GCCGUUAGGC UCCGGG ACCAUGUC | 4690 |
| 547 | AUCUGUUU G AAGCUACA | 1230 | UGUAGCUU GGA GCCGUUAGGC UCCCUUCAAGGA GCCGUUAGGC UCCGGG AAACAGAU | 4691 |
| 563 | AGGAAAGC G AUUUUAUU | 1231 | AAUAAAAU GGA GCCGUUAGGC UCCCUUCAAGGA GCCGUUAGGC UCCGGG GCUUUCCU | 4692 |
| 583 | AAAAUGUU G CCAUUUUG | 1232 | CAAAAUGG GGA GCCGUUAGGC UCCCUUCAAGGA GCCGUUAGGC UCCGGG AACAUUUU | 4693 |
| 591 | GCCAUUUU G AUUCCUGA | 1233 | UCAGGAAU GGA GCCGUUAGGC UCCCUUCAAGGA GCCGUUAGGC UCCGGG AAAAUGGC | 4694 |
| 598 | UGAUUCCU G AAACAUGG | 1234 | CCAUGUUU GGA GCCGUUAGGC UCCCUUCAAGGA GCCGUUAGGC UCCGGG AGGAAUCA | 4695 |
| 619 | CAAAGGCU G ACUAUGUG | 1235 | CACAUAGU GGA GCCGUUAGGC UCCCUUCAAGGA GCCGUUAGGC UCCGGG AGCCUUUG | 4696 |
| 627 | GACUAUGU G AGACCAAA | 1236 | UUUGGUCU GGA GCCGUUAGGC UCCCUUCAAGGA GCCGUUAGGC UCCGGG ACAUAGUC | 4697 |
| 640 | CAAAACUU G AGACCUAC | 1237 | GUAGGUCU GGA GCCGUUAGGC UCCCUUCAAGGA GCCGUUAGGC UCCGGG AAGUUUUG | 4698 |
| 655 | ACAAAAAU G CUGAUGUU | 1238 | AACAUCAG GGA GCCGUUAGGC UCCCUUCAAGGA GCCGUUAGGC UCCGGG AUUUUUGU | 4699 |
| 658 | AAAAUGCU G AUGUUCUG | 1239 | CAGAACAU GGA GCCGUUAGGC UCCCUUCAAGGA GCCGUUAGGC UCCGGG AGCAUUUU | 4700 |
| 670 | UUCUGGUU G CUGAGUCU | 1240 | AGACUCAG GGA GCCGUUAGGC UCCCUUCAAGGA GCCGUUAGGC UCCGGG AACCAGAA | 4701 |
| 673 | UGGUUGCU G AGUCUACU | 1241 | AGUAGACU GGA GCCGUUAGGC UCCCUUCAAGGA GCCGUUAGGC UCCGGG AGCAACCA | 4702 |
| 694 | CAGGUAAU G AUGAACCC | 1242 | GGGUUCAU GGA GCCGUUAGGC UCCCUUCAAGGA GCCGUUAGGC UCCGGG AUUACCUG | 4703 |
| 697 | GUAAUGAU G AACCCUAC | 1243 | GUAGGGUU GGA GCCGUUAGGC UCCCUUCAAGGA GCCGUUAGGC UCCGGG AUCAUUAC | 4704 |
| 709 | CCUACACU G AGCAGAUG | 1244 | CAUCUGCU GGA GCCGUUAGGC UCCCUUCAAGGA GCCGUUAGGC UCCGGG AGUGUAGG | 4705 |
| 739 | AGAAGGGU G AAAGGAUC | 1245 | GAUCCUUU GGA GCCGUUAGGC UCCCUUCAAGGA GCCGUUAGGC UCCGGG ACCCUUCU | 4706 |
| 760 | UCACUCCU G AUUUCAUU | 1246 | AAUGAAAU GGA GCCGUUAGGC UCCCUUCAAGGA GCCGUUAGGC UCCGGG AGGAGUGA | 4707 |
| 769 | AUUUCAUU G CAGGAAAA | 1247 | UUUUCCUG GGA GCCGUUAGGC UCCCUUCAAGGA GCCGUUAGGC UCCGGG AAUGAAAU | 4708 |
| 787 | AGUUAGCU G AAUAUGGA | 1248 | UCCAUAUU GGA GCCGUUAGGC UCCCUUCAAGGA GCCGUUAGGC UCCGGG AGCUAACU | 4709 |
| 820 | UUGUCCAU G AGUGGGCU | 1249 | AGCCCACU GGA GCCGUUAGGC UCCCUUCAAGGA GCCGUUAGGC UCCGGG AUGGACAA | 4710 |
| 836 | UCAUCUAC G AUGGGGAG | 1250 | CUCCCCAU GGA GCCGUUAGGC UCCCUUCAAGGA GCCGUUAGGC UCCGGG GUAGAUGA | 4711 |
| 850 | GAGUAUUU G ACGAGUAC | 1251 | GUACUCGU GGA GCCGUUAGGC UCCCUUCAAGGA GCCGUUAGGC UCCGGG AAAUACUC | 4712 |
| 853 | UAUUUGAC G AGUACAAU | 1252 | AUUGUACU GGA GCCGUUAGGC UCCCUUCAAGGA GCCGUUAGGC UCCGGG GUCAAAUA | 4713 |
| 865 | ACAAUAAU G AUGAGAAA | 1253 | UUUCUCAU GGA GCCGUUAGGC UCCCUUCAAGGA GCCGUUAGGC UCCGGG AUUAUUGU | 4714 |
| 868 | AUAAUGAU G AGAAAUUC | 1254 | GAAUUUCU GGA GCCGUUAGGC UCCCUUCAAGGA GCCGUUAGGC UCCGGG AUCAUUAU | 4715 |
| 980 | CAAAAGAU G CACAUUCA | 1255 | UGAAUGUG GGA GCCGUUAGGC UCCCUUCAAGGA GCCGUUAGGC UCCGGG AUCUUUUG | 4716 |
| 1009 | GACUCUAU G AAAAAGGA | 1256 | UCCUUUUU GGA GCCGUGAGGC UCCCUUCAAGGA GCCGUUAGGC UCCGGG AUAGAGUC | 4717 |
| 1021 | AAGGAUGU G AGUUUGUU | 1257 | AACAAACU GGA GCCGUUAGGC UCCCUUCAAGGA GCCGUUAGGC UCCGGG ACAUCCUU | 4718 |
| 1040 | CCAAUCCC G CCAGACGG | 1258 | CCGUCUGG GGA GCCGUUAGGC UCCCUUCAAGGA GCCGUUAGGC UCCGGG GGGAUUGG | 4719 |
| 1069 | UAAUGUUU G CACAACAU | 1259 | AUGUUGUG GGA GCCGUUAGGC UCCCUUCAAGGA GCCGUUAGGC UCCGGG AAACAUUA | 4720 |
| 1081 | AACAUGUU G AUUCUAUA | 1260 | UAUAGAAU GGA GCCGUUAGGC UCCCUUCAAGGA GCCGUUAGGC UCCGGG AACAUGUU | 4721 |
| 1093 | CUAUAGUU G AAUUCUGU | 1261 | ACAGAAUU GGA GCCGUUAGGC UCCCUUCAAGGA GCCGUUAGGC UCCGGG AACUAUAG | 4722 |
| 1151 | UCAAAAAU G CAAUCUCC | 1262 | GGAGAUUG GGA GCCGUUAGGC UCCCUUCAAGGA GCCGUUAGGC UCCGGG AUUUUUGA | 4723 |
| 1160 | CAAUCUCC G AAGCACAU | 1263 | AUGUGCUU GGA GCCGUUAGGC UCCCUUCAAGGA GCCGUUAGGC UCCGGG GGAGAUUG | 4724 |
| 1176 | UGGGAAGU G AUCCGUGA | 1264 | UCACGGAU GGA GCCGUUAGGC UCCCUUCAAGGA GCCGUUAGGC UCCGGG ACUUCCCA | 4725 |
| 1183 | UGAUCCGU G AUUCUGAG | 1265 | CUCAGAAU GGA GCCGUUAGGC UCCCUUCAAGGA GCCGUUAGGC UCCGGG ACGGAUCA | 4726 |
| 1189 | GUGAUUCU G AGGACUUU | 1266 | AAAGUCCU GGA GCCGUUAGGC UCCCUUCAAGGA GCCGUUAGGC UCCGGG AGAAUCAC | 4727 |
| 1215 | ACUCCUAU G ACAACACA | 1267 | UGUGUUGU GGA GCCGUGAGGC UCCCUUCAAGGA GCCGUUAGGC UCCGGG AUAGGAGU | 4728 |
| 1248 | UUCUCAUU G CUGCAGAU | 1268 | AUCUGCAG GGA GCCGUUAGGC UCCCUUCAAGGA GCCGUUAGGC UCCGGG AAUGAGAA | 4729 |
| 1251 | UCAUUGCU G CAGAUUGG | 1269 | CCAAUCUG GGA GCCGUUAGGC UCCCUUCAAGGA GCCGUUAGGC UCCGGG AGCAAUGA | 4730 |
| 1285 | UACUCCUU G ACAAAUCU | 1270 | AGAUUUGU GGA GCCGUUAGGC UCCCUUCAAGGA GCCGUUAGGC UCCGGG AAGGACUA | 4731 |
| 1305 | AGCAUGGC G ACUGGUAA | 1271 | UUACCAGU GGA GCCGUUAGGC UCCCUUCAAGGA GCCGUUAGGC UCCGGG GCCAUGCU | 4732 |
| 1316 | UGGUAACC G CCUCAAUC | 1272 | GAUUGAGG GGA GCCGUUAGGC UCCCUUCAAGGA GCCGUUAGGC UCCGGG GGUUACCA | 4733 |
| 1325 | CCUCAAUC G ACUGAAUC | 1273 | GAUUCAGU GGA GCCGUUAGGC UCCCUUCAAGGA GCCGUUAGGC UCCGGG GAUUGAGG | 4734 |
| 1329 | AAUCGACU G AAUCAAGC | 1274 | GCUUGAUU GGA GCCGUUAGGC UCCCUUCAAGGA GCCGUUAGGC UCCGGG AGUCGAUU | 4735 |
| 1353 | CUUUUCCU G CUGCAGAC | 1275 | GUCUGCAG GGA GCCGUUAGGC UCCCUUCAAGGA GCCGUUAGGC UCCGGG AGGAAAAG | 4736 |
| 1356 | UUCCUGCU G CAGACAGU | 1276 | ACUGUCUG GGA GCCGUUAGGC UCCCUUCAAGGA GCCGUUAGGC UCCGGG AGCAGGAA | 4737 |
| 1366 | AGACAGUU G AGCUGGGG | 1277 | CCCCAGCU GGA GCCGUUAGGC UCCCUUCAAGGA GCCGUUAGGC UCCGGG AACUGUCU | 4738 |
| 1392 | GGGAUGGU G ACAUUUGA | 1278 | UCAAAUGU GGA GCCGUUAGGC UCCCUUCAAGGA GCCGUUAGGC UCCGGG ACCAUCCC | 4739 |
| 1399 | UGACAUUU G ACAGUGCU | 1279 | AGCACUGU GGA GCCGUUAGGC UCCCUUCAAGGA GCCGUUAGGC UCCGGG AAAUGUCA | 4740 |
| 1405 | UUGACAGU G CUGCCCAU | 1280 | AUGGGCAG GGA GCCGUUAGGC UCCCUUCAAGGA GCCGUUAGGC UCCGGG ACUGUCAA | 4741 |
| 1408 | ACAGUGCU G CCCAUGUA | 1281 | UACAUGGG GGA GCCGUUAGGC UCCCUUCAAGGA GCCGUUAGGC UCCGGG AGCACUGU | 4742 |
| 1423 | UACAAAGU G AACUCAUA | 1282 | UAUGAGUU GGA GCCGUUAGGC UCCCUUCAAGGA GCCGUUAGGC UCCGGG ACUUUGUA | 4743 |
| 1450 | GUGGCAGU G ACAGGGAC | 1283 | GUCCCUGU GGA GCCGUUAGGC UCCCUUCAAGGA GCCGUUAGGC UCCGGG ACUGCCAC | 4744 |
| 1465 | ACACACUC G CCAAAAGA | 1284 | UCUUUUGG GGA GCCGUUAGGC UCCCUUCAAGGA GCCGUUAGGC UCCGGG GAGUGUGU | 4745 |
| 1480 | GAUUACCU G CAGCAGCU | 1285 | AGCUGCUG GGA GCCGUUAGGC UCCCUUCAAGGA GCCGUUAGGC UCCGGG AGGUAAUC | 4746 |
| 1508 | GUCCAUCU G CAGCGGGC | 1286 | GCCCGCUG GGA GCCGUUAGGC UCCCUUCAAGGA GCCGUUAGGC UCCGGG AGAUGGAC | 4747 |
| 1520 | CGGGCUUC G AUCGGCAU | 1287 | AUGCCGAU GGA GCCGUUAGGC UCCCUUCAAGGA GCCGUUAGGC UCCGGG GAAGCCCG | 4748 |
| 1536 | UUUACUGU G AUUAGGAA | 1288 | UUCCUAAU GGA GCCGUUAGGC UCCCUUCAAGGA GCCGUUAGGC UCCGGG ACAGUAAA | 4749 |
| 1558 | AUCCAACU G AUGGAUCU | 1289 | AGAUCCAU GGA GCCGUUAGGC UCCCUUCAAGGA GCCGUUAGGC UCCGGG AGUUGGAU | 4750 |
| 1567 | AUGGAUCU G AAAUUGUG | 1290 | CACAAUUU GGA GCCGUUAGGC UCCCUUCAAGGA GCCGUUAGGC UCCGGG AGAUCCAU | 4751 |
| 1575 | GAAAUUGU G CUGCUGAC | 1291 | GUCAGCAG GGA GCCGUUAGGC UCCCUUCAAGGA GCCGUUAGGC UCCGGG ACAAUUUC | 4752 |
| 1578 | AUUGUGCU G CUGACGGA | 1292 | UCCGUCAG GGA GCCGUUAGGC UCCCUUCAAGGA GCCGUUAGGC UCCGGG AGCACAAU | 4753 |
| 1581 | GUGCUGCU G ACGGAUGG | 1293 | CCAUCCGU GGA GCCGUUAGGC UCCCUUCAAGGA GCCGUUAGGC UCCGGG AGCAGCAC | 4754 |
| 1613 | AAGUGGGU G CUUUAACG | 1294 | CGUUAAAG GGA GCCGUUAGGC UCCCUUCAAGGA GCCGUUAGGC UCCGGG ACCCACUU | 4755 |
| 1621 | GCUUUAAC G AGGUCAAA | 1295 | UUUGACCU GGA GCCGUUAGGC UCCCUUCAAGGA GCCGUUAGGC UCCGGG GUUAAAGC | 4756 |
| 1639 | AAAGUGGU G CCAUCAUC | 1296 | GAUGAUGG GGA GCCGUUAGGC UCCCUUCAAGGA GCCGUUAGGC UCCGGG ACCACUUU | 4757 |
| 1657 | ACACAGUC G CUUUGGGG | 1297 | CCCCAAAG GGA GCCGUUAGGC UCCCUUCAAGGA GCCGUUAGGC UCCGGG GACUGUGU | 4758 |
| 1672 | GGCCCUCU G CAGCUCAA | 1298 | UUGAGCUG GGA GCCGUUAGGC UCCCUUCAAGGA GCCGUUAGGC UCCGGG AGAGGGCC | 4759 |
| 1704 | UCCAAAAU G ACAGGAGG | 1299 | CCUCCUGU GGA GCCGUUAGGC UCCCUUCAAGGA GCCGUUAGGC UCCGGG AUUUUGGA | 4760 |
| 1726 | AGACAUAU G CUUCAGAU | 1300 | AUCUGAAG GGA GCCGUUAGGC UCCCUUCAAGGA GCCGUUAGGC UCCGGG AUAUGUCU | 4761 |
| 1759 | GCCUCAUU G AUGCUUUU | 1301 | AAAAGCAU GGA GCCGUUAGGC UCCCUUCAAGGA GCCGUUAGGC UCCGGG AAUGAGGC | 4762 |
| 1762 | UCAUUGAU G CUUUUGGG | 1302 | CCCAAAAG GGA GCCGUUAGGC UCCCUUCAAGGA GCCGUUAGGC UCCGGG AUCAAUGA | 4763 |
| 1805 | CUCUCAGC G CUCCAUCC | 1303 | GGAUGGAG GGA GCCGUUAGGC UCCCUUCAAGGA GCCGUUAGGC UCCGGG GCUGAGAG | 4764 |
| 1819 | UCCAGCUU G AGAGUAAG | 1304 | CUUACUCU GGA GCCGUUAGGC UCCCUUCAAGGA GCCGUUAGGC UCCGGG AAGCUGGA | 4765 |
| 1857 | CAGUGGAU G AAUGGCAC | 1305 | GUGCCAUU GGA GCCGUUAGGC UCCCUUCAAGGA GCCGUUAGGC UCCGGG AUCCACUG | 4766 |
| 1869 | GGCACAGU G AUCGUGGA | 1306 | UCCACGAU GGA GCCGUUAGGC UCCCUUCAAGGA GCCGUUAGGC UCCGGG ACUGUGCC | 4767 |
| 1923 | UGGACAAC G CAGCCUCC | 1307 | GGAGGCUG GGA GCCGUUAGGC UCCCUUCAAGGA GCCGUUAGGC UCCGGG GUUGUCCA | 4768 |
| 2026 | CAGGCAUU G CUAAGGUU | 1308 | AACCUUAG GGA GCCGUUAGGC UCCCUUCAAGGA GCCGUUAGGC UCCGGG AAUGCCUG | 4769 |
| 2055 | UACAGUCU G CAAGCAAG | 1309 | CUUGCUUG GGA GCCGUUAGGC UCCCUUCAAGGA GCCGUUAGGC UCCGGG AGACUGUA | 4770 |
| 2076 | CAAACCUU G ACCCUGAC | 1310 | GUCAGGGU GGA GCCGUUAGGC UCCCUUCAAGGA GCCGUUAGGC UCCGGG AAGGUUUG | 4771 |
| 2082 | UUGACCCU G ACUGUCAC | 1311 | GUGACAGU GGA GCCGUUAGGC UCCCUUCAAGGA GCCGUUAGGC UCCGGG AGGGUCAA | 4772 |
| 2098 | CGUCCCGU G CGUCCAAU | 1312 | AUUGGACG GGA GCCGUUAGGC UCCCUUCAAGGA GCCGUUAGGC UCCGGG ACGGGACG | 4773 |
| 2107 | CGUCCAAU G CUACCCUG | 1313 | CAGGGUAG GGA GCCGUUAGGC UCCCUUCAAGGA GCCGUUAGGC UCCGGG AUUGGACG | 4774 |
| 2115 | GCUACCCU G CCUCCAAU | 1314 | AUUGGAGG GGA GCCGUUAGGC UCCCUUCAAGGA GCCGUUAGGC UCCGGG AGGGUAGC | 4775 |
| 2130 | AUUACAGU G ACUUCCAA | 1315 | UUGGAAGU GGA GCCGUUAGGC UCCCUUCAAGGA GCCGUUAGGC UCCGGG ACUGUAAU | 4776 |
| 2142 | UCCAAAAC G AACAAGGA | 1316 | UCCUUGUU GGA GCCGUUAGGC UCCCUUCAAGGA GCCGUUAGGC UCCGGG GUUUUGGA | 4777 |
| 2185 | UAGUUUAU G CAAAUAUU | 1317 | AAUAUUUG GGA GCCGUUAGGC UCCCUUCAAGGA GCCGUUAGGC UCCGGG AUAAACUA | 4778 |
| 2195 | AAAUAUUC G CCAAGGAG | 1318 | CUCCUUGG GGA GCCGUUAGGC UCCCUUCAAGGA GCCGUUAGGC UCCGGG GAAUAUUU | 4779 |
| 2238 | ACAGCCCU G AUUGAAUC | 1319 | GAUUCAAU GGA GCCGUUAGGC UCCCUUCAAGGA GCCGUUAGGC UCCGGG AGGGCUGU | 4780 |
| 2242 | CCCUGAUU G AAUCAGUG | 1320 | CACUGAUU GGA GCCGUUAGGC UCCCUUCAAGGA GCCGUUAGGC UCCGGG AAUCAGGG | 4781 |
| 2250 | GAAUCAGU G AAUGGAAA | 1321 | UUUCCAUU GGA GCCGUUAGGC UCCCUUCAAGGA GCCGUUAGGC UCCGGG ACUGAUUC | 4782 |
| 2296 | GAGCAGGU G CUGAUGCU | 1322 | AGCAUCAG GGA GCCGUUAGGC UCCCUUCAAGGA GCCGUUAGGC UCCGGG ACCUGCUC | 4783 |
| 2299 | CAGGUGCU G AUGCUACU | 1323 | AGUAGCAU GGA GCCGUUAGGC UCCCUUCAAGGA GCCGUUAGGC UCCGGG AGCACCUG | 4784 |
| 2302 | GUGCUGAU G CUACUAAG | 1324 | CUUAGUAG GGA GCCGUUAGGC UCCCUUCAAGGA GCCGUUAGGC UCCGGG AUCAGCAC | 4785 |
| 2314 | CUAAGGAU G ACGGUGUC | 1325 | GACACCGU GGA GCCGUUAGGC UCCCUUCAAGGA GCCGUUAGGC UCCGGG AUCCUUAG | 4786 |
| 2347 | CAACUUAU G ACACGAAU | 1326 | AUUCGUGU GGA GCCGUUAGGC UCCCUUCAAGGA GCCGUUAGGC UCCGGG AUAAGUUG | 4787 |
| 2352 | UAUGACAC G AAUGGUAG | 1327 | CUACCAUU GGA GCCGUUAGGC UCCCUUCAAGGA GCCGUUAGGC UCCGGG GUGUCAUA | 4788 |
| 2376 | GUAAAAGU G CGGGCUCU | 1328 | AGAGCCCG GGA GCCGUUAGGC UCCCUUCAAGGA GCCGUUAGGC UCCGGG ACUUUUAC | 4789 |
| 2398 | GAGUUAAC G CAGCCAGA | 1329 | UCUGGCUG GGA GCCGUUAGGC UCCCUUCAAGGA GCCGUUAGGC UCCGGG GUUAACUC | 4790 |
| 2415 | CGGAGAGU G AUACCCCA | 1330 | UGGGGUAU GGA GCCGUUAGGC UCCCUUCAAGGA GCCGUUAGGC UCCGGG ACUCUCCG | 4791 |
| 2458 | GCUGGAUU G AGAAUGAU | 1331 | AUCAUUCU GGA GCCGUUAGGC UCCCUUCAAGGA GCCGUUAGGC UCCGGG AAUCCAGC | 4792 |
| 2464 | UUGAGAAU G AUGAAAUA | 1332 | UAUUUCAU GGA GCCGUUAGGC UCCCUUCAAGGA GCCGUUAGGC UCCGGG AUUCUCAA | 4793 |
| 2467 | AGAAUGAU G AAAUACAA | 1333 | UUGUAUUU GGA GCCGUUAGGC UCCCUUCAAGGA GCCGUUAGGC UCCGGG AUCAUUCU | 4794 |
| 2494 | CAAGACCU G AAAUUAAU | 1334 | AUUAAUUU GGA GCCGUUAGGC UCCCUUCAAGGA GCCGUUAGGC UCCGGG AGGUCUUG | 4795 |
| 2509 | AUAAGGAU G AUGUUCAA | 1335 | UUGAACAU GGA GCCGUUAGGC UCCCUUCAAGGA GCCGUUAGGC UCCGGG AUCCUUAU | 4796 |
| 2572 | UGGCUUCU G AUGUCCCA | 1336 | UGGGACAU GGA GCCGUUAGGC UCCCUUCAAGGA GCCGUUAGGC UCCGGG AGAAGCCA | 4797 |
| 2584 | UCCCAAAU G CUCCCAUA | 1337 | UAUGGGAG GGA GCCGUUAGGC UCCCUUCAAGGA GCCGUUAGGC UCCGGG AUUUGGGA | 4798 |
| 2596 | CCAUACCU G AUCUCUUC | 1338 | GAAGAGAU GGA GCCGUUAGGC UCCCUUCAAGGA GCCGUUAGGC UCCGGG AGGUAUGG | 4799 |
| 2623 | AAAUCACC G ACCUGAAG | 1339 | CUUCAGGU GGA GCCGUUAGGC UCCCUUCAAGGA GCCGUUAGGC UCCGGG GGUGAUUU | 4800 |
| 2628 | ACCGACCU G AAGGCGGA | 1340 | UCCGCCUU GGA GCCGUUAGGC UCCCUUCAAGGA GCCGUUAGGC UCCGGG AGGUCGGU | 4801 |
| 2664 | AUUAAUCU G ACUUGGAC | 1341 | GUCCAAGU GGA GCCGUUAGGC UCCCUUCAAGGA GCCGUUAGGC UCCGGG AGAUUAAU | 4802 |
| 2686 | CUGGGGAU G AUUAUGAC | 1342 | GUCAUAAU GGA GCCGUUAGGC UCCCUUCAAGGA GCCGUUAGGC UCCGGG AUCCCCAG | 4803 |
| 2692 | AUGAUUAU G ACCAUGGA | 1343 | UCCAUGGU GGA GCCGUUAGGC UCCCUUCAAGGA GCCGUUAGGC UCCGGG AUAAUCAU | 4804 |
| 2723 | UAUCAUUC G AAUAAGUA | 1344 | UACUUAUU GGA GCCGUUAGGC UCCCUUCAAGGA GCCGUUAGGC UCCGGG GAAUGAUA | 4805 |
| 2743 | GUAUUCUU G AUCUCAGA | 1345 | UCUGAGAU GGA GCCGUUAGGC UCCCUUCAAGGA GCCGUUAGGC UCCGGG AAGAAUAC | 4806 |
| 2764 | AGUUCAAU G AAUCUCUU | 1346 | AAGAGAUU GGA GCCGUUAGGC UCCCUUCAAGGA GCCGUUAGGC UCCGGG AUUGAACU | 4807 |
| 2778 | CUUCAAGU G AAUACUAC | 1347 | GUAGUAUU GGA GCCGUUAGGC UCCCUUCAAGGA GCCGUUAGGC UCCGGG ACUUGAAG | 4808 |
| 2788 | AUACUACU G CUCUCAUC | 1348 | GAUGAGAG GGA GCCGUUAGGC UCCCUUCAAGGA GCCGUUAGGC UCCGGG AGUAGUAU | 4809 |
| 2815 | CCAACUCU G AGGAAGUC | 1349 | GACUUCCU GGA GCCGUUAGGC UCCCUUCAAGGA GCCGUUAGGC UCCGGG AGAGUUGG | 4810 |
| 2854 | UUACUUUU G AAAAUGGC | 1350 | GCCAUUUU GGA GCCGUUAGGC UCCCUUCAAGGA GCCGUUAGGC UCCGGG AAAAGUAA | 4811 |
| 2878 | UUUUCAUU G CUAUUCAG | 1351 | CUGAAUAG GGA GCCGUUAGGC UCCCUUCAAGGA GCCGUUAGGC UCCGGG AAUGAAAA | 4812 |
| 2893 | AGGCUGUU G AUAAGGUC | 1352 | GACCUUAU GGA GCCGUUAGGC UCCCUUCAAGGA GCCGUUAGGC UCCGGG AACAGCCU | 4813 |
| 2902 | AUAAGGUC G AUCUGAAA | 1353 | UUUCAGAU GGA GCCGUUAGGC UCCCUUCAAGGA GCCGUUAGGC UCCGGG GACCUUAU | 4814 |
| 2907 | GUCGAUCU G AAAUCAGA | 1354 | UCUGAUUU GGA GCCGUUAGGC UCCCUUCAAGGA GCCGUUAGGC UCCGGG AGAUCGAC | 4815 |
| 2929 | CCAACAUU G CACGAGUA | 1355 | UACUCGUG GGA GCCGUUAGGC UCCCUUCAAGGA GCCGUUAGGC UCCGGG AAUGUUGG | 4816 |
| 2933 | CAUUGCAC G AGUAUCUU | 1356 | AAGAUACU GGA GCCGUUAGGC UCCCUUCAAGGA GCCGUUAGGC UCCGGG GUGCAAUG | 4817 |
| 2964 | CAGACUCC G CCAGAGAC | 1357 | GUCUCUGG GGA GCCGUUAGGC UCCCUUCAAGGA GCCGUUAGGC UCCGGG GGAGUCUG | 4818 |
| 2983 | CUAGUCCU G AUGAAACG | 1358 | CGUUUCAU GGA GCCGUUAGGC UCCCUUCAAGGA GCCGUUAGGC UCCGGG AGGACUAG | 4819 |
| 2986 | GUCCUGAU G AAACGUCU | 1359 | AGACGUUU GGA GCCGUUAGGC UCCCUUCAAGGA GCCGUUAGGC UCCGGG AUCAGGAC | 4820 |
| 2995 | AAACGUCU G CUCCUUGU | 1360 | ACAAGGAG GGA GCCGUUAGGC UCCCUUCAAGGA GCCGUUAGGC UCCGGG AGACGUUU | 4821 |
| 3078 | GGAGAACU G CAGCUGUC | 1361 | GACAGCUG GGA GCCGUUAGGC UCCCUUCAAGGA GCCGUUAGGC UCCGGG AGUUCUCC | 4822 |
| 3101 | CUAGGGCU G AAUUUUUG | 1362 | CAAAAAUU GGA GCCGUUAGGC UCCCUUCAAGGA GCCGUUAGGC UCCGGG AGCCCUAG | 4823 |
| 3145 | CUUUUUUU G AUUAUAAA | 1363 | UUUAUAAU GGA GCCGUUAGGC UCCCUUCAAGGA GCCGUUAGGC UCCGGG AAAAAAAG | 4824 |
| 3191 | UAGGGGGC G AUAUACUA | 1364 | UAGUAUAU GGA GCCGUUAGGC UCCCUUCAAGGA GCCGUUAGGC UCCGGG GCCCCCUA | 4825 |
| 3244 | UAGGGGGC G AUAUACUA | 1364 | UAGUAUAU GGA GCCGUUAGGC UCCCUUCAAGGA GCCGUUAGGC UCCGGG GCCCCCUA | 4825 |
| 3281 | UAGGGGGC G AUAAAAUA | 1365 | UAUUUUAU GGA GCCGUUAGGC UCCCUUCAAGGA GCCGUUAGGC UCCGGG GCCCCCUA | 4826 |
| 3294 | AAUAAAAU G CUAAACAA | 1366 | UUGUUUAG GGA GCCGUUAGGC UCCCUUCAAGGA GCCGUUAGGC UCCGGG AUUUUAUU | 4827 |
| 27 | AAAUGGAU G UGGAAUAU | 1367 | AUAUUCCA GGA GCCGUUAGGC UCCCUUCAAGGA GCCGUUAGGC UCCGGG AUCCAUUU | 4828 |
| 52 | AUUUUCUU G UUUAAGGG | 1368 | CCCUUAAA GGA GCCGUUAGGC UCCCUUCAAGGA GCCGUUAGGC UCCGGG AAGAAAAU | 4829 |
| 75 | GAAGAGGU G UUGAGGUU | 1369 | AACCUCAA GGA GCCGUUAGGC UCCCUUCAAGGA GCCGUUAGGC UCCGGG ACCUCUUC | 4830 |
| 86 | GAGGUUAU G UCAAGCAU | 1370 | AUGCUUGA GGA GCCGUUAGGC UCCCUUCAAGGA GCCGUUAGGC UCCGGG AUAACCUC | 4831 |
| 155 | AAGAUAUU G UUAUCAUU | 1371 | AAUGAUAA GGA GCCGUUAGGC UCCCUUCAAGGA GCCGUUAGGC UCCGGG AAUAUCUU | 4832 |
| 221 | AAAGACCU G UGAUAAAC | 1372 | GUUUAUCA GGA GCCGUUAGGC UCCCUUCAAGGA GCCGUUAGGC UCCGGG AGGUCUUU | 4833 |
| 253 | GGAAACGU G UGUCUAUA | 1373 | UAUAGACA GGA GCCGUUAGGC UCCCUUCAAGGA GCCGUUAGGC UCCGGG ACGUUUCC | 4834 |
| 255 | AAACGUGU G UCUAUAUU | 1374 | AAUAUAGA GGA GCCGUUAGGC UCCCUUCAAGGA GCCGUUAGGC UCCGGG ACACGUUU | 4835 |
| 273 | UCAUAUCU G UAUAUAUA | 1375 | UAUAUAUA GGA GCCGUUAGGC UCCCUUCAAGGA GCCGUUAGGC UCCGGG AGAUAUGA | 4836 |
| 344 | AGGGAGAU G UACAGCAA | 1376 | UUGCUGUA GGA GCCGUUAGGC UCCCUUCAAGGA GCCGUUAGGC UCCGGG AUCUCCCU | 4837 |
| 373 | AGAGUUCU G UGUUCAUC | 1377 | GAUGAACA GGA GCCGUUAGGC UCCCUUCAAGGA GCCGUUAGGC UCCGGG AGAACUCU | 4838 |
| 375 | AGUUCUGU G UUCAUCUU | 1378 | AAGAUGAA GGA GCCGUUAGGC UCCCUUCAAGGA GCCGUUAGGC UCCGGG ACAGAACU | 4839 |
| 457 | AAGGCAUU G UCGUUGCA | 1379 | UGCAACGA GGA GCCGUUAGGC UCCCUUCAAGGA GCCGUUAGGC UCCGGG AAUGCCUU | 4840 |
| 478 | ACCCCAAU G UGCCAGAA | 1380 | UUCUGGCA GGA GCCGUUAGGC UCCCUUCAAGGA GCCGUUAGGC UCCGGG AUUGGGGU | 4841 |
| 537 | GCAUCUCU G UAUCUGUU | 1381 | AACAGAUA GGA GCCGUUAGGC UCCCUUCAAGGA GCCGUUAGGC UCCGGG AGAGAUGC | 4842 |
| 543 | CUGUAUCU G UUUGAAGC | 1382 | GCUUCAAA GGA GCCGUUAGGC UCCCUUCAAGGA GCCGUUAGGC UCCGGG AGAUACAG | 4843 |
| 580 | UCAAAAAU G UUGCCAUU | 1383 | AAUGGCAA GGA GCCGUUAGGC UCCCUUCAAGGA GCCGUUAGGC UCCGGG AUUUUUGA | 4844 |
| 625 | CUGACUAU G UGAGACCA | 1384 | UGGUCUCA GGA GCCGUUAGGC UCCCUUCAAGGA GCCGUUAGGC UCCGGG AUAGUCAG | 4845 |
| 661 | AUGCUGAU G UUCUGGUU | 1385 | AACCAGAA GGA GCCGUUAGGC UCCCUUCAAGGA GCCGUUAGGC UCCGGG AUCAGCAU | 4846 |
| 725 | GGGCAACU G UGGAGAGA | 1386 | UCUCUCCA GGA GCCGUUAGGC UCCCUUCAAGGA GCCGUUAGGC UCCGGG AGUUGCCC | 4847 |
| 814 | AGGCAUUU G UCCAUGAG | 1387 | CUCAUGGA GGA GCCGUUAGGC UCCCUUCAAGGA GCCGUUAGGC UCCGGG AAAUGCCU | 4848 |
| 911 | AGUAAGAU G UUCAGCAG | 1388 | CUGCUGAA GGA GCCGUUAGGC UCCCUUCAAGGA GCCGUUAGGC UCCGGG AUCUUACU | 4849 |
| 937 | GUACAAAU G UAGUAAAG | 1389 | CUUUACUA GGA GCCGUUAGGC UCCCUUCAAGGA GCCGUUAGGC UCCGGG AUUUGUAC | 4850 |
| 950 | AAAGAAGU G UCAGGGAG | 1390 | CUCCCUGA GGA GCCGUUAGGC UCCCUUCAAGGA GCCGUUAGGC UCCGGG ACUUCUUU | 4851 |
| 965 | AGGCAGCU G UUACACCA | 1391 | UGGUGUAA GGA GCCGUUAGGC UCCCUUCAAGGA GCCGUUAGGC UCCGGG AGCUGCCU | 4852 |
| 1019 | AAAAGGAU G UGAGUUUG | 1392 | CAAACUCA GGA GCCGUUAGGC UCCCUUCAAGGA GCCGUUAGGC UCCGGG AUCCUUUU | 4853 |
| 1027 | GUGAGUUU G UUCUCCAA | 1393 | UUGGAGAA GGA GCCGUUAGGC UCCCUUCAAGGA GCCGUUAGGC UCCGGG AAACUCAC | 4854 |
| 1065 | UCUAUAAU G UUUGCACA | 1394 | UGUGCAAA GGA GCCGUUAGGC UCCCUUCAAGGA GCCGUUAGGC UCCGGG AUUAUAGA | 4855 |
| 1078 | CACAACAU G UUGAUUCU | 1395 | AGAAUCAA GGA GCCGUUAGGC UCCCUUCAAGGA GCCGUUAGGC UCCGGG AUGUUGUG | 4856 |
| 1100 | UGAAUUCU G UACAGAAC | 1396 | GUUCUGUA GGA GCCGUUAGGC UCCCUUCAAGGA GCCGUUAGGC UCCGGG AGAAUUCA | 4857 |
| 1270 | AAAGAAUU G UGUGUUUA | 1397 | UAAACACA GGA GCCGUUAGGC UCCCUUCAAGGA GCCGUUAGGC UCCGGG AAUUCUUU | 4858 |
| 1272 | AGAAUUGU G UGUUUAGU | 1398 | ACUAAACA GGA GCCGUUAGGC UCCCUUCAAGGA GCCGUUAGGC UCCGGG ACAAUUCU | 4859 |
| 1274 | AAUUGUGU G UUUAGUCC | 1399 | GGACUAAA GGA GCCGUUAGGC UCCCUUCAAGGA GCCGUUAGGC UCCGGG ACACAAUU | 4860 |
| 1414 | CUGCCCAU G UACAAAGU | 1400 | ACUUUGUA GGA GCCGUUAGGC UCCCUUCAAGGA GCCGUUAGGC UCCGGG AUGGGCAG | 4861 |
| 1534 | CAUUUACU G UGAUUAGG | 1401 | CCUAAUCA GGA GCCGUUAGGC UCCCUUCAAGGA GCCGUUAGGC UCCGGG AGUAAAUG | 4862 |
| 1573 | CUGAAAUU G UGCUGCUG | 1402 | CAGCAGCA GGA GCCGUUAGGC UCCCUUCAAGGA GCCGUUAGGC UCCGGG AAUUUCAG | 4863 |
| 1695 | GAGGAGCU G UCCAAAAU | 1403 | AUUUUGGA GGA GCCGUUAGGC UCCCUUCAAGGA GCCGUUAGGC UCCGGG AGCUCCUC | 4864 |
| 1795 | AUGGAGCU G UCUCUCAG | 1404 | CUGAGAGA GGA GCCGUUAGGC UCCCUUCAAGGA GCCGUUAGGC UCCGGG AGCUCCAU | 4865 |
| 1902 | GACACUUU G UUUCUUAU | 1405 | AUAAGAAA GGA GCCGUUAGGC UCCCUUCAAGGA GCCGUUAGGC UCCGGG AAAGUGUC | 4866 |
| 1978 | GUGGCUUU G UAGUGGAC | 1406 | GUCCACUA GGA GCCGUUAGGC UCCCUUCAAGGA GCCGUUAGGC UCCGGG AAAGCCAC | 4867 |
| 2086 | CCCUGACU G UCACGUCC | 1407 | GGACGUGA GGA GCCGUUAGGC UCCCUUCAAGGA GCCGUUAGGC UCCGGG AGUCAGGG | 4868 |
| 2227 | GGGCCAGU G UCACAGCC | 1408 | GGCUGUGA GGA GCCGUUAGGC UCCCUUCAAGGA GCCGUUAGGC UCCGGG ACUGGCCC | 4869 |
| 2320 | AUGACGGU G UCUACUCA | 1409 | UGAGUAGA GGA GCCGUUAGGC UCCCUUCAAGGA GCCGUUAGGC UCCGGG ACCGUCAU | 4870 |
| 2368 | GAUACAGU G UAAAAGUG | 1410 | CACUUUUA GGA GCCGUUAGGC UCCCUUCAAGGA GCCGUUAGGC UCCGGG ACUGUAUC | 4871 |
| 2439 | GGAGCACU G UACAUACC | 1411 | GGUAUGUA GGA GCCGUUAGGC UCCCUUCAAGGA GCCGUUAGGC UCCGGG AGUGCUCC | 4872 |
| 2512 | AGGAUGAU G UUCAACAC | 1412 | GUGUUGAA GGA GCCGUUAGGC UCCCUUCAAGGA GCCGUUAGGC UCCGGG AUCAUCCU | 4873 |
| 2529 | AAGCAAGU G UGUUUCAG | 1413 | CUGAAACA GGA GCCGUUAGGC UCCCUUCAAGGA GCCGUUAGGC UCCGGG ACUUGCUU | 4874 |
| 2531 | GCAAGUGU G UUUCAGCA | 1414 | UGCUGAAA GGA GCCGUUAGGC UCCCUUCAAGGA GCCGUUAGGC UCCGGG ACACUUGC | 4875 |
| 2563 | GCUCAUUU G UGGCUUCU | 1415 | AGAAGCCA GGA GCCGUUAGGC UCCCUUCAAGGA GCCGUUAGGC UCCGGG AAAUGAGC | 4876 |
| 2575 | CUUCUGAU G UCCCAAAU | 1416 | AUUUGGGA GGA GCCGUUAGGC UCCCUUCAAGGA GCCGUUAGGC UCCGGG AUCAGAAG | 4877 |
| 2829 | GUCUUUUU G UUUAAACC | 1417 | GGUUUAAA GGA GCCGUUAGGC UCCCUUCAAGGA GCCGUUAGGC UCCGGG AAAAAGAC | 4878 |
| 2890 | UUCAGGCU G UUGAUAAG | 1418 | CUUAUCAA GGA GCCGUUAGGC UCCCUUCAAGGA GCCGUUAGGC UCCGGG AGCCUGAA | 4879 |
| 2943 | GUAUCUUU G UUUAUUCC | 1419 | GGAAUAAA GGA GCCGUUAGGC UCCCUUCAAGGA GCCGUUAGGC UCCGGG AAAGAUAC | 4880 |
| 3002 | UGCUCCUU G UCCUAAUA | 1420 | UAUUAGGA GGA GCCGUUAGGC UCCCUUCAAGGA GCCGUUAGGC UCCGGG AAGGAGCA | 4881 |
| 3057 | AAAAUUAU G UGGAAGUG | 1421 | CACUUCCA GGA GCCGUUAGGC UCCCUUCAAGGA GCCGUUAGGC UCCGGG AUAAUUUU | 4882 |
| 3084 | CUGCAGCU G UCAAUAGC | 1422 | GCUAUUGA GGA GCCGUUAGGC UCCCUUCAAGGA GCCGUUAGGC UCCGGG AGCUGCAG | 4883 |
| 3109 | GAAUUUUU G UCAGAUAA | 1423 | UUAUCUGA GGA GCCGUUAGGC UCCCUUCAAGGA GCCGUUAGGC UCCGGG AAAAAUUC | 4884 |
| 3166 | UCUAAAAU G UAUUUUAG | 1424 | CUAAAAUA GGA GCCGUUAGGC UCCCUUCAAGGA GCCGUUAGGC UCCGGG AUUUUAGA | 4885 |
| 3182 | GACUUCCU G UAGGGGGC | 1425 | GCCCCCUA GGA GCCGUUAGGC UCCCUUCAAGGA GCCGUUAGGC UCCGGG AGGAAGUC | 4886 |
| 3272 | GACUUCCU G UAGGGGGC | 1425 | GCCCCCUA GGA GCCGUUAGGC UCCCUUCAAGGA GCCGUUAGGC UCCGGG AGGAAGUC | 4886 |
| 3203 | UACUAAAU G UAUAUAGU | 1426 | ACUAUAUA GGA GCCGUUAGGC UCCCUUCAAGGA GCCGUUAGGC UCCGGG AUUUAGUA | 4887 |
| 3227 | UACUAAAU G UAUUCCUG | 1427 | CAGGAAUA GGA GCCGUUAGGC UCCCUUCAAGGA GCCGUUAGGC UCCGGG AUUUAGUA | 4888 |
| 3235 | GUAUUCCU G UAGGGGGC | 1428 | GCCCCCUA GGA GCCGUUAGGC UCCCUUCAAGGA GCCGUUAGGC UCCGGG AGGAAUAC | 4889 |
| 3256 | UACUAAAU G UAUUUUAG | 1429 | CUAAAAUA GGA GCCGUUAGGC UCCCUUCAAGGA GCCGUUAGGC UCCGGG AUUUAGUA | 4890 |
| 15 | UGCUUUUG G UACAAAUG | 1430 | CAUUUGUA GGA GCCGUUAGGC UCCCUUCAAGGA GCCGUUAGGC UCCGGG CAAAAGCA | 4891 |
| 63 | UAAGGGGA G CAUGAAGA | 1431 | UCUUCAUG GGA GCCGUUAGGC UCCCUUCAAGGA GCCGUUAGGC UCCGGG UCCCCUUA | 4892 |
| 73 | AUGAAGAG G UGUUGAGG | 1432 | CCUCAACA GGA GCCGUUAGGC UCCCUUCAAGGA GCCGUUAGGC UCCGGG CUCUUCAU | 4893 |
| 81 | GUGUUGAG G UUAUGUCA | 1433 | UGACAUAA GGA GCCGUUAGGC UCCCUUCAAGGA GCCGUUAGGC UCCGGG CUCAACAC | 4894 |
| 91 | UAUGUCAA G CAUCUGGC | 1434 | GCCAGAUG GGA GCCGUUAGGC UCCCUUCAAGGA GCCGUUAGGC UCCGGG UUGACAUA | 4895 |
| 98 | AGCAUCUG G CACAGCUG | 1435 | CAGCUGUG GGA GCCGUUAGGC UCCCUUCAAGGA GCCGUUAGGC UCCGGG CAGAUGCU | 4896 |
| 103 | CUGGCACA G CUGAAGGC | 1436 | GCCUUCAG GGA GCCGUUAGGC UCCCUUCAAGGA GCCGUUAGGC UCCGGG UGUGCCAG | 4897 |
| 110 | AGCUGAAG G CAGAUGGA | 1437 | UCCAUCUG GGA GCCGUUAGGC UCCCUUCAAGGA GCCGUUAGGC UCCGGG CUUCAGCU | 4898 |
| 130 | AUUUACAA G UACGCAAU | 1438 | AUUGCGUA GGA GCCGUUAGGC UCCCUUCAAGGA GCCGUUAGGC UCCGGG UUGUAAAU | 4899 |
| 182 | AGACAAGA G CAAUAGUA | 1439 | UACUAUUG GGA GCCGUUAGGC UCCCUUCAAGGA GCCGUUAGGC UCCGGG UCUUGUCU | 4900 |
| 188 | GAGCAAUA G UAAAACAC | 1440 | GUGUUUUA GGA GCCGUUAGGC UCCCUUCAAGGA GCCGUUAGGC UCCGGG UAUUGCUC | 4901 |
| 202 | CACAUCAG G UCAGGGGG | 1441 | CCCCCUGA GGA GCCGUUAGGC UCCCUUCAAGGA GCCGUUAGGC UCCGGG CUGAUGUG | 4902 |
| 210 | GUCAGGGG G UUAAAGAC | 1442 | GUCUUUAA GGA GCCGUUAGGC UCCCUUCAAGGA GCCGUUAGGC UCCGGG CCCCUGAC | 4903 |
| 242 | UCCGAUAA G UUGGAAAC | 1443 | GUUUCCAA GGA GCCGUUAGGC UCCCUUCAAGGA GCCGUUAGGC UCCGGG UUAUCGGA | 4904 |
| 251 | UUGGAAAC G UGUGUCUA | 1444 | UAGACACA GGA GCCGUUAGGC UCCCUUCAAGGA GCCGUUAGGC UCCGGG GUUUCCAA | 4905 |
| 287 | AUAUAAUG G UAAAGAAA | 1445 | UUUCUUUA GGA GCCGUUAGGC UCCCUUCAAGGA GCCGUUAGGC UCCGGG CAUUAUAU | 4906 |
| 305 | ACACCUUC G UAACCCGC | 1446 | GCGGGUUA GGA GCCGUUAGGC UCCCUUCAAGGA GCCGUUAGGC UCCGGG GAAGGUGU | 4907 |
| 349 | GAUGUACA G CAAUGGGG | 1447 | CCCCAUUG GGA GCCGUUAGGC UCCCUUCAAGGA GCCGUUAGGC UCCGGG UGUACAUC | 4908 |
| 357 | GCAAUGGG G CCAUUUAA | 1448 | UUAAAUGG GGA GCCGUUAGGC UCCCUUCAAGGA GCCGUUAGGC UCCGGG CCCAUUGC | 4909 |
| 368 | AUUUAAGA G UUCUGUGU | 1449 | ACACAGAA GGA GCCGUUAGGC UCCCUUCAAGGA GCCGUUAGGC UCCGGG UCUUAAAU | 4910 |
| 406 | UAGAAGGG G CCCUGAGU | 1450 | ACUCAGGG GGA GCCGUUAGGC UCCCUUCAAGGA GCCGUUAGGC UCCGGG CCCUUCUA | 4911 |
| 413 | GGCCCUGA G UAAUUCAC | 1451 | GUGAAUUA GGA GCCGUUAGGC UCCCUUCAAGGA GCCGUUAGGC UCCGGG UCAGGGCC | 4912 |
| 429 | CUCAUUCA G CUGAACAA | 1452 | UUGUUCAG GGA GCCGUUAGGC UCCCUUCAAGGA GCCGUUAGGC UCCGGG UGAAUGAG | 4913 |
| 443 | CAACAAUG G CUAUGAAG | 1453 | CUUCAUAG GGA GCCGUUAGGC UCCCUUCAAGGA GCCGUUAGGC UCCGGG CAUUGUUG | 4914 |
| 452 | CUAUGAAG G CAUUGUCG | 1454 | CGACAAUG GGA GCCGUUAGGC UCCCUUCAAGGA GCCGUUAGGC UCCGGG CUUCAUAG | 4915 |
| 460 | GCAUUGUC G UUGCAAUC | 1455 | GAUUGCAA GGA GCCGUUAGGC UCCCUUCAAGGA GCCGUUAGGC UCCGGG GACAAUGC | 4916 |
| 520 | AGGACAUG G UGACCCAG | 1456 | CUGGGUCA GGA GCCGUUAGGC UCCCUUCAAGGA GCCGUUAGGC UCCGGG CAUGUCCU | 4917 |
| 529 | UGACCCAG G CAUCUCUG | 1457 | CAGAGAUG GGA GCCGUUAGGC UCCCUUCAAGGA GCCGUUAGGC UCCGGG CUGGGUCA | 4918 |
| 550 | UGUUUGAA G CUACAGGA | 1458 | UCCUGUAG GGA GCCGUUAGGC UCCCUUCAAGGA GCCGUUAGGC UCCGGG UUCAAACA | 4919 |
| 561 | ACAGGAAA G CGAUUUUA | 1459 | UAAAAUCG GGA GCCGUUAGGC UCCCUUCAAGGA GCCGUUAGGC UCCGGG UUUCCUGU | 4920 |
| 616 | AGACAAAG G CUGACUAU | 1460 | AUAGUCAG GGA GCCGUUAGGC UCCCUUCAAGGA GCCGUUAGGC UCCGGG CUUUGUCU | 4921 |
| 667 | AUGUUCUG G UUGCUGAG | 1461 | CUCAGCAA GGA GCCGUUAGGC UCCCUUCAAGGA GCCGUUAGGC UCCGGG CAGAACAU | 4922 |
| 675 | GUUGCUGA G UCUACUCC | 1462 | GGACUAGA GGA GCCGUUAGGC UCCCUUCAAGGA GCCGUUAGCC UCCGGG UCAGCAAC | 4923 |
| 689 | UCCUCCAG G UAAUGAUG | 1463 | CAUCAUUA GGA GCCGUUAGGC UCCCUUCAAGGA GCCGUUAGGC UCCGGG CUGGAGGA | 4924 |
| 711 | UACACUGA G CAGAUGGG | 1464 | CCCAUCUG GGA GCCGUUAGGC UCCCUUCAAGGA GCCGUUAGGC UCCGGG UCAGUGUA | 4925 |
| 719 | GCAGAUGG G CAACUGUG | 1465 | CACAGUUG GGA GCCGUUAGGC UCCCUUCAAGGA GCCGUUAGGC UCCGGG CCAUCUGC | 4926 |
| 737 | AGAGAAGG G UGAAAGGA | 1466 | UCCUUUCA GGA GCCGUUAGGC UCCCUUCAAGGA GCCGUUAGGC UCCGGG CCUUCUCU | 4927 |
| 780 | GGAAAAAA G UUAGCUGA | 1467 | UCAGCUAA GGA GCCGUUAGGC UCCCUUCAAGGA GCCGUUAGGC UCCGGG UUUUUUCC | 4928 |
| 784 | AAAAGUUA G CUGAAUAU | 1468 | AUAUUCAG GGA GCCGUUAGGC UCCCUUCAAGGA GCCGUUAGGC UCCGGG UAACUUUU | 4929 |
| 803 | ACCACAAG G UAAGGCAU | 1469 | AUGCCUUA GGA GCCGUUAGGC UCCCUUCAAGGA GCCGUUAGGC UCCGGG CUUGUGGU | 4930 |
| 808 | AAGGUAAG G CAUUUGUC | 1470 | GACAAAUG GGA GCCGUUAGGC UCCCUUCAAGGA GCCGUUAGGC UCCGGG CUUACCUU | 4931 |
| 822 | GUCCAUGA G UGGGCUCA | 1471 | UGAGCCCA GGA GCCGUUAGGC UCCCUUCAAGGA GCCGUUAGGC UCCGGG UCAUGGAC | 4932 |
| 826 | AUGAGUGG G CUCAUCUA | 1472 | UAGAUGAG GGA GCCGUUAGGC UCCCUUCAAGGA GCCGUUAGGC UCCGGG CCACUCAU | 4933 |
| 844 | GAUGGGGA G UAUUUGAC | 1473 | GUCAAAUA GGA GCCGUUAGGC UCCCUUCAAGGA GCCGUUAGGC UCCGGG UCCCCAUC | 4934 |
| 855 | UUUGACGA G UACAAUAA | 1474 | UUAUUGUA GGA GCCGUUAGGC UCCCUUCAAGGA GCCGUUAGGC UCCGGG UCGUCAAA | 4935 |
| 901 | GAAUACAA G CAGUAAGA | 1475 | UCUUACUG GGA GCCGUUAGGC UCCCUUCAAGGA GCCGUUAGGC UCCGGG UUGUAUUC | 4936 |
| 904 | UACAAGCA G UAAGAUGU | 1476 | ACAUCUUA GGA GCCGUUAGGC UCCCUUCAAGGA GCCGUUAGGC UCCGGG UGCUUGUA | 4937 |
| 916 | GAUGUUCA G CAGGUAUU | 1477 | AAUACCUG GGA GCCGUUAGGC UCCCUUCAAGGA GCCGUUAGGC UCCGGG UGAACAUC | 4938 |
| 920 | UUCAGCAG G UAUUACUG | 1478 | CAGUAAUA GGA GCCGUUAGGC UCCCUUCAAGGA GCCGUUAGGC UCCGGG CUGCUGAA | 4939 |
| 929 | UAUUACUG G UACAAAUG | 1479 | CAUUUGUA GGA GCCGUUAGGC UCCCUUCAAGGA GCCGUUAGGC UCCGGG CAGUAAUA | 4940 |
| 940 | CAAAUGUA G UAAAGAAG | 1480 | CUUCUUUA GGA GCCGUUAGGC UCCCUUCAAGGA GCCGUUAGGC UCCGGG UACAUUUG | 4941 |
| 948 | GUAAAGAA G UGUCAGGG | 1481 | CCCUGACA GGA GCCGUUAGGC UCCCUUCAAGGA GCCGUUAGGC UCCGGG UUCUUUAC | 4942 |
| 959 | UCAGGCAC G CAGCUGUU | 1482 | AACAGCUG GGA GCCGUUAGGC UCCCUUCAAGGA GCCGUUAGGC UCCGGG CUCCCUGA | 4943 |
| 962 | GGGAGCCA G CUGUUACA | 1483 | UGUAACAG GGA GCCGUUAGGC UCCCUUCAAGGA GCCGUUAGGC UCCGGG UGCCUCCC | 4944 |
| 994 | UCAAUAAA G UUACAGGA | 1484 | UCCUGUAA GGA GCCGUUAGGC UCCCUUCAAGGA GCCGUUAGGC UCCGGG UUUAUUGA | 4945 |
| 1023 | GGAUGUGA G UUUGUUCU | 1485 | AGAACAAA GGA GCCGUUAGGC UCCCUUCAAGGA GCCGUUAGGC UCCGGG UCACAUCC | 4946 |
| 1054 | CGGAGAAG G CUUCUAUA | 1486 | UAUAGAAG GGA GCCGUUAGGC UCCCUUCAAGGA GCCGUUAGGC UCCGGG CUUCUCCG | 4947 |
| 1090 | AUUCUAUA G UUGAAUUC | 1487 | GAAUUCAA GGA GCCGUUAGGC UCCCUUCAAGGA GCCGUUAGGC UCCGGG UAUAGAAU | 4948 |
| 1126 | ACAAAGAA G CUCCAAAC | 1488 | GUUUGGAG GGA GCCGUUAGGC UCCCUUCAAGGA GCCGUUAGGC UCCGGG UUCUUUGU | 4949 |
| 1137 | CCAAACAA G CAAAAUCA | 1489 | UGAUUUUG GGA GCCGUUAGGC UCCCUUCAAGGA GCCGUUAGGC UCCGGG UUGUUUGG | 4950 |
| 1163 | UCUCCGAA G CACAUGGG | 1490 | CCCAUGUG GGA GCCGUUAGGC UCCCUUCAAGGA GCCGUUAGGC UCCGGG UUCGGAGA | 4951 |
| 1174 | CAUGGGAA G UGAUCCGU | 1491 | ACGGAUCA GGA GCCGUUAGGC UCCCUUCAAGGA GCCGUUAGGC UCCGGG UUCCCAUG | 4952 |
| 1181 | AGUGAUCC G UGAUUCUG | 1492 | CAGAAUCA GGA GCCGUUAGGC UCCCUUCAAGGA GCCGUUAGGC UCCGGG GGAUCACU | 4953 |
| 1224 | ACAACACA G CCACCAAA | 1493 | UUUGGUGG GGA GCCGUUAGGC UCCCUUCAAGGA GCCGUUAGGC UCCGGG UGUGUUGU | 4954 |
| 1279 | UGUGUUUA G UCCUUGAC | 1494 | GUCAAGGA GGA GCCGUUAGGC UCCCUUCAAGGA GCCGUUAGGC UCCGGG UAAACACA | 4955 |
| 1298 | AUCUGGAA G CAUGGCGA | 1495 | UCGCCAUG GGA GCCGUUAGGC UCCCUUCAAGGA GCCGUUAGGC UCCGGG UUCCAGAU | 4956 |
| 1303 | GAAGCAUG G CGACUGGU | 1496 | ACCAGUCG GGA GCCGUUAGGC UCCCUUCAAGGA GCCGUUAGGC UCCGGG CAUGCUUC | 4957 |
| 1310 | GGCGACUG G UAACCGCC | 1497 | GGCGGUUA GGA GCCGUUAGGC UCCCUUCAAGGA GCCGUUAGGC UCCGGG CAGUCGCC | 4958 |
| 1336 | UGAAUCAA G CAGGCCAG | 1498 | CUGGCCUG GGA GCCGUUAGGC UCCCUUCAAGGA GCCGUUAGGC UCCGGG UUGAUUCA | 4959 |
| 1340 | UCAAGCAG G CCAGCUUU | 1499 | AAAGCUGG GGA GCCGUUAGGC UCCCUUCAAGGA GCCGUUAGGC UCCGGG CUGCUUGA | 4960 |
| 1344 | GCAGGCCA G CUUUUCCU | 1500 | AGGAAAAG GGA GCCGUUAGGC UCCCUUCAAGGA GCCGUUAGGC UCCGGG UGGCCUGC | 4961 |
| 1363 | UGCAGACA G UUGAGCUG | 1501 | CAGCUCAA GGA GCCGUUAGGC UCCCUUCAAGGA GCCGUUAGGC UCCGGG UGUCUGCA | 4962 |
| 1368 | ACAGUUGA G CUGGGGUC | 1502 | GACCCCAG GGA GCCGUUAGGC UCCCUUCAAGGA GCCGUUAGGC UCCGGG UCAACUGU | 4963 |
| 1374 | GAGCUGGG G UCCUGGGU | 1503 | ACCCAGGA GGA GCCGUUAGGC UCCCUUCAAGGA GCCGUUAGGC UCCGGG CCCAGCUC | 4964 |
| 1381 | GGUCCUGG G UUGGGAUG | 1504 | CAUCCCAA GGA GCCGUUAGGC UCCCUUCAAGGA GCCGUUAGGC UCCGGG CCAGGACC | 4965 |
| 1390 | UUGGGAUG G UGACAUUU | 1505 | AAAUGUCA GGA GCCGUUAGGC UCCCUUCAAGGA GCCGUUAGGC UCCGGG CAUCCCAA | 4966 |
| 1403 | AUUUGACA G UGCUGCCC | 1506 | GGGCAGCA GGA GCCGUUAGGC UCCCUUCAAGGA GCCGUUAGGC UCCGGG UGUCAAAU | 4967 |
| 1421 | UGUACAAA G UGAACUCA | 1507 | UGAGUUCA GGA GCCGUUAGGC UCCCUUCAAGGA GCCGUUAGGC UCCGGG UUUGUACA | 4968 |
| 1442 | GAUAAACA G UGGCAGUG | 1508 | CACUGCCA GGA GCCGUUAGGC UCCCUUCAAGGA GCCGUUAGGC UCCGGG UGUUUAUC | 4969 |
| 1445 | AAACAGUG G CAGUGACA | 1509 | UGUCACUG GGA GCCGUUAGGC UCCCUUCAAGGA GCCGUUAGGC UCCGGG CACUGUUU | 4970 |
| 1448 | CAGUGGCA G UGACAGGG | 1510 | CCCUGUCA GGA GCCGUUAGGC UCCCUUCAAGGA GCCGUUAGGC UCCGGG UGCCACUG | 4971 |
| 1483 | UACCUGCA G CAGCUUCA | 1511 | UGAAGCUG GGA GCCGUUAGGC UCCCUUCAAGGA GCCGUUAGGC UCCGGG UGCAGGUA | 4972 |
| 1486 | CUGCAGCA G CUUCAGGA | 1512 | UCCUGAAG GGA GCCGUUAGGC UCCCUUCAAGGA GCCGUUAGGC UCCGGG UGCUGCAG | 4973 |
| 1500 | GGAGGGAC G UCCAUCUG | 1513 | CAGAUGGA GGA GCCGUUAGGC UCCCUUCAAGGA GCCGUUAGGC UCCGGG GUCCCUCC | 4974 |
| 1511 | CAUCUGCA G CGGGCUUC | 1514 | GAAGCCCG GGA GCCGUUAGGC UCCCUUCAAGGA GCCGUUAGGC UCCGGG UGCAGAUG | 4975 |
| 1515 | UGCAGCGG G CUUCGAUC | 1515 | GAUCGAAG GGA GCCGUUAGGC UCCCUUCAAGGA GCCGUUAGGC UCCGGG CCGCUGCA | 4976 |
| 1525 | UUCGAUCG G CAUUUACU | 1516 | AGUAAAUG GGA GCCGUUAGGC UCCCUUCAAGGA GCCGUUAGGC UCCGGG CGAUCGAA | 4977 |
| 1607 | CACUAUAA G UGGGUGCU | 1517 | AGCACCCA GGA GCCGUUAGGC UCCCUUCAAGGA GCCGUUAGGC UCCGGG UUAUAGUG | 4978 |
| 1611 | AUAAGUGG G UGCUUUAA | 1518 | UUAAAGCA GGA GCCGUUAGGC UCCCUUCAAGGA GCCGUUAGGC UCCGGG CCACUUAU | 4979 |
| 1624 | UUAACGAG G UCAAACAA | 1519 | UUGUUUGA GGA GCCGUUAGGC UCCCUUCAAGGA GCCGUUAGGC UCCGGG CUCGUUAA | 4980 |
| 1634 | CAAACAAA G UGGUGCCA | 1520 | UGGCACCA GGA GCCGUUAGGC UCCCUUCAAGGA GCCGUUAGGC UCCGGG UUUGUUUG | 4981 |
| 1637 | ACAAAGUG G UGCCAUCA | 1521 | UGAUGGCA GGA GCCGUUAGGC UCCCUUCAAGGA GCCGUUAGGC UCCGGG CACUUUGU | 4982 |
| 1654 | UCCACACA G UCGCUUUG | 1522 | CAAAGCGA GGA GCCGUUAGGC UCCCUUCAAGGA GCCGUUAGGC UCCGGG UGUGUGGA | 4983 |
| 1665 | GCUUUGGG G CCCUCUGC | 1523 | GCAGAGGG GGA GCCGUUAGGC UCCCUUCAAGGA GCCGUUAGGC UCCGGG CCCAAAGC | 4984 |
| 1675 | CCUCUGCA G CUCAAGAA | 1524 | UUCUUGAG GGA GCCGUUAGGC UCCCUUCAAGGA GCCGUUAGGC UCCGGG UGCAGAGG | 4985 |
| 1692 | CUAGAGGA G CUGUCCAA | 1525 | UUGGACAG GGA GCCGUUAGGC UCCCUUCAAGGA GCCGUUAGGC UCCGGG UCCUCUAG | 4986 |
| 1712 | GACAGGAG G UUUACAGA | 1526 | UCUGUAAA GGA GCCGUUAGGC UCCCUUCAAGGA GCCGUUAGGC UCCGGG CUCCUGUC | 4987 |
| 1738 | CAGAUCAA G UUCAGAAC | 1527 | GUUCUGAA GGA GCCGUUAGGC UCCCUUCAAGGA GCCGUUAGGC UCCGGG UUGAUCUG | 4988 |
| 1751 | GAACAAUG G CCUCAUUG | 1528 | CAAUGAGG GGA GCCGUUAGGC UCCCUUCAAGGA GCCGUUAGGC UCCGGG CAUUGUUC | 4989 |
| 1771 | CUUUUGGG G CCCUUUCA | 1529 | UGAAAGGG GGA GCCGUUAGGC UCCCUUCAAGGA GCCGUUAGGC UCCGGG CCCAAAAG | 4990 |
| 1792 | GAAAUGGA G CUGUCUCU | 1530 | AGAGACAG GGA GCCGUUAGGC UCCCUUCAAGGA GCCGUUAGGC UCCGGG UCCAUUUC | 4991 |
| 1803 | GUCUCUCA G CGCUCCAU | 1531 | AUGGAGCG GGA GCCGUUAGGC UCCCUUCAAGGA GCCGUUAGGC UCCGGG UGAGAGAC | 4992 |
| 1815 | UCCAUCCA G CUUGAGAG | 1532 | CUCUCAAG GGA GCCGUUAGGC UCCCUUCAAGGA GCCGUUAGGC UCCGGG UGGAUGGA | 4993 |
| 1823 | GCUUGAGA G UAAGGGAU | 1533 | AUCCCUUA GGA GCCGUUAGGC UCCCUUCAAGGA GCCGUUAGGC UCCGGG UCUCAAGC | 4994 |
| 1847 | CCAGAACA G CCAGUGGA | 1534 | UCCACUGG GGA GCCGUUAGGC UCCCUUCAAGGA GCCGUUAGGC UCCGGG UGUUCUGG | 4995 |
| 1851 | AACAGCCA G UGGAUGAA | 1535 | UUCAUCCA GGA GCCGUUAGGC UCCCUUCAAGGA GCCGUUAGGC UCCGGG UGGCUGUU | 4996 |
| 1862 | GAUGAAUG G CACAGUGA | 1536 | UCACUGUG GGA GCCGUUAGGC UCCCUUCAAGGA GCCGUUAGGC UCCGGG CAUUCAUC | 4997 |
| 1867 | AUGGCACA G UGAUCGUG | 1537 | CACGAUCA GGA GCCGUUAGGC UCCCUUCAAGGA GCCGUUAGGC UCCGGG UGUGCCAU | 4998 |
| 1873 | CAGUGAUC G UGGACAGC | 1538 | GCUGUCCA GGA GCCGUUAGGC UCCCUUCAAGGA GCCGUUAGGC UCCGGG GAUCACUG | 4999 |
| 1880 | CGUGGACA G CACCGUGG | 1539 | CCACGGUG GGA GCCGUUAGGC UCCCUUCAAGGA GCCGUUAGGC UCCGGG UGUCCACG | 5000 |
| 1885 | ACAGCACC G UGGGAAAG | 1540 | CUUUCCCA GGA GCCGUUAGGC UCCCUUCAAGGA GCCGUUAGGC UCCGGG GGUGCUGU | 5001 |
| 1926 | ACAACGCA G CCUCCCCA | 1541 | UGGGGAGG GGA GCCGUUAGGC UCCCUUCAAGGA GCCGUUAGGC UCCGGG UGCGUUGU | 5002 |
| 1955 | GGAUCCCA G UGGACAGA | 1542 | UCUGUCCA GGA GCCGUUAGGC UCCCUUCAAGGA GCCGUUAGGC UCCGGG UGGGAUCC | 5003 |
| 1965 | GGACAGAA G CAAGGUGG | 1543 | CCACCUUG GGA GCCGUUAGGC UCCCUUCAAGGA GCCGUUAGGC UCCGGG UUCUGUCC | 5004 |
| 1970 | GAAGCAAG G UGGCUUUG | 1544 | CAAAGCCA GGA GCCGUUAGGC UCCCUUCAAGGA GCCGUUAGGC UCCGGG CUUGCUUC | 5005 |
| 1973 | GCAAGGUG G CUUUGUAG | 1545 | CUACAAAG GGA GCCGUUAGGC UCCCUUCAAGGA GCCGUUAGGC UCCGGG CACCUUGC | 5006 |
| 1981 | GCUUUGUA G UGGACAAA | 1546 | UUUGUCCA GGA GCCGUUAGGC UCCCUUCAAGGA GCCGUUAGGC UCCGGG UACAAAGC | 5007 |
| 2002 | CCAAAAUG G CCUACCUC | 1547 | GAGGUAGG GGA GCCGUUAGGC UCCCUUCAAGGA GCCGUUAGGC UCCGGG CAUUUUGG | 5008 |
| 2021 | AAUCCCAG G CAUUGCUA | 1548 | UAGCAAUG GGA GCCGUUAGGC UCCCUUCAAGGA GCCGUUAGGC UCCGGG CUGGGAUU | 5009 |
| 2032 | UUGCUAAG G UUGGCACU | 1549 | AGUGCCAA GGA GCCGUUAGGC UCCCUUCAAGGA GCCGUUAGGC UCCGGG CUUAGCAA | 5010 |
| 2036 | UAAGGUUG G CACUUGGA | 1550 | UCCAAGUG GGA GCCGUUAGGC UCCCUUCAAGGA GCCGUUAGGC UCCGGG CAACCUUA | 5011 |
| 2051 | GAAAUACA G UCUGCAAG | 1551 | CUUGCAGA GGA GCCGUUAGGC UCCCUUCAAGGA GCCGUUAGGC UCCGGG UGUAUUUC | 5012 |
| 2059 | GUCUGCAA G CAAGCUCA | 1552 | UGAGCUUG GGA GCCGUUAGGC UCCCUUCAAGGA GCCGUUAGGC UCCGGG UUGCAGAC | 5013 |
| 2063 | GCAAGCAA G CUCACAAA | 1553 | UUUGUGAG GGA GCCGUUAGGC UCCCUUCAAGGA GCCGUUAGGC UCCGGG UUGCUUGC | 5014 |
| 2091 | ACUGUCAC G UCCCGUGC | 1554 | GCACGGGA GGA GCCGUUAGGC UCCCUUCAAGGA GCCGUUAGGC UCCGGG GUGACAGU | 5015 |
| 2096 | CACGUCCC G UGCGUCCA | 1555 | UGGACGCA GGA GCCGUUAGGC UCCCUUCAAGGA GCCGUUAGGC UCCGGG GGGACGUG | 5016 |
| 2100 | UCCCGUGC G UCCAAUGC | 1556 | GCAUUGGA GGA GCCGUUAGGC UCCCUUCAAGGA GCCGUUAGGC UCCGGG GCACGGGA | 5017 |
| 2128 | CAAUUACA G UGACUUCC | 1557 | GGAAGUCA GGA GCCGUUAGGC UCCCUUCAAGGA GCCGUUAGGC UCCGGG UGUAAUUG | 5018 |
| 2156 | GGACACCA G CAAAUUCC | 1558 | GGAAUUUG GGA GCCGUUAGGC UCCCUUCAAGGA GCCGUUAGGC UCCGGG UGGUGUCC | 5019 |
| 2168 | AUUCCCCA G CCCUCUGG | 1559 | CCAGAGGG GGA GCCGUUAGGC UCCCUUCAAGGA GCCGUUAGGC UCCGGG UGGGGAAU | 5020 |
| 2176 | GCCCUCUG G UAGUUUAU | 1560 | AUAAACUA GGA GCCGUUAGGC UCCCUUCAAGGA GCCGUUAGGC UCCGGG CAGAGGGC | 5021 |
| 2179 | CUCUGGUA G UUUAUGCA | 1561 | UGCAUAAA GGA GCCGUUAGGC UCCCUUCAAGGA GCCGUUAGGC UCCGGG UACCAGAG | 5022 |
| 2203 | GCCAAGGA G CCUCCCCA | 1562 | UGGGGAGG GGA GCCGUUAGGC UCCCUUCAAGGA GCCGUUAGGC UCCGGG UCCUUGGC | 5023 |
| 2221 | UUCUCAGG G CCAGUGUC | 1563 | GACACUGG GGA GCCGUUAGGC UCCCUUCAAGGA GCCGUUAGGC UCCGGG CCUGAGAA | 5024 |
| 2225 | CAGGGCCA G UGUCACAG | 1564 | CUGUGACA GGA GCCGUUAGGC UCCCUUCAAGGA GCCGUUAGGC UCCGGG UGGCCCUG | 5025 |
| 2233 | GUGUCACA G CCCUGAUU | 1565 | AAUCAGGG GGA GCCGUUAGGC UCCCUUCAAGGA GCCGUUAGGC UCCGGG UGUGACAC | 5026 |
| 2248 | UUGAAUCA G UGAAUGGA | 1566 | UCCAUUCA GGA GCCGUUAGGC UCCCUUCAAGGA GCCGUUAGGC UCCGGG UGAUUCAA | 5027 |
| 2263 | GAAAAACA G UUACCUUG | 1567 | CAAGGUAA GGA GCCGUUAGGC UCCCUUCAAGGA GCCGUUAGGC UCCGGG UGUUUUUC | 5028 |
| 2290 | AUAAUGGA G CAGGUGCU | 1568 | AGCACCUG GGA GCCGUUAGGC UCCCUUCAAGGA GCCGUUAGGC UCCGGG UCCAUUAU | 5029 |
| 2294 | UGGAGCAG G UGCUGAUG | 1569 | CAUCAGCA GGA GCCGUUAGGC UCCCUUCAAGGA GCCGUUAGGC UCCGGG CUGCUCCA | 5030 |
| 2318 | GGAUGACG G UGUCUACU | 1570 | AGUAGACA GGA GCCGUUAGGC UCCCUUCAAGGA GCCGUUAGGC UCCGGG CGUCAUCC | 5031 |
| 2331 | UACUCAAG G UAUUUCAC | 1571 | GUGAAAUA GGA GCCGUUAGGC UCCCUUCAAGGA GCCGUUAGGC UCCGGG CUUGAGUA | 5032 |
| 2357 | CACGAAUG G UAGAUACA | 1572 | UGUAUCUA GGA GCCGUUAGGC UCCCUUCAAGGA GCCGUUAGGC UCCGGG CAUUCGUG | 5033 |
| 2366 | UAGAUACA G UGUAAAAG | 1573 | CUUUUACA GGA GCCGUUAGGC UCCCUUCAAGGA GCCGUUAGGC UCCGGG UGUAUCUA | 5034 |
| 2374 | GUGUAAAA G UGCGGGCU | 1574 | AGCCCGCA GGA GCCGUUAGGC UCCCUUCAAGGA GCCGUUAGGC UCCGGG UUUUACAC | 5035 |
| 2380 | AAGUGCGG G CUCUGGGA | 1575 | UCCCAGAG GGA GCCGUUAGGC UCCCUUCAAGGA GCCGUUAGGC UCCGGG CCGCACUU | 5036 |
| 2392 | UGGGAGGA G UUAACGCA | 1576 | UGCGUUAA GGA GCCGUUAGGC UCCCUUCAAGGA GCCGUUAGGC UCCGGG UCCUCCCA | 5037 |
| 2401 | UUAACGCA G CCAGACGG | 1577 | CCGUCUGG GGA GCCGUUAGGC UCCCUUCAAGGA GCCGUUAGGC UCCGGG UGCGUUAA | 5038 |
| 2413 | GACGGAGA G UGAUACCC | 1578 | GGGUAUCA GGA GCCGUUAGGC UCCCUUCAAGGA GCCGUUAGGC UCCGGG UCUCCGUC | 5039 |
| 2424 | AUACCCCA G CAGAGUGG | 1579 | CCACUCUG GGA GCCGUUAGGC UCCCUUCAAGGA GCCGUUAGGC UCCGGG UGGGGUAU | 5040 |
| 2429 | CCAGCAGA G UGGAGCAC | 1580 | GUGCUCCA GGA GCCGUUAGGC UCCCUUCAAGGA GCCGUUAGGC UCCGGG UCUGCUGG | 5041 |
| 2434 | AGAGUGGA G CACUGUAC | 1581 | GUACAGUG GGA GCCGUUAGGC UCCCUUCAAGGA GCCGUUAGGC UCCGGG UCCACUCU | 5042 |
| 2450 | CAUACCUG G CUGGAUUG | 1582 | CAAUCCAG GGA GCCGUUAGGC UCCCUUCAAGGA GCCGUUAGGC UCCGGG CAGGUAUG | 5043 |
| 2523 | CAACACAA G CAAGUGUG | 1583 | CACACUUG GGA GCCGUUAGGC UCCCUUCAAGGA GCCGUUAGGC UCCGGG UUGUGUUG | 5044 |
| 2527 | ACAAGCAA G UGUGUUUC | 1584 | GAAACACA GGA GCCGUUAGGC UCCCUUCAAGGA GCCGUUAGGC UCCGGG UUGCUUGU | 5045 |
| 2537 | GUGUUUCA G CAGAACAU | 1585 | AUGUUCUG GGA GCCGUUAGGC UCCCUUCAAGGA GCCGUUAGGC UCCGGG UGAAACAC | 5046 |
| 2555 | CUCGGGAG G CUCAUUUG | 1586 | CAAAUGAG GGA GCCGUUAGGC UCCCUUCAAGGA GCCGUUAGGC UCCGGG CUCCCGAG | 5047 |
| 2566 | CAUUUGUG G CUUCUGAU | 1587 | AUCAGAAG GGA GCCGUUAGGC UCCCUUCAAGGA GCCGUUAGGC UCCGGG CACAAAUG | 5048 |
| 2612 | CCCACCUG G CCAAAUCA | 1588 | UGAUUUGG GGA GCCGUUAGGC UCCCUUCAAGGA GCCGUUAGGC UCCGGG CAGGUGGG | 5049 |
| 2632 | ACCUGAAG G CGGAAAUU | 1589 | AAUUUCCG GGA GCCGUUAGGC UCCCUUCAAGGA GCCGUUAGGC UCCGGG CUUCAGGU | 5050 |
| 2648 | UCACGGGG G CAGUCUCA | 1590 | UGAGACUG GGA GCCGUUAGGC UCCCUUCAAGGA GCCGUUAGGC UCCGGG CCCCGUGA | 5051 |
| 2651 | CGGGGGCA G UCUCAUUA | 1591 | UAAUGAGA GGA GCCGUUAGGC UCCCUUCAAGGA GCCGUUAGGC UCCGGG UGCCCCCG | 5052 |
| 2674 | CUUGGACA G CUCCUGGG | 1592 | CCCAGGAG GGA GCCGUUAGGC UCCCUUCAAGGA GCCGUUAGGC UCCGGG UGUCCAAG | 5053 |
| 2704 | AUGGAACA G CUCACAAG | 1593 | CUUGUGAG GGA GCCGUUAGGC UCCCUUCAAGGA GCCGUUAGGC UCCGGG UGUUCCAU | 5054 |
| 2712 | GCUCACAA G UAUAUCAU | 1594 | AUGAUAUA GGA GCCGUUAGGC UCCCUUCAAGGA GCCGUUAGGC UCCGGG UUGUGAGC | 5055 |
| 2729 | UCGAAUAA G UACAAGUA | 1595 | UACUUGUA GGA GCCGUUAGGC UCCCUUCAAGGA GCCGUUAGGC UCCGGG UUAUUCGA | 5056 |
| 2735 | AAGUACAA G UAUUCUUG | 1596 | CAAGAAUA GGA GCCGUUAGGC UCCCUUCAAGGA GCCGUUAGGC UCCGGG UUGUACUU | 5057 |
| 2757 | AGAGACAA G UUCAAUGA | 1597 | UCAUUGAA GGA GCCGUUAGGC UCCCUUCAAGGA GCCGUUAGGC UCCGGG UUGUCUCU | 5058 |
| 2776 | CUCUUCAA G UGAAUACU | 1598 | AGUAUUCA GGA GCCGUUAGGC UCCCUUCAAGGA GCCGUUAGGC UCCGGG UUGAAGAG | 5059 |
| 2806 | CAAAGGAA G CCAACUCU | 1599 | AGAGUUGG GGA GCCGUUAGGC UCCCUUCAAGGA GCCGUUAGGC UCCGGG UUCCUUUG | 5060 |
| 2821 | CUGAGGAA G UCUUUUUG | 1600 | CAAAAAGA GGA GCCGUUAGGC UCCCUUCAAGGA GCCGUUAGGC UCCGGG UUCCUCAG | 5061 |
| 2861 | UGAAAAUG G CACAGAUC | 1601 | GAUCUGUG GGA GCCGUUAGGC UCCCUUCAAGGA GCCGUUAGGC UCCGGG CAUUUUCA | 5062 |
| 2887 | CUAUUCAG G CUGUUGAU | 1602 | AUCAACAG GGA GCCGUUAGGC UCCCUUCAAGGA GCCGUUAGGC UCCGGG CUGAAUAG | 5063 |
| 2899 | UUGAUAAG G UCGAUCUG | 1603 | CAGAUCGA GGA GCCGUUAGGC UCCCUUCAAGGA GCCGUUAGGC UCCGGG CUUAUCAA | 5064 |
| 2935 | UUGCACGA G UAUCUUUG | 1604 | CAAAGAUA GGA GCCGUUAGGC UCCCUUCAAGGA GCCGUUAGGC UCCGGG UCGUGCAA | 5065 |
| 2978 | GACACCUA G UCCUGAUG | 1605 | CAUCAGGA GGA GCCGUUAGGC UCCCUUCAAGGA GCCGUUAGGC UCCGGG UAGGUGUC | 5066 |
| 2991 | GAUGAAAC G UCUGCUCC | 1606 | GGAGCAGA GGA GCCGUUAGGC UCCCUUCAAGGA GCCGUUAGGC UCCGGG GUUUCAUC | 5067 |
| 3023 | UAUCAACA G CACCAUUC | 1607 | GAAUGGUG GGA GCCGUUAGGC UCCCUUCAAGGA GCCGUUAGGC UCCGGG UGUUGAUA | 5068 |
| 3035 | CAUUCCUG G CAUUCACA | 1608 | UGUGAAUG GGA GCCGUUAGGC UCCCUUCAAGGA GCCGUUAGGC UCCGGG CAGGAAUG | 5069 |
| 3063 | AUGUGGAA G UGGAUAGG | 1609 | CCUAUCCA GGA GCCGUUAGGC UCCCUUCAAGGA GCCGUUAGGC UCCGGG UUCCACAU | 5070 |
| 3081 | GAACUGCA G CUGUCAAU | 1610 | AUUGACAG GGA GCCGUUAGGC UCCCUUCAAGGA GCCGUUAGGC UCCGGG UGCAGUUC | 5071 |
| 3091 | UGUCAAUA G CCUAGGGC | 1611 | GCCCUAGG GGA GCCGUUAGGC UCCCUUCAAGGA GCCGUUAGGC UCCGGG UAUUGACA | 5072 |
| 3098 | AGCCUAGG G CUGAAUUU | 1612 | AAAUUCAG GGA GCCGUUAGGC UCCCUUCAAGGA GCCGUUAGGC UCCGGG CCUAGGCU | 5073 |
| 3189 | UGUAGGGG G CGAUAUAC | 1613 | GUAUAUCG GGA GCCGUUAGGC UCCCUUCAAGGA GCCGUUAGGC UCCGGG CCCCUACA | 5074 |
| 3242 | UGUAGGGG G CGAUAUAC | 1613 | GUAUAUCG GGA GCCGUUAGGC UCCCUUCAAGGA GCCGUUAGGC UCCGGG CCCCUACA | 5074 |
| 3210 | UGUAUAUA G UACAUUUA | 1614 | UAAAUGUA GGA GCCGUUAGGC UCCCUUCAAGGA GCCGUUAGGC UCCGGG UAUAUACA | 5075 |
| 3279 | UGUAGGGG G CGAUAAAA | 1615 | UUUUAUCG GGA GCCGUUAGGC UCCCUUCAAGGA GCCGUUAGGC UCCGGG CCCCUACA | 5076 |
| 14 | AUGCUUUU G GUACAAAU | 1868 | AUUUGUAC GGA GCCGUUAGGC UCCCUUCAAGGA GCCGUUAGGC UCCGGG AAAAGCAU | 5077 |
| 23 | GUACAAAU G GAUGUGGA | 1869 | UCCACAUC GGA GCCGUUAGGC UCCCUUCAAGGA GCCGUUAGGC UCCGGG AUUUGUAC | 5078 |
| 24 | UACAAAUG G AUGUGGAA | 1870 | UUCCACAU GGA GCCGUUAGGC UCCCUUCAAGGA GCCGUUAGGC UCCGGG CAUUUGUA | 5079 |
| 29 | AUGGAUGU G GAAUAUAA | 1871 | UUAUAUUC GGA GCCGUUAGGC UCCCUUCAAGGA GCCGUUAGGC UCCGGG ACAUCCAU | 5080 |
| 30 | UGGAUGUG G AAUAUAAU | 1872 | AUUAUAUU GGA GCCGUUAGGC UCCCUUCAAGGA GCCGUUAGGC UCCGGG CACAUCCA | 5081 |
| 58 | UUGUUUAA G GGGAGCAU | 1873 | AUGCUCCC GGA GCCGUUAGGC UCCCUUCAAGGA GCCGUUAGGC UCCGGG UUAAACAA | 5082 |
| 59 | UGUUUAAG G GGAGCAUG | 1874 | CAUGCUCC GGA GCCGUUAGGC UCCCUUCAAGGA GCCGUUAGGC UCCGGG CUUAAACA | 5083 |
| 60 | GUUUAAGG G GAGCAUGA | 1875 | UCAUGCUC GGA GCCGUUAGGC UCCCUUCAAGGA GCCGUUAGGC UCCGGG CCUUAAAC | 5084 |
| 61 | UUUAAGGG G AGCAUGAA | 1876 | UUCAUGCU GGA GCCGUUAGGC UCCCUUCAAGGA GCCGUUAGGC UCCGGG CCCUUAAA | 5085 |
| 70 | AGCAUGAA G AGGUGUUG | 1877 | CAACACCU GGA GCCGUUAGGC UCCCUUCAAGGA GCCGUUAGGC UCCGGG UUCAUGCU | 5086 |
| 72 | CAUGAAGA G GUGUUGAG | 1878 | CUCAACAC GGA GCCGUUAGGC UCCCUUCAAGGA GCCGUUAGGC UCCGGG UCUUCAUG | 5087 |
| 80 | GGUGUUGA G GUUAUGUC | 1879 | GACAUAAC GGA GCCGUUAGGC UCCCUUCAAGGA GCCGUUAGGC UCCGGG UCAACACC | 5088 |
| 97 | AAGCAUCU G GCACAGCU | 1880 | AGCUGUGC GGA GCCGUUAGGC UCCCUUCAAGGA GCCGUUAGGC UCCGGG AGAUGCUU | 5089 |
| 109 | CAGCUGAA G GCAGAUGG | 1881 | CCAUCUGC GGA GCCGUUAGGC UCCCUUCAAGGA GCCGUUAGGC UCCGGG UUCAGCUG | 5090 |
| 113 | UGAAGGCA G AUGGAAAU | 1882 | AUUUCCAU GGA GCCGUUAGGC UCCCUUCAAGGA GCCGUUAGGC UCCGGG UGCCUUCA | 5091 |
| 116 | AGGCAGAU G GAAAUAUU | 1883 | AAUAUUUC GGA GCCGUUAGGC UCCCUUCAAGGA GCCGUUAGGC UCCGGG AUCUGCCU | 5092 |
| 117 | GGCAGAUG G AAAUAUUU | 1884 | AAAUAUUU GGA GCCGUUAGGC UCCCUUCAAGGA GCCGUUAGGC UCCGGG CAUCUGCC | 5093 |
| 143 | CAAUUUGA G ACUAAGAU | 1885 | AUCUUAGU GGA GCCGUUAGGC UCCCUUCAAGGA GCCGUUAGGC UCCGGG UCAAAUUG | 5094 |
| 149 | GAGACUAA G AUAUUGUU | 1886 | AACAAUAU GGA GCCGUUAGGC UCCCUUCAAGGA GCCGUUAGGC UCCGGG UUAGUCUC | 5095 |
| 175 | CUAUUGAA G ACAAGAGC | 1887 | GCUCUUGU GGA GCCGUUAGGC UCCCUUCAAGGA GCCGUUAGGC UCCGGG UUCAAUAG | 5096 |
| 180 | GAAGACAA G AGCAAUAG | 1888 | CUAUUGCU GGA GCCGUUAGGC UCCCUUCAAGGA GCCGUUAGGC UCCGGG UUGUCUUC | 5097 |
| 201 | ACACAUCA G GUCAGGGG | 1889 | CCCCUGAC GGA GCCGUUAGGC UCCCUUCAAGGA GCCGUUAGGC UCCGGG UGAUGUGU | 5098 |
| 206 | UCAGGUCA G GGGGUUAA | 1890 | UUAACCCC GGA GCCGUUAGGC UCCCUUCAAGGA GCCGUUAGGC UCCGGG UGACCUGA | 5099 |
| 207 | CAGGUCAG G GGGUUAAA | 1891 | UUUAACCC GGA GCCGUUAGGC UCCCUUCAAGGA GCCGUUAGGC UCCGGG CUGACCUG | 5100 |
| 208 | AGGUCAGG G GGUUAAAG | 1892 | CUUUAACC GGA GCCGUUAGGC UCCCUUCAAGGA GCCGUUAGGC UCCGGG CCUGACCU | 5101 |
| 209 | GGUCAGGG G GUUAAAGA | 1893 | UCUUUAAC GGA GCCGUUAGGC UCCCUUCAAGGA GCCGUUAGGC UCCGGG CCCUGACC | 5102 |
| 216 | GGGUUAAA G ACCUGUGA | 1894 | UCACAGGU GGA GCCGUUAGGC UCCCUUCAAGGA GCCGUUAGGC UCCGGG UUUAACCC | 5103 |
| 245 | GAUAAGUU G GAAACGUG | 1895 | CACGUUUC GGA GCCGUUAGGC UCCCUUCAAGGA GCCGUUAGGC UCCGGG AACUUAUC | 5104 |
| 246 | AUAAGUUG G AAACGUGU | 1896 | ACACGUUU GGA GCCGUUAGGC UCCCUUCAAGGA GCCGUUAGGC UCCGGG CAACUUAU | 5105 |
| 286 | UAUAUAAU G GUAAAGAA | 1897 | UUCUUUAC GGA GCCGUUAGGC UCCCUUCAAGGA GCCGUUAGGC UCCGGG AUUAUAUA | 5106 |
| 292 | AUGGUAAA G AAAGACAC | 1898 | GUGUCUUU GGA GCCGUUAGGC UCCCUUCAAGGA GCCGUUAGGC UCCGGG UUUACCAU | 5107 |
| 296 | UAAAGAAA G ACACCUUC | 1899 | GAAGGUGU GGA GCCGUUAGGC UCCCUUCAAGGA GCCGUUAGGC UCCGGG UUUCUUUA | 5108 |
| 324 | UUUCCAAA G AGAGGAAU | 1900 | AUUCCUCU GGA GCCGUUAGGC UCCCUUCAAGGA GCCGUUAGGC UCCGGG UUUGGAAA | 5109 |
| 326 | UCCAAAGA G AGGAAUCA | 1901 | UGAUUCCU GGA GCCGUUAGGC UCCCUUCAAGGA GCCGUUAGGC UCCGGG UCUUUGGA | 5110 |
| 328 | CAAAGAGA G GAAUCACA | 1902 | UGUGAUUC GGA GCCGUUAGGC UCCCUUCAAGGA GCCGUUAGGC UCCGGG UCUCUUUG | 5111 |
| 329 | AAAGAGAG G AAUCACAG | 1903 | CUGUGAUU GGA GCCGUUAGGC UCCCUUCAAGGA GCCGUUAGGC UCCGGG CUCUCUUU | 5112 |
| 337 | GAAUCACA G GGAGAUGU | 1904 | ACAUCUCC GGA GCCGUUAGGC UCCCUUCAAGGA GCCGUUAGGC UCCGGG UGUGAUUC | 5113 |
| 338 | AAUCACAG G GAGAUGUA | 1905 | UACAUCUC GGA GCCGUUAGGC UCCCUUCAAGGA GCCGUUAGGC UCCGGG CUGUGAUU | 5114 |
| 339 | AUCACAGG G AGAUGUAC | 1906 | GUACAUCU GGA GCCGUUAGGC UCCCUUCAAGGA GCCGUUAGGC UCCGGG CCUGUGAU | 5115 |
| 341 | CACAGGGA G AUGUACAG | 1907 | CUGUACAU GGA GCCGUUAGGC UCCCUUCAAGGA GCCGUUAGGC UCCGGG UCCCUGUG | 5116 |
| 354 | ACAGCAAU G GGGCCAUU | 1908 | AAUGGCCC GGA GCCGUUAGGC UCCCUUCAAGGA GCCGUUAGGC UCCGGG AUUGCUGU | 5117 |
| 355 | CAGCAAUG G GGCCAUUU | 1909 | AAAUGGCC GGA GCCGUUAGGC UCCCUUCAAGGA GCCGUUAGGC UCCGGG CAUUGCUG | 5118 |
| 356 | AGCAAUGG G GCCAUUUA | 1910 | UAAAUGGC GGA GCCGUUAGGC UCCCUUCAAGGA GCCGUUAGGC UCCGGG CCAUUGCU | 5119 |
| 366 | CCAUUUAA G AGUUCUGU | 1911 | ACAGAACU GGA GCCGUUAGGC UCCCUUCAAGGA GCCGUUAGGC UCCGGG UUAAAUGG | 5120 |
| 400 | ACCUUCUA G AAGGGGCC | 1912 | GGCCCCUU GGA GCCGUUAGGC UCCCUUCAAGGA GCCGUUAGGC UCCGGG UAGAAGGU | 5121 |
| 403 | UUCUAGAA G GGGCCCUG | 1913 | CAGGGCCC GGA GCCGUUAGGC UCCCUUCAAGGA GCCGUUAGGC UCCGGG UUCUAGAA | 5122 |
| 404 | UCUAGAAG G GGCCCUGA | 1914 | UCAGGGCC GGA GCCGUUAGGC UCCCUUCAAGGA GCCGUUAGGC UCCGGG CUUCUAGA | 5123 |
| 405 | CUAGAAGG G GCCCUGAG | 1915 | CUCAGGGC GGA GCCGUUAGGC UCCCUUCAAGGA GCCGUUAGGC UCCGGG CCUUCUAG | 5124 |
| 442 | ACAACAAU G GCUAUGAA | 1916 | UUCAUAGC GGA GCCGUUAGGC UCCCUUCAAGGA GCCGUUAGGC UCCGGG AUUGUUGU | 5125 |
| 451 | GCUAUGAA G GCAUUGUC | 1917 | GACAAUGC GGA GCCGUUAGGC UCCCUUCAAGGA GCCGUUAGGC UCCGGG UUCAUAGC | 5126 |
| 484 | AUGUGCCA G AAGAUGAA | 1918 | UUCAUCUU GGA GCCGUUAGGC UCCCUUCAAGGA GCCGUUAGGC UCCGGG UGGCACAU | 5127 |
| 487 | UGCCAGAA G AUGAAACA | 1919 | UGUUUCAU GGA GCCGUUAGGC UCCCUUCAAGGA GCCGUUAGGC UCCGGG UUCUGGCA | 5128 |
| 513 | CAAAUAAA G GACAUGGU | 1920 | ACCAUGUC GGA GCCGUUAGGC UCCCUUCAAGGA GCCGUUAGGC UCCGGG UUUAUUUG | 5129 |
| 514 | AAAUAAAG G ACAUGGUG | 1921 | CACCAUGU GGA GCCGUUAGGC UCCCUUCAAGGA GCCGUUAGGC UCCGGG CUUUAUUU | 5130 |
| 519 | AAGGACAU G GUGACCCA | 1922 | UGGGUCAC GGA GCCGUUAGGC UCCCUUCAAGGA GCCGUUAGGC UCCGGG AUGUCCUU | 5131 |
| 528 | GUGACCCA G GCAUCUCU | 1923 | AGAGAUGC GGA GCCGUUAGGC UCCCUUCAAGGA GCCGUUAGGC UCCGGG UGGGUCAC | 5132 |
| 556 | AAGCUACA G GAAAGCGA | 1924 | UCGCUUUC GGA GCCGUUAGGC UCCCUUCAAGGA GCCGUUAGGC UCCGGG UGUAGCUU | 5133 |
| 557 | AGCUACAG G AAAGCGAU | 1925 | AUCGCUUU GGA GCCGUUAGGC UCCCUUCAAGGA GCCGUUAGGC UCCGGG CUGUAGCU | 5134 |
| 605 | UGAAACAU G GAAGACAA | 1926 | UUGUCUUC GGA GCCGUUAGGC UCCCUUCAAGGA GCCGUUAGGC UCCGGG AUGUUUCA | 5135 |
| 606 | GAAACAUG G AAGACAAA | 1927 | UUUGUCUU GGA GCCGUUAGGC UCCCUUCAAGGA GCCGUUAGGC UCCGGG CAUGUUUC | 5136 |
| 609 | ACAUGGAA G ACAAAGGC | 1928 | GCCUUUGU GGA GCCGUUAGGC UCCCUUCAAGGA GCCGUUAGGC UCCGGG UUCCAUGU | 5137 |
| 615 | AAGACAAA G GCUGACUA | 1929 | UAGUCAGC GGA GCCGUUAGGC UCCCUUCAAGGA GCCGUUAGGC UCCGGG UUUGUCUU | 5138 |
| 629 | CUAUGUGA G ACCAAAAC | 1930 | GUUUUGGU GGA GCCGUUAGGC UCCCUUCAAGGA GCCGUUAGGC UCCGGG UCACAUAG | 5139 |
| 642 | AAACUUGA G ACCUACAA | 1931 | UUGUAGGU GGA GCCGUUAGGC UCCCUUCAAGGA GCCGUUAGGC UCCGGG UCAAGUUU | 5140 |
| 666 | GAUGUUCU G GUUGCUGA | 1932 | UCAGCAAC GGA GCCGUUAGGC UCCCUUCAAGGA GCCGUUAGGC UCCGGG AGAACAUC | 5141 |
| 688 | CUCCUCCA G GUAAUGAU | 1933 | AUCAUUAC GGA GCCGUUAGGC UCCCUUCAAGGA GCCGUUAGGC UCCGGG UGGAGGAG | 5142 |
| 714 | ACUGAGCA G AUGGGCAA | 1934 | UUGCCCAU GGA GCCGUUAGGC UCCCUUCAAGGA GCCGUUAGGC UCCGGG UGCUCAGU | 5143 |
| 717 | GAGCAGAU G GGCAACUG | 1935 | CAGUUGCC GGA GCCGUUAGGC UCCCUUCAAGGA GCCGUUAGGC UCCGGG AUCUGCUC | 5144 |
| 718 | AGCAGAUG G GCAACUGU | 1936 | ACAGUUGC GGA GCCGUUAGGC UCCCUUCAAGGA GCCGUUAGGC UCCGGG CAUCUGCU | 5145 |
| 727 | GCAACUGU G GAGAGAAG | 1937 | CUUCUCUC GGA GCCGUUAGGC UCCCUUCAAGGA GCCGUUAGGC UCCGGG ACAGUUGC | 5146 |
| 728 | CAACUGUG G AGAGAAGG | 1938 | CCUUCUCU GGA GCCGUUAGGC UCCCUUCAAGGA GCCGUUAGGC UCCGGG CACAGUUG | 5147 |
| 730 | ACUGUGGA G AGAAGGGU | 1939 | ACCCUUCU GGA GCCGUUAGGC UCCCUUCAAGGA GCCGUUAGGC UCCGGG UCCACAGU | 5148 |
| 732 | UGUGGAGA G AAGGGUGA | 1940 | UCACCCUU GGA GCCGUUAGGC UCCCUUCAAGGA GCCGUUAGGC UCCGGG UCUCCACA | 5149 |
| 735 | GGAGAGAA G GGUGAAAG | 1941 | CUUUCACC GGA GCCGUUAGGC UCCCUUCAAGGA GCCGUUAGGC UCCGGG UUCUCUCC | 5150 |
| 736 | GAGAGAAG G GUGAAAGG | 1942 | CCUUUCAC GGA GCCGUUAGGC UCCCUUCAAGGA GCCGUUAGGC UCCGGG CUUCUCUC | 5151 |
| 743 | GGGUGAAA G GAUCCACC | 1943 | GGUGGAUC GGA GCCGUUAGGC UCCCUUCAAGGA GCCGUUAGGC UCCGGG UUUCACCC | 5152 |
| 744 | GGUGAAAG G AUCCACCU | 1944 | AGGUGGAU GGA GCCGUUAGGC UCCCUUCAAGGA GCCGUUAGGC UCCGGG CUUUCACC | 5153 |
| 772 | UCAUUGCA G GAAAAAAG | 1945 | CUUUUUUC GGA GCCGUUAGGC UCCCUUCAAGGA GCCGUUAGGC UCCGGG UGCAAUGA | 5154 |
| 773 | CAUUGCAG G AAAAAAGU | 1946 | ACUUUUUU GGA GCCGUUAGGC UCCCUUCAAGGA GCCGUUAGGC UCCGGG CUGCAAUG | 5155 |
| 793 | CUGAAUAU G GACCACAA | 1947 | UUGUGGUC GGA GCCGUUAGGC UCCCUUCAAGGA GCCGUUAGGC UCCGGG AUAUUCAG | 5156 |
| 794 | UGAAUAUG G ACCACAAG | 1948 | CUUGUGGU GGA GCCGUUAGGC UCCCUUCAAGGA GCCGUUAGGC UCCGGG CAUAUUCA | 5157 |
| 802 | GACCACAA G GUAAGGCA | 1949 | UGCCUUAC GGA GCCGUUAGGC UCCCUUCAAGGA GCCGUUAGGC UCCGGG UUGUGGUC | 5158 |
| 807 | CAAGGUAA G GCAUUUGU | 1950 | ACAAAUGC GGA GCCGUUAGGC UCCCUUCAAGGA GCCGUUAGGC UCCGGG UUACCUUG | 5159 |
| 824 | CCAUGAGU G GGCUCAUC | 1951 | GAUGAGCC GGA GCCGUUAGGC UCCCUUCAAGGA GCCGUUAGGC UCCGGG ACUCAUGG | 5160 |
| 825 | CAUGAGUG G GCUCAUCU | 1952 | AGAUGAGC GGA GCCGUUAGGC UCCCUUCAAGGA GCCGUUAGGC UCCGGG CACUCAUG | 5161 |
| 839 | UCUACGAU G GGGAGUAU | 1953 | AUACUCCC GGA GCCGUUAGGC UCCCUUCAAGGA GCCGUUAGGC UCCGGG AUCGUAGA | 5162 |
| 840 | CUACGAUG G GGAGUAUU | 1954 | AAUACUCC GGA GCCGUUAGGC UCCCUUCAAGGA GCCGUUAGGC UCCGGG CAUCGUAG | 5163 |
| 841 | UACGAUGG G GAGUAUUU | 1955 | AAAUACUC GGA GCCGUUAGGC UCCCUUCAAGGA GCCGUUAGGC UCCGGG CCAUCGUA | 5164 |
| 842 | ACGAUGGG G AGUAUUUG | 1956 | CAAAUACU GGA GCCGUUAGGC UCCCUUCAAGGA GCCGUUAGGC UCCGGG CCCAUCGU | 5165 |
| 870 | AAUGAUGA G AAAUUCUA | 1957 | UAGAAUUU GGA GCCGUUAGGC UCCCUUCAAGGA GCCGUUAGGC UCCGGG UCAUCAUU | 5166 |
| 889 | UAUCCAAU G GAAGAAUA | 1958 | UAUUCUUC GGA GCCGUUAGGC UCCCUUCAAGGA GCCGUUAGGC UCCGGG AUUGGAUA | 5167 |
| 890 | AUCCAAUG G AAGAAUAC | 1959 | GUAUUCUU GGA GCCGUUAGGC UCCCUUCAAGGA GCCGUUAGGC UCCGGG CAUUGGAU | 5168 |
| 893 | CAAUGGAA G AAUACAAG | 1960 | CUUGUAUU GGA GCCGUUAGGC UCCCUUCAAGGA GCCGUUAGGC UCCGGG UUCCAUUG | 5169 |
| 908 | AGCAGUAA G AUGUUCAG | 1961 | CUGAACAU GGA GCCGUUAGGC UCCCUUCAAGGA GCCGUUAGGC UCCGGG UUACUGCU | 5170 |
| 919 | GUUCAGCA G GUAUUACU | 1962 | AGUAAUAC GGA GCCGUUAGGC UCCCUUCAAGGA GCCGUUAGGC UCCGGG UGCUGAAC | 5171 |
| 928 | GUAUUACU G GUACAAAU | 1963 | AUUUGUAC GGA GCCGUUAGGC UCCCUUCAAGGA GCCGUUAGGC UCCGGG AGUAAUAC | 5172 |
| 945 | GUAGUAAA G AAGUGUCA | 1964 | UGACACUU GGA GCCGUUAGGC UCCCUUCAAGGA GCCGUUAGGC UCCGGG UUUACUAC | 5173 |
| 954 | AAGUGUCA G GGAGGCAG | 1965 | CUGCCUCC GGA GCCGUUAGGC UCCCUUCAAGGA GCCGUUAGGC UCCGGG UGACACUU | 5174 |
| 955 | AGUGUCAG G GAGGCAGC | 1966 | GCUGCCUC GGA GCCGUUAGGC UCCCUUCAAGGA GCCGUUAGGC UCCGGG CUGACACU | 5175 |
| 956 | GUGUCAGG G AGGCAGCU | 1967 | AGCUGCCU GGA GCCGUUAGGC UCCCUUCAAGGA GCCGUUAGGC UCCGGG CCUGACAC | 5176 |
| 958 | GUCAGGGA G GCAGCUGU | 1968 | ACAGCUGC GGA GCCGUUAGGC UCCCUUCAAGGA GCCGUUAGGC UCCGGG UCCCUGAC | 5177 |
| 977 | CACCAAAA G AUGCACAU | 1969 | AUGUGCAU GGA GCCGUUAGGC UCCCUUCAAGGA GCCGUUAGGC UCCGGG UUUUGGUG | 5178 |
| 1000 | AAGUUACA G GACUCUAU | 1970 | AUAGAGUC GGA GCCGUUAGGC UCCCUUCAAGGA GCCGUUAGGC UCCGGG UGUAACUU | 5179 |
| 1001 | AGUUACAG G ACUCUAUG | 1971 | CAUAGAGU GGA GCCGUUAGGC UCCCUUCAAGGA GCCGUUAGGC UCCGGG CUGUAACU | 5180 |
| 1015 | AUGAAAAA G GAUGUGAG | 1972 | CUCACAUC GGA GCCGUUAGGC UCCCUUCAAGGA GCCGUUAGGC UCCGGG UUUUUCAU | 5181 |
| 1016 | UGAAAAAG G AUGUGAGU | 1973 | ACUCACAU GGA GCCGUUAGGC UCCCUUCAAGGA GCCGUUAGGC UCCGGG CUUUUUCA | 5182 |
| 1044 | UCCCGCCA G ACGGAGAA | 1974 | UUCUCCGU GGA GCCGUUAGGC UCCCUUCAAGGA GCCGUUAGGC UCCGGG UGGCGGGA | 5183 |
| 1047 | CGCCAGAC G GAGAAGGC | 1975 | GCCUUCUC GGA GCCGUUAGGC UCCCUUCAAGGA GCCGUUAGGC UCCGGG GUCUGGCG | 5184 |
| 1048 | GCCAGACG G AGAAGGCU | 1976 | AGCCUUCU GGA GCCGUUAGGC UCCCUUCAAGGA GCCGUUAGGC UCCGGG CGUCUGGC | 5185 |
| 1050 | CAGACGGA G AAGGCUUC | 1977 | GAAGCCUU GGA GCCGUUAGGC UCCCUUCAAGGA GCCGUUAGGC UCCGGG UCCGUCUG | 5186 |
| 1053 | ACGGAGAA G GCUUCUAU | 1978 | AUAGAAGC GGA GCCGUUAGGC UCCCUUCAAGGA GCCGUUAGGC UCCGGG UUCUCCGU | 5187 |
| 1105 | UCUGUACA G AACAAAAC | 1979 | GUUUUGUU GGA GCCGUUAGGC UCCCUUCAAGGA GCCGUUAGGC UCCGGG UGUACAGA | 5188 |
| 1123 | ACAACAAA G AAGCUCCA | 1980 | UGGAGCUU GGA GCCGUUAGGC UCCCUUCAAGGA GCCGUUAGGC UCCGGG UUUGUUGU | 5189 |
| 1169 | AAGCACAU G GGAAGUGA | 1981 | UCACUUCC GGA GCCGUUAGGC UCCCUUCAAGGA GCCGUUAGGC UCCGGG AUGUGCUU | 5190 |
| 1170 | AGCACAUG G GAAGUGAU | 1982 | AUCACUUC GGA GCCGUUAGGC UCCCUUCAAGGA GCCGUUAGGC UCCGGG CAUGUGCU | 5191 |
| 1171 | GCACAUGG G AAGUGAUC | 1983 | GAUCACUU GGA GCCGUUAGGC UCCCUUCAAGGA GCCGUUAGGC UCCGGG CCAUGUGC | 5192 |
| 1191 | GAUUCUGA G GACUUUAA | 1984 | UUAAAGUC GGA GCCGUUAGGC UCCCUUCAAGGA GCCGUUAGGC UCCGGG UCAGAAUC | 5193 |
| 1192 | AUUCUGAG G ACUUUAAG | 1985 | CUUAAAGU GGA GCCGUUAGGC UCCCUUCAAGGA GCCGUUAGGC UCCGGG CUCAGAAU | 5194 |
| 1200 | GACUUUAA G AAAACCAC | 1986 | GUGGUUUU GGA GCCGUUAGGC UCCCUUCAAGGA GCCGUUAGGC UCCGGG UUAAAGUC | 5195 |
| 1254 | UUGCUGCA G AUUGGACA | 1987 | UGUCCAAU GGA GCCGUUAGGC UCCCUUCAAGGA GCCGUUAGGC UCCGGG UGCAGCAA | 5196 |
| 1258 | UGCAGAUU G GACAAAGA | 1988 | UCUUUGUC GGA GCCGUUAGGC UCCCUUCAAGGA GCCGUUAGGC UCCGGG AAUCUGCA | 5197 |
| 1259 | GCAGAUUG G ACAAAGAA | 1989 | UUCUUUGU GGA GCCGUUAGGC UCCCUUCAAGGA GCCGUUAGGC UCCGGG CAAUCUGC | 5198 |
| 1265 | UGGACAAA G AAUUGUGU | 1990 | ACACAAUU GGA GCCGUUAGGC UCCCUUCAAGGA GCCGUUAGGC UCCGGG UUUGUCCA | 5199 |
| 1294 | ACAAAUCU G GAAGCAUG | 1991 | CAUGCUUC GGA GCCGUUAGGC UCCCUUCAAGGA GCCGUUAGGC UCCGGG AGAUUUGU | 5200 |
| 1295 | CAAAUCUG G AAGCAUGG | 1992 | CCAUGCUU GGA GCCGUUAGGC UCCCUUCAAGGA GCCGUUAGGC UCCGGG CAGAUUUG | 5201 |
| 1302 | GGAAGCAU G GCGACUGG | 1993 | CCAGUCGC GGA GCCGUUAGGC UCCCUUCAAGGA GCCGUUAGGC UCCGGG AUGCUUCC | 5202 |
| 1309 | UGGCGACU G GUAACCGC | 1994 | GCGGUUAC GGA GCCGUUAGGC UCCCUUCAAGGA GCCGUUAGGC UCCGGG AGUCGCCA | 5203 |
| 1339 | AUCAAGCA G GCCAGCUU | 1995 | AAGCUGGC GGA GCCGUUAGGC UCCCUUCAAGGA GCCGUUAGGC UCCGGG UGCUUGAU | 5204 |
| 1359 | CUGCUGCA G ACAGUUGA | 1996 | UCAACUGU GGA GCCGUUAGGC UCCCUUCAAGGA GCCGUUAGGC UCCGGG UGCAGCAG | 5205 |
| 1371 | GUUGAGCU G GGGUCCUG | 1997 | CAGGACCC GGA GCCGUUAGGC UCCCUUCAAGGA GCCGUUAGGC UCCGGG AGCUCAAC | 5206 |
| 1372 | UUGAGCUG G GGUCCUGG | 1998 | CCAGGACC GGA GCCGUUAGGC UCCCUUCAAGGA GCCGUUAGGC UCCGGG CAGCUCAA | 5207 |
| 1373 | UGAGCUGG G GUCCUGGG | 1999 | CCCAGGAC GGA GCCGUUAGGC UCCCUUCAAGGA GCCGUUAGGC UCCGGG CCAGCUCA | 5208 |
| 1379 | GGGGUCCU G GGUUGGGA | 2000 | UCCCAACC GGA GCCGUUAGGC UCCCUUCAAGGA GCCGUUAGGC UCCGGG AGGACCCC | 5209 |
| 1380 | GGGUCCUG G GUUGGGAU | 2001 | AUCCCAAC GGA GCCGUUAGGC UCCCUUCAAGGA GCCGUUAGGC UCCGGG CAGGACCC | 5210 |
| 1384 | CCUGGGUU G GGAUGGUG | 2002 | CACCAUCC GGA GCCGUUAGGC UCCCUUCAAGGA GCCGUUAGGC UCCGGG AACCCAGG | 5211 |
| 1385 | CUGGGUUG G GAUGGUGA | 2003 | UCACCAUC GGA GCCGUUAGGC UCCCUUCAAGGA GCCGUUAGGC UCCGGG CAACCCAG | 5212 |
| 1386 | UGGGUUGG G AUGGUGAC | 2004 | GUCACCAU GGA GCCGUUAGGC UCCCUUCAAGGA GCCGUUAGGC UCCGGG CCAACCCA | 5213 |
| 1389 | GUUGGGAU G GUGACAUU | 2005 | AAUGUCAC GGA GCCGUUAGGC UCCCUUCAAGGA GCCGUUAGGC UCCGGG AUCCCAAC | 5214 |
| 1434 | CUCAUACA G AUAAACAG | 2006 | CUGUUUAU GGA GCCGUUAGGC UCCCUUCAAGGA GCCGUUAGGC UCCGGG UGUAUGAG | 5215 |
| 1444 | UAAACAGU G GCAGUGAC | 2007 | GUCACUGC GGA GCCGUUAGGC UCCCUUCAAGGA GCCGUUAGGC UCCGGG ACUGUUUA | 5216 |
| 1454 | CAGUGACA G GGACACAC | 2008 | GUGUGUCC GGA GCCGUUAGGC UCCCUUCAAGGA GCCGUUAGGC UCCGGG UGUCACUG | 5217 |
| 1455 | AGUGACAG G GACACACU | 2009 | AGUGUGUC GGA GCCGUUAGGC UCCCUUCAAGGA GCCGUUAGGC UCCGGG CUGUCACU | 5218 |
| 1456 | GUGACAGG G ACACACUC | 2010 | GAGUGUGU GGA GCCGUUAGGC UCCCUUCAAGGA GCCGUUAGGC UCCGGG CCUGUCAC | 5219 |
| 1472 | CGCCAAAA G AUUACCUG | 2011 | CAGGUAAU GGA GCCGUUAGGC UCCCUUCAAGGA GCCGUUAGGC UCCGGG UUUUGGCG | 5220 |
| 1492 | CAGCUUCA G GAGGGACG | 2012 | CGUCCCUC GGA GCCGUUAGGC UCCCUUCAAGGA GCCGUUAGGC UCCGGG UGAAGCUG | 5221 |
| 1493 | AGCUUCAG G AGGGACGU | 2013 | ACGUCCCU GGA GCCGUUAGGC UCCCUUCAAGGA GCCGUUAGGC UCCGGG CUGAAGCU | 5222 |
| 1495 | CUUCAGGA G GGACGUCC | 2014 | GGACGUCC GGA GCCGUUAGGC UCCCUUCAAGGA GCCGUUAGGC UCCGGG UCCUGAAG | 5223 |
| 1496 | UUCAGGAG G GACGUCCA | 2015 | UGGACGUC GGA GCCGUUAGGC UCCCUUCAAGGA GCCGUUAGGC UCCGGG CUCCUGAA | 5224 |
| 1497 | UCAGGAGG G ACGUCCAU | 2016 | AUGGACGU GGA GCCGUUAGGC UCCCUUCAAGGA GCCGUUAGGC UCCGGG CCUCCUGA | 5225 |
| 1513 | UCUGCAGC G GGCUUCGA | 2017 | UCGAAGCC GGA GCCGUUAGGC UCCCUUCAAGGA GCCGUUAGGC UCCGGG GCUGCAGA | 5226 |
| 1514 | CUGCAGCG G GCUUCGAU | 2018 | AUCGAAGC GGA GCCGUUAGGC UCCCUUCAAGGA GCCGUUAGGC UCCGGG CGCUGCAG | 5227 |
| 1524 | CUUCGAUC G GCAUUUAC | 2019 | GUAAAUGC GGA GCCGUUAGGC UCCCUUCAAGGA GCCGUUAGGC UCCGGG GAUCGAAG | 5228 |
| 1541 | UGUGAUUA G GAAGAAAU | 2020 | AUUUCUUC GGA GCCGUUAGGC UCCCUUCAAGGA GCCGUUAGGC UCCGGG UAAUCACA | 5229 |
| 1542 | GUGAUUAG G AAGAAAUA | 2021 | UAUUUCUU GGA GCCGUUAGGC UCCCUUCAAGGA GCCGUUAGGC UCCGGG CUAAUCAC | 5230 |
| 1545 | AUUAGGAA G AAAUAUCC | 2022 | GGAUAUUU GGA GCCGUUAGGC UCCCUUCAAGGA GCCGUUAGGC UCCGGG UUCCUAAU | 5231 |
| 1561 | CAACUGAU G GAUCUGAA | 2023 | UUCAGAUC GGA GCCGUUAGGC UCCCUUCAAGGA GCCGUUAGGC UCCGGG AUCAGUUG | 5232 |
| 1562 | AACUGAUG G AUCUGAAA | 2024 | UUUCAGAU GGA GCCGUUAGGC UCCCUUCAAGGA GCCGUUAGGC UCCGGG CAUCAGUU | 5233 |
| 1584 | CUGCUGAC G GAUGGGGA | 2025 | UCCCCAUC GGA GCCGUUAGGC UCCCUUCAAGGA GCCGUUAGGC UCCGGG GUCAGCAG | 5234 |
| 1585 | UGCUGACG G AUGGGGAA | 2026 | UUCCCCAU GGA GCCGUUAGGC UCCCUUCAAGGA GCCGUUAGGC UCCGGG CGUCAGCA | 5235 |
| 1588 | UGACGGAU G GGGAAGAC | 2027 | GUCUUCCC GGA GCCGUUAGGC UCCCUUCAAGGA GCCGUUAGGC UCCGGG AUCCGUCA | 5236 |
| 1589 | GACGGAUG G GGAAGACA | 2028 | UGUCUUCC GGA GCCGUUAGGC UCCCUUCAAGGA GCCGUUAGGC UCCGGG CAUCCGUC | 5237 |
| 1590 | ACGGAUGG G GAAGACAA | 2029 | UUGUCUUC GGA GCCGUUAGGC UCCCUUCAAGGA GCCGUUAGGC UCCGGG CCAUCCGU | 5238 |
| 1591 | CGGAUGGG G AAGACAAC | 2030 | GUUGUCUU GGA GCCGUUAGGC UCCCUUCAAGGA GCCGUUAGGC UCCGGG CCCAUCCG | 5239 |
| 1594 | AUGGGGAA G ACAACACU | 2031 | AGUGUUGU GGA GCCGUUAGGC UCCCUUCAAGGA GCCGUUAGGC UCCGGG UUCCCCAU | 5240 |
| 1609 | CUAUAAGU G GGUGCUUU | 2032 | AAAGCACC GGA GCCGUUAGGC UCCCUUCAAGGA GCCGUUAGGC UCCGGG ACUUAUAG | 5241 |
| 1610 | UAUAAGUG G GUGCUUUA | 2033 | UAAAGCAC GGA GCCGUUAGGC UCCCUUCAAGGA GCCGUUAGGC UCCGGG CACUUAUA | 5242 |
| 1623 | UUUAACGA G GUCAAACA | 2034 | UGUUUGAC GGA GCCGUUAGGC UCCCUUCAAGGA GCCGUUAGGC UCCGGG UCGUUAAA | 5243 |
| 1636 | AACAAAGU G GUGCCAUC | 2035 | GAUGGCAC GGA GCCGUUAGGC UCCCUUCAAGGA GCCGUUAGGC UCCGGG ACUUUGUU | 5244 |
| 1662 | GUCGCUUU G GGGCCCUC | 2036 | GAGGGCCC GGA GCCGUUAGGC UCCCUUCAAGGA GCCGUUAGGC UCCGGG AAAGCGAC | 5245 |
| 1663 | UCGCUUUG G GGCCCUCU | 2037 | AGAGGGCC GGA GCCGUUAGGC UCCCUUCAAGGA GCCGUUAGGC UCCGGG CAAAGCGA | 5246 |
| 1664 | CGCUUUGG G GCCCUCUG | 2038 | CAGAGGGC GGA GCCGUUAGGC UCCCUUCAAGGA GCCGUUAGGC UCCGGG CCAAAGCG | 5247 |
| 1681 | CAGCUCAA G AACUAGAG | 2039 | CUCUAGUU GGA GCCGUUAGGC UCCCUUCAAGGA GCCGUUAGGC UCCGGG UUGAGCUG | 5248 |
| 1687 | AAGAACUA G AGGAGCUG | 2040 | CAGCUCCU GGA GCCGUUAGGC UCCCUUCAAGGA GCCGUUAGGC UCCGGG UAGUUCUU | 5249 |
| 1689 | GAACUAGA G GAGCUGUC | 2041 | GACAGCUC GGA GCCGUUAGGC UCCCUUCAAGGA GCCGUUAGGC UCCGGG UCUAGUUC | 5250 |
| 1690 | AACUAGAG G AGCUGUCC | 2042 | GGACAGCU GGA GCCGUUAGGC UCCCUUCAAGGA GCCGUUAGGC UCCGGG CUCUAGUU | 5251 |
| 1708 | AAAUGACA G GAGGUUUA | 2043 | UAAACCUC GGA GCCGUUAGGC UCCCUUCAAGGA GCCGUUAGGC UCCGGG UGUCAUUU | 5252 |
| 1709 | AAUGACAG G AGGUUUAC | 2044 | GUAAACCU GGA GCCGUUAGGC UCCCUUCAAGGA GCCGUUAGGC UCCGGG CUGUCAUU | 5253 |
| 1711 | UGACAGGA G GUUUACAG | 2045 | CUGUAAAC GGA GCCGUUAGGC UCCCUUCAAGGA GCCGUUAGGC UCCGGG UCCUGUCA | 5254 |
| 1719 | GGUUUACA G ACAUAUGC | 2046 | GCAUAUGU GGA GCCGUUAGGC UCCCUUCAAGGA GCCGUUAGGC UCCGGG UGUAAACC | 5255 |
| 1732 | AUGCUUCA G AUCAAGUU | 2047 | AACUUGAU GGA GCCGUUAGGC UCCCUUCAAGGA GCCGUUAGGC UCCGGG UGAAGCAU | 5256 |
| 1743 | CAAGUUCA G AACAAUGG | 2048 | CCAUUGUU GGA GCCGUUAGGC UCCCUUCAAGGA GCCGUUAGGC UCCGGG UGAACUUG | 5257 |
| 1750 | AGAACAAU G GCCUCAUU | 2049 | AAUGAGGC GGA GCCGUUAGGC UCCCUUCAAGGA GCCGUUAGGC UCCGGG AUUGUUCU | 5258 |
| 1768 | AUGCUUUU G GGGCCCUU | 2050 | AAGGGCCC GGA GCCGUUAGGC UCCCUUCAAGGA GCCGUUAGGC UCCGGG AAAAGCAU | 5259 |
| 1769 | UGCUUUUG G GGCCCUUU | 2051 | AAAGGGCC GGA GCCGUUAGGC UCCCUUCAAGGA GCCGUUAGGC UCCGGG CAAAAGCA | 5260 |
| 1770 | GCUUUUGG G GCCCUUUC | 2052 | GAAAGGGC GGA GCCGUUAGGC UCCCUUCAAGGA GCCGUUAGGC UCCGGG CCAAAAGC | 5261 |
| 1783 | UUUCAUCA G GAAAUGGA | 2053 | UCCAUUUC GGA GCCGUUAGGC UCCCUUCAAGGA GCCGUUAGGC UCCGGG UGAUGAAA | 5262 |
| 1784 | UUCAUCAG G AAAUGGAG | 2054 | CUCCAUUU GGA GCCGUUAGGC UCCCUUCAAGGA GCCGUUAGGC UCCGGG CUGAUGAA | 5263 |
| 1789 | CAGGAAAU G GAGCUGUC | 2055 | GACAGCUC GGA GCCGUUAGGC UCCCUUCAAGGA GCCGUUAGGC UCCGGG AUUUCCUG | 5264 |
| 1790 | AGGAAAUG G AGCUGUCU | 2056 | AGACAGCU GGA GCCGUUAGGC UCCCUUCAAGGA GCCGUUAGGC UCCGGG CAUUUCCU | 5265 |
| 1821 | CAGCUUGA G AGUAAGGG | 2057 | CCCUUACU GGA GCCGUUAGGC UCCCUUCAAGGA GCCGUUAGGC UCCGGG UCAAGCUG | 5266 |
| 1827 | GAGAGUAA G GGAUUAAC | 2058 | GUUAAUCC GGA GCCGUUAGGC UCCCUUCAAGGA GCCGUUAGGC UCCGGG UUACUCUC | 5267 |
| 1828 | AGAGUAAG G GAUUAACC | 2059 | GGUUAUUC GGA GCCGUUAGGC UCCCUUCAAGGA GCCGUUAGGC UCCGGG CUUACUCU | 5268 |
| 1829 | GAGUAAGG G AUUAACCC | 2060 | GGGUUAAU GGA GCCGUUAGGC UCCCUUCAAGGA GCCGUUAGGC UCCGGG CCUUACUC | 5269 |
| 1842 | ACCCUCCA G AACAGCCA | 2061 | UGGCUGUU GGA GCCGUUAGGC UCCCUUCAAGGA GCCGUUAGGC UCCGGG UGGAGGGU | 5270 |
| 1853 | CAGCCAGU G GAUGAAUG | 2062 | CAUUCAUC GGA GCCGUUAGGC UCCCUUCAAGGA GCCGUUAGGC UCCGGG ACUGGCUG | 5271 |
| 1854 | AGCCAGUG G AUGAAUGG | 2063 | CCAUUCAU GGA GCCGUUAGGC UCCCUUCAAGGA GCCGUUAGGC UCCGGG CACUGGCU | 5272 |
| 1861 | GGAUGAAU G GCACAGUG | 2064 | CACUGUGC GGA GCCGUUAGGC UCCCUUCAAGGA GCCGUUAGGC UCCGGG AUUCAUCC | 5273 |
| 1875 | GUGAUCGU G GACAGCAC | 2065 | GUGCUGUC GGA GCCGUUAGGC UCCCUUCAAGGA GCCGUUAGGC UCCGGG ACGAUCAC | 5274 |
| 1876 | UGAUCGUG G ACAGCACC | 2066 | GGUGCUGU GGA GCCGUUAGGC UCCCUUCAAGGA GCCGUUAGGC UCCGGG CACGAUCA | 5275 |
| 1887 | AGCACCGU G GGAAAGGA | 2067 | UCCUUUCC GGA GCCGUUAGGC UCCCUUCAAGGA GCCGUUAGGC UCCGGG ACGGUGCU | 5276 |
| 1888 | GCACCGUG G GAAAGGAC | 2068 | GUCCUUUC GGA GCCGUUAGGC UCCCUUCAAGGA GCCGUUAGGC UCCGGG CACGGUGC | 5277 |
| 1889 | CACCGUGG G AAAGGACA | 2069 | UGUCCUUU GGA GCCGUUAGGC UCCCUUCAAGGA GCCGUUAGGC UCCGGG CCACGGUG | 5278 |
| 1893 | GUGGGAAA G GACACUUU | 2070 | AAAGUGUC GGA GCCGUUAGGC UCCCUUCAAGGA GCCGUUAGGC UCCGGG UUUCCCAC | 5279 |
| 1894 | UGGGAAAG G ACACUUUG | 2071 | CAAAGUGU GGA GCCGUUAGGC UCCCUUCAAGGA GCCGUUAGGC UCCGGG CUUUCCCA | 5280 |
| 1916 | UAUCACCU G GACAACGC | 2072 | GCGUUGUC GGA GCCGUUAGGC UCCCUUCAAGGA GCCGUUAGGC UCCGGG AGGUGAUA | 5281 |
| 1917 | AUCACCUG G ACAACGCA | 2073 | UGCGUUGU GGA GCCGUUAGGC UCCCUUCAAGGA GCCGUUAGGC UCCGGG CAGGUGAU | 5282 |
| 1946 | CCUUCUCU G GGAUCCCA | 2074 | UGGGAUCC GGA GCCGUUAGGC UCCCUUCAAGGA GCCGUUAGGC UCCGGG AGAGAAGG | 5283 |
| 1947 | CUUCUCUG G GAUCCCAG | 2075 | CUGGGAUC GGA GCCGUUAGGC UCCCUUCAAGGA GCCGUUAGGC UCCGGG CAGAGAAG | 5284 |
| 1948 | UUCUCUGG G AUCCCAGU | 2076 | ACUGGGAU GGA GCCGUUAGGC UCCCUUCAAGGA GCCGUUAGGC UCCGGG CCAGAGAA | 5285 |
| 1957 | AUCCCAGU G GACAGAAG | 2077 | CUUCUGUC GGA GCCGUUAGGC UCCCUUCAAGGA GCCGUUAGGC UCCGGG ACUGGGAU | 5286 |
| 1958 | UCCCAGUG G ACAGAAGC | 2078 | GCUUCUGU GGA GCCGUUAGGC UCCCUUCAAGGA GCCGUUAGGC UCCGGG CACUGGGA | 5287 |
| 1962 | AGUGGACA G AAGCAAGG | 2079 | CCUUGCUU GGA GCCGUUAGGC UCCCUUCAAGGA GCCGUUAGGC UCCGGG UGUCCACU | 5288 |
| 1969 | AGAAGCAA G GUGGCUUU | 2080 | AAAGCCAC GGA GCCGUUAGGC UCCCUUCAAGGA GCCGUUAGGC UCCGGG UUGCUUCU | 5289 |
| 1972 | AGCAAGGU G GCUUUGUA | 2081 | UACAAAGC GGA GCCGUUAGGC UCCCUUCAAGGA GCCGUUAGGC UCCGGG ACCUUGCU | 5290 |
| 1983 | UUUGUAGU G GACAAAAA | 2082 | UUUUUGUC GGA GCCGUUAGGC UCCCUUCAAGGA GCCGUUAGGC UCCGGG ACUACAAA | 5291 |
| 1984 | UUGUAGUG G ACAAAAAC | 2083 | GUUUUUGU GGA GCCGUUAGGC UCCCUUCAAGGA GCCGUUAGGC UCCGGG CACUACAA | 5292 |
| 2001 | ACCAAAAU G GCCUACCU | 2084 | AGGUAGGC GGA GCCGUUAGGC UCCCUUCAAGGA GCCGUUAGGC UCCGGG AUUUUGGU | 5293 |
| 2020 | AAAUCCCA G GCAUUGCU | 2085 | AGCAAUGC GGA GCCGUUAGGC UCCCUUCAAGGA GCCGUUAGGC UCCGGG UGGGAUUU | 5294 |
| 2031 | AUUGCUAA G GUUGGCAC | 2086 | GUGCCAAC GGA GCCGUUAGGC UCCCUUCAAGGA GCCGUUAGGC UCCGGG UUAGCAAU | 5295 |
| 2035 | CUAAGGUU G GCACUUGG | 2087 | CCAAGUGC GGA GCCGUUAGGC UCCCUUCAAGGA GCCGUUAGGC UCCGGG AACCUUAG | 5296 |
| 2042 | UGGCACUU G GAAAUACA | 2088 | UGUAUUUC GGA GCCGUUAGGC UCCCUUCAAGGA GCCGUUAGGC UCCGGG AAGUGCCA | 5297 |
| 2043 | GGCACUUG G AAAUACAG | 2089 | CUGUAUUU GGA GCCGUUAGGC UCCCUUCAAGGA GCCGUUAGGC UCCGGG CAAGUGCC | 5298 |
| 2148 | ACGAACAA G GACACCAG | 2090 | CUGGUGUC GGA GCCGUUAGGC UCCCUUCAAGGA GCCGUUAGGC UCCGGG UUGUUCGU | 5299 |
| 2149 | CGAACAAG G ACACCAGC | 2091 | GCUGGUGU GGA GCCGUUAGGC UCCCUUCAAGGA GCCGUUAGGC UCCGGG CUUGUUCG | 5300 |
| 2175 | AGCCCUCU G GUAGUUUA | 2092 | UAAACUAC GGA GCCGUUAGGC UCCCUUCAAGGA GCCGUUAGGC UCCGGG AGAGGGCU | 5301 |
| 2200 | UUCGCCAA G GAGCCUCC | 2093 | GGAGGCUC GGA GCCGUUAGGC UCCCUUCAAGGA GCCGUUAGGC UCCGGG UUGGCGAA | 5302 |
| 2201 | UCGCCAAG G AGCCUCCC | 2094 | GGGAGGCU GGA GCCGUUAGGC UCCCUUCAAGGA GCCGUUAGGC UCCGGG CUUGGCGA | 5303 |
| 2219 | AAUUCUCA G GGCCAGUG | 2095 | CACUGGCC GGA GCCGUUAGGC UCCCUUCAAGGA GCCGUUAGGC UCCGGG UGAGAAUU | 5304 |
| 2220 | AUUCUCAG G GCCAGUGU | 2096 | ACACUGGC GGA GCCGUUAGGC UCCCUUCAAGGA GCCGUUAGGC UCCGGG CUGAGAAU | 5305 |
| 2254 | CAGUGAAU G GAAAAACA | 2097 | UGUUUUUC GGA GCCGUUAGGC UCCCUUCAAGGA GCCGUUAGGC UCCGGG AUUCACUG | 5306 |
| 2255 | AGUGAAUG G AAAAACAG | 2098 | CUGUUUUU GGA GCCGUUAGGC UCCCUUCAAGGA GCCGUUAGGC UCCGGG CAUUCACU | 5307 |
| 2271 | GUUACCUU G GAACUACU | 2099 | AGUAGUUC GGA GCCGUUAGGC UCCCUUCAAGGA GCCGUUAGGC UCCGGG AAGGUAAC | 5308 |
| 2272 | UUACCUUG G AACUACUG | 2100 | CAGUAGUU GGA GCCGUUAGGC UCCCUUCAAGGA GCCGUUAGGC UCCGGG CAAGGUAA | 5309 |
| 2280 | GAACUACU G GAUAAUGG | 2101 | CCAUUAUC GGA GCCGUUAGGC UCCCUUCAAGGA GCCGUUAGGC UCCGGG AGUAGUUC | 5310 |
| 2281 | AACUACUG G AUAAUGGA | 2102 | UCCAUUAU GGA GCCGUUAGGC UCCCUUCAAGGA GCCGUUAGGC UCCGGG CAGUAGUU | 5311 |
| 2287 | UGGAUAAU G GAGCAGGU | 2103 | ACCUGCUC GGA GCCGUUAGGC UCCCUUCAAGGA GCCGUUAGGC UCCGGG AUUAUCCA | 5312 |
| 2288 | GGAUAAUG G AGCAGGUG | 2104 | CACCUGCU GGA GCCGUUAGGC UCCCUUCAAGGA GCCGUUAGGC UCCGGG CAUUAUCC | 5313 |
| 2293 | AUGGAGCA G GUGCUGAU | 2105 | AUCAGCAC GGA GCCGUUAGGC UCCCUUCAAGGA GCCGUUAGGC UCCGGG UGCUCCAU | 5314 |
| 2310 | GCUACUAA G GAUGACGG | 2106 | CCGUCAUC GGA GCCGUUAGGC UCCCUUCAAGGA GCCGUUAGGC UCCGGG UUAGUAGC | 5315 |
| 2311 | CUACUAAG G AUGACGGU | 2107 | ACCGUCAU GGA GCCGUUAGGC UCCCUUCAAGGA GCCGUUAGGC UCCGGG CUUAGUAG | 5316 |
| 2317 | AGGAUGAC G GUGUCUAC | 2108 | GUAGACAC GGA GCCGUUAGGC UCCCUUCAAGGA GCCGUUAGGC UCCGGG GUCAUCCU | 5317 |
| 2330 | CUACUCAA G GUAUUUCA | 2109 | UGAAAUAC GGA GCCGUUAGGC UCCCUUCAAGGA GCCGUUAGGC UCCGGG UUGAGUAG | 5318 |
| 2356 | ACACGAAU G GUAGAUAC | 2110 | GUAUCUAC GGA GCCGUUAGGC UCCCUUCAAGGA GCCGUUAGGC UCCGGG AUUCGUGU | 5319 |
| 2360 | GAAUGGUA G AUACAGUG | 2111 | CACUGUAU GGA GCCGUUAGGC UCCCUUCAAGGA GCCGUUAGGC UCCGGG UACCAUUC | 5320 |
| 2378 | AAAAGUGC G GGCUCUGG | 2112 | CCAGAGCC GGA GCCGUUAGGC UCCCUUCAAGGA GCCGUUAGGC UCCGGG GCACUUUU | 5321 |
| 2379 | AAAGUGCG G GCUCUGGG | 2113 | CCCAGAGC GGA GCCGUUAGGC UCCCUUCAAGGA GCCGUUAGGC UCCGGG CGCACUUU | 5322 |
| 2385 | CGGGCUCU G GGAGGAGU | 2114 | ACUCCUCC GGA GCCGUUAGGC UCCCUUCAAGGA GCCGUUAGGC UCCGGG AGAGCCCG | 5323 |
| 2386 | GGGCUCUG G GAGGAGUU | 2115 | AACUCCUC GGA GCCGUUAGGC UCCCUUCAAGGA GCCGUUAGGC UCCGGG CAGAGCCC | 5324 |
| 2387 | GGCUCUGG G AGGAGUUA | 2116 | UAACUCCU GGA GCCGUUAGGC UCCCUUCAAGGA GCCGUUAGGC UCCGGG CCAGAGCC | 5325 |
| 2389 | CUCUGGGA G GAGUUAAC | 2117 | GUUAACUC GGA GCCGUUAGGC UCCCUUCAAGGA GCCGUUAGGC UCCGGG UCCCAGAG | 5326 |
| 2390 | UCUGGGAG G AGUUAACG | 2118 | CGUUAACU GGA GCCGUUAGGC UCCCUUCAAGGA GCCGUUAGGC UCCGGG CUCCCAGA | 5327 |
| 2405 | CGCAGCCA G ACGGAGAG | 2119 | CUCUCCGU GGA GCCGUUAGGC UCCCUUCAAGGA GCCGUUAGGC UCCGGG UGGCUGCG | 5328 |
| 2408 | AGCCAGAC G GAGAGUGA | 2120 | UCACUCUC GGA GCCGUUAGGC UCCCUUCAAGGA GCCGUUAGGC UCCGGG GUCUGGCU | 5329 |
| 2409 | GCCAGACG G AGAGUGAU | 2121 | AUCACUCU GGA GCCGUUAGGC UCCCUUCAAGGA GCCGUUAGGC UCCGGG CGUCUGGC | 5330 |
| 2411 | CAGACGGA G AGUGAUAC | 2122 | GUAUCACU GGA GCCGUUAGGC UCCCUUCAAGGA GCCGUUAGGC UCCGGG UCCGUCUG | 5331 |
| 2427 | CCCCAGCA G AGUGGAGC | 2123 | GCUCCACU GGA GCCGUUAGGC UCCCUUCAAGGA GCCGUUAGGC UCCGGG UGCUGGGG | 5332 |
| 2431 | AGCAGAGU G GAGCACUG | 2124 | CAGUGCUC GGA GCCGUUAGGC UCCCUUCAAGGA GCCGUUAGGC UCCGGG ACUCUGCU | 5333 |
| 2432 | GCAGAGUG G AGCACUGU | 2125 | ACAGUGCU GGA GCCGUUAGGC UCCCUUCAAGGA GCCGUUAGGC UCCGGG CACUCUGC | 5334 |
| 2449 | ACAUACCU G GCUGGAUU | 2126 | AAUCCAGC GGA GCCGUUAGGC UCCCUUCAAGGA GCCGUUAGGC UCCGGG AGGUAUGU | 5335 |
| 2453 | ACCUGGCU G GAUUGAGA | 2127 | UCUCAAUC GGA GCCGUUAGGC UCCCUUCAAGGA GCCGUUAGGC UCCGGG AGCCAGGU | 5336 |
| 2454 | CCUGGCUG G AUUGAGAA | 2128 | UUCUCAUU GGA GCCGUUAGGC UCCCUUCAAGGA GCCGUUAGGC UCCGGG CAGCCAGG | 5337 |
| 2460 | UGGAUUGA G AAUGAUGA | 2129 | UCAUCAUU GGA GCCGUUAGGC UCCCUUCAAGGA GCCGUUAGGC UCCGGG UCAAUCCA | 5338 |
| 2477 | AAUACAAU G GAAUCCAC | 2130 | GUGGAUUC GGA GCCGUUAGGC UCCCUUCAAGGA GCCGUUAGGC UCCGGG AUUGUAUU | 5339 |
| 2478 | AUACAAUG G AAUCCACC | 2131 | GGUGGAUU GGA GCCGUUAGGC UCCCUUCAAGGA GCCGUUAGGC UCCGGG CAUUGUAU | 5340 |
| 2489 | UCCACCAA G ACCUGAAA | 2132 | UUUCAGGU GGA GCCGUUAGGC UCCCUUCAAGGA GCCGUUAGGC UCCGGG UUGGUGGA | 5341 |
| 2505 | AUUAAUAA G GAUGAUGU | 2133 | ACAUCAUC GGA GCCGUUAGGC UCCCUUCAAGGA GCCGUUAGGC UCCGGG UUAUUAAU | 5342 |
| 2506 | UUAAUAAG G AUGAUGUU | 2134 | AACAUCAU GGA GCCGUUAGGC UCCCUUCAAGGA GCCGUUAGGC UCCGGG CUUAUUAA | 5343 |
| 2540 | UUUCAGCA G AACAUCCU | 2135 | AGGAUGUU GGA GCCGUUAGGC UCCCUUCAAGGA GCCGUUAGGC UCCGGG UGCUGAAA | 5344 |
| 2550 | ACAUCCUC G GGAGGCUC | 2136 | GAGCCUCC GGA GCCGUUAGGC UCCCUUCAAGGA GCCGUUAGGC UCCGGG GAGGAUGU | 5345 |
| 2551 | CAUCCUCG G GAGGCUCA | 2137 | UGAGCCUC GGA GCCGUUAGGC UCCCUUCAAGGA GCCGUUAGGC UCCGGG CGAGGAUG | 5346 |
| 2552 | AUCCUCGG G AGGCUCAU | 2138 | AUGAGCCU GGA GCCGUUAGGC UCCCUUCAAGGA GCCGUUAGGC UCCGGG CCGAGGAU | 5347 |
| 2554 | CCUCGGGA G GCUCAUUU | 2139 | AAAUGAGC GGA GCCGUUAGGC UCCCUUCAAGGA GCCGUUAGGC UCCGGG UCCCGAGG | 5348 |
| 2565 | UCAUUUGU G GCUUCUGA | 2140 | UCAGAAGC GGA GCCGUUAGGC UCCCUUCAAGGA GCCGUUAGGC UCCGGG ACAAAUGA | 5349 |
| 2611 | UCCCACCU G GCCAAAUC | 2141 | GAUUUGGC GGA GCCGUUAGGC UCCCUUCAAGGA GCCGUUAGGC UCCGGG AGGUGGGA | 5350 |
| 2631 | GACCUGAA G GCGGAAAU | 2142 | AUUUCCGC GGA GCCGUUAGGC UCCCUUCAAGGA GCCGUUAGGC UCCGGG UUCAGGUC | 5351 |
| 2634 | CUGAAGGC G GAAAUUCA | 2143 | UGAAUUUC GGA GCCGUUAGGC UCCCUUCAAGGA GCCGUUAGGC UCCGGG GCCUUCAG | 5352 |
| 2635 | UGAAGGCG G AAAUUCAC | 2144 | GUGAAUUU GGA GCCGUUAGGC UCCCUUCAAGGA GCCGUUAGGC UCCGGG CGCCUUCA | 5353 |
| 2644 | AAAUUCAC G GGGGCAGU | 2145 | ACUGCCCC GGA GCCGUUAGGC UCCCUUCAAGGA GCCGUUAGGC UCCGGG GUGAAUUU | 5354 |
| 2645 | AAUUCACG G GGGCAGUC | 2146 | GACUGCCC GGA GCCGUUAGGC UCCCUUCAAGGA GCCGUUAGGC UCCGGG CGUGAAUU | 5355 |
| 2646 | AUUCACGG G GGCAGUCU | 2147 | AGACUGCC GGA GCCGUUAGGC UCCCUUCAAGGA GCCGUUAGGC UCCGGG CCGUGAAU | 5356 |
| 2647 | UUCACGGG G GCAGUCUC | 2148 | GAGACUGC GGA GCCGUUAGGC UCCCUUCAAGGA GCCGUUAGGC UCCGGG CCCGUGAA | 5357 |
| 2669 | UCUGACUU G GACAGCUC | 2149 | GAGCUGUC GGA GCCGUUAGGC UCCCUUCAAGGA GCCGUUAGGC UCCGGG AAGUCAGA | 5358 |
| 2670 | CUGACUUG G ACAGCUCC | 2150 | GGAGCUGU GGA GCCGUUAGGC UCCCUUCAAGGA GCCGUUAGGC UCCGGG CAAGUCAG | 5359 |
| 2680 | CAGCUCCU G GGGAUGAU | 2151 | AUCAUCCC GGA GCCGUUAGGC UCCCUUCAAGGA GCCGUUAGGC UCCGGG AGGAGCUG | 5360 |
| 2681 | AGCUCCUG G GGAUGAUU | 2152 | AAUCAUCC GGA GCCGUUAGGC UCCCUUCAAGGA GCCGUUAGGC UCCGGG CAGGAGCU | 5361 |
| 2682 | GCUCCUGG G GAUGAUUA | 2153 | UAAUCAUC GGA GCCGUUAGGC UCCCUUCAAGGA GCCGUUAGGC UCCGGG CCAGGAGC | 5362 |
| 2683 | CUCCUGGG G AUGAUUAU | 2154 | AUAAUCAU GGA GCCGUUAGGC UCCCUUCAAGGA GCCGUUAGGC UCCGGG CCCAGGAG | 5363 |
| 2698 | AUGACCAU G GAACAGCU | 2155 | AGCUGUUC GGA GCCGUUAGGC UCCCUUCAAGGA GCCGUUAGGC UCCGGG AUGGUCAU | 5364 |
| 2699 | UGACCAUG G AACAGCUC | 2156 | GAGCUGUU GGA GCCGUUAGGC UCCCUUCAAGGA GCCGUUAGGC UCCGGG CAUGGUCA | 5365 |
| 2750 | UGAUCUCA G AGACAAGU | 2157 | ACUUGUCU GGA GCCGUUAGGC UCCCUUCAAGGA GCCGUUAGGC UCCGGG UGAGAUCA | 5366 |
| 2752 | AUCUCAGA G ACAAGUUC | 2158 | GAACUUGU GGA GCCGUUAGGC UCCCUUCAAGGA GCCGUUAGGC UCCGGG UCUGAGAU | 5367 |
| 2802 | AUCCCAAA G GAAGCCAA | 2159 | UUGGCUUC GGA GCCGUUAGGC UCCCUUCAAGGA GCCGUUAGGC UCCGGG UUUGGGAU | 5368 |
| 2803 | UCCCAAAG G AAGCCAAC | 2160 | GUUGGCUU GGA GCCGUUAGGC UCCCUUCAAGGA GCCGUUAGGC UCCGGG CUUUGGGA | 5369 |
| 2817 | AACUCUGA G GAAGUCUU | 2161 | AAGACUUC GGA GCCGUUAGGC UCCCUUCAAGGA GCCGUUAGGC UCCGGG UCAGAGUU | 5370 |
| 2818 | ACUCUGAG G AAGUCUUU | 2162 | AAAGACUU GGA GCCGUUAGGC UCCCUUCAAGGA GCCGUUAGGC UCCGGG CUCAGAGU | 5371 |
| 2839 | UUAAACCA G AAAACAUU | 2163 | AAUGUUUU GGA GCCGUUAGGC UCCCUUCAAGGA GCCGUUAGGC UCCGGG UGGUUUAA | 5372 |
| 2860 | UUGAAAAU G GCACAGAU | 2164 | AUCUGUGC GGA GCCGUUAGGC UCCCUUCAAGGA GCCGUUAGGC UCCGGG AUUUUCAA | 5373 |
| 2866 | AUGGCACA G AUCUUUUC | 2165 | GAAAAGAU GGA GCCGUUAGGC UCCCUUCAAGGA GCCGUUAGGC UCCGGG UGUGCCAU | 5374 |
| 2886 | GCUAUUCA G GCUGUUGA | 2166 | UCAACAGC GGA GCCGUUAGGC UCCCUUCAAGGA GCCGUUAGGC UCCGGG UGAAUAGC | 5375 |
| 2898 | GUUGAUAA G GUCGAUCU | 2167 | AGAUCGAC GGA GCCGUUAGGC UCCCUUCAAGGA GCCGUUAGGC UCCGGG UUAUCAAC | 5376 |
| 2914 | UGAAAUCA G AAAUAUCC | 2168 | GGAUAUUU GGA GCCGUUAGGC UCCCUUCAAGGA GCCGUUAGGC UCCGGG UGAUUUCA | 5377 |
| 2958 | CCUCCACA G ACUCCGCC | 2169 | GGCGGAGU GGA GCCGUUAGGC UCCCUUCAAGGA GCCGUUAGGC UCCGGG UGUGGAGG | 5378 |
| 2968 | CUCCGCCA G AGACACCU | 2170 | AGGUGUCU GGA GCCGUUAGGC UCCCUUCAAGGA GCCGUUAGGC UCCGGG UGGCGGAG | 5379 |
| 2970 | CCGCCAGA G ACACCUAG | 2171 | CUAGGUGU GGA GCCGUUAGGC UCCCUUCAAGGA GCCGUUAGGC UCCGGG UCUGGCGG | 5380 |
| 3034 | CCAUUCCU G GCAUUCAC | 2172 | GUGAAUGC GGA GCCGUUAGGC UCCCUUCAAGGA GCCGUUAGGC UCCGGG AGGAAUGG | 5381 |
| 3059 | AAUUAUGU G GAAGUGGA | 2173 | UCCACUUC GGA GCCGUUAGGC UCCCUUCAAGGA GCCGUUAGGC UCCGGG ACAUAAUU | 5382 |
| 3060 | AUUAUGUG G AAGUGGAU | 2174 | AUCCACUU GGA GCCGUUAGGC UCCCUUCAAGGA GCCGUUAGGC UCCGGG CACAUAAU | 5383 |
| 3065 | GUGGAAGU G GAUAGGAG | 2175 | CUCCUAUC GGA GCCGUUAGGC UCCCUUCAAGGA GCCGUUAGGC UCCGGG ACUUCCAC | 5384 |
| 3066 | UGGAAGUG G AUAGGAGA | 2176 | UCUCCUAU GGA GCCGUUAGGC UCCCUUCAAGGA GCCGUUAGGC UCCGGG CACUUCCA | 5385 |
| 3070 | AGUGGAUA G GAGAACUG | 2177 | CAGUUCUC GGA GCCGUUAGGC UCCCUUCAAGGA GCCGUUAGGC UCCGGG UAUCCACU | 5386 |
| 3071 | GUGGAUAG G AGAACUGC | 2178 | GCAGUUCU GGA GCCGUUAGGC UCCCUUCAAGGA GCCGUUAGGC UCCGGG CUAUCCAC | 5387 |
| 3073 | GGAUAGGA G AACUGCAG | 2179 | CUGCAGUU GGA GCCGUUAGGC UCCCUUCAAGGA GCCGUUAGGC UCCGGG UCCUAUCC | 5388 |
| 3096 | AUAGCCUA G GGCUGAAU | 2180 | AUUCAGCC GGA GCCGUUAGGC UCCCUUCAAGGA GCCGUUAGGC UCCGGG UAGGCUAU | 5389 |
| 3097 | UAGCCUAG G GCUGAAUU | 2181 | AAUUCAGC GGA GCCGUUAGGC UCCCUUCAAGGA GCCGUUAGGC UCCGGG CUAGGCUA | 5390 |
| 3113 | UUUUGUCA G AUAAAUAA | 2182 | UUAUUUAU GGA GCCGUUAGGC UCCCUUCAAGGA GCCGUUAGGC UCCGGG UGACAAAA | 5391 |
| 3174 | GUAUUUUA G ACUUCCUG | 2183 | CAGGAAGU GGA GCCGUUAGGC UCCCUUCAAGGA GCCGUUAGGC UCCGGG UAAAAUAC | 5392 |
| 3264 | GUAUUUUA G ACUUCCUG | 2183 | CAGGAAGU GGA GCCGUUAGGC UCCCUUCAAGGA GCCGUUAGGC UCCGGG UAAAAUAC | 5392 |
| 3185 | UUCCUGUA G GGGGCGAU | 2184 | AUCGCCCC GGA GCCGUUAGGC UCCCUUCAAGGA GCCGUUAGGC UCCGGG UACAGGAA | 5393 |
| 3238 | UUCCUGUA G GGGGCGAU | 2184 | AUCGCCCC GGA GCCGUUAGGC UCCCUUCAAGGA GCCGUUAGGC UCCGGG UACAGGAA | 5393 |
| 3275 | UUCCUGUA G GGGGCGAU | 2184 | AUCGCCCC GGA GCCGUUAGGC UCCCUUCAAGGA GCCGUUAGGC UCCGGG UACAGGAA | 5393 |
| 3186 | UCCUGUAG G GGGCGAUA | 2185 | UAUCGCCC GGA GCCGUUAGGC UCCCUUCAAGGA GCCGUUAGGC UCCGGG CUACAGGA | 5394 |
| 3239 | UCCUGUAG G GGGCGAUA | 2185 | UAUCGCCC GGA GCCGUUAGGC UCCCUUCAAGGA GCCGUUAGGC UCCGGG CUACAGGA | 5394 |
| 3276 | UCCUGUAG G GGGCGAUA | 2185 | UAUCGCCC GGA GCCGUUAGGC UCCCUUCAAGGA GCCGUUAGGC UCCGGG CUACAGGA | 5394 |
| 3187 | CCUGUAGG G GGCGAUAU | 2186 | AUAUCGCC GGA GCCGUUAGGC UCCCUUCAAGGA GCCGUUAGGC UCCGGG CCUACAGG | 5395 |
| 3240 | CCUGUAGG G GGCGAUAU | 2186 | AUAUCGCC GGA GCCGUUAGGC UCCCUUCAAGGA GCCGUUAGGC UCCGGG CCUACAGG | 5395 |
| 3188 | CUGUAGGG G GCGAUAUA | 2187 | UAUAUCGC GGA GCCGUUAGGC UCCCUUCAAGGA GCCGUUAGGC UCCGGG CCCUACAG | 5396 |
| 3241 | CUGUAGGG G GCGAUAUA | 2187 | UAUAUCGC GGA GCCGUUAGGC UCCCUUCAAGGA GCCGUUAGGC UCCGGG CCCUACAG | 5396 |
| 3277 | CCUGUAGG G GGCGAUAA | 2188 | UUAUCGCC GGA GCCGUUAGGC UCCCUUCAAGGA GCCGUUAGGC UCCGGG CCUACAGG | 5397 |
| 3278 | CUGUAGGG G GCGAUAAA | 2189 | UUUAUCGC GGA GCCGUUAGGC UCCCUUCAAGGA GCCGUUAGGC UCCGGG CCCUACAG | 5398 |

Input Sequence = NM_001285. Cut Site = G/.
Arm Length = 8. Core Sequence = GGAGGAAACUCC CU UCAAGGACAUCGUCCGGG
Underlined region can be any X sequence or linker, as described herein.
NM_001285 (Homo sapiens chloride channel, calcium activated, 1 (CLCA1) mRNA, 3311 bp)

TABLE IX
Human CLCA1 GeneBloc and Target Sequence 249.021

| | | Substrate | | | | Rz |
|---|---|---|---|---|---|---|
| Pos | Substrate | Seq ID No. | RPI# | Alias | GeneBloc | Seq ID No. |
| 821 | CAAGGUAAGGCAUUUGUCCAUGA | 5399 | 19843 | hCLCA1:821L23 GB3.3 | B ucauggaCSASASASTSGSCSCSTSuaccuug B | 5417 |
| 1141 | CAAAGAAGCUCCAAACAAGCAAA | 5400 | 19837 | hCLCA1:1141L23 GB3.3 | B uuugcuuGSTSTSTSGSGSASGSCSuucuuug B | 5418 |
| 1646 | GUCAAACAAAGUGGUGCCAUCAU | 5401 | 19841 | hCLCA1:1646L23 GB3.3 | B augauggCSASCSCSASCSTSTSTSguuugac B | 5419 |
| 2464 | CAUACCUGGCUGGAUUGAGAAUG | 5402 | 19836 | hCLCA1:2464L23 GB3.3 | B cauucucASASTSCSCSASGSCSCSagguaug B | 5420 |
| 2542 | CAAGCAAGUGUGUUUCAGCAGAA | 5403 | 19839 | hCLCA1:2542L23 GB3.3 | B uucugcuGSASASASCSASCSASCSuugcuug B | 5421 |
| 2577 | GCUCAUUUGUGGCUUCUGAUGUC | 5404 | 19840 | hCLCA1:2577L23 GB3.3 | B gacaucaGSASASGSCSCSASCSASaaugagc B | 5422 |
| 2711 | UAUGACCAUGGAACAGCUCACAA | 5405 | 19842 | hCLCA1:2711L23 GB3.3 | B uugugagCSTSGSTSTSCSCSASTSggucaua B | 5423 |
| 3087 | GGAUAGGAGAACUGCAGCUGUCA | 5406 | 19838 | hCLCA1:3087L23 GB3.3 | B ugacagcTSGSCSASGSTSTSCSTSccuaucc B | 5424 |
| 69 | TCTTGATTCTTCACC | 5407 | 20960 | hCLCA1-69 Rz-7 allyl | gSgSuSgSaag cUGAuGaggccguuaggccGaa Aucaaga B | 5425 |
| | | | | stab1e | | |
| 70 | CTTGATTCTTCACCT | 5408 | 20961 | hCLCA1-70 Rz-7 allyl | aSgSgSuSgaa cUGAuGaggccguuaggccGaa Aaucaag B | 5426 |
| | | | | stab1e | | |
| 71 | TTGATTCTTCACCTT | 5409 | 20968 | hCLCA1-71 CHz-7 allyl | aSaSgSgSuga cUGAuGaggccguuaggccGaa Iaaucaa B | 5427 |
| | | | | stab1e | | |
| 72 | TGATTCTTCACCTTC | 5410 | 20962 | hCLCA1-72 Rz-7 allyl | gSaSaSgSgug cUGAuGaggccguuaggccGaa Agaauca B | 5428 |
| | | | | stab1e | | |
| 73 | GATTCTTCACCTTCT | 5411 | 20963 | hCLCA1-73 Rz-7 allyl | aSgSaSaSggu cUGAuGaggccguuaggccGaa Aagaauc B | 5429 |
| | | | | stab1e | | |
| 445 | TCCTGATTTCATTGC | 5412 | 20964 | hCLCA1-445 Rz-7 allyl | gScSaSaSuga cUGAuGaggccguuaggccGaa Aucagga B | 5430 |
| | | | | stab1e | | |
| 446 | CCTGATTTCATTGCA | 5413 | 20965 | hCLCA1-446 Rz-7 allyl | uSgScSaSaug cUGAuGaggccguuaggccGaa Aaucagg B | 5431 |
| | | | | stab1e | | |
| 447 | CTGATTTCATTGCAG | 5414 | 20966 | hCLCA1-447 Rz-7 allyl | cSuSgScSaau cUGAuGaggccguuaggccGaa Aaaucag B | 5432 |
| | | | | stab1e | | |
| 448 | TGATTTCATTGCAGG | 5415 | 20969 | hCLCA1-448 CHz7 | cScSuSgScaa cUGAuGaggccguuaggccGaa Iaaauca B | 5433 |
| | | | | allyl staB1e | | |
| 450 | ATTTCATTGCAGGAA | 5416 | 20967 | hCLCA1-450 Rz-7 allyl | uSuScScSugc cUGAuGaggccguuaggccGaa Augaaau B | 5434 |
| | | | | stab1e | | |

lower case = 2′OMe; A = riBo A
Upper Case = DeoxyriBose (DNA)
s = phosphorothioate linkages
B = inverted aBasic
U = 2′-C-allyl Uridine
G = riBo G

TABLE X
PCR Primers 249.021

| PCR primer | Seq ID No |
|---|---|
| CGAAATCTCGAGCAGACTTGTGGGAGAAGCTC | 5435 |
| AGCACACTGCAGAGTTGCTGGCCAGCTTACCTCC | 5436 |

What is claimed is:

1. A nucleic acid molecule that down regulates expression of CLCA1 (Chloride Channel Calcium Activated) gene.

2. The nucleic acid molecule of claim 1, wherein said nucleic acid molecule is used to treat conditions selected from the group consisting of Chronic Obstructive Pulmonary Disease (COPD), chronic bronchitis, asthma, cystic fibrosis, and obstructive bowel syndrome.

3. The nucleic acid molecule of claim 1, wherein said nucleic acid molecule is an enzymatic nucleic acid molecule.

4. The nucleic acid molecule of claim 3, wherein a binding arm of said enzymatic nucleic acid molecule comprise sequences complementary to any of sequences having SEQ ID NOs:1-2189 and 5399-5416.

5. The nucleic acid molecule of claim 3, wherein said enzymatic nucleic acid molecule comprises any of sequences having SEQ ID NOs:2190-5398 and 5425-5434.

6. The nucleic acid molecule of claim 1, wherein said nucleic acid molecule is an antisense nucleic acid molecule.

7. The nucleic acid molecule of claim 6, wherein said antisense nucleic acid molecule comprises sequences complementary to any of sequences having SEQ ID NOs:1-2189 and 5399-5416.

8. The nucleic acid molecule of claim 6, wherein said antisense nucleic acid molecule comprise any of sequences having SEQ ID NOs:5417-5424.

9. The nucleic acid molecule of claim 3, wherein said enzymatic nucleic acid molecule is in a hammerhead (HH) motif.

10. The nucleic acid molecule of claim 3, wherein said enzymatic nucleic acid molecule is in a hairpin, hepatitis Delta virus, group I intron, VS nucleic acid, amberzyme, zinzyme or RNAse P nucleic acid motif.

11. The nucleic acid molecule of claim 3, wherein said enzymatic nucleic acid molecule is in an Inozyme motif.

12. The nucleic acid molecule of claim 3, wherein said enzymatic nucleic acid molecule is in a G-cleaver motif.

13. The nucleic acid molecule of claim 3, wherein said enzymatic nucleic acid molecule is a DNAzyme.

14. The nucleic acid molecule of claims 3 or 6, wherein said nucleic acid molecule comprises between 12 and 100 bases complementary to RNA of a CLCA1 gene.

15. The nucleic acid molecule of claims 3 or 6, wherein said nucleic acid molecule comprises between 14 and 24 bases complementary to RNA of a CLCA1 gene.

16. The nucleic acid molecule of claim 1, wherein said nucleic acid molecule is chemically synthesized.

17. The nucleic acid molecule of claim 1, wherein said nucleic acid molecule comprises at least one 2′-sugar modification.

18. The nucleic acid molecule of claim 1, wherein said nucleic acid molecule comprises at least one nucleic acid base modification.

19. The nucleic acid molecule of claim 1, wherein said nucleic acid molecule comprises at least one phosphate backbone modification.

20. A mammalian cell comprising the nucleic acid molecule of claim 1.

21. The mammalian cell of claim 20, wherein said mammalian cell is a human cell.

22. A method of reducing CLCA1 activity in a cell, comprising the step of contacting said cell with the nucleic acid molecule of claim 1, under conditions suitable for said reduction of CLCA1 activity.

23. A method of treatment of a patient having a condition associated with the level of CLCA1, comprising contacting cells of said patient with the nucleic acid molecule of claim 1, under conditions suitable for said treatment.

24. The method of claim 23 further comprising the use of one or more therapies under conditions suitable for said treatment.

25. A method of cleaving RNA of a CLCA1 gene, comprising contacting the nucleic acid molecule of claim 3 with said RNA under conditions suitable for the cleavage of said RNA.

26. The method of claim 25, wherein said cleavage is carried out in the presence of a divalent cation.

27. The method of claim 26, wherein said divalent cation is Mg²⁺.

28. The nucleic acid molecule of claim 1, wherein said nucleic acid comprises a cap structure, wherein the cap structure is at the 5′-end or 3′-end or both the 5′-end and the 3′-end.

29. The enzymatic nucleic acid molecule of claim 9, wherein said hammerhead motif comprises sequences complementary to any of sequences having SEQ ID NOs: 1-575.

30. The enzymatic nucleic acid molecule of claim 11, wherein said Inozyme motif comprises sequences complementary to any of sequences having SEQ ID NOs:576-1210.

31. The enzymatic nucleic acid molecule of claim 12, wherein said G-cleaver motif comprises sequences complementary to any of sequences having SEQ ID NOs:1211-1429.

32. The enzymatic nucleic acid molecule of claim 13, wherein said DNAzyme comprises sequences complementary to any sequence shown as substrate sequences in Table VII.

33. The enzymatic nucleic acid molecule of claim 10, wherein said zinzyme comprises sequences complementary to any sequence shown as substrate sequences in Table VI.

34. The enzymatic nucleic acid molecule of claim 10, wherein said amberzyme comprises sequences complementary to any sequence shown as substrate sequences in Table VIU.

35. An expression vector comprising at least one nucleic acid molecule of claim 1, in a manner that allows expression of the nucleic acid molecule.

36. A mammalian cell comprising an expression vector of claim 35.

37. The mammalian cell of claim 36, wherein said mammalian cell is a human cell.

38. The expression vector of claim 35, wherein said nucleic acid molecule is an enzymatic nucleic acid molecule.

39. The expression vector of claim 35, wherein said expression vector further comprises an antisense nucleic acid molecule complementary to RNA of a CLCA1 gene.

40. The expression vector of claim 35, wherein said expression vector comprises at least two said nucleic acid molecules.

41. The expression vector of claim 40, wherein the at least two nucleic acid molecules are the same.

42. The expression vector of claim 40, wherein the at least two nucleic acid molecules are different.

43. The expression vector of claim 40, wherein one said expression vector further comprises an antisense nucleic acid molecule complementary to RNA of a CLCA1 gene.

44. The expression vector of claim 40, wherein one said expression vector further comprises an enzymatic nucleic acid molecule complementary to RNA of a CLCA1 gene.

45. A method for treatment of chronic obstructive pulmonary disease comprising the step of administering to a patient the nucleic acid molecule of claim 1 under conditions suitable for said treatment.

46. A method for treatment of cystic fibrosis comprising the step of administering to a patient the nucleic acid molecule of claim 1 under conditions suitable for said treatment.

47. An enzymatic nucleic acid molecule that cleaves RNA derived from a CLCA1 gene.

48. The enzymatic nucleic acid molecule of claim 47, wherein said enzymatic nucleic acid molecule is selected from the group consisting of Hammerhead, Hairpin, Inozyme, G-cleaver, DNAzyme, Amberzyme and Zinzyme.

49. The method of claims 45 or 46, wherein said method further comprises administering to said patient the nucleic acid molecule of claim 1 in conjunction with one or more other therapies.

50. The method of claim 49, wherein said other therapies are therapies selected from the group consisting of oxygen therapy, bronchodilators, corticosteroids, antibacterials, vaccinations, acetylcysteine, mucokinetic agents, and DNase (Pulmozyme) treatments.

51. The nucleic acid molecule of claim 9, wherein said nucleic acid molecule comprises at least five ribose residues, at least ten 2′-O-methyl modifications, and a 3′-end modification.

52. The nucleic acid molecule of claim 51, wherein said nucleic acid molecule further comprises a phosphorothioate core with both 3′ and 5′ -end modifications.

53. The nucleic acid molecule of claims 51 or 52, wherein said 3′ and/or 5′-end modification is 3′-3′ inverted abasic moiety.

54. The nucleic acid molecule of claim 3, wherein said nucleic acid molecule comprises at least five ribose residues; at least ten 2′-O-methyl modifications, and a 3′-end modification.

55. The nucleic acid molecule of claim 54, wherein said nucleic acid molecule further comprises phosphorothioate linkages on at least three of the 5′ terminal nucleotides.

56. The nucleic acid molecule of claim 54, wherein said 3′-end modification is 3′-3′ inverted abasic moiety.

57. The enzymatic nucleic acid molecule of claim 13, wherein said DNAzyme comprises at least ten 2′-O-methyl modifications.

58. The enzymatic nucleic acid molecule of claim 57, wherein said DNAzyme further comprises phosphorothioate linkages on at least three of the 5′ terminal nucleotides.

59. The enzymatic nucleic acid molecule of claim 57, wherein said DNAzyme further comprises a 3′-end modification.

60. The enzymatic nucleic acid molecule of claim 59, wherein said 3′-end modification is 3′-3′ inverted abasic moiety.
